(19) United States
(12) Patent Application Publication
(10) Pub. No.: US 2021/0199660 A1
(43) Pub. Date: Jul. 1, 2021

(54) BIOMARKERS OF BREAST CANCER

(71) Applicant: 10x Genomics, Inc., Pleasanton, CA (US)

(72) Inventors: Stephen R. Williams, Pleasanton, CA (US); Cedric Uytingco, Pleasanton, CA (US); Zachary Bent, Pleasanton, CA (US); Jennifer Chew, Pleasanton, CA (US)

(21) Appl. No.: 17/100,106

(22) Filed: Nov. 20, 2020

Related U.S. Application Data
(60) Provisional application No. 63/035,324, filed on Jun. 5, 2020.
(60) Provisional application No. 62/980,116, filed on Feb. 21, 2020.
(60) Provisional application No. 62/979,681, filed on Feb. 21, 2020.
(60) Provisional application No. 62/939,517, filed on Nov. 22, 2019.

(51) Int. Cl. G01N 33/574; A61K 45/06; A61K 31/704; A61K 31/337

(52) U.S. Cl. CPC G01N 33/57415; A61K 45/06; G01N 2496/00; A61K 31/337; A61K 31/704

(57) ABSTRACT

Provided herein are methods of determining a location of one or more analytes of interest in a cancer sample. Also provided herein are methods of detecting expression of one or more analytes in a cancer (e.g., breast cancer) sample. Further provided are methods of diagnosing and treating breast cancer.

### CROSS-REFERENCE TO RELATED APPLICATIONS

This application claims priority and benefit from U.S. Provisional Patent Application 62/939,517, filed Nov. 22, 2019, U.S. Provisional Patent Application 62/979,681, filed Feb. 21, 2020, U.S. Provisional Patent Application 62/980,116, filed Feb. 21, 2020, and U.S. Provisional Patent Application 63/035,324, filed Jun. 5, 2020, the contents and disclosures of which are incorporated herein by reference in their entireties.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

Tumors can be heterogeneous, with different regions within a tumor sample demonstrating different gene expression. A particular cancer, triple negative breast cancer (TNBC), accounts for 10-20% of all diagnosed breast cancer cases in the United States. TNBC is aggressive and exhibits poor prognosis due to resistance to traditional therapies and lack of identification of therapeutic targets. The lack of therapeutic targets, as well as the complexity of the disease, make it critical to further investigate the underlying biology in order to improve diagnosis and therapeutic outcome. TNBC is extremely complex, making it critical to understand the underlying biology to improve outcomes.

### SEQUENCE LISTING

The instant application contains a Sequence Listing which has been submitted on a compact disc and is hereby incorporated by reference in its entirety. Said compact disc, created on Nov. 18, 2020, is includes a txt file named Sequence_Listing_47706-0199001.ba which is 308.869 megabytes in size. 3 copies of the compact discs are submitted.

### INCORPORATION BY REFERENCE OF TABLES SUBMITTED AS TEXT FILE VIA EFS-WEB

The instant application contains Tables 1a-4a, which have been submitted via EFS-Web and are hereby incorporated in their entirety by reference herein. The text files, creation date of Feb. 20, 2020, are named 47706_0198P01_0199P01_Table_1_pan_cancer.txt (referred to in the present disclosure as “Table 1a”), 47706_0198P01_0199P01_Table_2_immunology.txt (referred to in the present disclosure as “Table 2a”), 47706_0198P01_0199P01_Table_3_pathway.txt (referred to in the present disclosure as “Table 3a”), and 47706_0198P01_0199P01_Table_4_neuro.txt (referred to in the present disclosure as “Table 4a”), and are respectively 1818 kilobytes, 1034 kilobytes, 1448 kilobytes, and 1724 kilobytes in size.

LENGTHY TABLES
The patent application contains a lengthy table section. A copy of the table is available in electronic form from the USPTO web site (https://seqdata.uspto.gov/?pageRequest=docDetail&DocID=US20210199660A1). An electronic copy of the table will also be available from the USPTO upon request and payment of the fee set forth in 37 CFR 1.19(b)(3).

### SUMMARY

Featured herein is a method of assessing expression levels in a subject having breast cancer. In some instances, the method includes obtaining a biological sample from the subject; and determining an expression level of one or more analytes selected from the group consisting of centromere protein W (CENPW), alpha-2-macroglobulin like 1 (A2ML1), very low density lipoprotein receptor (VLDLR), Scrapie-responsive protein 1 (SCRG1), RNA 3′-terminal phosphate cyclase-like protein (RCL1), fatty acid binding protein 7 (FABP7), cyclin E1 (CCNE1), migration and invasion enhancer 1 (MIEN1), cell division cycle 37 like 1 (CDC37L1), endoplasmic reticulum metallopeptidase 1 (ERMP1), tumor necrosis factor (ligand) superfamily, member 10 (TNFSF10), actin gamma 2, smooth muscle (ACTG2), dermokine (DMKN), calmodulin like 3 (CALML3), collagen type XVII alpha 1 chain (COL17A1), Melanoma-associated antigen D1 (MAGED1), pleiotrophin (PTN), transmembrane protein 98 (TMEM98), lymphocyte antigen 6 family member D (LY6D), tenascin C (TNC), and reticulon 4 interacting protein 1 (RTN4IP1), immunoglobulin lambda constant 2 (IGLC2), immunoglobulin heavy constant gamma 3 (IGHG), immunoglobulin kappa constant (IGKC), immunoglobulin heavy constant gamma 1 (IGHG1), immunoglobulin lambda constant 3 (IGLC3), immunoglobulin heavy constant alpha 1 (IGHA1), Immunoglobulin Heavy Constant Gamma 2 (G2m Marker) (IGHG2), Immunoglobulin Heavy Constant Mu (IGHM), Immunoglobulin Heavy Constant Gamma 4 (G4m Marker) (IGHG4), Joining Chain Of Multimeric IgA And IgM (JCHAIN), Inhibitor Of DNA Binding 3, HLH Protein (ID3), Class II Major Histocompatibility Complex Transactivator (CIITA), Cystatin F (CST7), Interferon Alpha Inducible Protein 27 Like 2 (IFI27L2), FYN Proto-Oncogene, Src Family Tyrosine Kinase (FYN), Microtubule Associated Monooxygenase, Calponin And LIM Domain Containing (MICAL1), Heme Oxygenase 1 (HMOX1), CD7 Molecule (CD7), Rho Guanine Nucleotide Exchange Factor 1 (ARHGEF1), and Complement Factor H (CFH), Metastasis Associated Lung Adenocarcinoma Transcript 1 (MALAT1), Cathepsin D (CTSD), Thymidine Phosphorylase (TYMP), SAM and HD Domain Containing Deoxynucleoside Triphosphate Triphosphohydrolase 1 (SAMHD1), Cytochrome B-245 Alpha Chain (CYBA), ISG15 Ubiquitin Like Modifier (ISG15), Complement C1q A Chain (C1QA), Ribosomal Protein S9 (RPS9), H2A.J Histone (H2AFJ), Adipogenesis Regulatory Factor (ADIRF), Rho GDP Dissociation Inhibitor Alpha (ARHGDIA), Adenine Phosphoribosyltransferase (APRT), AE Binding Protein 1 (AEBP1), Plectin (PLEC), Apolipoprotein E (APOE), Fc Fragment Of IgG Receptor And Transporte (FCGRT), NADH:Ubiquinone Oxidoreductase Subunit B7 (NDUFB7), Methyl-CpG Binding Domain Protein 3 (MBD3), Elastin Microfibril Interfacer 1 (EMILIN1), and GADD45G Interacting Protein 1 (GADD45GIP1), C—X—C Motif Chemokine Ligand 14 (CXCL14), Tubulin Tyrosine Ligase Like 12 (TTLL12), GDNF Family Receptor Alpha 1 (GFRA1), Delta 4-Desaturase, Sphingolipid 1 (DEGS1), Anterior Gradient 2, Protein Disulphide Isomerase Family Member (AGR2), Acidic Residue Methyltransferase 1 (ARMT1), Cyclin D1 (CCND1), CAMP Regulated Phosphoprotein 21 (ARPP21), Carnitine 0-Acetyltransferase (CRAT), Protein Kinase CAMP-Activated Catalytic Subunit Beta (PRKACB), Nuclear Receptor Binding SET Domain Protein 3 (NSD3), Plasminogen Activator, Urokinase Receptor (PLAUR), Cyclin Dependent Kinase Inhibitor 2C (CDKN2C), Factor Interacting With PAPOLA And CPSF1 (F1P1L1), Transmembrane Protein 159 (TMEM159), Transmembrane Protein 141 (TMEM141), Lin-7 Homolog C, Crumbs Cell Polarity Complex Component (LIN7C), Rho Guanine Nucleotide Exchange Factor 39 (ARHGEF39), ARFGEF Family Member 3 (ARFGEF3), and Epithelial Membrane Protein 2 (EMP2), Carboxypeptidase B1 (CPB1), Fc Fragment Of IgG Receptor IIIb (FCGR3B), Kelch Domain Containing 7B (KLHDC7B), Secretoglobin Family 1D Member 2 (SCGB1D2), Signal Peptide, CUB Domain And EGF Like Domain Containing 3 (SCUBE3), C—X—C Motif Chemokine Ligand 9 (CXCL9), Cytochrome C Oxidase Subunit 6C (COX6C), Complement Factor B (CFB), Secretoglobin Family 2A Member 2 (SCGB2A2), Neuropeptide Y Receptor Y1 (NPY1R), ENSG00000262580 (AC087741.1), Guanylate Binding Protein 5 (GBP5), Intraflagellar Transport 27 (IFT27), Neuralized E3 Ubiquitin Protein Ligase 4 (NEURL4), Empty Spiracles Homeobox 1 (EMX1), Solute Carrier Family 13 Member 2 (SLC13A2), Family With Sequence Similarity 110 Member A (FAM110A), Signal Peptidase Complex Subunit 1 (SPCS1), Homogentisate 1,2-Dioxygenase (HGD), and Zinc Finger Protein 587 (ZNF587), Cysteine Rich Secretory Protein 3 (CRISP3), SLIT And NTRK Like Family Member 6 (SLITRK6), Chromosome 6 Open Reading Frame 141 (C6orf141), V-Set Domain Containing T Cell Activation Inhibitor 1 (VTCN1), Serine Hydrolase Like 2 (SERHL2), CEA Cell Adhesion Molecule 6 (CEACAM6), ATP Binding Cassette Subfamily C Member 11 (ABCC11), Shisa Family Member 2 (SHISA2), Chromosome 2 open reading frame 54 (C2orf54), PDZ and LIM Domain 1 (PDLIM1), Zinc Finger CCCH-Type Containing 12A (ZC3H12A), VPS37B Subunit Of ESCRT-I (VPS37B), Interferon Regulatory Factor 2 Binding Protein 2 (IRF2BP2), Ras Association (RalGDS/AF-6) And Pleckstrin Homology Domains 1 (RAPH1), NFKB Inhibitor Alpha (NFKBIA), Eukaryotic Translation Initiation Factor 2 Alpha Kinase 1 (EIF2AK1), Tripartite Motif Containing 33 (TRIM33), Splicing Factor Proline And Glutamine Rich (SFPQ), Coagulation Factor VII (F7), and Trafficking Protein Particle Complex 3 (TRAPPC3), Long Intergenic Non-Protein Coding RNA 52 (LINC00052), Cytochrome C Oxidase Subunit 6C (COX6C), Synuclein Gamma (SNCG), WAP Four-Disulfide Core Domain 2 (WFDC2), Solute Carrier Family 39 Member 6 (SLC39A6), Microsomal Glutathione S-Transferase 1 (MGST1), Mitochondrial Coiled-Coil Domain 1 (MCCD1), Cystatin A (CSTA), Phosphodiesterase 5A (PDE5A), Mitochondrially Encoded NADH:Ubiquinone Oxidoreductase Core Subunit 1 (MT-ND1), Signal Recognition Particle 14 (SRP14), Trafficking Protein Particle Complex 1 (TRAPPC1), Small Nuclear Ribonucleoprotein D3 Polypeptide (SNRPD3), Methionine Adenosyltransferase 2 (MAT2A), Solute Carrier Family 7 Member 8 (SLC7A8), RNA Polymerase II Subunit K (POLR2K), Transmembrane BAX Inhibitor Motif Containing 6 (TMBIM6), OCIA Domain Containing 1 (OCIAD1), Exosome Component 3 (EXOSC3), and Carbonic Anhydrase 1 (CA14), Atypical Chemokine Receptor 1 (Duffy Blood Group) (ACKR1), Insulin Like Growth Factor Binding Protein 7 (IGFBP7), Aquaporin 1 (Colton Blood Group) (AQP1), Von Willebrand Factor (VWF), Metastasis Associated Lung Adenocarcinoma Transcript 1 (MALAT1), SPARC Like 1 (SPARCL1), Transgelin (TAGLN), C—C Motif Chemokine Ligand 21 (CCL21), Actin Alpha 2, Smooth Muscle (ACTA2), Coiled-Coil Domain Containing 80 (CCDC₈₀), Cytochrome B5 Reductase 3 (CYB5R3), Plexin D1 (PLXND1), Dual Specificity Phosphatase 1 (DUSP1), Ribosomal Protein S6 (RPS6), Complement C1q A Chain (C1QA), Heart Development Protein with EGF Like Domains 1 (HEG1), ETS Proto-Oncogene 2, Transcription Factor (ETS2), A-Kinase Anchoring Protein 9 (AKAP9), Cell Division Cycle and Apoptosis Regulator 1 (CCAR1), and Tripartite Motif Containing 47 (TRIM47), Albumin (ALB), Matrix Gla Protein (MGP), ZNF350 Antisense RNA 1 (ZNF350-AS1), S100 Calcium Binding Protein G (S100G), Stanniocalcin 2 (STC2), CART Prepropeptide (CARTPT), Uncharacterized LOC102724957 (AC087379.2), Glypican 3 (GPC3), Endoplasmic Reticulum Protein 27 (ERP27), and Apolipoprotein D (APOD), CDV3 Homolog (CDV3), Triosephosphate Isomerase 1 (TPI1), TSPY Like 5 (TSPYL5), Phosphofructokinase, Platelet (PFKP), Cysteine Rich Transmembrane BMP Regulator 1 (CRIM1), Palmitoyl-Protein Thioesterase 1 (PPT1), ENSG00000259457 (AC100826.1), Aldolase, Fructose-Bisphosphate A (ALDOA), Leucine Rich Repeat Containing G Protein-Coupled Receptor 4 (LGR4), and Glutaredoxin 2 (GLRX2), Uncharacterized LOC102724957 (AC087379.2), S100 Calcium Binding Protein G (S100G), Secretoglobin Family 2A Member 2 (SCGB2A2), PGM5 Antisense RNA 1 (PGM5-AS1), Heme Binding Protein 1 (HEBP1), Adhesion Molecule With Ig Like Domain 2 (AMIGO2), PC-Esterase Domain Containing 1B (PCED1B), Secretoglobin Family 1D Member 2 (SCGB1D2), Inositol 1,4,5-Trisphosphate Receptor Type 1 (ITPR1), Intraflagellar Transport 122 (IFT122); GABA Type A Receptor Associated Protein Like 1 (GABARAPL1), Glucose-6-Phosphatase Catalytic Subunit 3 (G6PC3), Inositol Polyphosphate-5-Phosphatase K (INPP5K), Stanniocalcin 2 (STC2), HEXIM P-TEFb Complex Subunit 2 (HEXIM2), Reactive Intermediate Imine Deaminase A Homolog (RIDA), LDL Receptor Related Protein 2 (LRP2), Hexokinase 2 (HK2), Interleukin 1 Receptor Type 2 (IL1R2), and Glutamic-Oxaloacetic Transaminase 2 (GOT2), Albumin (ALB), Mitochondrially Encoded NADH:Ubiquinone Oxidoreductase Core Subunit 2 (MT-ND2), Mitochondrially Encoded NADH:Ubiquinone Oxidoreductase Core Subunit 1 (MT-ND1), Mitochondrially Encoded NADH:Ubiquinone Oxidoreductase Core Subunit 3 (MT-ND3), Mitochondrially Encoded ATP Synthase Membrane Subunit 6 (MT-ATP6), Mitochondrially Encoded NADH:Ubiquinone Oxidoreductase Core Subunit 4 (MT-ND4), Mitochondrially Encoded Cytochrome C Oxidase I (MT-CO1), Mitochondrially Encoded Cytochrome C Oxidase III (MT-CO3), Mitochondrially Encoded ATP Synthase Membrane Subunit 8 (MT-ATPS), Mitochondrially Encoded NADH:Ubiquinone Oxidoreductase Core Subunit 5 (MT-ND5), Glyoxalase I (GLO1), Abhydrolase Domain Containing 2 (ABHD2), Long Intergenic Non-Protein Coding RNA 52 (LINC00052), RAS Like Estrogen Regulated Growth Inhibitor (RERG), Stanniocalcin 2 (STC2), Metastasis Associated Lung Adenocarcinoma Transcript 1 (MALAT1), SRY-Box Transcription Factor 4 (SOX4), Carbonic Anhydrase 12 (CA12), Zinc Finger Protein 703 (ZNF703), and Mitochondrial Coiled-Coil Domain 1 (MCCD1), Long Intergenic Non-Protein Coding RNA 645 (LINC00645), Solute Carrier Family 30 Member 8 (SLC30A8), Mucin 5B, Oligomeric Mucus/Gel-Forming (MUC5B), Collectin Subfamily Member 12 (COLEC12), Parvalbumin (PVALB), Carboxypeptidase B1 (CPB1), Exocyst Complex Component 2 (EXOC2), ENSG00000278621 (AC037198.2), V-Set And Transmembrane Domain Containing 2A (VSTM2A), Fibrous Sheath Interacting Protein 1 (FSIP1); Kinesin Family Member 16B (KIF16B), Spermatogenesis Associated 20 (SPATA20), Tetraspanin 9 (TSPAN9), Calcium Voltage-Gated Channel Auxiliary Subunit Beta 3 (CACNB3), Calcium/Calmodulin Dependent Protein Kinase II Inhibitor 1 (CAMK2N1), Intraflagellar Transport 27 (IFT27), Neuralized E3 Ubiquitin Protein Ligase 1 (NEURL1), Tripartite Motif Containing 3 (TRIM3), Solute Carrier Family 46 Member 1 (SLC46A1), and Enoyl-CoA Delta Isomerase 2 (ECI2), Matrix Gla Protein (MGP), Trefoil Factor 1 (TFF1), Keratin 14 (KRT14), S100 Calcium Binding Protein A9 (S100A9), Keratin 17 (KRT17), S100 Calcium Binding Protein G (S100G), S100 Calcium Binding Protein A2 (S100A2), ZNF350 Antisense RNA 1 (ZNF350-AS1), Keratin 5 (KRT5), S100 Calcium Binding Protein A8 (S100A8), MN1 Proto-Oncogene, Transcriptional Regulator (MN1), Transmembrane Protein 45A (TMEM45A), Dynein Axonemal Light Intermediate Chain 1 (DNALI1), Chromosome 3 Open Reading Frame 14 (C3orf14), TSR1 Ribosome Maturation Factor (TSR1), ENSG00000233461 (AL445524.1) Semaphorin 3C (SEMA3C), NADH:Ubiquinone Oxidoreductase Complex Assembly Factor 2 (NDUFAF2), Achaete-Scute Family BHLH Transcription Factor 1 (ASCL1), and Grainyhead Like Transcription Factor 2 (GRHL2), Serum Amyloid A1 (SAA1), Fatty Acid Binding Protein 4 (FABP4), Glutathione Peroxidase 3 (GPX3), Pleckstrin Homology Domain Containing A8 (PIP), Alcohol Dehydrogenase 1B (Class I), Beta Polypeptide (ADH1B), Perilipin 1 (PLIN1), Collagen Type II Alpha 1 Chain (COL2A1), SH3 Domain Binding Glutamate Rich Protein Like (SH3BGRL), Adiponectin, C1Q And Collagen Domain Containing (ADIPOQ), Perilipin 4 (PLIN4), Heterogeneous Nuclear Ribonucleoprotein A0 (HNRNPA0), Ribosomal Protein L11 (RPL11), Solute Carrier Family 40 Member 1 (SLC40A1), Ribosomal Protein Lateral Stalk Subunit P2 (RPLP2), CTD Small Phosphatase 2 (CTDSP2), Sortilin Related Receptor 1 (SORL1), Ribosomal Protein L31 (RPL31), Endothelin Converting Enzyme 1 (ECE1), Secreted Frizzled Related Protein 2 (SFRP2), and Cyclin D1 (CCND1), Phosphodiesterase 5A (PDE5A), Matrix Gla Protein (MGP), WAP Four-Disulfide Core Domain 2 (WFDC2), MRPS30 Divergent Transcript (MRPS30-DT), RNA Binding Motif Protein 20 (RBM20), Mitochondrial Ribosomal Protein S30 (MRPS30), Autocrine Motility Factor Receptor (AMFR), Stanniocalcin 2 (STC2), Potassium Voltage-Gated Channel Subfamily E Regulatory Subunit 4 (KCNE4), Discoidin Domain Receptor Tyrosine Kinase 1 (DDR1), Methylcrotonoyl-CoA Carboxylase 1 (MCCC1), Pyruvate Dehydrogenase Phosphatase Regulatory Subunit (PDPR), Archaelysin Family Metallopeptidase 2 (AMZ2), Chromosome 5 Open Reading Frame 15 (C5orf15), TBC1 Domain Family Member 9 (TBC1D9), SRSF Protein Kinase 1 (SRPK1), Long Intergenic Non-Protein Coding RNA 1488 (LINC01488), Tumor Susceptibility 101 (TSG101), Cytochrome C Oxidase Assembly Factor 3 (COA3), and NFKB Inhibitor Epsilon (NFKBIE), immunoglobulin lambda constant 2 (IGLC2), C—C motif chemokine ligand 19 (CCL19), immunoglobulin heavy constant alpha 2 (A2m marker) (IGHA2), migration and invasion enhancer 1 (MIEN1), secretory leukocyte peptidase inhibitor (SLPI), Suppressor Of Cytokine Signaling 2 (SOCS2), BMP And Activin Membrane Bound Inhibitor (BAMBI), WD Repeat Domain, Phosphoinositide Interacting 1 (WIPI1), Prolactin Receptor (PRLR), B-cell lymphoma 2 (BCL2), Zinc finger protein 3 (ZNF03), Interleukin 6 signal transducer (IL6ST), Secreted Phosphoprotein 1 (SPP1), Fibroblast Growth Factor Receptor 1 (FGFR1), Vascular Endothelial Growth Factor A (VEGFA), fibronectin-1 (FN-1), hairy and enhancer of split 6 (1-1ES6), 5100 Calcium Binding Protein A11 (S100A11), and H2A histone family member X (H2AFX), and byproducts, precursors, and degradation products thereof, in the biological sample obtained from the subject. In some instances, the method includes obtaining a biological sample from the subject; and determining an expression level of one or more analytes selected from the group consisting of IGLC2, CCL19, IGHA2, MIEN1, SLPI, SOCS2), BAMBI, WIPI1, PRLR, BCL2, ZNF03, IL6ST, SPP1, FGFR1, VEGFA, FN-1, HES6, S100A11, and H2AFX, and byproducts, precursors, and degradation products thereof, in the biological sample obtained from the subject.

In some instances, the one or more biomarkers is centromere protein W (CENPW), alpha-2-macroglobulin like 1 (A2ML1), very low density lipoprotein receptor (VLDLR), Scrapie-responsive protein 1 (SCRG1), RNA 3′-terminal phosphate cyclase-like protein (RCL1), fatty acid binding protein 7 (FABP7), cyclin E1 (CCNE1), migration and invasion enhancer 1 (MIEN1), cell division cycle 37 like 1 (CDC37L1), endoplasmic reticulum metallopeptidase 1 (ERMP1), actin gamma 2, smooth muscle (ACTG2), dermokine (DMKN), calmodulin like 3 (CALML3), collagen type XVII alpha 1 chain (COL17A1), Melanoma-associated antigen D1 (MAGED1), pleiotrophin (PTN), transmembrane protein 98 (TMEM98), lymphocyte antigen 6 family member D (LY6D), tenascin C (TNC), and reticulon 4 interacting protein 1 (RTN4IP1), byproducts, precursors, and degradation products thereof, or any combination thereof.

In some instances, the one or more biomarkers is immunoglobulin lambda constant 2 (IGLC2), immunoglobulin heavy constant gamma 3 (IGHG), immunoglobulin kappa constant (IGKC), immunoglobulin heavy constant gamma 1 (IGHG1), immunoglobulin lambda constant 3 (IGLC3), immunoglobulin heavy constant alpha 1 (IGHA1), Immunoglobulin Heavy Constant Gamma 2 (G2m Marker) (IGHG2), Immunoglobulin Heavy Constant Mu (IGHM), Immunoglobulin Heavy Constant Gamma 4 (G4m Marker) (IGHG4), Joining Chain Of Multimeric IgA And IgM (JCHAIN), Inhibitor Of DNA Binding 3, HLH Protein (ID3), Class II Major Histocompatibility Complex Transactivator (CIITA), Cystatin F (CST7), Interferon Alpha Inducible Protein 27 Like 2 (IFI27L2), FYN Proto-Oncogene, Src Family Tyrosine Kinase (FYN), Microtubule Associated Monooxygenase, Calponin And LIM Domain Containing (MICAL1), Heme Oxygenase 1 (HMOX1), CD7 Molecule (CD7), Rho Guanine Nucleotide Exchange Factor 1 (ARHGEF1), Complement Factor H (CFH), Metastasis Associated Lung Adenocarcinoma Transcript 1 (MALAT1), Cathepsin D (CTSD), Thymidine Phosphorylase (TYMP), SAM and HD Domain Containing Deoxynucleoside Triphosphate Triphosphohydrolase 1 (SAMHD1), Cytochrome B-245 Alpha Chain (CYBA), ISG15 Ubiquitin Like Modifier (ISG15), Complement C1q A Chain (C1QA), Ribosomal Protein S9 (RPS9), H2A.J Histone (H2AFJ), Adipogenesis Regulatory Factor (ADIRF), Rho GDP Dissociation Inhibitor Alpha (ARHGDIA), Adenine Phosphoribosyltransferase (APRT), AE Binding Protein 1 (AEBP1), Plectin (PLEC), Apolipoprotein E (APOE), Fc Fragment Of IgG Receptor And Transporte (FCGRT), NADH:Ubiquinone Oxidoreductase Subunit B7 (NDUFB7), Methyl-CpG Binding Domain Protein 3 (MBD3), Elastin Microfibril Interfacer 1 (EMILIN1), GADD45G Interacting Protein 1 (GADD45GIP1), C—X—C Motif Chemokine Ligand 14 (CXCL14), Tubulin Tyrosine Ligase Like 12 (TTLL12), GDNF Family Receptor Alpha 1 (GFRA1), Delta 4-Desaturase, Sphingolipid 1 (DEGS1), Anterior Gradient 2, Protein Disulphide Isomerase Family Member (AGR2), Acidic Residue Methyltransferase 1 (ARMT1), Cyclin D1 (CCND1), CAMP Regulated Phosphoprotein 21 (ARPP21), Carnitine 0-Acetyltransferase (CRAT), Protein Kinase CAMP-Activated Catalytic Subunit Beta (PRKACB), Nuclear Receptor Binding SET Domain Protein 3 (NSD3), Plasminogen Activator, Urokinase Receptor (PLAUR), Cyclin Dependent Kinase Inhibitor 2C (CDKN2C), Factor Interacting With PAPOLA And CPSF1 (FIP1L1), Transmembrane Protein 159 (TMEM159), Transmembrane Protein 141 (TMEM141), Lin-7 Homolog C, Crumbs Cell Polarity Complex Component (LIN7C), Rho Guanine Nucleotide Exchange Factor 39 (ARHGEF39), ARFGEF Family Member 3 (ARFGEF3), Epithelial Membrane Protein 2 (EMP2), Carboxypeptidase B1 (CPB1), Fc Fragment Of IgG Receptor IIIb (FCGR3B), Kelch Domain Containing 7B (KLHDC7B), Secretoglobin Family 1D Member 2 (SCGB1D2), Signal Peptide, CUB Domain And EGF Like Domain Containing 3 (SCUBE3), C—X—C Motif Chemokine Ligand 9 (CXCL9), Cytochrome C Oxidase Subunit 6C (COX6C), Complement Factor B (CFB), Secretoglobin Family 2A Member 2 (SCGB2A2), Neuropeptide Y Receptor Y1 (NPY1R), ENSG00000262580 (AC087741.1), Guanylate Binding Protein 5 (GBP5), Intraflagellar Transport 27 (IFT27), Neuralized E3 Ubiquitin Protein Ligase 4 (NEURL4), Empty Spiracles Homeobox 1 (EMX1), Solute Carrier Family 13 Member 2 (SLC13A2), Family With Sequence Similarity 110 Member A (FAM110A), Signal Peptidase Complex Subunit 1 (SPCS1), Homogentisate 1,2-Dioxygenase (HGD), Zinc Finger Protein 587 (ZNF587), Cysteine Rich Secretory Protein 3 (CRISP3), SLIT And NTRK Like Family Member 6 (SLITRK6), Chromosome 6 Open Reading Frame 141 (C6orf141), V-Set Domain Containing T Cell Activation Inhibitor 1 (VTCN1), Serine Hydrolase Like 2 (SERHL2), CEA Cell Adhesion Molecule 6 (CEACAM6), ATP Binding Cassette Subfamily C Member 11 (ABCC11), Shisa Family Member 2 (SHISA2), Chromosome 2 open reading frame 54 (C2orf54), PDZ and LIM Domain 1 (PDLIM1), Zinc Finger CCCH-Type Containing 12A (ZC3H12A), VPS37B Subunit Of ESCRT-I (VPS37B), Interferon Regulatory Factor 2 Binding Protein 2 (IRF2BP2), Ras Association (RalGDS/AF-6) And Pleckstrin Homology Domains 1 (RAPH1), NFKB Inhibitor Alpha (NFKBIA), Eukaryotic Translation Initiation Factor 2 Alpha Kinase 1 (EIF2AK1), Tripartite Motif Containing 33 (TRIM33), Splicing Factor Proline And Glutamine Rich (SFPQ), Coagulation Factor VII (F7), Trafficking Protein Particle Complex 3 (TRAPPC3), Long Intergenic Non-Protein Coding RNA 52 (LINC00052), Cytochrome C Oxidase Subunit 6C (COX6C), Synuclein Gamma (SNCG), WAP Four-Disulfide Core Domain 2 (WFDC2), Solute Carrier Family 39 Member 6 (SLC39A6), Microsomal Glutathione S-Transferase 1 (MGST1), Mitochondrial Coiled-Coil Domain 1 (MCCD1), Cystatin A (CSTA), Phosphodiesterase 5A (PDE5A), Mitochondrially Encoded NADH:Ubiquinone Oxidoreductase Core Subunit 1 (MT-ND1), Signal Recognition Particle 14 (SRP14), Trafficking Protein Particle Complex 1 (TRAPPC1), Small Nuclear Ribonucleoprotein D3 Polypeptide (SNRPD3), Methionine Adenosyltransferase 2 (MAT2A), Solute Carrier Family 7 Member 8 (SLC7A8), RNA Polymerase II Subunit K (POLR2K), Transmembrane BAX Inhibitor Motif Containing 6 (TMBIM6), OCIA Domain Containing 1 (OCIAD1), Exosome Component 3 (EXOSC3), Carbonic Anhydrase 1 (CA14), Atypical Chemokine Receptor 1 (Duffy Blood Group) (ACKR1), Insulin Like Growth Factor Binding Protein 7 (IGFBP7), Aquaporin 1 (Colton Blood Group) (AQP1), Von Willebrand Factor (VWF), Metastasis Associated Lung Adenocarcinoma Transcript 1 (MALAT1), SPARC Like 1 (SPARCL1), Transgelin (TAGLN), C—C Motif Chemokine Ligand 21 (CCL21), Actin Alpha 2, Smooth Muscle (ACTA2), Coiled-Coil Domain Containing 80 (CCDC₈₀), Cytochrome B5 Reductase 3 (CYB5R3), Plexin D1 (PLXND1), Dual Specificity Phosphatase 1 (DUSP1), Ribosomal Protein S6 (RPS6), Complement C1q A Chain (C1QA), Heart Development Protein with EGF Like Domains 1 (HEG1), ETS Proto-Oncogene 2, Transcription Factor (ETS2), A-Kinase Anchoring Protein 9 (AKAP9), Cell Division Cycle and Apoptosis Regulator 1 (CCAR1), Tripartite Motif Containing 47 (TRIM47), Albumin (ALB), Matrix Gla Protein (MGP), ZNF350 Antisense RNA 1 (ZNF350-AS1), 5100 Calcium Binding Protein G (S100G), Stanniocalcin 2 (STC2), CART Prepropeptide (CARTPT), Uncharacterized LOC102724957 (AC087379.2), Glypican 3 (GPC3), Endoplasmic Reticulum Protein 27 (ERP27), and Apolipoprotein D (APOD), CDV3 Homolog (CDV3), Triosephosphate Isomerase 1 (TPI1), TSPY Like 5 (TSPYL5), Phosphofructokinase, Platelet (PFKP), Cysteine Rich Transmembrane BMP Regulator 1 (CRIM1), Palmitoyl-Protein Thioesterase 1 (PPT1), ENSG00000259457 (AC100826.1), Aldolase, Fructose-Bisphosphate A (ALDOA), Leucine Rich Repeat Containing G Protein-Coupled Receptor 4 (LGR4), Glutaredoxin 2 (GLRX2), Uncharacterized LOC102724957 (AC087379.2), S100 Calcium Binding Protein G (S100G), Secretoglobin Family 2A Member 2 (SCGB2A2), PGM5 Antisense RNA 1 (PGM5-AS1), Heme Binding Protein 1 (HEBP1), Adhesion Molecule With Ig Like Domain 2 (AMIGO2), PC-Esterase Domain Containing 1B (PCED1B), Secretoglobin Family 1D Member 2 (SCGB1D2), Inositol 1,4,5-Trisphosphate Receptor Type 1 (ITPR1), Intraflagellar Transport 122 (IFT122); GABA Type A Receptor Associated Protein Like 1 (GABARAPL1), Glucose-6-Phosphatase Catalytic Subunit 3 (G6PC3), Inositol Polyphosphate-5-Phosphatase K (INPP5K), Stanniocalcin 2 (STC2), HEXIM P-TEFb Complex Subunit 2 (HEXIM2), Reactive Intermediate Imine Deaminase A Homolog (RIDA), LDL Receptor Related Protein 2 (LRP2), Hexokinase 2 (HK2), Interleukin 1 Receptor Type 2 (IL1R2), and Glutamic-Oxaloacetic Transaminase 2 (GOT2), Albumin (ALB), Mitochondrially Encoded NADH:Ubiquinone Oxidoreductase Core Subunit 2 (MT-ND2), Mitochondrially Encoded NADH:Ubiquinone Oxidoreductase Core Subunit 1 (MT-ND1), Mitochondrially Encoded NADH:Ubiquinone Oxidoreductase Core Subunit 3 (MT-ND3), Mitochondrially Encoded ATP Synthase Membrane Subunit 6 (MT-ATP6), Mitochondrially Encoded NADH:Ubiquinone Oxidoreductase Core Subunit 4 (MT-ND4), Mitochondrially Encoded Cytochrome C Oxidase I (MT-CO1), Mitochondrially Encoded Cytochrome C Oxidase III (MT-CO3), Mitochondrially Encoded ATP Synthase Membrane Subunit 8 (MT-ATPS), Mitochondrially Encoded NADH:Ubiquinone Oxidoreductase Core Subunit 5 (MT-ND5), Glyoxalase I (GLO1), Abhydrolase Domain Containing 2 (ABHD2), Long Intergenic Non-Protein Coding RNA 52 (LINC00052), RAS Like Estrogen Regulated Growth Inhibitor (RERG), Stanniocalcin 2 (STC2), Metastasis Associated Lung Adenocarcinoma Transcript 1 (MALAT1), SRY-Box Transcription Factor 4 (SOX4), Carbonic Anhydrase 12 (CA12), Zinc Finger Protein 703 (ZNF703), Mitochondrial Coiled-Coil Domain 1 (MCCD1), Long Intergenic Non-Protein Coding RNA 645 (LINC00645), Solute Carrier Family 30 Member 8 (SLC30A8), Mucin 5B, Oligomeric Mucus/Gel-Forming (MUC5B), Collectin Subfamily Member 12 (COLEC12), Parvalbumin (PVALB), Carboxypeptidase B1 (CPB1), Exocyst Complex Component 2 (EXOC2), ENSG00000278621 (AC037198.2), V-Set And Transmembrane Domain Containing 2A (VSTM2A), Fibrous Sheath Interacting Protein 1 (FSIP1); Kinesin Family Member 16B (KIF16B), Spermatogenesis Associated 20 (SPATA20), Tetraspanin 9 (TSPAN9), Calcium Voltage-Gated Channel Auxiliary Subunit Beta 3 (CACNB3), Calcium/Calmodulin Dependent Protein Kinase II Inhibitor 1 (CAMK2N1), Intraflagellar Transport 27 (IFT27), Neuralized E3 Ubiquitin Protein Ligase 1 (NEURL1), Tripartite Motif Containing 3 (TRIM3), Solute Carrier Family 46 Member 1 (SLC46A1), Enoyl-CoA Delta Isomerase 2 (ECI2), Matrix Gla Protein (MGP), Trefoil Factor 1 (TFF1), Keratin 14 (KRT14), S100 Calcium Binding Protein A9 (S100A9), Keratin 17 (KRT17), 5100 Calcium Binding Protein G (S100G), 5100 Calcium Binding Protein A2 (S100A2), ZNF350 Antisense RNA 1 (ZNF350-AS1), Keratin 5 (KRT5), S100 Calcium Binding Protein A8 (S100A8), MN1 Proto-Oncogene, Transcriptional Regulator (MN1), Transmembrane Protein 45A (TMEM45A), Dynein Axonemal Light Intermediate Chain 1 (DNALI1), Chromosome 3 Open Reading Frame 14 (C3orf14), TSR1 Ribosome Maturation Factor (TSR1), ENSG00000233461 (AL445524.1) Semaphorin 3C (SEMA3C), NADH:Ubiquinone Oxidoreductase Complex Assembly Factor 2 (NDUFAF2), Achaete-Scute Family BHLH Transcription Factor 1 (ASCL1), Grainyhead Like Transcription Factor 2 (GRHL2), Serum Amyloid A1 (SAA1), Fatty Acid Binding Protein 4 (FABP4), Glutathione Peroxidase 3 (GPX3), Pleckstrin Homology Domain Containing A8 (PIP), Alcohol Dehydrogenase 1B (Class I), Beta Polypeptide (ADH1B), Perilipin 1 (PLIN1), Collagen Type II Alpha 1 Chain (COL2A1), SH3 Domain Binding Glutamate Rich Protein Like (SH3BGRL), Adiponectin, C1Q And Collagen Domain Containing (ADIPOQ), Perilipin 4 (PLIN4), Heterogeneous Nuclear Ribonucleoprotein A0 (HNRNPA0), Ribosomal Protein L11 (RPL11), Solute Carrier Family 40 Member 1 (SLC40A1), Ribosomal Protein Lateral Stalk Subunit P2 (RPLP2), CTD Small Phosphatase 2 (CTDSP2), Sortilin Related Receptor 1 (SORL1), Ribosomal Protein L31 (RPL31), Endothelin Converting Enzyme 1 (ECE1), Secreted Frizzled Related Protein 2 (SFRP2), Cyclin D1 (CCND1), Phosphodiesterase 5A (PDE5A), Matrix Gla Protein (MGP), WAP Four-Disulfide Core Domain 2 (WFDC2), MRPS30 Divergent Transcript (MRPS30-DT), RNA Binding Motif Protein 20 (RBM20), Mitochondrial Ribosomal Protein S30 (MRPS30), Autocrine Motility Factor Receptor (AMFR), Stanniocalcin 2 (STC2), Potassium Voltage-Gated Channel Subfamily E Regulatory Subunit 4 (KCNE4), Discoidin Domain Receptor Tyrosine Kinase 1 (DDR1), Methylcrotonoyl-CoA Carboxylase 1 (MCCC1), Pyruvate Dehydrogenase Phosphatase Regulatory Subunit (PDPR), Archaelysin Family Metallopeptidase 2 (AMZ2), Chromosome 5 Open Reading Frame 15 (C5orf15), TBC1 Domain Family Member 9 (TBC1D9), SRSF Protein Kinase 1 (SRPK1), Long Intergenic Non-Protein Coding RNA 1488 (LINC01488), Tumor Susceptibility 101 (TSG101), Cytochrome C Oxidase Assembly Factor 3 (COA3), NFKB Inhibitor Epsilon (NFKBIE), byproducts, precursors, and degradation products thereof, or any combination thereof.

In some instances, the one or more biomarkers is immunoglobulin lambda constant 2 (IGLC2), C—C motif chemokine ligand 19 (CCL19), immunoglobulin heavy constant alpha 2 (A2m marker) (IGHA2), migration and invasion enhancer 1 (MIEN1), secretory leukocyte peptidase inhibitor (SLPI), Suppressor Of Cytokine Signaling 2 (SOCS2), BMP And Activin Membrane Bound Inhibitor (BAMBI), WD Repeat Domain, Phosphoinositide Interacting 1 (WIPI1), Prolactin Receptor (PRLR), B-cell lymphoma 2 (BCL2), Zinc finger protein 3 (ZNF03), Interleukin 6 signal transducer (IL6ST), Secreted Phosphoprotein 1 (SPP1), Fibroblast Growth Factor Receptor 1 (FGFR1), Vascular Endothelial Growth Factor A (VEGFA), fibronectin-1 (FN-1), hairy and enhancer of split 6 (HES6), S100 Calcium Binding Protein A11 (S100A11), and H2A histone family member X (H2AFX), and byproducts, precursors, and degradation products thereof, or any combination thereof.

In some instances, the method further includes serially obtaining a biological sample from the subject at a plurality of time points. In some instances, the method also includes determining the expression levels of the one or more analytes in the serially obtained biological samples from the subject.

Also featured herein is a method of determining the expression levels of the one or more analytes wherein the determining step comprises: (a) contacting the biological sample with an substrate comprising a plurality of attached capture probes, wherein a capture probe of the plurality comprises (i) the spatial barcode and (ii) a capture domain that binds specifically to a sequence present in the one or more analytes; and (b) hybridizing an analyte of the one or more analytes to the capture domain. In some instances, the method further comprises extending a 3′ end of the capture probe using the analyte that is specifically bound to the capture domain as a template to generate an extended capture probe. In some instances, the method further comprises amplifying the extended capture probe to produce a nucleic acid. In some embodiments, the determining step comprises determining (i) all or a portion of the sequence of the spatial barcode or the complement thereof, and (ii) all or a portion of the sequence of the analyte from the biological sample, and using the determined sequences of (i) and (ii) to identify the location of the analyte in the biological sample.

In some instances, the analytes is determined by in situ sequencing. The in situ sequencing is selected from the group consisting of sequencing-by-synthesis (SBS), sequential fluorescence hybridization, sequencing by ligation, nucleic acid hybridization, hybridization chain reaction amplification, sequencing via rolling circle amplification, and high-throughput digital sequencing techniques.

Also disclosed herein is a method for identifying a location of an analyte in a biological sample comprising breast tissue from a subject diagnosed or suspected of having a breast cancer, the method comprising: (a) contacting a plurality of nucleic acids with a plurality of bait oligonucleotides, wherein: a nucleic acid of the plurality of nucleic acids comprises (i) a spatial barcode or a complement thereof, and (ii) a portion of a sequence of an analyte from a biological sample, or a complement thereof; and a bait oligonucleotide of the plurality of bait oligonucleotides comprises: a domain that binds specifically to (i) all or a portion of the spatial barcode or a complement thereof, and/or (ii) all or a portion of the sequence of the analyte from the biological sample, or a complement thereof, and a molecular tag; (b) enriching a complex of the bait oligonucleotide specifically bound to the nucleic acid using a substrate comprising an agent that binds specifically to the molecular tag; and (c) determining (i) all or a portion of the sequence of the spatial barcode or the complement thereof, and (ii) all or a portion of the sequence of the analyte from the biological sample, and using the determined sequences of (i) and (ii) to identify the location of the analyte in the biological sample. In some embodiments, the determining step comprises in situ sequencing. In some embodiments, the in situ sequencing is selected from the group consisting of sequencing-by-synthesis (SBS), sequential fluorescence hybridization, sequencing by ligation, nucleic acid hybridization, hybridization chain reaction amplification, sequencing via rolling circle amplification, and high-throughput digital sequencing techniques.

In some instances, the plurality of nucleic acids in (a) are generated by: (1) contacting the biological sample with an substrate comprising a plurality of attached capture probes, wherein a capture probe of the plurality comprises (i) the spatial barcode and (ii) a capture domain that binds specifically to a sequence present in the one or more analytes; and (2) hybridizing an analyte of the one or more analytes to the capture domain. In some embodiments, the method further comprises extending a 3′ end of the capture probe using the analyte that is specifically bound to the capture domain as a template to generate an extended capture probe. In some embodiments, the method further comprises amplifying the extended capture probe to produce the nucleic acid.

In some embodiments, the domain of the bait oligonucleotide binds specifically to all or a portion of the spatial barcode or a complement thereof. In some embodiments, the domain of the bait oligonucleotide binds specifically to all or a portion of the sequence of the analyte from the biological sample. In some embodiments, the domain of the bait oligonucleotide binds specifically to a 3′ portion of the sequence of the analyte from the biological sample or a complement thereof. In some embodiments, the domain of the bait oligonucleotide binds specifically to a 5′ portion of the sequence of the analyte from the biological sample or a complement thereof. In some embodiments, the domain of the bait oligonucleotide binds specifically to an intron in the sequence of the analyte from the biological sample or a complement thereof. In some embodiments, the domain of the bait oligonucleotide binds specifically to an exon in the sequence of the analyte from the biological sample or a complement thereof. In some embodiments, the domain of the bait oligonucleotide binds specifically to an untranslated 3′ region of the analyte from the biological sample or a complement thereof. In some embodiments, the domain of the bait oligonucleotide binds specifically to an untranslated 5′ region of the analyte from the biological sample or a complement thereof. In some embodiments, the analyte from the biological sample is associated with a disease or condition. In some embodiments, the analyte from the biological sample comprises a mutation. In some embodiments, the analyte from the biological sample comprises a single nucleotide polymorphism (SNP). In some embodiments, the analyte from the biological sample comprises a trinucleotide repeat. In some embodiments, the domain of the bait oligonucleotide comprises a total of about 10 nucleotides to about 300 nucleotides.

In some embodiments, the molecular tag comprises a moiety. In some embodiments, the moiety is streptavidin, avidin, biotin, or a fluorophore. In some embodiments, the molecular tag comprises a streptavidin molecule, an avidin molecule, a biotin molecule, or a fluorophore and/or a small molecule, a nucleic acid, a carbohydrate, or a combination thereof. In some embodiments, the molecular tag is positioned 5′ to the domain in the bait oligonucleotide. In some embodiments, the molecular tag is position 3′ to the domain in the bait oligonucleotide. In some embodiments, the agent that binds specifically to the molecular tag comprises a protein. In some embodiments, the protein is an antibody. In some embodiments, the agent that binds specifically to the molecular tag comprises a nucleic acid. In some embodiments, the agent that binds specifically to the molecular tag comprises a small molecule. In some embodiments, the agent that binds specifically to the molecular tag is attached to a substrate. In some embodiments, the substrate is a bead. In some embodiments, the substrate is a well. In some embodiments, the substrate is a slide. In some embodiments, the nucleic acid is DNA.

In some embodiments, the nucleic acid further comprises a functional sequence, wherein the functional sequence is a primer sequence or a complement thereof. In some embodiments, the nucleic acid further comprises a unique molecular sequence or a complement thereof. In some embodiments, the nucleic acid further comprises an additional primer binding sequence or a complement thereof. In some embodiments, the nucleic acid further comprises one or more functional domains, a unique molecular identifier, a cleavage domain, and combinations thereof.

In some embodiments, the biological sample is a tissue sample. In some embodiments, the biological sample is a solid tissue sample. In some embodiments, the tissue sample is a formalin-fixed, paraffin-embedded (FFPE) tissue sample or a frozen tissue sample. In some embodiments, the biological sample was previously stained using a detectable label. In some embodiments, the biological sample was previously stained using hematoxylin and eosin (H&E). In some embodiments, the biological sample is a breast tissue sample. In some embodiments, the biological sample is a permeabilized biological sample. In some embodiments, the permeabilized biological sample has been permeabilized with a permeabilization agent. In some embodiments, the permeabilization agent is selected from an organic solvent, a cross-linking agent, a detergent, and an enzyme, or a combination thereof. In some embodiments, the permeabilization agent is selected from an organic solvent, a cross-linking agent, a detergent, and an enzyme, or a combination thereof. In some embodiments, the analyte is an RNA molecule. In some embodiments, the RNA molecule is an mRNA molecule. In some embodiments, the analyte is a protein.

In some embodiments, the analyte from the one or more analytes is detected by the steps comprising: attaching the biological sample with a plurality of analyte capture agents, wherein an analyte capture agent of the plurality of analyte capture agents comprises: (i) an analyte binding moiety that binds specifically to the analyte; (ii) an analyte binding moiety barcode; and (iii) an analyte capture sequence, wherein the analyte capture sequence binds specifically to a capture domain; contacting the biological sample with a substrate, wherein the substrate comprises a plurality of capture probes, wherein a capture probe of the plurality of capture probes comprises (i) the capture domain and (ii) a spatial barcode; hybridizing the analyte to the capture probe; and determining (i) all or a part of a sequence corresponding to the analyte, and (ii) all or a part of a sequence corresponding to the spatial barcode, or a complement thereof, and using the determined sequence of (i) and (ii) to identify the abundance and/or spatial location of the analyte in the biological sample. In some embodiments, the determining step further comprises sequencing (i) all or a part of a sequence corresponding to the analyte, and (ii) all or a part of a sequence corresponding to the spatial barcode, or a complement thereof, and using the determined sequence of (i) and (ii) to identify the abundance and/or spatial location of the analyte in the biological sample. In some instances, the analyte binding moiety is an antibody or antigen-binding fragment thereof, a cell surface receptor binding molecule, a receptor ligand, a small molecule, a T-cell receptor engager, a B-cell receptor engager, a pro-body, an aptamer, a monobody, an affimer, or a darpin.

In some instances of any of the above methods, the analyte is detected using an antibody and/or contacting the biological sample with one or more stains. In some instances, the one or more stains comprises hematoxylin and eosin. In some instances, the one or more stains comprise one or more optical labels selected from the group consisting of: fluorescent, radioactive, chemiluminescent, calorimetric, or colorimetric labels. In some instances of any of the above methods, the biological sample is imaged.

In some embodiments, the determining in step (c) comprises sequencing (i) all or a portion of the sequence of the spatial barcode or the complement thereof, and (ii) all or a portion of the sequence of the analyte from the biological sample. In some embodiments, the sequencing is high throughput sequencing. In some embodiments, the sequencing comprises ligating an adapter to the nucleic acid.

In some embodiments, the method further comprises generating the plurality of nucleic acids comprises: (a) contacting the biological sample with an substrate comprising a plurality of attached capture probes, wherein a capture probe of the plurality comprises (i) the spatial barcode and (ii) a capture domain that binds specifically to a sequence present in the analyte; (b) extending a 3′ end of the capture probe using the analyte that is specifically bound to the capture domain as a template to generate an extended capture probe; and (c) amplifying the extended capture probe to produce the nucleic acid. In some embodiments, the amplifying is isothermal. In some embodiments, the produced nucleic acid is released from the extended capture probe.

In some embodiments, the breast cancer is ductal carcinoma in situ (DCIS). In some embodiments, the breast cancer is carcinoma. In some embodiments, the breast cancer is invasive carcinoma. In some embodiments, the breast cancer is triple negative breast cancer. In some embodiments, the breast cancer is an estrogen receptor positive (ER+), progesterone receptor negative (PR−), human epidermal growth factor receptor 2 positive (HER2+) breast cancer. In some embodiments, the plurality of capture probes comprise capture domains that bind specifically to nucleic acid analytes associated with breast cancer. In some embodiments, the methods further include diagnosing or confirming a diagnosis of breast cancer in the subject based on the location and optionally the abundance of the analyte in the biological sample. In some embodiments, the methods further comprise determining a location of a cancer cell in the biological sample based on the location and optionally the abundance of the analyte, in the biological sample. In some embodiments, the methods further comprise comparing the location and optionally the abundance of the analyte in the biological sample with a control sample.

In some embodiments, the capture probe binds specifically to CENPW, A2ML1, VLDLR, SCRG1, RCL1, FABP7, CCNE1, MIEN1, CDC37L1, ERMP1, TNFSF10, ACTG2, DMKN, CALML3, COL17A1, MAGED1, PTN, TMEM98, LY6D, TNC, RTN4IP1, IGLC2, IGHG, IGKC, IGHG1, IGLC3, IGHA1, G2m Marker, IGHG2, IGHM, G4m Marker, IGHG4, JCHAIN, ID3, CIITA, CST7, IFI27L2, FYN, MICAL1, HMOX1, CD7, ARHGEF1, CFH, MALAT1, CTSD, TYMP, SAMHD1, CYBA, ISG15, C1QA, RPS9, H2AFJ, ADIRF, ARHGDIA, APRT, AEBP1, PLEC, APOE, FCGRT, NDUFB7, MBD3, EMILIN1, GADD45G, GADD45GIP1, CXCL14, TTLL12, GFRA1, DEGS1, AGR2, ARMT1, CCND1, CAMP, ARPP21, CRAT, PRKACB, NSD3, PLAUR, CDKN2C, FIP1L1, TMEM159, TMEM141, LIN7C, ARHGEF39, ARFGEF3, EMP2, CPB1, FCGR3B, KLHDC7B, SCGB1D2, SCUBE3, CXCL9, COX6C, CFB, SCGB2A2, NPY1R, AC087741.1, GBP5, IFT27, NEURL4, EMX1, SLC13A2, FAM110A, SPCS1, HGD, ZNF587, CRISP3, SLITRK6, C6orf141, VTCN1, SERHL2, CEACAM6, ABCC11, SHISA2, C2orf54, PDLIM1, ZC3H12A, VPS37B, IRF2BP2, RalGDS/AF-6, RAPH1, NFKBIA, EIF2AK1, TRIM33, SFPQ, F7, TRAPPC3, LINC00052, COX6C, SNCG, WFDC2, SLC39A6, MGST1, MCCD1, CSTA, PDE5A, MT-ND1, SRP14, TRAPPC1, SNRPD3, MAT2A, SLC7A8, POLR2K, TMBIM6, OCIAD1, EXOSC3, CA14, ACKR1, IGFBP7, AQP1, VWF, MALAT1, SPARCL1, TAGLN, CCL21, ACTA2, CCDC₈₀, CYB5R3, PLXND1, DUSP1, RPS6, C1QA, HEG1, ETS2, AKAP9, CCAR1, TRIM47, ALB, MGP, ZNF350-AS1, S100G, STC2, CARTPT, AC087379.2, GPC3, ERP27, APOD, CDV3, TPI1, TSPYL5, PFKP, CRIM1, PPT1, AC100826.1, ALDOA, LGR4, GLRX2, AC087379.2, S100G, SCGB2A2, PGM5-AS1, HEBP1, AMIGO2, PCED1B, SCGB1D2, ITPR1, IFT122, GABARAPL1, G6PC3, INPP5K, STC2, HEXIM2, RIDA, LRP2, HK2, IL1R2, GOT2, ALB, MT-ND2, MT-ND1, MT-ND3, MT-ATP6, MT-ND4, MT-CO1, MT-CO3, MT-ATPS, MT-ND5), GLO1, ABHD2, LINC00052, RERG, STC2, MALAT1, SOX4, CA12, ZNF703, MCCD1, LINC00645, SLC30A8, MUC5B, COLEC12, PVALB, CPB1, EXOC2, AC037198.2, VSTM2A), FSIP1, KIF16B, SPATA20, TSPAN9, CACNB3, CAMK2N1, IFT27, NEURL1, TRIM3, SLC46A1, ECI2, MGP, TFF1, KRT14, S100A9, KRT17, S100G, S100A2, ZNF350-AS1, KRT5, S100A8, MN1, TMEM45A, DNALI1, C3orf14, TSR1, AL445524.1, SEMA3C, NDUFAF2, ASCL1, GRHL2, SAA1, FABP4, GPX3, PIP, ADH1B, PLIN1, COL2A1, SH3BGRL, ADIPOQ, PLIN, HNRNPA0, RPL11, SLC40A1, RPLP2, CTDSP2, SORL1, RPL31, ECE1, SFRP2, CCND1, PDE5A, MGP, WFDC2, MRPS30-DT, RBM20, MRPS30, AMFR, STC2, KCNE4, DDR1, MCCC1, PDPR, AMZ2, C5orf15, TBC1D9, SRPK1, LINC01488, TSG101, COA3, NFKBIE, IGLC2, CCL19, A2m marker, IGHA2, MIEN1, SLPI, BAMBI, WIPI1, PRLR, BCL2, ZNF03, IL6ST, SPP1, FGFR1, FN-1, HES6, S100A11, H2AFX or a byproduct, precursor, or degradation product thereof.

In some embodiments, the capture probe binds specifically to IGLC2, CCL19, A2m marker, IGHA2, MIEN1, SLPI, BAMBI, WIPI1, PRLR, BCL2, ZNF03, IL6ST, SPP1, FGFR1, VEGFA, FN-1, HES6, S100A11, or H2AFX or a byproduct, precursor, or degradation product thereof.

In some embodiments, the capture domain binds specifically to centromere protein W (CENPW), alpha-2-macroglobulin like 1 (A2ML1), very low density lipoprotein receptor (VLDLR), Scrapie-responsive protein 1 (SCRG1), RNA 3′-terminal phosphate cyclase-like protein (RCL1), fatty acid binding protein 7 (FABP7), cyclin E1 (CCNE1), migration and invasion enhancer 1 (MIEN1), cell division cycle 37 like 1 (CDC37L1), endoplasmic reticulum metallopeptidase 1 (ERMP1), actin gamma 2, smooth muscle (ACTG2), dermokine (DMKN), calmodulin like 3 (CALML3), collagen type XVII alpha 1 chain (COL17A1), Melanoma-associated antigen D1 (MAGED1), pleiotrophin (PTN), transmembrane protein 98 (TMEM98), lymphocyte antigen 6 family member D (LY6D), tenascin C (TNC), or reticulon 4 interacting protein 1 (RTN4IP1).

In some embodiments, the capture domain binds specifically to ALB, SOCS2, CSTA, BAMBI, WIPI1, PRLR, BCL2, ZNF03, IL6ST, PRKACB, SPP1, CCND1, FGFR1, VEGFA, FN1, HES6, S100A11, or H2AFX, or a byproduct, precursor, or degradation product thereof.

In some embodiments, the plurality of capture probes comprise capture domains that bind specifically to two or more of: CENPW, A2ML1, VLDLR, SCRG1, RCL1, FABP7, CCNE1, MIEN1, CDC37L1, ERMP1, ACTG2, DMKN, CALML3, COL17A1, MAGED1, PTN, TMEM98, LY6D, TNC, or RTN4IP1. In some embodiments, the plurality of capture probes comprise capture domains that bind specifically to ten or more of: CENPW, A2ML1, VLDLR, SCRG1, RCL1, FABP7, CCNE1, MIEN1, CDC37L1, ERMP1, ACTG2, DMKN, CALML3, COL17A1, MAGED1, PTN, TMEM98, LY6D, TNC, or RTN4IP1. In some embodiments, the plurality of capture probes comprise capture domains that bind specifically to two or more of: ALB, SOCS2, CSTA, BAMBI, WIPI1, PRLR, BCL2, ZNF03, IL6ST, PRKACB, SPP1, CCND1, FGFR1, VEGFA, FN1, HES6, S100A11, or H2AFX. In some embodiments, the plurality of capture probes comprise capture domains that bind specifically to ten or more of: ALB, SOCS2, CSTA, BAMBI, WIPI1, PRLR, BCL2, ZNF03, IL6ST, PRKACB, SPP1, CCND1, FGFR1, VEGFA, FN1, HES6, S100A11, or H2AFX.

In some embodiments, the plurality of capture probes comprise capture domains that bind specifically to nucleic acid analytes associated immune cells or immune function. In some embodiments, the methods further comprise determining a location of an immune cell based on the location and optionally the abundance of the analyte in the biological sample. In some embodiments, the immune cell is selected from a T-cell, a B-cell, a macrophage, a dendritic cell, or a NK cell. In some embodiments, the methods further comprise comparing the location and optionally the abundance of the analyte in the biological sample with a control sample. In some embodiments, the plurality of capture probes comprise capture domains that bind specifically to nucleic acid analytes encoding transcription factors. In some embodiments, the methods further comprise identifying a cell having aberrant cell signaling or gene expression based on the location and optionally the abundance of the analyte in the biological sample.

In some embodiments, the cell having aberrant cell signaling or gene expression is a cancer cell. In some embodiments, the methods further comprise comparing the location and optionally the abundance of the analyte in the biological sample with a control sample. In some embodiments, the methods further comprise determining one or more cellular areas of the biological sample. In some embodiments, the methods further comprise determining one or more non-cellular areas of the biological sample. In some embodiments, the one or more non-cellular areas include fat, fibrous tissue, glands, or a combination thereof.

In some embodiments, the methods further comprise imaging the tissue sample. In some embodiments, the imaging comprises the use of immunofluorescence microscopy. In some embodiments, the imaging comprises the use of immunohistochemistry. In some embodiments, the analyte is a nucleic acid. In some embodiments, the nucleic acid is DNA. In some embodiments, the nucleic acid is RNA. In some embodiments, the RNA is mRNA.

In some embodiments, disclosed herein is a substrate comprising a plurality of capture probes, wherein a capture probe of the plurality comprises (i) a capture domain that binds specifically to an analyte in the biological sample, and (ii) a spatial barcode; wherein capture probes of the plurality comprise capture domains that bind specifically to two or more of CENPW, A2ML1, VLDLR, SCRG1, RCL1, FABP7, CCNE1, MIEN1, CDC37L1, ERMP1, ACTG2, DMKN, CALML3, COL17A1, MAGED1, PTN, TMEM98, LY6D, TNC, or RTN4IP1.

In some embodiments, the capture probes of the plurality comprise capture domains that bind specifically to ten or more of CENPW, A2ML1, VLDLR, SCRG1, RCL1, FABP7, CCNE1, MIEN1, CDC37L1, ERMP1, ACTG2, DMKN, CALML3, COL17A1, MAGED1, PTN, TMEM98, LY6D, TNC, or RTN4IP1. In some embodiments, the capture probes of the plurality comprise capture domains that bind specifically to CENPW, A2ML1, VLDLR, SCRG1, RCL1, FABP7, CCNE1, MIEN1, CDC37L1, ERMP1, ACTG2, DMKN, CALML3, COL17A1, MAGED1, PTN, TMEM98, LY6D, TNC, or RTN4IP1. In some embodiments, the nucleic acid further comprises a functional sequence, wherein the functional sequence is a primer sequence or a complement thereof.

In some embodiments, disclosed herein is a method of diagnosing a subject as having breast cancer, wherein the method comprises: (a) determining an abundance of one or more of:

(1) CENPW, or a byproduct or precursor or degradation product thereof;

(2) A2ML1, or a byproduct or precursor or degradation product thereof;

(3) VLDLR, or a byproduct or precursor or degradation product thereof;

(4) SCRG1, or a byproduct or precursor or degradation product thereof;

(5) RCL1, or a byproduct or precursor or degradation product thereof;

(6) FABP7, or a byproduct or precursor or degradation product thereof;

(7) CCNE1, or a byproduct or precursor or degradation product thereof;

(8) MIEN1, or a byproduct or precursor or degradation product thereof;

(9) CDC37L1, or a byproduct or precursor or degradation product thereof;

(10) ERMP1, or a byproduct or precursor or degradation product thereof;

(11) TNFSF10, or a byproduct or precursor or degradation product thereof;

(12) ACTG2, or a byproduct or precursor or degradation product thereof;

(13) DMKN, or a byproduct or precursor or degradation product thereof;

(14) CALML3, or a byproduct or precursor or degradation product thereof;

(15) COL17A1, or a byproduct or precursor or degradation product thereof;

(16) MAGED1, or a byproduct or precursor or degradation product thereof;

(17) PTN, or a byproduct or precursor or degradation product thereof;

(18) TMEM98, or a byproduct or precursor or degradation product thereof;

(19) LY6D, or a byproduct or precursor or degradation product thereof;

(20) TNC, or a byproduct or precursor or degradation product thereof; and

(21) RTN4IP1, or a byproduct or precursor or degradation product thereof; and

(b) identifying a subject having increased abundance(s) of one or more of (1)-(21), in the biological sample as compared to reference sample(s) of the one or more of (1)-(21), as having breast cancer. In some embodiments, the method further comprises confirming a diagnosis of breast cancer in the subject by obtaining an image of the subject's breast. In some embodiments, the method further comprises administering a treatment of breast cancer to the subject.

In some embodiments, the subject is diagnosed as having invasive carcinoma, when the subject has increased abundance(s) of one or more analytes (1)-(11) and byproducts, precursors, and degradation products thereof. In some embodiments, the subject is diagnosed as having ductal carcinoma, when the subject has increased abundance(s) of one or more analytes (12)-(21) and byproducts, precursors, and degradation products thereof.

In some embodiments, disclosed herein is a method of diagnosing a subject as having breast cancer, wherein the method comprises: (a) determining an abundance of one or more analytes selected from the group consisting of: ALB, SOCS2, CSTA, BAMBI, WIPI1, PRLR, BCL2, ZNF03, IL6ST, PRKACB, SPP1, CCND1, FGFR1, VEGFA, FN1, HES6, S100A11, H2AFX, and byproducts, precursors, and degradation products thereof; and (b) identifying a subject having increased abundance(s) of one or more analytes ALB, SOCS2, CSTA, BAMBI, WIPI1, PRLR, BCL2, ZNF03, IL6ST, PRKACB, SPP1, CCND1, FGFR1, VEGFA, FN1, HES6, S100A11, H2AFX, and byproducts, precursors, and degradation products thereof, in the biological sample as compared to reference abundance(s) of the one or more analytes ALB, SOCS2, CSTA, BAMBI, WIPI1, PRLR, BCL2, ZNF03, IL6ST, PRKACB, SPP1, CCND1, FGFR1, VEGFA, FN1, HES6, S100A11, H2AFX, and byproducts, precursors, and degradation products thereof, as having breast cancer.

In some embodiments, the subject is diagnosed as having ductal carcinoma, when the subject has increased abundance(s) of one or more analytes ALB, SOCS2, CSTA, BAMBI, WIPI1, PRLR, BCL2, ZNF03, IL6ST and byproducts, precursors, and degradation products thereof. In some embodiments, the subject is diagnosed as having invasive carcinoma, when the subject has increased abundance(s) of one or more analytes PRKACB, SPP1, CCND1, FGFR1, VEGFA, FN1, HES6, S100A11, and H2AFX, and byproducts, precursors, and degradation products thereof.

Also featured herein is a method of diagnosing a subject as having breast cancer or having an increased likelihood of developing breast cancer, wherein the method comprises: (a) determining an abundance of one or more of

(1) CENPW, or a byproduct or precursor or degradation product thereof;

(2) A2ML1, or a byproduct or precursor or degradation product thereof;

(3) VLDLR, or a byproduct or precursor or degradation product thereof;

(4) SCRG1, or a byproduct or precursor or degradation product thereof;

(5) RCL1, or a byproduct or precursor or degradation product thereof;

(6) FABP7, or a byproduct or precursor or degradation product thereof;

(7) CCNE1, or a byproduct or precursor or degradation product thereof;

(8) MIEN1, or a byproduct or precursor or degradation product thereof;

(9) CDC37L1, or a byproduct or precursor or degradation product thereof;

(10) ERMP1, or a byproduct or precursor or degradation product thereof;

(11) TNFSF10, or a byproduct or precursor or degradation product thereof;

(12) ACTG2, or a byproduct or precursor or degradation product thereof;

(13) DMKN, or a byproduct or precursor or degradation product thereof;

(14) CALML3, or a byproduct or precursor or degradation product thereof;

(15) COL17A1, or a byproduct or precursor or degradation product thereof;

(16) MAGED1, or a byproduct or precursor or degradation product thereof;

(17) PTN, or a byproduct or precursor or degradation product thereof;

(18) TMEM98, or a byproduct or precursor or degradation product thereof;

(19) LY6D, or a byproduct or precursor or degradation product thereof;

(20) TNC, or a byproduct or precursor or degradation product thereof; and

(21) RTN4IP1, or a byproduct or precursor or degradation product thereof; and

(b) identifying a subject having increased abundance(s) of one or more of (1)-(21), in the biological sample as compared to reference abundance(s) of the one or more of (1)-(21), as having breast cancer or an increased likelihood of developing breast cancer.

Also disclosed herein is a method of diagnosing a subject as having breast cancer, wherein the method comprises (a) determining an abundance of one or more of: (i) one or more analytes selected from the group consisting of IGLC2, IGHG3, IGKC, IGHG1, IGLC3, IGHA1, IGHG2, IGHM, IGHG4, JCHAIN, ID3, CITTA, CST7, IFI27L2, FYN, MICAL1, HMOX1, CD7, ARHGEF1, and CFH and byproducts, precursors, and degradation products thereof; (ii) one or more analytes selected from the group consisting of MALAT1, CTSD, TYMP, SAMHD1, CYBA, ISG15, C1QA, RPS9, H2AFJ, ADIRF, ARHGDIA, APRT, AEBP1, PLEC, APOE, FCGRT, NDUFB7, MBD3, EMILIN1, and GADD45GIP1 and byproducts, precursors, and degradation products thereof; (iii) one or more analytes selected from the group consisting of CXCL14, TTLL12, GFRA1, DEGS1, AGR2, ARMT1, CCND1, ARPP21, CRAT, PRKACB, NSD3, PLAUR, CDKN2C, FIP1L1, TMEM159, TMEM141, LIN7C, ARHGEF39, ARFGEF3, and EMP2 and byproducts, precursors, and degradation products thereof; (iv) one or more analytes selected from the group consisting of CPB1, FCGR3B, KLHDC7B, SCGB1D2, SCUBE3, CXCL9, COX6C, CFB, SCGB2A2, NPY1R, AC087741.1, GBP5, IFT27, NEURL4, EMX1, SLC13A2, FAM110A, SPCS1, HGD, and ZNF587 and byproducts, precursors, and degradation products thereof; (v) one or more analytes selected from the group consisting of CRISP3, SLITRK6, C6orf141, VTCN1, SERHL2, CEACAM6, ABCC11, SHISA2, C2orf54, PDLIM1, ZC3H12A, VPS37B, IRF2BP2, RAPH1, NFKBIA, EIF2AK1, TRIM33, SFPQ, F7, and TRAPPC3 and byproducts, precursors, and degradation products thereof; (vi) one or more analytes selected from the group consisting of LINC00052, COX6C, SNCG, WFDC2, SLC39A6, MGST1, MCCD1, CSTA, PDE5A, MT-ND1, SRP14, TRAPPC1, SNRPD3, MAT2A, SLC7A8, POLR2K, TMBIM6, OCIAD1, EXOSC3, and CA14 and byproducts, precursors, and degradation products thereof; (vii) one or more analytes selected from the group consisting of ACKR1, IGFBP7, AQP1, VWF, MALAT1, SPARCL1, TAGLN, CCL21, ACTA2, CCDC₈₀, CYB5R3, PLXND1, DUSP1, RPS6, C1QA, HEG1, ETS2, AKAP9, CCAR1, and TRIM47 and byproducts, precursors, and degradation products thereof; (viii) one or more analytes selected from the group consisting of ALB, MGP, ZNF350-AS1, S100G, STC2, CARTPT, AC087379.2, GPC3, ERP27, APOD, CDV3, TPI1, TSPYL5, PFKP, CRIM1, PPT1, AC100826.1, ALDOA, LGR4, and GLRX2 and byproducts, precursors, and degradation products thereof; (ix) one or more analytes selected from the group consisting of AC087379.2, S100G, SCGB2A2, PGM5-AS1, HEBP1, AMIGO2, PCED1B, SCGB1D2, ITPR1, IFT122, GABARAPL1, G6PC3, INPP5K, STC2, HEXIM2, RIDA, LRP2, HK2, IL1R2, and GOT2 and byproducts, precursors, and degradation products thereof; (x) one or more analytes selected from the group consisting of ALB, MT-ND2, MT-ND1, MT-ND3, MT-ATP6, MT-ND4, MT-CO1, MT-CO3, MT-ATPS, MT-ND5, GLO1, ABHD2, LINC00052, RERG, STC2, MALAT1, SOX4, CA12, ZNF703, and MCCD1 and byproducts, precursors, and degradation products thereof; (xi) one or more analytes selected from the group consisting of LINC00645, SLC30A8, MUC5B, COLEC12, PVALB, CPB1, EXOC2, AC037198.2, VSTM2A, FSIP1, KIF16B, SPATA20, TSPAN9, CACNB3, CAMK2N1, IFT27, NEURL1, TRIM3, SLC46A1, and ECI2 and byproducts, precursors, and degradation products thereof; (xii) one or more analytes selected from the group consisting of MGP, TFF1, KRT14, S100A9, KRT17, S100G, S100A2, ZNF350-AS1, KRT5, S100A8, MN1, TMEM45A, DNALI1, C3orf14, TSR1, AL445524.1, SEMA3C, NDUFAF2, ASCL1, and GRHL2 and byproducts, precursors, and degradation products thereof; (xiii) one or more analytes selected from the group consisting of SAA1, FABP4, GPX3, PIP, ADH1B, PLIN1, COL2A1, SH3BGRL, ADIPOQ, PLIN4, HNRNPA0, RPL11, SLC40A1, RPLP2, CTDSP2, SORL1, RPL31, ECE1, SFRP2, and CCND1 and byproducts, precursors, and degradation products thereof; or (xiv) one or more analytes selected from the group consisting of PDE5A, MGP, WFDC2, MRPS30-DT, RBM20, MRPS30, AMFR, STC2, KCNE4, DDR1, MCCC1, PDPR, AMZ2, C5orf15, TBC1D9, SRPK1, LINC01488, TSG101, COA3, and NFKBIE and byproducts, precursors, and degradation products thereof; and (b) identifying a subject having dysregulated abundance(s) of one or more analytes (i)-(xiv) and byproducts, precursors, and degradation products thereof, in the biological sample as compared to reference abundance(s) of the one or more analytes (i)-(xiv) and byproducts, precursors, and degradation products thereof, as having breast cancer.

In some embodiments, the method comprises: (a) determining an abundance of one or more of: (i) one or more analytes selected from the group consisting of IGLC2, IGHG3, IGKC, IGHG1, IGLC3, IGHA1, IGHG2, IGHM, IGHG4, JCHAIN, and byproducts, precursors, and degradation products thereof; (ii) one or more analytes selected from the group consisting of MALAT1, CTSD, TYMP, SAMHD1, CYBA, ISG15, C1QA, RPS9, H2AFJ, ADIRF, and byproducts, precursors, and degradation products thereof; (iii) one or more analytes selected from the group consisting of CXCL14, TTLL12, GFRA1, DEGS1, AGR2, ARMT1, CCND1, ARPP21, CRAT, PRKACB, and byproducts, precursors, and degradation products thereof; (iv) one or more analytes selected from the group consisting of CPB1, FCGR3B, KLHDC7B, SCGB1D2, SCUBE3, CXCL9, COX6C, CFB, SCGB2A2, NPY1R, and byproducts, precursors, and degradation products thereof; (v) one or more analytes selected from the group consisting of CRISP3, SLITRK6, C6orf141, VTCN1, SERHL2, CEACAM6, ABCC11, SHISA2, C2orf54, PDLIM1, and byproducts, precursors, and degradation products thereof; (vi) one or more analytes selected from the group consisting of LINC00052, COX6C, SNCG, WFDC2, SLC39A6, MGST1, MCCD1, CSTA, PDE5A, MT-ND1, and byproducts, precursors, and degradation products thereof; (vii) one or more analytes selected from the group consisting of ACKR1, IGFBP7, AQP1, VWF, MALAT1, SPARCL1, TAGLN, CCL21, ACTA2, CCDC₈₀, and byproducts, precursors, and degradation products thereof; (viii) one or more analytes selected from the group consisting of ALB, MGP, ZNF350-AS1, S100G, STC2, CARTPT, AC087379.2, GPC3, ERP27, APOD, and byproducts, precursors, and degradation products thereof; (ix) one or more analytes selected from the group consisting of AC087379.2, S100G, SCGB2A2, PGM5-AS1, HEBP1, AMIGO2, PCED1B, SCGB1D2, ITPR1, IFT122, and byproducts, precursors, and degradation products thereof; (x) one or more analytes selected from the group consisting of ALB, MT-ND2, MT-ND1, MT-ND3, MT-ATP6, MT-ND4, MT-CO1, MT-CO3, MT-ATPS, MT-ND5, and byproducts, precursors, and degradation products thereof; (xi) one or more analytes selected from the group consisting of LINC00645, SLC30A8, MUC5B, COLEC12, PVALB, CPB1, EXOC2, AC037198.2, VSTM2A, FSIP1, and byproducts, precursors, and degradation products thereof; (xii) one or more analytes selected from the group consisting of MGP, TFF1, KRT14, S100A9, KRT17, S100G, S100A2, ZNF350-AS1, KRT5, S100A8, and byproducts, precursors, and degradation products thereof; (xiii) one or more analytes selected from the group consisting of SAA1, FABP4, GPX3, PIP, ADH1B, PLIN1, COL2A1, SH3BGRL, ADIPOQ, PLIN4, and byproducts, precursors, and degradation products thereof; or (xiv) one or more analytes selected from the group consisting of PDE5A, MGP, WFDC2, MRPS30-DT, RBM20, MRPS30, AMFR, STC2, KCNE4, DDR1, and byproducts, precursors, and degradation products thereof; and (b) identifying a subject having increased abundance(s) of one or more analytes (i)-(xiv) and byproducts, precursors, and degradation products thereof, in the biological sample as compared to reference abundance(s) of the one or more analytes (i)-(xiv) and byproducts, precursors, and degradation products thereof, as having breast cancer.

In some embodiments, the method comprises: (a) determining an abundance of one or more of: (i) one or more analytes selected from the group consisting of ID3, CIITA, CST7, IFI27L2, FYN, MICAL1, HMOX1. CD7. ARHGEF1, and CFH and byproducts, precursors, and degradation products thereof; (ii) one or more analytes selected from the group consisting of ARHGDIA, APRT, AEBP1, PLEC, APOE, FCGRT, NDUFB7, MBD3, EMILIN1, and GADD45GIP1 and byproducts, precursors, and degradation products thereof; (iii) one or more analytes selected from the group consisting of NSD3, PLAUR, CDKN2C, FIP1L1, TMEM159, TMEM141, LIN7C, ARHGEF39, ARFGEF3, and EMP2 and byproducts, precursors, and degradation products thereof; (iv) one or more analytes selected from the group consisting of AC087741.1, GBP5, IFT27, NEURL4, EMX1, SLC13A2, FAM110A, SPCS1, HGD, and ZNF587 and byproducts, precursors, and degradation products thereof; (v) one or more analytes selected from the group consisting of ZC3H12A, VPS37B, IRF2BP2, RAPH1, NFKBIA, EIF2AK1, TRIM33, SFPQ, F7, and TRAPPC3 and byproducts, precursors, and degradation products thereof; (vi) one or more analytes selected from the group consisting of SRP14, TRAPPC1, SNRPD3, MAT2A, SLC7A8, POLR2K, TMBIM6, OCIAD1, EXOSC3, and CA14 and byproducts, precursors, and degradation products thereof; (vii) one or more analytes selected from the group consisting of CYB5R3, PLXND1, DUSP1, RPS6, C1QA, HEG1, ETS2, AKAP9, CCAR1, and TRIM47 and byproducts, precursors, and degradation products thereof; (viii) one or more analytes selected from the group consisting of CDV3, TPI1, TSPYL5, PFKP, CRIM1, PPT1, AC100826.1, ALDOA, LGR4, and GLRX2 and byproducts, precursors, and degradation products thereof; (ix) one or more analytes selected from the group consisting of GABARAPL1, G6PC3, INPP5K, STC2, HEXIM2, RIDA, LRP2, HK2, IL1R2, and GOT2 and byproducts, precursors, and degradation products thereof; (x) one or more analytes selected from the group consisting of GLO1, ABHD2, LINC00052, RERG, STC2, MALAT1, SOX4, CA12, ZNF703, and MCCD1 and byproducts, precursors, and degradation products thereof; (xi) one or more analytes selected from the group consisting of KIF16B, SPATA20, TSPAN9, CACNB3, CAMK2N1, IFT27, NEURL1, TRIMS, SLC46A1, and ECI2 and byproducts, precursors, and degradation products thereof; (xii) one or more analytes selected from the group consisting of MN1, TMEM45A, DNALI1, C3orf14, TSR1, AL445524.1, SEMA3C, NDUFAF2, ASCL1, and GRHL2 and byproducts, precursors, and degradation products thereof; (xiii) one or more analytes selected from the group consisting of HNRNPA0, RPL11, SLC40A1, RPLP2, CTDSP2, SORL1, RPL31, ECE1, SFRP2, and CCND1 and byproducts, precursors, and degradation products thereof; or (xiv) one or more analytes selected from the group consisting of MCCC1, PDPR, AMZ2, C5orf15, TBC1D9, SRPK1, LINC01488, TSG101, COA3, and NFKBIE and byproducts, precursors, and degradation products thereof; and (b) identifying a subject having decreased abundance(s) of one or more analytes (i)-(xiv) and byproducts, precursors, and degradation products thereof, in the biological sample as compared to reference abundance(s) of the one or more analytes (i)-(xiv) and byproducts, precursors, and degradation products thereof, as having breast cancer.

In some embodiments, the methods disclosed herein further comprise obtaining an image of the biological sample. In some instances, the methods further comprises obtaining a biological sample from the subject.

In some embodiments, the methods further comprise administering a treatment of breast cancer to the subject, adjusting a dosage of a treatment of breast cancer for the subject, or adjusting a treatment of breast cancer for the subject. In some instances, the treatment comprises administering one or more therapies selected from the group consisting of an endocrine therapy, a chemotherapy a hormonal therapy, and a surgical resection. In some instances, endocrine therapy is one or more agents selected from the group consisting of tamoxifen, raloxifene, megestrol, toremifene, and an aromatase inhibitor. In some instances, the aromatase inhibitor is one or more agents selected from the group consisting of anastrozole, letrozole, or exemestane. In some instances, the chemotherapy is neoadjuvant chemotherapy. In some instances, the neoadjuvant chemotherapy is one or more agents selected from a taxane derivative, an anthracycline derivative, and a topoisomerase inhibitors. In some instances, the taxane derivative is one or more agents selected from docetaxel and paclitaxel; and the anthracycline derivative is doxorubicin. In some instances, the surgical resection is surgery for breast tissue and/or lymph node tissue. In some instances, the breast tissue surgery is selected from the group comprising lumpectomy, quadrantectomy, partial mastectomy, segmental mastectomy, complete mastectomy, and the lymph node tissue surgery is selected from the group consisting of sentinel lymph node biopsy and axillary lymph node dissection.

In some embodiments, the methods further comprise monitoring the identified subject for the development of symptoms of breast cancer. In some embodiments, the methods further comprise recording in the identified subject's clinical record that the subject has an increased likelihood of developing breast cancer. In some embodiments, the methods further comprise notifying the subject's family that the subject has an increased likelihood of developing breast cancer. In some embodiments, the methods further comprise administering to the subject a treatment for decreasing the likelihood of developing breast cancer. In some embodiments, the methods further comprise obtaining the biological sample from the subject. In some embodiments, the abundance is an abundance of protein or a byproduct or precursor or degradation product thereof. In some embodiments, the abundance is an abundance of mRNA or a fragment thereof. All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The patent or application file contains at least one drawing executed in color. Copies of this patent or patent application publication with color drawing(s) will be provided by the Office upon request and payment of the necessary fee.

The following drawings illustrate certain embodiments of the features and advantages of this disclosure. These embodiments are not intended to limit the scope of the appended claims in any manner. Like reference symbols in the drawings indicate like elements.

FIG. 1 shows an exemplary spatial analysis workflow.

FIG. 2 shows an exemplary spatial analysis workflow.

FIG. 3 shows an exemplary spatial analysis workflow.

FIG. 4 shows an exemplary spatial analysis workflow.

FIG. 5 shows an exemplary spatial analysis workflow.

FIG. 6 is a schematic diagram showing an example of a barcoded capture probe, as described herein.

FIG. 7 is a schematic illustrating a cleavable capture probe, wherein the cleaved capture probe can enter into a non-permeabilized cell and bind to target analytes within the sample.

FIG. 8 is a schematic diagram of an exemplary multiplexed spatially-barcoded feature.

FIG. 9 is a schematic diagram of an exemplary analyte capture agent.

FIG. 10 is a schematic diagram depicting an exemplary interaction between a feature-immobilized capture probe 1024 and an analyte capture agent 1026.

FIGS. 11A, 11B, and 11C are schematics illustrating how streptavidin cell tags can be utilized in an array-based system to produce a spatially-barcoded cells or cellular contents.

FIG. 12 is a schematic showing the arrangement of barcoded features within an array.

FIG. 13 is a schematic illustrating a side view of a diffusion-resistant medium, e.g., a lid.

FIGS. 14A and 14B are schematics illustrating expanded FIG. 14A and side views FIG. 14B of an electrophoretic transfer system configured to direct transcript analytes toward a spatially-barcoded capture probe array.

FIG. 15 is a schematic illustrating an exemplary workflow protocol utilizing an electrophoretic transfer system.

FIG. 16 shows an example of a microfluidic channel structure 1600 for partitioning dissociated sample (e.g., biological particles or individual cells from a sample).

FIG. 17A shows an example of a microfluidic channel structure 1700 for delivering spatial barcode carrying beads to droplets.

FIG. 17B shows a cross-section view of another example of a microfluidic channel structure 1750 with a geometric feature for controlled partitioning.

FIG. 17C shows an example of a workflow schematic.

FIG. 18 is a schematic depicting cell tagging using either covalent conjugation of the analyte binding moiety to the cell surface or non-covalent interactions with cell membrane elements.

FIG. 19 is a schematic depicting cell tagging using either cell-penetrating peptides or delivery systems.

FIG. 20A is a workflow schematic illustrating exemplary, non-limiting, non-exhaustive steps for “pixelating” a sample, wherein the sample is cut, stamped, microdissected, or transferred by hollow-needle or microneedle, moving a small portion of the sample into an individual partition or well.

FIG. 20B is a schematic depicting multi-needle pixilation, wherein an array of needles punched through a sample on a scaffold and into nanowells containing gel beads and reagents below. Once the needle is in the nanowell, the cell(s) are ejected.

FIG. 21 shows a workflow schematic illustrating exemplary, non-limiting, non-exhaustive steps for dissociating a spatially-barcoded sample for analysis via droplet or flow cell analysis methods.

FIG. 22A is a schematic diagram showing an example sample handling apparatus that can be used to implement various steps and methods described herein.

FIG. 22B is a schematic diagram showing an example imaging apparatus that can be used to obtain images of biological samples, analytes, and arrays of features.

FIG. 22C is a schematic diagram of an example of a control unit of the apparatus of FIGS. 22A and 22B.

FIG. 23A shows a histological section of an invasive ductal carcinoma annotated by a pathologist.

FIG. 23B shows a tissue plot with spots colored by unsupervised clustering.

FIG. 23C is a tSNE plot of spots colored by unsupervised clustering.

FIG. 23D shows a gene expression heat map of the most variable genes between 9 clusters.

FIG. 23E shows the expression levels of genes corresponding to human epidermal growth factor receptor 2 (Her2), estrogen receptor (ER), and progesterone receptor (PR) in the tissue section.

FIG. 23F shows the expression levels of genes of top differentially expressed genes from each of the 9 clusters on individual plots.

FIG. 23G shows the expression levels of genes of top differentially expressed genes from each of the 9 clusters on a single plot.

FIG. 23H is a plot of the expression levels of the top differentially expressed genes from each of the 9 clusters in invasive ductal cell carcinoma (IDC) and normal breast tissue.

FIG. 23I shows the expression of KRT14 in IDC and match normal tissue.

FIG. 23J is a plot of the expression levels of extracellular matrix genes in IDC and normal tissue.

FIG. 24A shows a schematic of an example analytical workflow in which electrophoretic migration of analytes is performed after permeabilization.

FIG. 24B shows a schematic of an example analytical workflow in which electrophoretic migration of analytes and permeabilization are performed simultaneously.

FIG. 25A shows an example perpendicular, single slide configuration for use during electrophoresis.

FIG. 25B shows an example parallel, single slide configuration for use during electrophoresis

FIG. 25C shows an example multi-slide configuration for use during electrophoresis.

FIG. 26A shows detection of cancer-related genes using probes for the entire transcriptome in a triple-negative breast cancer sample.

FIG. 26B shows detection of cancer-related genes using probes from a cancer-specific panel in the triple-negative breast cancer sample as in FIG. 26A.

FIG. 26C shows the correlation of expression detection using probes for the entire transcriptome from FIG. 26A (x-axis) versus probes from a cancer-specific panel from FIG. 26B (y-axis) in the triple-negative breast cancer sample.

FIG. 27A shows detection of immune-related genes using probes for the entire transcriptome in a triple-negative breast cancer sample.

FIG. 27B shows detection of immune-related genes using probes from an immune-specific panel in the triple-negative breast cancer sample as in FIG. 27A.

FIG. 27C shows the correlation of expression detection using probes for the entire transcriptome from FIG. 27A (x-axis) versus probes from an immune-specific panel from FIG. 27B (y-axis) in the triple-negative breast cancer sample.

FIG. 28A shows detection of pathway-related genes using probes for the entire transcriptome in a triple-negative breast cancer sample.

FIG. 28B shows detection of immune-related genes using probes from a pathway-specific panel in the triple-negative breast cancer sample as in FIG. 28A.

FIG. 28C shows the correlation of expression detection using probes for the entire transcriptome from FIG. 28A (x-axis) versus probes from a pathway-specific panel from FIG. 28B (y-axis) in the triple-negative breast cancer sample.

FIGS. 29A and 29B show identification of cellular and non-cellular areas of a triple negative breast cancer tissue sample using Soup or Cell Cluster Assignment.

FIGS. 30A and 30B show identification of cellular and non-cellular areas of a triple negative breast cancer tissue sample by examining the inflammation signal in each area.

FIGS. 30C and 30D show quantification of the inflammation score for each gene cluster.

FIG. 31 shows identification of areas of a triple negative breast cancer tissue sample using panel data.

FIGS. 32A and 32B show identification of B-cells in a triple negative breast cancer tissue sample.

FIGS. 33A and 33B show infiltration of particular cells in a triple negative breast cancer tissue sample.

FIGS. 34A-34C show immunohistochemical detection of CD3 (FIG. 34B) and intensity of CD3 protein expression (FIG. 34C). FIG. 34A: merged image.

FIG. 34D shows mRNA quantification of T-cells.

FIG. 34E shows protein quantification of CD3.

FIG. 34F shows mRNA quantification of T-cells.

FIG. 34G shows spots clustered on CD3 protein expression fluorescence intensity (High>0.3<Low).

FIG. 34H shows differential expression CD3 High vs Low (log fold change).

FIG. 35A shows a pathologist annotation of areas of a breast cancer sample, identifying areas having invasive cancer (IC), fibrous tissue, or ductal cancer in situ (DCIS).

FIG. 35B shows a Uniform Manifold Approximation and Projection (UMAP) graph of 14 clusters (clusters 0-13) identified using Seurat.

FIG. 35C shows spatial expression of 14 clusters (clusters 0-13) identified using Seurat.

FIG. 36 shows localized expression of the most upregulated individual genes in the breast cancer sample of FIG. 35A.

FIG. 37 shows localized expression of the most downregulated individual genes in the breast cancer sample of FIG. 35A.

FIG. 38A shows a brightfield image showing H&E staining of TNBC tissue section.

FIG. 38B shows a pathologist annotations of TNBC tissue section overlaid on H&E staining.

FIG. 38C shows classification as defined by pathologist annotations were assigned to each barcoded spot and overlaid on H&E staining of the tissue section.

FIG. 38D shows an overlay of H&E staining and graph-based cluster assignment.

FIG. 38E shows a UMAP projection of graph-based clustering of the section in FIG. 38A.

FIG. 38F shows targeted gene expression analysis of a second section of the same TNBC sample with the cancer panel.

FIG. 39A shows a UMAP projection of graph-based clustering of the single cell sample.

FIG. 39B shows regions of specific T-cell subtype infiltration in the section.

FIG. 39C shows localization of mammary stem cells, identified by CD39f^{hi} expression in snRNA-seq data and visualized over an H&E image.

FIG. 39D shows an image inferring each spot as either tumor or normal.

FIGS. 39E, 39F, and 39G show mutant variable load in three serial sections of a TNBC sample.

FIG. 40A shows a UMAP projection of graph-based clustering of expression of IGLC2.

FIG. 40B shows spatial expression of IGLC2 in a TNBC sample.

FIG. 40C shows spatial expression of CCL19 in a TNBC sample.

FIG. 40D shows spatial expression of IGHA2 in a TNBC sample.

FIG. 40E shows spatial expression of MIEN1 in a TNBC sample.

FIG. 40F shows spatial expression of SLPI in a TNBC sample.

FIG. 41 shows regions of specific T-cell subtype infiltration in the section.

FIG. 42 shows regions of specific B-cell subtype infiltration in the section.

FIG. 43 shows regions of specific tumor cell infiltration in the section.

FIG. 44 shows localization of mammary stem cells, identified by CD39f^{hi} expression in snRNA-seq data and visualized over an H&E image.

FIG. 45 shows regions of luminal progenitor cells (i.e., cancer stem cells (“csc”)) in the section.

FIG. 46A shows targeted gene expression (pan-cancer markers) analysis of a breast invasive ductal carcinoma (IDC) tissue section overlaid on a brightfield image showing H&E staining of the same tissue section.

FIG. 46B shows targeted gene expression (immunology markers) analysis of a breast IDC tissue section overlaid on a brightfield image showing H&E staining of the same tissue section.

FIG. 46C shows targeted gene expression (gene-signature (i.e., transcription factor panel) markers) analysis of a breast IDC tissue section overlaid on a brightfield image showing H&E staining of the same tissue section.

FIG. 46D shows targeted gene expression (unbiased markers) analysis of a breast IDC tissue section overlaid on a brightfield image showing H&E staining of the same tissue section.

FIG. 47A shows a graph of universal molecular identifiers (UMIs) per gene in a parent sample (x axis) vs UMIs per gene in a targeted sample (y axis) for the pan-cancer markers.

FIG. 47B shows a graph of universal molecular identifiers (UMIs) per gene in a parent sample (x axis) vs UMIs per gene in a targeted sample (y axis) for the immunology markers.

FIG. 47C shows a graph of universal molecular identifiers (UMIs) per gene in a parent sample (x axis) vs UMIs per gene in a targeted sample (y axis) for the gene signature markers.

FIG. 48A shows identification of areas of a breast IDC tissue sample using panel data.

FIG. 48B shows multi-section aggregation of detected analytes.

FIG. 49A shows targeted gene expression (B-cells, immunology targeted) analysis of a breast IDC tissue section overlaid on a brightfield image showing H&E staining of the same tissue section.

FIG. 49B shows targeted gene expression (monocytes, immunology targeted) analysis of a breast IDC tissue section overlaid on a brightfield image showing H&E staining of the same tissue section.

FIG. 49C shows targeted gene expression (T-cells, immunology targeted) analysis of a breast IDC tissue section overlaid on a brightfield image showing H&E staining of the same tissue section.

FIG. 49D shows targeted gene expression (lymphocytes, immunology targeted) analysis of a breast IDC tissue section overlaid on a brightfield image showing H&E staining of the same tissue section.

### DETAILED DESCRIPTION

### I. Introduction

Spatial analysis methodologies can provide a vast amount of analyte level and/or expression data for a variety of multiple analytes within a sample at high spatial resolution, e.g., while retaining the native spatial context. Spatial analysis methods can include, e.g., the use of a capture probe including a spatial barcode (e.g., a nucleic acid sequence that provides information as to the position of the capture probe within a cell or a tissue sample (e.g., mammalian cell or a mammalian tissue sample) and a capture domain that is capable of binding to an analyte (e.g., a protein and/or nucleic acid) produced by and/or present in a cell.

Tissues and cells can be obtained from any source. For example, tissues and cells can be obtained from single-cell or multicellular organisms (e.g., a mammal). Tissues and cells obtained from a mammal, e.g., a human, often have varied analyte levels (e.g., gene and/or protein expression) which can result in differences in cell morphology and/or function. The position of a cell or a subset of cells (e.g., neighboring cells and/or non-neighboring cells) within a tissue can affect, e.g., the cell's fate, behavior, morphology, and signaling and cross-talk with other cells in the tissue. Information regarding the differences in analyte levels (gene and/or protein expression) within different cells in a tissue of a mammal can also help physicians select or administer a treatment that will be effective and can allow researchers to identify and elucidate differences in cell morphology and/or cell function in the single-cell or multicellular organisms (e.g., a mammal) based on the detected differences in analyte levels within different cells in the tissue. Differences in analyte levels within different cells in a tissue of a mammal can also provide information on how tissues (e.g., healthy and diseased tissues) function and/or develop.

Non-limiting aspects of spatial analysis methodologies are described in WO 2011/127099, WO 2014/210233, WO 2014/210225, WO 2016/162309, WO 2018/091676, WO 2012/140224, WO 2014/060483, WO 2020/176788 U.S. Pat. Nos. 10,002,316, 9,727,810, U.S. Patent Application Publication No. 2020/0277663, U.S. Patent Application Publication No. 2017/0016053, Rodrigues et al., Science 363(6434):1463-1467, 2019; WO 2018/045186, Lee et al., Nat. Protoc. 10(3):442-458, 2015; WO 2016/007839, WO 2018/045181, WO 2014/163886, Trejo et al., PLoS ONE 14(2):e0212031, 2019, U.S. Patent Application Publication No. 2018/0245142, Chen et al., Science 348(6233):aaa6090, 2015, Gao et al., BMC Biol. 15:50, 2017, WO 2017/144338, WO 2018/107054, WO 2017/222453, WO 2019/068880, WO 2011/094669, U.S. Pat. Nos. 7,709,198, 8,604,182, 8,951,726, 9,783,841, 10,041,949, WO 2016/057552, WO 2017/147483, WO 2018/022809, WO 2016/166128, WO 2017/027367, WO 2017/027368, WO 2018/136856, WO 2019/075091, U.S. Pat. No. 10,059,990, WO 2018/057999, WO 2015/161173, and Gupta et al., Nature Biotechnol. 36:1197-1202, 2018, each of which is incorporated by reference in its entirety, and can be used herein in any combination. Further non-limiting aspects of spatial analysis methodologies are described herein.

Some general terminology that may be used in this disclosure can be found in Section (I)(b) of WO 2020/176788 and/or U.S. Patent Application Publication No. 2020/0277663, each of which is incorporated by reference in its entirety. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest. Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663, each of which is incorporated by reference in its entirety. In some embodiments, an analyte is a biomarker disclosed herein.

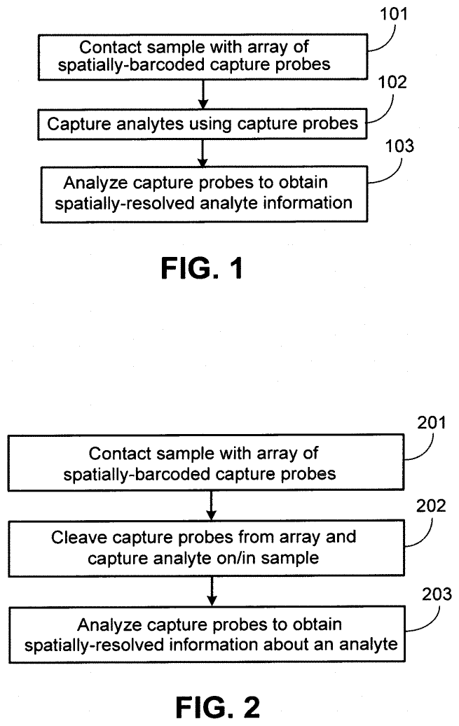

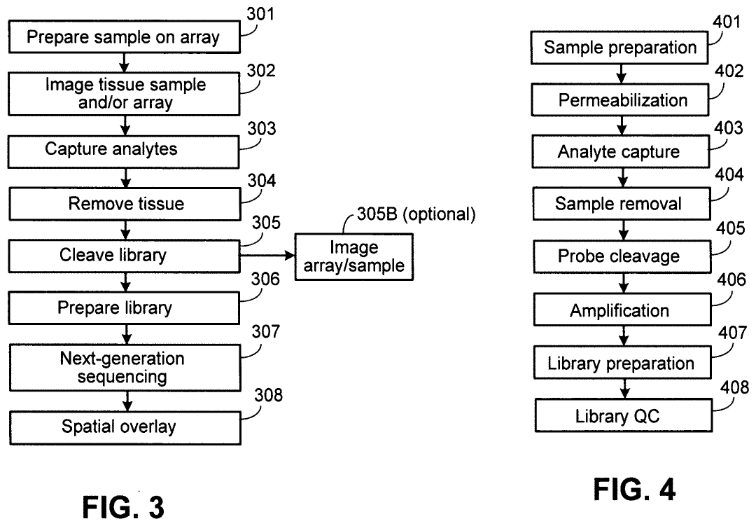

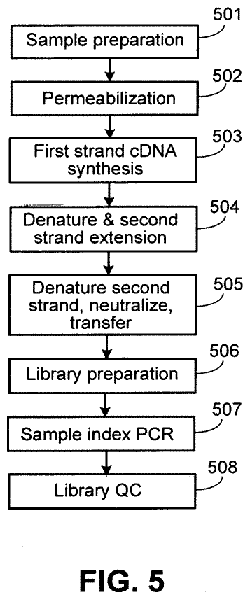

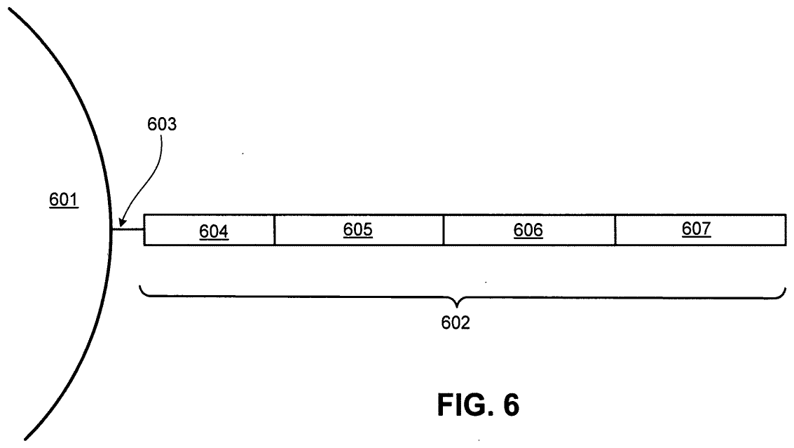

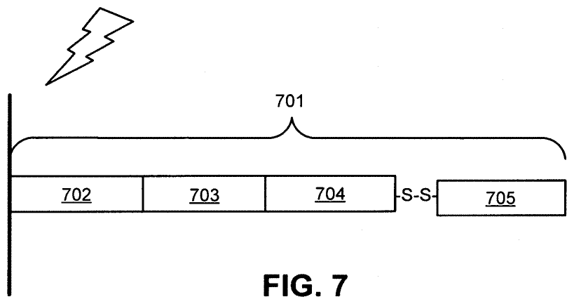

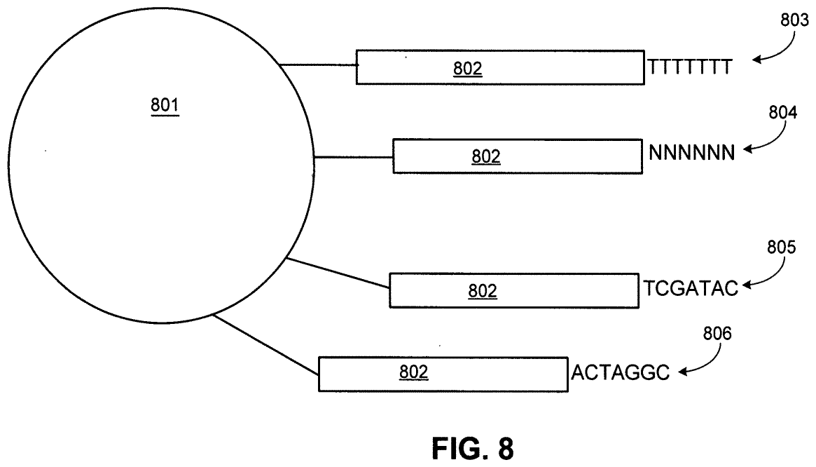

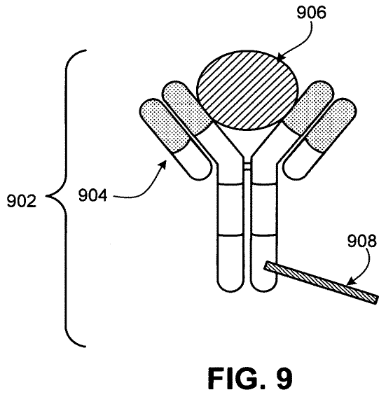

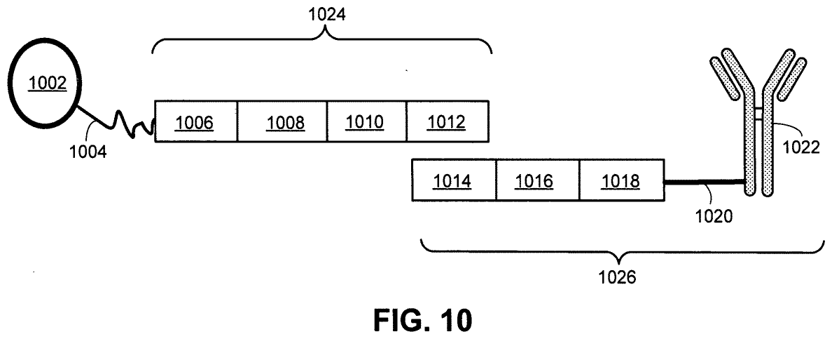

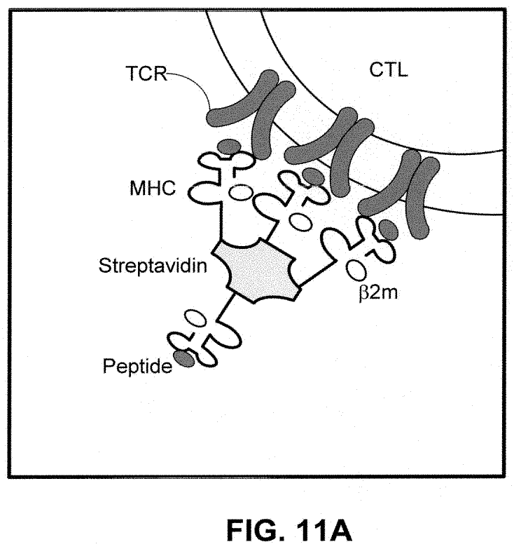

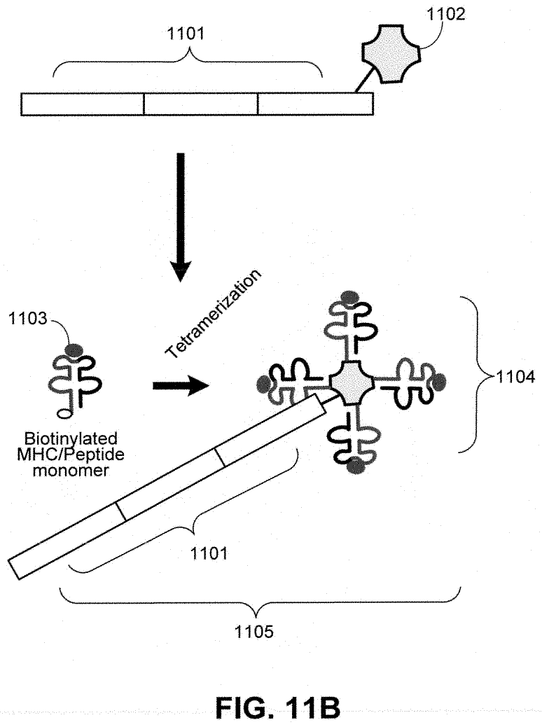

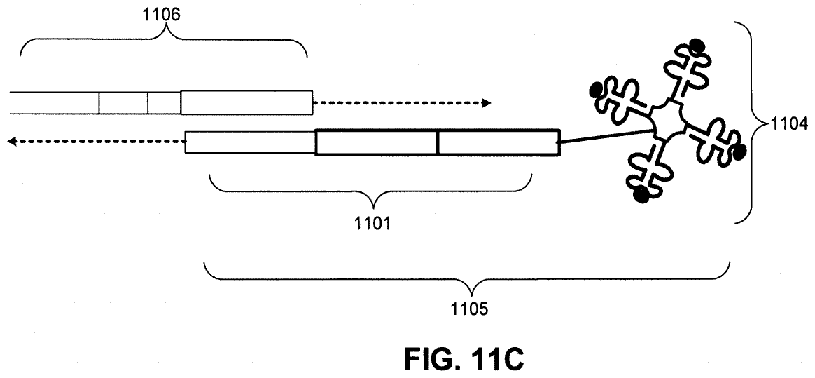

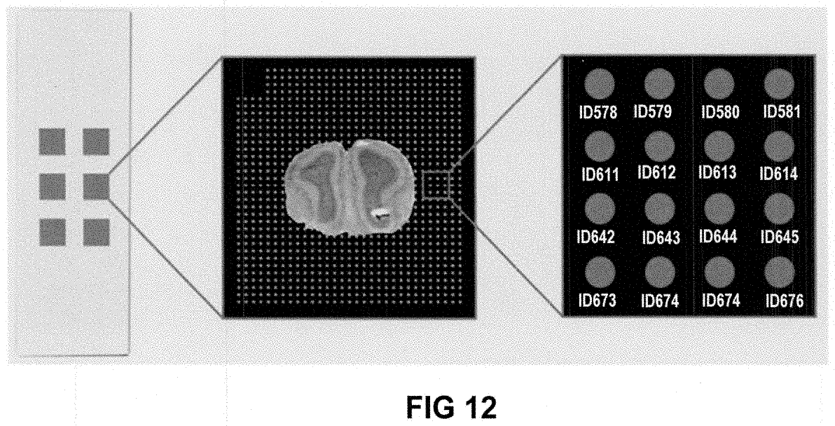

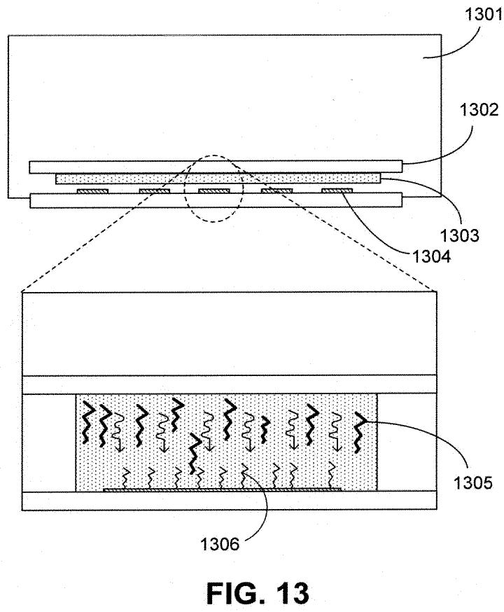

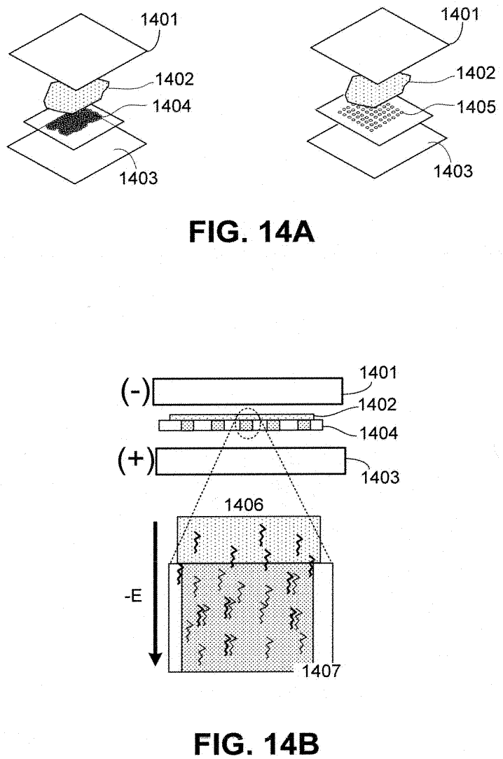

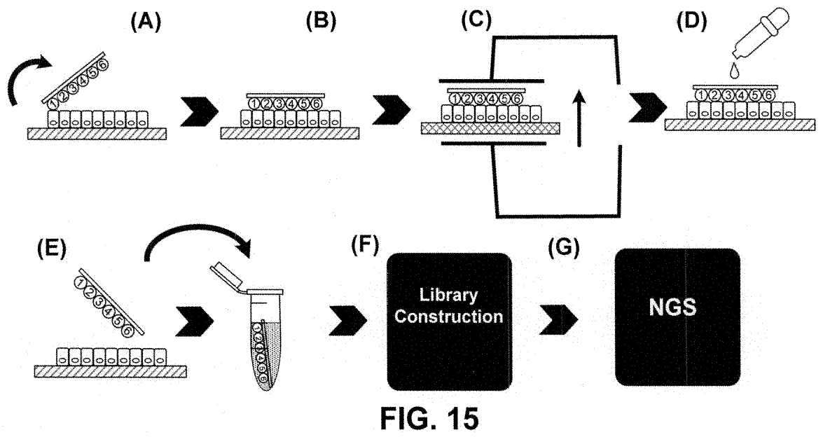

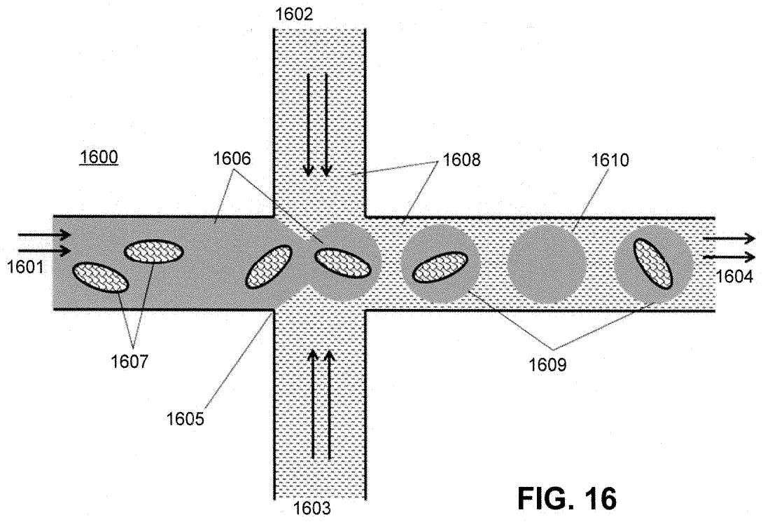

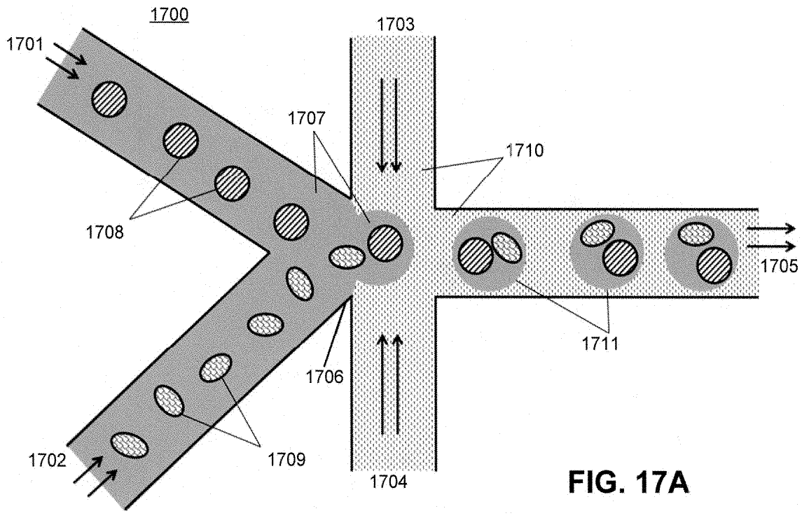

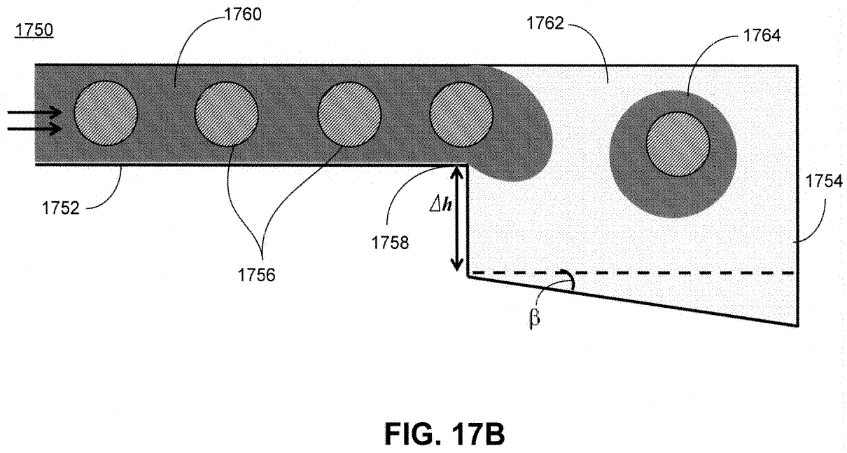

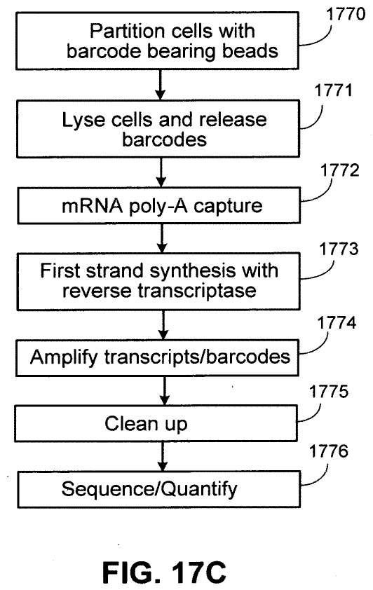

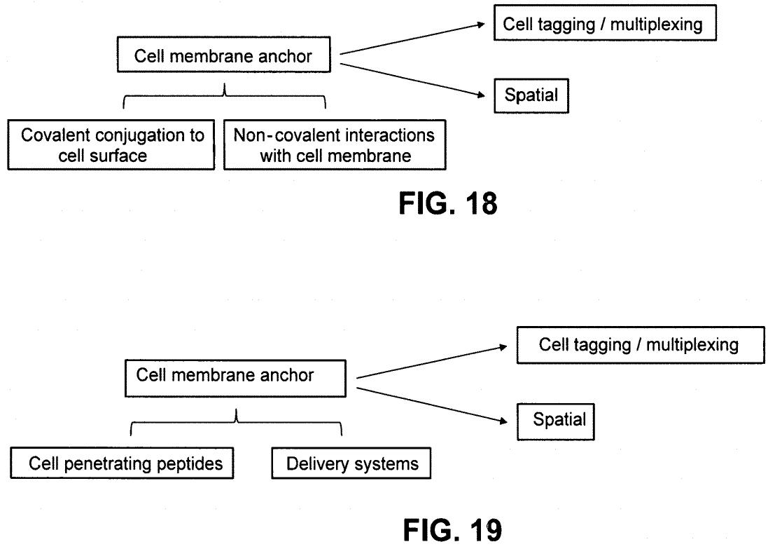

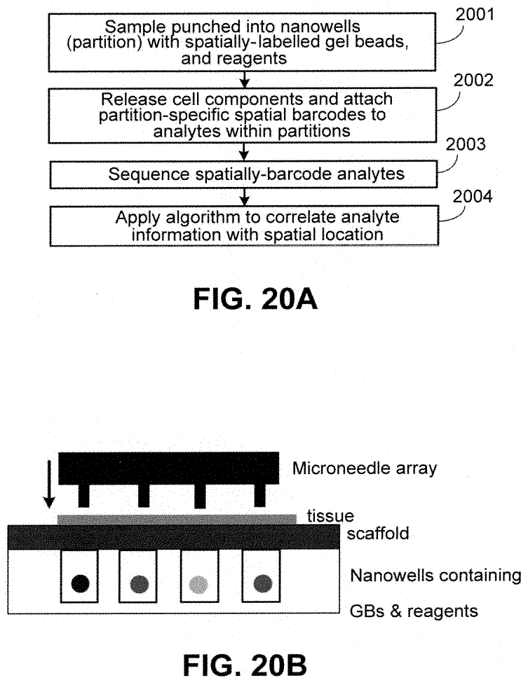

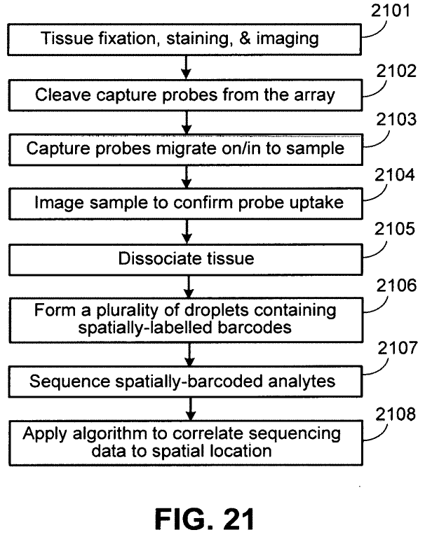

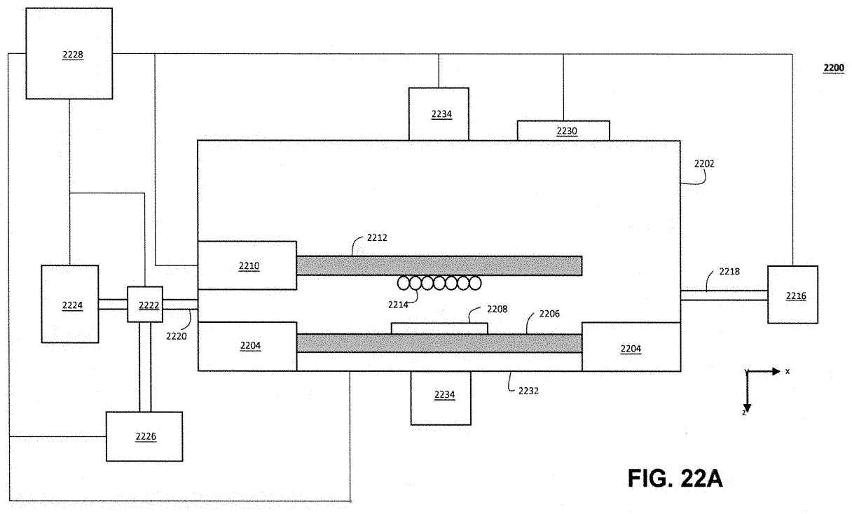

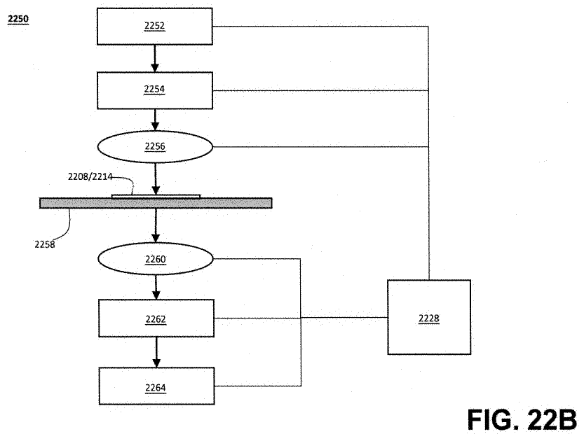

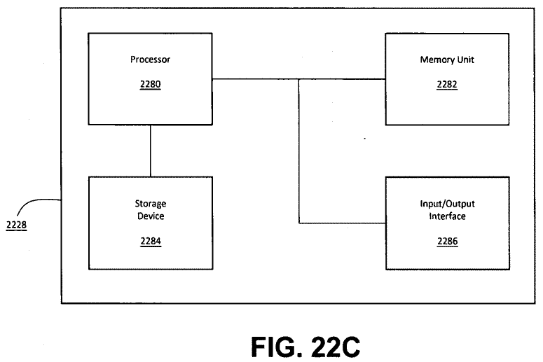

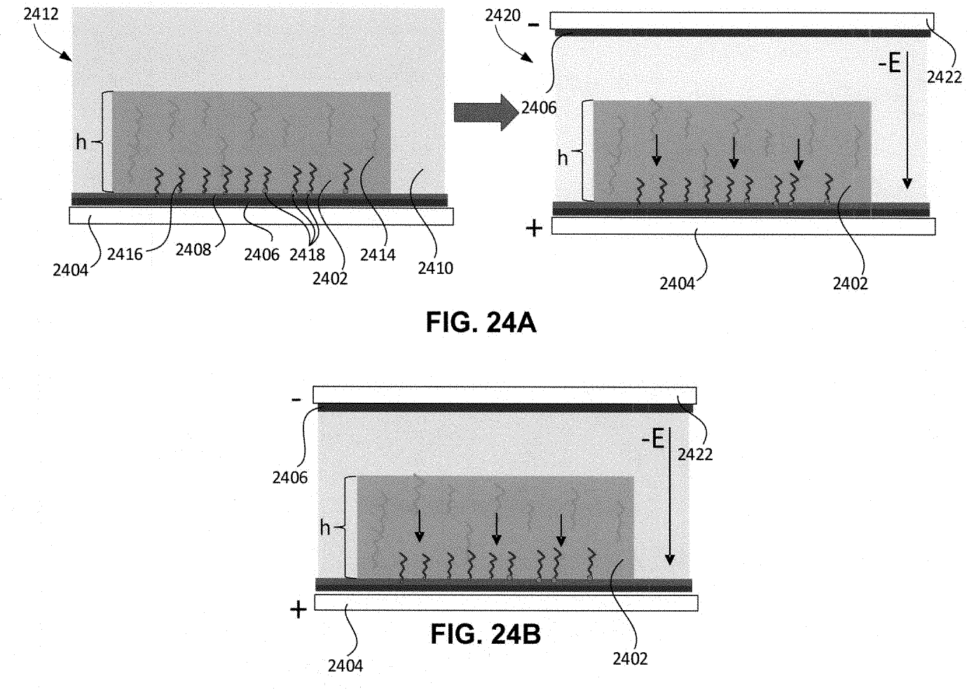

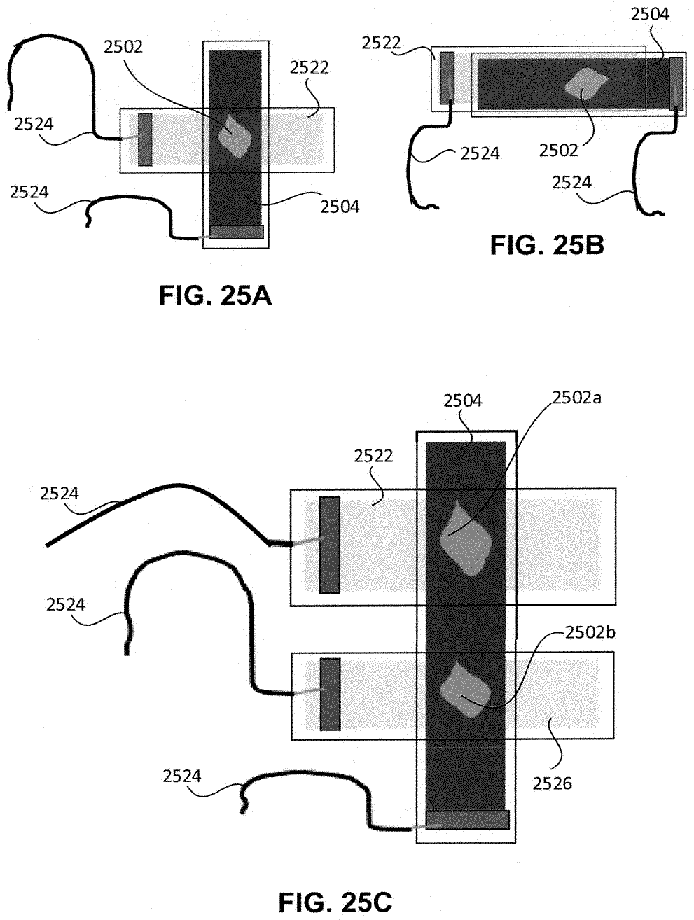

Spatial detection compositions and methods are disclosed in FIGS. 1-22C and 24A-25C, which are described in further detail in priority documents U.S. Ser. Nos. 62/979,681; 62/980,116; and 63/035,324, each of which is incorporated by reference in its entirety.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). In some embodiments biological sample can be a cell culture sample. In some embodiments, a biological sample can be nervous tissue, blood, serum, plasma, cerebrospinal fluid, or bone marrow aspirate. Biological samples are also described in Section (I)(d) of WO 2020/176788 and/or U.S. Patent Application Publication No. 2020/0277663, each of which is incorporated by reference in its entirety.

In addition, spatial analysis methods can be performed on various types of samples, including tissues (e.g., tissue slices) or single cells (e.g., cultured cells). Exemplary methods and compositions relating to tissue or single-cell spatial analysis is found at least in WO 2020/176788 and/or U.S. Patent Application Publication No. 2020/0277663, each of which is incorporated by reference in its entirety. In some instances, one biological sample can be used for tissue and single cell analysis. For example, multiple serial slices (e.g., 10 μm in thickness) of a tissue can be cut. A first slice can be placed on an array and analyte capture as described herein can be performed. In some instances, a second slice of tissue can further undergo cellular dissociation, creating a sample with isolated cells that can be analyzed using spatial analysis methods. Briefly, in some instances, a tissue is minced into small pieces and treated with lysis buffer to homogenize the sample. The homogenous resultant can be filtered and centrifuged to collect a pellet of nuclei. The nuclei can be resuspended and used for single cell analysis methods described herein. Data captured from the second slice (i.e., the single nuclei data) could then be combined with the data from the first slice (i.e., the whole tissue data) to gain a higher cell type understanding and potentially deconvolve the cell type identity within each spot on the array. Additional methods of single cell isolation is found in Hu et al., Mol Cell. 2017 Dec. 7; 68(5):1006-1015.e7; Habib et al., Science, 2016 Aug. 26; 353(6302):925-8; Habib et al., Nat Methods, 2017 October; 14(10):955-958; Lake et al., Science, 2016 Jun. 24; 352(6293):1586-90; and Lacar et al., Nat Commun, 2016 Apr. 19; 7:11022; each of which is incorporated by reference in its entirety.

In another embodiment, two different samples are collected, whereby one sample is analyzed with intact tissue and a second tissue undergoes cell dissociation. Results from each biological sample can be compared to gain a higher cell type understanding and potentially deconvolve the cell type identity within each spot on the array. Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of each analyte within the biological sample. The spatial location of each analyte within the biological sample is determined based on the feature to which each analyte is bound on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain. In some instances, the capture probe can include functional sequences that are useful for subsequent processing, such as functional sequence 604, which can include a sequencer specific flow cell attachment sequence, e.g., a P5 or P7 sequence, as well as functional sequence 606, which can include sequencing primer sequences, e.g., a R1 primer binding site, a R2 primer binding site. In some embodiments, sequence 604 is a P7 sequence and sequence 606 is a R2 primer binding site. Additional features of capture probes are described in Section (II)(b) of WO 2020/176788 and/or U.S. Patent Application Publication No. 2020/0277663, each of which is incorporated by reference in its entirety. Generation of capture probes can be achieved by any appropriate method, including those described in Section (II)(d)(ii) of WO 2020/176788 and/or U.S. Patent Application Publication No. 2020/0277663, each of which is incorporated by reference in its entirety.

In some embodiments, more than one analyte type from a biological sample can be detected (e.g., simultaneously or sequentially) using any appropriate multiplexing technique, such as those described in Section (IV) of WO 2020/176788 and/or U.S. Patent Application Publication No. 2020/0277663, each of which is incorporated by reference in its entirety.

In some embodiments, detection of one or more analytes (e.g., protein analytes) can be performed using one or more analyte capture agents. As used herein, an “analyte capture agent” refers to an agent that interacts with an analyte (e.g., an analyte in a sample) and with a capture probe (e.g., a capture probe attached to a substrate) to identify the analyte. In some embodiments, the analyte capture agent includes an analyte binding moiety and a capture agent barcode domain. Additional description of analyte capture agents can be found in Section (II)(b)(viii) of WO 2020/176788 and/or U.S. Patent Application Publication No. 2020/0277663, each of which is incorporated by reference in its entirety.

There are at least two general methods to associate a spatial barcode with one or more neighboring cells, such that the spatial barcode identifies the one or more cells, and/or contents of the one or more cells, as associated with a particular spatial location. One general method is to promote analytes out of a cell and towards a spatially-barcoded array (e.g., including spatially-barcoded capture probes). Another general method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template), or derivatives thereof (see, e.g., Section (II)(b)(ii) of WO 2020/176788 and/or U.S. Patent Application Publication No. 2020/0277663 regarding extended capture probes, the references of which are incorporated by reference in their entireties.). For example, in some cases, the capture probes may include mRNA specific priming sequences, e.g., poly-T primer segments that allow priming and replication of mRNA in a reverse transcription reaction or other targeted priming sequences. Alternatively or additionally, random RNA priming may be carried out using random N-mer primer segments of the barcoded oligonucleotides. Reverse transcriptases (RTs) can use an RNA template and a primer complementary to the 3′ end of the RNA template to direct the synthesis of the first strand complementary DNA (cDNA). Additional variants of spatial analysis methods, including in some embodiment's, an imaging step, are described in Section (II)(a) of WO 2020/176788 and/or U.S. Patent Application Publication No. 2020/0277663, each of which is incorporated by reference in its entirety. Analysis of captured analytes, for example, including sample removal, extension of capture probes, sequencing (e.g., of a cleaved extended capture probe and/or a cDNA molecule complementary to an extended capture probe), sequencing on the array (e.g., using in situ hybridization approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663, each of which is incorporated by reference in its entirety.

Typically, for spatial array-based analytical methods, a substrate functions as a support for direct or indirect attachment of capture probes to features of the array. A “feature” is an entity that acts as a support or repository for various molecular entities used in sample analysis. In some embodiments, some or all of the features in an array are functionalized for analyte capture. Exemplary substrates are described in Section (II)(c) of WO 2020/176788 and/or U.S. Patent Application Publication No. 2020/0277663, each of which is incorporated by reference in its entirety. Exemplary features and geometric attributes of an array can be found in Sections (II)(d)(i), (II)(d)(iii), and (II)(d)(iv) of WO 2020/176788 and/or U.S. Patent Application Publication No. 2020/0277663, each of which is incorporated by reference in its entirety.

Generally, analytes can be captured when contacting a biological sample with a substrate including capture probes (e.g., substrate with capture probes embedded, spotted, printed on the substrate or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663, each of which is incorporated by reference in its entirety.

In some cases, a spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be separated into single cells or cell groups for analysis. Some such methods of spatial analysis are described in Section (III) of WO 2020/176788 and/or U.S. Patent Application Publication No. 2020/0277663, each of which is incorporated by reference in its entirety.

Some exemplary particular spatial analysis workflows are described in the Exemplary Embodiments section of WO 2020/176788 and/or U.S. Patent Application Publication No. 2020/0277663, each of which is incorporated by reference in its entirety.

In some embodiments, a spatial analysis can be performed using dedicated hardware and/or software, such as any of the systems described in Sections (II)(e)(ii) and/or (V) of WO 2020/176788 and/or U.S. Patent Application Publication No. 2020/0277663, each of which is incorporated by reference in its entirety.

### II. Targeted Spatial Gene Expression Profiling by Hybridization and Capture of Spatial cDNA

Disclosed herein are methods to enhance detection of one or more target analytes. The methods disclosed herein may combine methods of spatial detection while enhancing detection of particular analytes of interest. In some embodiments, the methods of spatial detection include contacting a capture probe comprising a spatial barcode and a capture domain (e.g., an analyte-binding sequence) to an analyte. In some embodiments, the capture probe hybridized to the analyte can be extended using a polymerase (e.g., a reverse transcriptase) using the hybridized analyte as a template, to generate an extended capture probe. In some embodiments, a 3′ end of the capture probe hybridized to the analyte can be extended using a polymerase (e.g., a reverse transcriptase) using the hybridized analyte as a template, to generate an extended capture probe. In some embodiments, a 5′ end of the capture probe hybridized to the analyte can be extended using a polymerase (e.g., a reverse transcriptase) using the hybridized analyte as a template, to generate an extended capture probe. The extended capture probe can be amplified (e.g., via second strand synthesis) to generate a single-stranded nucleic acid comprising a sequence that is complementary to the extended capture probe. The single-stranded nucleic acid comprising a sequence that is complementary to the extended capture probe can be used to generate or can be a part of a nucleic acid library. A subset of nucleic acids in the nucleic acid library can be enriched using any of the exemplary enrichment methods described herein (e.g., hybridization using any of the oligonucleotides including a bait domain described herein). Compared to methods involving whole genome analysis (e.g., whole transcriptome sequencing), targeted spatial gene expression profiling as described herein can, in some cases, reduce sequencing costs by avoiding the sequencing and/or detection of analytes that are not of interest (e.g., ribosomal, mitochondrial, or house-keeping transcripts).

The methods disclosed herein include identifying a location of an analyte in a biological sample. In some embodiments, the methods include (a) contacting a plurality of nucleic acids with a plurality of bait oligonucleotides, where a nucleic acid of the plurality of nucleic acids comprises (i) a spatial barcode or a complement thereof, and (ii) a portion of a sequence of an analyte from a biological sample, or a complement thereof; and a bait oligonucleotide of the plurality of bait oligonucleotides comprises a domain that binds specifically to (i) all or a portion of the spatial barcode or a complement thereof, and/or (ii) all or a portion of the sequence of the analyte from the biological sample, or a complement thereof, and a molecular tag; (b) enriching a complex of the bait oligonucleotide specifically bound to the nucleic acid using a substrate comprising an agent that binds specifically to the molecular tag; and (c) determining (i) all or a portion of the sequence of the spatial barcode or the complement thereof, and (ii) all or a portion of the sequence of the analyte from the biological sample, and using the determined sequences of (i) and (ii) to identify the location of the analyte in the biological sample. In some embodiments, the methods further include generating the plurality of nucleic acids prior to the steps of (a)-(c) above. In some embodiments, for example, the methods further include generating the plurality of nucleic acids by contacting the biological sample with a substrate comprising a plurality of attached capture probes, wherein a capture probe of the plurality comprises (i) the spatial barcode and (ii) a capture domain that binds specifically to a sequence present in the analyte. Then, in some embodiments, the capture probe is extended using the analyte that is specifically bound to the capture domain as a template to generate an extended capture probe. In some embodiments, the 3′ end of the capture probe is extended using the analyte that is specifically bound to the capture domain as a template to generate an extended capture probe. In some embodiments, the 5′ end of the capture probe is extended using the analyte that is specifically bound to the capture domain as a template to generate an extended capture probe. In some embodiments, the extended capture probe is amplified to produce the nucleic acid.

(a) Nucleic Acid Detection and Library Preparation

Also disclosed herein are methods of preparing a library of nucleic acids (e.g., a plurality of nucleic acids). In some embodiments, the library of nucleic acids (e.g., a plurality of nucleic acids) include one or more nucleic acids of interest, whose detection can be enhanced using hybridization methods. Some embodiments of any of the methods described herein can include contacting a biological sample with a substrate comprising a plurality of attached capture probes, wherein a capture probe of the plurality comprises (i) a spatial barcode and (ii) a capture domain that binds specifically to a sequence present in a analyte; extending a 3′ end of the capture probe using the analyte that is specifically bound to the capture domain as a template to generate an extended capture probe; and amplifying the extended capture probe to produce a nucleic acid.

In some embodiments, the biological sample is a tissue sample. In some embodiments, the biological sample is a section of a tissue sample. In some embodiments, the biological sample is a fresh tissue sample. In some embodiments, the biological sample is a fresh-frozen tissue sample. In some embodiments, the biological sample is a tissue sample that has been formalin-fixed and paraffin-embedded (FFPE) (i.e., an FFPE sample). In some embodiments, the biological sample is a tissue sample embedded in optimal cutting temperature (OCT) compound. In some embodiments, the biological sample has been previously stained (e.g., immunohistochemistry (IHC) or histological staining) and imaged, and optionally, destained. In some embodiments, the analyte is a nucleic acid. In some embodiments, the nucleic acid is DNA (e.g., genomic DNA, mitochondrial DNA, or exosomal DNA). In some embodiments, the nucleic acid is RNA. In some embodiments, the RNA is mRNA. In some embodiments, the nucleic acid comprises DNA.

In some embodiments, the biological sample is permeabilized. In some examples, a biological sample is permeabilized to allow analytes to be released from one or more cells within the biological sample. In some embodiments, analytes released from the one or more cells can be amplified before they are contacted with a capture probe.

In some embodiments, the amplification of the extended capture probe results in the production of a single-stranded nucleic acid including a sequence that is complementary to all or a portion of the sequence of the extended capture probe. In some examples, the single-stranded nucleic acid can be denatured or dissociated from the extended capture probe.

In some embodiments, the biological sample is affixed to a slide. In some embodiments, the sample is stained prior to creation of the library of nucleic acids (e.g., plurality of nucleic acids). In some embodiments, the biological sample is stained while the biological sample is on the slide. In some embodiments, the stained biological sample is imaged prior to creation of the library of nucleic acids (e.g., plurality of nucleic acids).

In some embodiments, staining includes biological staining techniques such as H&E staining. In some embodiments, staining includes identifying analytes using fluorescently conjugated antibodies (e.g., immunofluorescence). In some embodiments, a biological sample is stained using two or more different types of stains, or two or more different staining techniques (e.g., IF, IHC, and/or H&E staining). For example, a biological sample can be prepared by staining and imaging using one technique (e.g., H&E staining and bright field imaging), destained (e.g., quenching or photobleaching), followed by staining and imaging using another technique (e.g., IHC/IF staining and fluorescence microscopy) for the same biological sample.

In some embodiments, biological samples can be destained prior to creation of the spatial library of nucleic acids. Methods of destaining or discoloring a biological sample are known in the art, and generally depend on the nature of the stain(s) applied to the biological sample.

In some embodiments, after creation of the library of nucleic acids (e.g., plurality of nucleic acids), one or more bait oligonucleotides (e.g., from one or more panels as described herein) are hybridized to a plurality of nucleic acids. In some embodiments, the nucleic acids include all or a portion of a sequence of an analyte of interest or a complement thereof and/or include all or a portion of a spatial barcode of interest or a complement thereof. In some embodiments, one or more bait oligonucleotides are hybridized to the nucleic acid including all or a portion of a sequence of analyte of interest or a complement thereof, and/or all or a portion of a spatial barcode of interest, or a complement thereof, before additional sequences, such as an adaptor (or a complement thereof), a primer binding site (or a complement thereof), and/or a poly(G/I) sequence are ligated or added to the nucleic acid (e.g., using a splint oligonucleotide).

In other embodiments, the library, the nucleic acid(s), or the enriched nucleic acid(s) can be quantified using quantitative PCT (qPCR). In some embodiments, the library, the nucleic acid(s), or the enriched nucleic acid(s) can be fragmented. In some embodiments, the library, the nucleic acid(s), or the enriched nucleic acid(s) can be fragmented by enzyme-based methods (e.g., by restriction enzymes, nicking enzymes and/or transposases). In some embodiments, the library, the nucleic acid(s), or the enriched nucleic acid(s) can be fragmented by endonucleases. In some embodiments, the library, the nucleic acid(s), or the enriched nucleic acid(s) can be fragmented by mechanical shearing (e.g., acoustic shearing, hydrodynamic shearing, and/or nebulization). In some embodiments, the library, the nucleic acid(s), or the enriched nucleic acid(s) can be fragmented by combined enzyme-based methods and mechanical shearing. In some embodiments, the library, the nucleic acid(s), or the enriched nucleic acid(s) can be further processed via end-repair, poly-A tailing, or a combination thereof. In some embodiments, adaptors are ligated to each nucleic acid or enriched nucleic acid sequence. In some embodiments, the adaptors can be ligated to the 3′ end of the nucleic acid or enriched nucleic acid sequence. In some embodiments, the adaptors can be ligated to the 5′ end of the nucleic acid sequence or enriched nucleic acid sequence. In some embodiments, the adaptors can be nucleic acid sequences that add a function, e.g., spacer sequences, primer sequences/sites, barcode sequences, unique molecular identifier (UMI) sequences, linkers, and/or sequencing adaptors.

In some embodiments, the methods disclosed herein include sample index (SI) PCR, which adds nucleic acid sequences (e.g., barcodes) to the 5′ and/or 3′ ends of a nucleic acid sequence or an enriched nucleic acid sequence. In some embodiments, SI-PCR is a PCR reaction that introduces sample index sequences (e.g., i5 and i7) to the 5′ and/or 3′ ends of a nucleic acid sequence or an enriched nucleic acid sequence. In some embodiments, methods for SI-PCR add the i5 sample index sequence. In some embodiments, methods for SI-PCR add the i7 sample index sequence. In some embodiments, a P5 adapter is added to a nucleic acid sequence or an enriched nucleic acid sequence. In some embodiments, a P7 adapter is added to a nucleic acid sequence or an enriched nucleic acid sequence. In some embodiments, SI-PCR is performed before bait oligonucleotide enrichment of a nucleic acid of interest. In some embodiments, SI-PCR is performed after bait oligonucleotide enrichment of a nucleic acid of interest.

In some embodiments, the nucleic acid of interest (before or after enrichment using a bait oligonucleotide) or a library generated from the same can be dried. In some embodiments, drying includes a dehydrating process such as heat, a vacuum, lyophilization, desiccation, filtration, and air-drying. In some embodiments, drying can be performed for at least 1 hour, at least 2 hours, at least 3 hours or at least 4 hours. In some embodiments, the nucleic acid of interest (before or after enrichment using a bait oligonucleotide) or a library generated from the same is not dried.

In alternative embodiments, combined with (1) capture of an analyte on an array and/or (2) target enrichment, or independent of (i.e., methods do not include) capture and target enrichment methods, additional methods are provided herein for analyzing an analyte using in situ methods. In some instances, the methods include amplifying an analyte using in situ methods. For instance, in some embodiments where an analyte is amplified, the amplification is performed by rolling circle amplification. In some embodiments, the capture probe to be amplified includes sequences (e.g., docking sequences, functional sequences, and/or primer sequences) that enable rolling circle amplification. In one example, the capture probe can include a functional sequence that is capable of binding to a primer used for amplification. In another example, the capture probe can include one or more docking sequences (e.g., a first docking sequence and a second docking sequence) that can hybridize to one or more oligonucleotides (e.g., a padlock probe(s)) used for rolling circle amplification. In some embodiments, additional probes are affixed to the substrate, where the additional probes include sequences (e.g., a docking sequence(s), a functional sequence(s), and/or a primer sequence(s)) that enable rolling circle amplification. In some embodiments, the spatial array is contacted with an oligonucleotide (e.g., a padlock probe). As used herein, a “padlock probe” refers to an oligonucleotide that has, at its 5′ and 3′ ends, sequences that are complementary to adjacent or nearby target sequences (e.g., docking sequences) on a capture probe. Upon hybridization to the target sequences (e.g., docking sequences), the two ends of the padlock probe are either brought into contact or an end is extended until the two ends are brought into contact, allowing circularization of the padlock probe by ligation (e.g., ligation using any of the methods described herein). In some embodiments, after circularization of the oligonucleotide, rolling circle amplification can be used to amplify the ligation product, which includes at least a capture domain and a spatial barcode from the capture probe. In some embodiments, amplification of the capture probe using a padlock oligonucleotide and rolling circle amplification increases the number of capture domains and the number of spatial barcodes on the spatial array. Padlock probes are disclosed in U.S. Pat. No. 8,551,710 and US-2020-0224244-A1, each of which is incorporated by reference in its entirety.

In some embodiments, the one or more additional biomarkers are detected using in situ sequencing. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (i.e., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363, the entire contents of each of which are incorporated herein by reference.

In addition, examples of methods and systems for performing in situ sequencing are described in PCT Patent Application Publication Nos. WO2014/163886, WO2018/045181, WO2018/045186, and in U.S. Pat. Nos. 10,138,509 and 10,179,932, the entire contents of each of which are incorporated herein by reference. Exemplary techniques for in situ sequencing include, but are not limited to, STARmap (described for example in Wang et al., (2018) Science, 361(6499) 5691), MERFISH (described for example in 2017/0220733 and in Moffitt, (2016) Methods in Enzymology, 572, 1-49), SeqFISH (described for example in U.S. Pat. No. 10,457,980), hybridization chain reaction amplification (described in U.S. Pat. No. 8,507,204) and FISSEQ (described for example in U.S. Patent Application Publication No. 2019/0032121). The entire contents of each of the foregoing references are incorporated herein by reference.

(b) Targeted Capture of Analytes Using Hybridization of Bait Oligonucleotides

(i) Design of Bait Oligonucleotides

In some embodiments, bait oligonucleotide sets are designed to target and hybridize to a plurality of nucleic acids (e.g., to prepared spatial libraries; e.g., to prepared cDNA libraries). In some embodiments, bait oligonucleotide sets hybridize to targeted nucleic acids (e.g., cDNA) in a library. In some embodiments, the hybridized product (e.g., the bait oligonucleotide and hybridized nucleic acid) are then captured by avidin beads. In some embodiments, the hybridized product (e.g., the bait oligonucleotide and hybridized nucleic acid) are then captured by streptavidin beads. Unhybridized nucleic acids are washed away. Then, the targeted product is reamplified and sequenced. In some embodiments, the reamplifed targeted product can be fragmented, ligated with adaptor sequences, and amplified by SI-PCR.

In some embodiments, disclosed herein are methods to design and test candidate bait oligonucleotide sequences. Candidate bait oligonucleotide sequences are designed so that each bait oligonucleotide sequence theoretically hybridizes to a unique target of interest. Accordingly, in some embodiment, the designed bait oligonucleotides are at least 40 nucleotides in length. To identify a bait oligonucleotide of interest, at unique 40 nucleotide sections of the human transcriptome are identified and aligned to the genome using an aligner designed for aligning RNA-seq data. In some embodiments, bait oligonucleotides are designed to hybridize to a particular exon. In some embodiments, bait oligonucleotides are designed to span an exon-exon junction. In some embodiments, bait oligonucleotides can hybridize to a target, allowing for identification of splicing and alternative splicing transcripts in the transcriptome. Using the alignments, sequences that align to the genome one or more times are identified and cataloged. Each bait designed can be tested against (i.e., compared to) a sequence identified in the genome. If the sequences in the bait oligonucleotide and in the genome do not match, then the bait can be tested and/or in one or more panels as disclosed herein.

However, if the sequences in the bait oligonucleotide and in the genome match, then in some embodiments, a modified bait is designed. To prepare a modified bait, one can slide the initial sequence+/−40 bp from the original position to identify a potentially new bait oligonucleotide. With each design, the new bait oligonucleotide is tested against the genome. After all such candidates are cataloged, the bait oligonucleotides that are ultimately included in one or more panels described herein are ordered (i.e., ranked) based on the bait oligonucleotide length (i.e. a longer bait is prioritized) and then by distance to the original intended position (with priority to sequences closer to the original intended position). However, if no bait meets the required criteria, that bait is dropped and no bait is designed at that position.

In some embodiments, the bait oligonucleotide sequence is 40 nucleotides long. In some embodiments, the bait oligonucleotide sequence is between 40 and 160 nucleotides long. In some embodiments, the bait oligonucleotide sequence is between 40 and 120 nucleotides long. In some embodiments, the bait oligonucleotide sequence is about 40, about 41, about 42, about 43, about 44, about 45, about 46, about 47, about 48, about 49, about 50, about 51, about 52 about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, or about 160 nucleotides long.

In some embodiments, a bait oligonucleotide of the plurality of bait oligonucleotides includes a domain that binds specifically to all or a portion of the spatial barcode or a complement thereof. In some embodiments, a bait oligonucleotide of the plurality of bait oligonucleotides includes a domain that binds specifically to all or a portion of the sequence of the analyte from the biological sample, or a complement thereof. In some embodiments, a bait oligonucleotide of the plurality of bait oligonucleotides includes a domain that binds specifically to all or a portion of the spatial barcode or a complement thereof and all or a portion of the sequence of the analyte from the biological sample, or a complement thereof. In some embodiments, the domain of the bait oligonucleotide hybridizes to an analyte of interest. In some embodiments, the domain of the bait oligonucleotide binds specifically to an analyte of interest. In some embodiments, the domain of the bait oligonucleotide binds specifically to all or a portion of the spatial barcode or a complement thereof. In some embodiments, the domain of the bait oligonucleotide binds specifically to all or a portion of the sequence of the analyte from the biological sample. In some embodiments, the domain of the bait oligonucleotide binds specifically to a 3′ portion of the sequence of the analyte from the biological sample or a complement thereof. In some embodiments, the domain of the bait oligonucleotide binds specifically to a 5′ portion of the sequence of the analyte from the biological sample or a complement thereof. In some embodiments, the domain of the bait oligonucleotide binds specifically to an intron in the sequence of the analyte from the biological sample or a complement thereof. In some embodiments, the domain of the bait oligonucleotide binds specifically to an exon in the sequence of the analyte from the biological sample or a complement thereof. In some embodiments, the domain of the bait oligonucleotide binds specifically to an untranslated 3′ region of the analyte from the biological sample or a complement thereof. In some embodiments, the domain of the bait oligonucleotide binds specifically to an untranslated 5′ region of the analyte from the biological sample or a complement thereof.

In some embodiments, the domain of the bait oligonucleotide sequence is 40 nucleotides long. In some embodiments, the domain of the bait oligonucleotide sequence is between 40 and 160 nucleotides long. In some embodiments, the domain of the bait oligonucleotide sequence is between 40 and 120 nucleotides long. In some embodiments, the domain of the bait oligonucleotide sequence is about 40, about 41, about 42, about 43, about 44, about 45, about 46, about 47, about 48, about 49, about 50, about 51, about 52 about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, or about 160 nucleotides long.

In some embodiments, the analyte from the biological sample is associated with a disease or condition. In some embodiments, the analyte from the biological sample comprises a mutation. In some embodiments, the analyte from the biological sample comprises a single nucleotide polymorphism (SNP). In some embodiments, the analyte from the biological sample comprises a trinucleotide repeat.

In some embodiments, the domain of the bait oligonucleotide hybridizes to a particular exon of a transcript (i.e., an mRNA molecule). For example, a transcript can be processed such that an exon that would otherwise be excised during a normal setting is included in the mature mRNA product in a different setting (e.g., a pathological setting such as cancer). In some embodiments, the domain of the bait oligonucleotide identifies (e.g., hybridizes to) one or more isoforms or an analyte, but not others. In some embodiments, for example, a bait oligonucleotide hybridizes to a particular exon that is detected in a pathological setting (e.g., cancer).

In some embodiments, there is more than one bait oligonucleotide for a particular analyte of interest in a panel. For example, an analyte can undergo alternate splicing (e.g., as in an mRNA molecule). In some embodiments, different baits could detect and enhance particular transcripts, thereby detecting whether a particular exon is included in an analyte. Thus, in some embodiments, a panel will include at least 2, at least 3, at least 4, at least 5, at least 6, at least 7, at least 8, at least 9, at least 10, or more bait oligonucleotides for one analyte.

In some embodiments, a bait oligonucleotide is fully complementary (i.e., 100% complementary) to a portion of a target analyte. In some embodiments, a bait oligonucleotide is partially complementary (i.e., less than 100% complementary) to a portion of a target analyte. In some embodiments, a bait oligonucleotide has at least 75%, at least 80%, at least 85%, at least 90%, at least 91%, at least 92%, at least 93%, at least 94%, at least 95%, at least 96°/o, at least 97%, at least 98%, or at least 99% sequence identity to a portion of a target analyte.

In some embodiments, a bait oligonucleotide is partially complementary (i.e., less than 100% complementary) to a portion of a target analyte. In some embodiments, a bait oligonucleotide is partially complementary (i.e., less than 100% complementary) to a portion of a target analyte. In some embodiments, part of the bait oligonucleotide hybridizes to a portion of a target analyte. In some embodiments, part of the bait oligonucleotide has at least 75%, at least 80%, at least 85%, at least 90%, at least 91%, at least 92%, at least 93%, at least 94%, at least 95%, at least 96%, at least 97%, at least 98%, or at least 99% sequence identity to a portion of a target analyte.

Bait oligonucleotides are included in one or more panels (e.g., cohorts). Panels include groups of bait oligonucleotides that target a particular cohort of analytes. For example, a panel can include a set of bait oligonucleotides that is particular to a pathological setting. In some embodiments, more than one panel (e.g., set of bait oligonucleotides) can be used to enhance detection of analytes. For example, in some embodiments, at least 1, at least 2, at least 3, at least 4, at least 5, or more panels (e.g., set of bait oligonucleotides) can be used to enhance detection of analytes. The bait oligonucleotides and target analytes for all panels disclosed are provided in the accompanying sequencing listing disclosed herein.

In some embodiments, a panel includes bait oligonucleotides that hybridize and enhance detection of analytes dysregulated in cancer (e.g., a cancer panel). In some embodiments, the cancer panel allows for quantitative analysis of analytes aberrantly expressed in the cancer transcriptome while avoiding the excess costs and time associated with sequencing an entire transcriptome. The bait oligonucleotides and target analytes for an exemplary cancer panel are provided in Table 1a and in the accompanying sequencing listing disclosed herein. In some embodiments, the bait oligonucleotides in the cancer panel can include detection of dysregulated analytes associated with biological processes such as apoptosis, metabolism, cell cycle (e.g., checkpoint analytes), DNA damage and repair, hypoxia, and stress toxicity. More specifically, the bait oligonucleotides in the cancer panel target cancer-specific analytes such as analytes that function in pathways that include but are not limited to the Myc pathway, Hippo pathway, RTK/RAS pathway, TP53 and TP53-associate pathway, TFGβ pathway, and Wnt pathway. The bait oligonucleotides in the cancer panel also can detect cancer hot spots, tumor suppressor analytes, and immune-dysregulated analytes. The bait oligonucleotides and target analytes for an exemplary cancer panel were disclosed in U.S. Appl. No. 62/970,066 (Titled “Capturing Targeted Genetic Targets Using A Hybridization/Capture Approach”) and U.S. Appl. No. 62/929,686, (Titled “Capturing Targeted Genetic Targets Using A Hybridization/Capture Approach”), each of which is incorporated by reference in its entirety.

In some embodiments, a panel includes bait oligonucleotides that hybridize and enhance detection of analytes associated with immunological dysregulation (e.g., an immunology panel). In some embodiments, the immunology panel allows for quantitative analysis of analytes associated with immune-dysregulation while avoiding the excess costs and time associated with sequencing an entire transcriptome. The bait oligonucleotides and target analytes for an exemplary immunology panel are provided in Table 2a and in the accompanying sequencing listing disclosed herein. In some embodiments, the bait oligonucleotides in the immunology panel can include detection of dysregulated analytes associated with biological processes such as B cell function, T cell function, cell cycle, cell signaling, interleukin signaling, and metabolism. More specifically, bait oligonucleotides target analytes that include but are not limited to transcription factors, T cell activation markers, antigen presentation genes, metabolic genes, and SIRP family members. In some embodiments, the immunology panel has applications to detect associated with immunological dysregulation to examine innate immunity, adaptive immunity, one or more inflammatory responses, detection of one or more infectious diseases (e.g., a bacterial infection; a viral infection), and immune response in a transplant recipient. More specifically, the bait oligonucleotides in the immunology panel target immuno-specific biomarkers that include but are not limited to lineage markers, tissue markers, and cancer markers. In some embodiments, the bait oligonucleotides enhance detection of analytes expressed in bone marrow, gut, lung, salivary gland, intestine, lymph node, stem cells, or a combination thereof. The bait oligonucleotides and target analytes for an exemplary immunology panel were disclosed in U.S. Appl. No. 62/970,066 (Titled “Capturing Targeted Genetic Targets Using A Hybridization/Capture Approach”) and U.S. Appl. No. 62/929,686, (Titled “Capturing Targeted Genetic Targets Using A Hybridization/Capture Approach”), each of which is incorporated by reference in its entirety.

In some embodiments, a panel includes bait oligonucleotides that hybridize and enhance detection of analytes that detect pathway dysregulation (e.g., a pathway panel). In some embodiments, the pathway panel allows for quantitative analysis of analytes associated with pathway dysregulation while avoiding the excess costs and time associated with sequencing an entire transcriptome. The bait oligonucleotides and target analytes for an exemplary pathway panel are provided in Table 3a and in the accompanying sequencing listing disclosed herein. In some embodiments, the bait oligonucleotides in the pathway panel can include detection of dysregulated analytes associated with complex signal transduction pathways. The target analytes include analytes specific to disease or drug targets; G-protein coupled receptors (GPCRs), one or more kinases; one or more epigenetic markers; or one or more checkpoint analytes. Analytes detected by bait oligonucleotides in the pathway panel include but are not limited to tissue markers of cancer dysregulation, central nervous system dysregulation, inflammation dysregulation, metabolic dysregulation, cardiovascular dysregulation, respiratory dysregulation, and reproductive dysregulation. The bait oligonucleotides and target analytes for an exemplary pathway panel were disclosed in U.S. Appl. No. 62/970,066 (Titled “Capturing Targeted Genetic Targets Using A Hybridization/Capture Approach”) and U.S. Appl. No. 62/929,686, (Titled “Capturing Targeted Genetic Targets Using A Hybridization/Capture Approach”), each of which is incorporated by reference in its entirety.

In some embodiments, a panel includes bait oligonucleotides that hybridize and enhance detection of analytes that detect neurological development and/or dysregulation. In some embodiments, a panel includes bait oligonucleotides that hybridize and enhance detection of analytes that detect neurological development. In some embodiments, a panel includes bait oligonucleotides that hybridize and enhance detection of analytes that detect neurological dysregulation. In some embodiments, a panel includes bait oligonucleotides that hybridize and enhance detection of analytes that detect neurological development and dysregulation. In some embodiments, the neurological panel allows for quantitative analysis of analytes associated with pathway dysregulation while avoiding the excess costs and time associated with sequencing an entire transcriptome. The bait oligonucleotides and target analytes for an exemplary neurological panel are provided in Table 4a and in the accompanying sequencing listing disclosed herein. In some embodiments, the bait oligonucleotides in the neurological panel can include detection of dysregulated analytes associated with axonal targeting, hypoxia, and glioblastoma. In some embodiments, the bait oligonucleotides in the neurological panel can include detection of dysregulated analytes associated with axonal targeting. In some embodiments, the bait oligonucleotides in the neurological panel can include detection of dysregulated analytes associated with hypoxia. In some embodiments, the bait oligonucleotides in the neurological panel can include detection of dysregulated analytes associated with glioblastoma. In some embodiments, the bait oligonucleotides in the neurological panel can include detection of mitochondrial protein-coding genes. In some embodiments, the bait oligonucleotides in the neurological panel can include detection of mitochondrial protein-coding genes in order to evaluate energy metabolism.

In some embodiments, any one of the panels disclosed herein include bait oligonucleotides that can enhance detection of at least about 100 analytes, about 200 analytes, about 300 analytes, about 400 analytes, about 500 analytes, about 600 analytes, about 700 analytes, about 800 analytes, about 900 analytes, about 1000 analytes, about 1100 analytes, about 1200 analytes, about 1300 analytes, about 1400 analytes, about 1500 analytes, about 1600 analytes, about 1700 analytes, about 1800 analytes, about 1900 analytes, about 2000 analytes or more.

In some embodiments, a bait oligonucleotide enhances detection of an analyte of interest by about 1.5 fold, about 2 fold, about 3 fold, about 4 fold, about 5 fold, about 6 fold, about 7 fold, about 8 fold, about 9 fold, about 10 fold, about 20 fold, about 50 fold, about 100 fold, 500 fold, 1000 fold, or greater in comparison to analytes that are not detected using a bait oligonucleotide.

In some embodiments, a bait oligonucleotide includes a molecular tag. As disclosed herein, a molecular tag of a bait oligonucleotide is affixed to (e.g., conjugated to) the nucleic acid sequence of the bait oligonucleotide. In some embodiments, the molecular tag includes one or more moieties. In some embodiments, the moiety includes a label as described herein. The label can allow detection of the hybridization of a bait oligonucleotide to an analyte. In some embodiments, the label is directly associated with (i.e., conjugated to) the bait oligonucleotide. The detectable label can be directly detectable by itself (e.g., radioisotope labels or fluorescent labels) or, in the case of an enzymatic label, can be indirectly detectable, e.g., by catalyzing chemical alterations of a chemical substrate compound or composition, which chemical substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

In some embodiments, the molecular tag includes a small molecule. In some embodiments, the molecular tag includes a nucleic acid. In some embodiments, the nucleic acid is single-stranded. In some embodiments, the nucleic acid is double-stranded. In some embodiments, the nucleic acid is RNA. In some embodiments, the nucleic acid is DNA. In some embodiments, the molecular tag includes a carbohydrate. In some embodiments, the molecular tag is positioned 5′ to the domain in the bait oligonucleotide. In some embodiments, the molecular tag is position 3′ to the domain in the bait oligonucleotide.

In some embodiments, the agent that binds specifically to the molecular tag includes a protein. In some embodiments, the protein is an antibody. In some embodiments, the agent that binds specifically to the molecular tag comprises a nucleic acid. In some embodiments, the agent that binds specifically to the molecular tag comprises a small molecule. In some embodiments, the agent that binds specifically to the molecular tag is attached to a substrate. In some embodiments, the substrate is a bead. In some embodiments, the substrate is a well. In some embodiments, the substrate is a slide.

In some embodiments, the moiety is biotin. In some embodiments, a biotin molecule is directly associated with (i.e., conjugated to) the bait oligonucleotide at the 3′ end. In some embodiments, a biotin molecule is directly associated with (i.e., conjugated to) the bait oligonucleotide at the 5′ end. In some embodiments, and as disclosed below, the biotin molecule can be associated to (e.g. conjugated to) an avidin molecule, allowing pulldown of an analyte. In some embodiments, and as disclosed below, the biotin molecule can be associated to (e.g. conjugated to) a streptavidin molecule, allowing pulldown of an analyte.

In some embodiments, the bait oligonucleotide does not include a moiety affixed to the sequence (i.e., the bait oligonucleotide is a naked bait oligonucleotide).

(ii) Hybridization Methods

(1) Hybridization of Bait Oligonucleotides Directly

After creation of the nucleic acid (e.g., cDNA) library as disclosed above, the methods disclosed herein include denaturation of the nucleic acid (e.g., cDNA) strands, creating single-stranded nucleic acid (e.g., cDNA) molecules. In some embodiments, the nucleic acid (e.g., cDNA) library includes transcripts from the entire transcriptome.

In some embodiments, bait oligonucleotides are added to a plurality of nucleic acids (e.g., a library of nucleic acids). In some embodiments, the plurality of nucleic acids includes nucleic acids that have a spatial barcode or a complement thereof, and a portion of a sequence of an analyte from a biological sample, or a complement thereof. In some embodiments, the spatial barcode includes a sequence that corresponds to a region of interest in the biological sample. In some embodiments, the spatial barcode allows detection of and association with a particular region of interest in the biological sample. In some embodiments, the methods disclosed herein include identifying the region of interest. In some embodiments, the spatial barcode provides information about the location of an analyte in a biological sample. In some embodiments, the methods disclosed herein include identifying the location and/or abundance of an analyte in the biological sample.

In some embodiments, the bait oligonucleotides are added to a plurality of nucleic acids. In some embodiments, bait oligonucleotides include a domain that binds specifically to all or a portion of a spatial barcode or a complement thereof, and/or all or a portion of a sequence of an analyte or a complement thereof. In some embodiments, a complex of a bait oligonucleotide specifically bound to the nucleic acid can be enriched. For example, a bait oligonucleotide can include a molecular tag, and a complex of a bait oligonucleotide specifically bound to the nucleic acid can be enriched using an agent that binds specifically to the molecular tag. In some embodiments, the molecular tag can be attached (directly or indirectly) to a substrate (e.g., a slide, a well, or a bead). In some embodiments, a molecular tag can include a protein, a nucleic acid, a carbohydrate, a small molecule, or any combination thereof. In some embodiments, an agent that binds specifically to a molecular tag can be a protein, a nucleic acid, a carbohydrate, a small molecule, or any combination thereof. In some embodiments, a molecular tag can be avidin and an agent that binds specifically to the molecular tag can be biotin. In some embodiments, a molecular tag can be streptavidin and an agent that binds specifically to the molecular tag can be biotin.

In some embodiments, a molecular tag can be biotin and an agent that binds specifically to the molecular tag can be streptavidin (e.g., streptavidin attached to a bead). In some embodiments, where the molecular tag is biotin and the method includes the use of streptavidin beads to enrich a nucleic acid complexed with a bait oligonucleotide, the streptavidin beads can be washed using any method known in the art. In some embodiments, the streptavidin bead can be washed for 1 time, 2 times, 3 times, 4 times, 5 times, 6 times, 7 times or more. In some embodiments, the streptavidin beads can be stringently washed for one time, 2 times, 3 times, 4 times, 5 times, 6 times, 7 times, or more. In some embodiments, the streptavidin beads can be washed at about 15° C., about 20° C., about 25° C., about 30° C., about 35° C., about 40° C., about 45° C., about 48° C., about 50° C., or more. In some embodiments, after one or more wash steps, the nucleic acid(s) hybridized to the bait oligonucleotide(s) can be recovered and are enriched.

In some embodiments, a molecular tag can be biotin and an agent that binds specifically to the molecular tag can be avidin (e.g., avidin attached to a bead). In some embodiments, where the molecular tag is biotin and the method includes the use of avidin beads to enrich a nucleic acid complexed with a bait oligonucleotide, the avidin beads can be washed using any method known in the art. In some embodiments, the avidin bead can be washed for 1 time, 2 times, 3 times, 4 times, 5 times, 6 times, 7 times or more. In some embodiments, the avidin beads can be stringently washed for one time, 2 times, 3 times, 4 times, 5 times, 6 times, 7 times, or more. In some embodiments, the avidin beads can be washed at about 15° C., about 20° C., about 25° C., about 30° C., about 35° C., about 40° C., about 45° C., about 48° C., about 50° C., or more. In some embodiments, after one or more wash steps, the nucleic acid(s) hybridized to the bait oligonucleotide(s) can be recovered and are enriched.

In some embodiments, the recovered nucleic acids can be released from the bait oligonucleotide(s) and purified to remove avidin and biotin (or any other molecular tag and agent that binds specifically to the molecular tag). In some embodiments, the recovered nucleic acids can be released from the bait oligonucleotide(s) and purified to remove avidin and biotin (or any other molecular tag and agent that binds specifically to the molecular tag). In some embodiments, the recovered nucleic acids can be released from the bait oligonucleotide(s) and purified to remove streptavidin and biotin (or any other molecular tag and agent that binds specifically to the molecular tag).

In some embodiments, the methods disclosed herein include an enrichment method to selectively increase one or more particular targets. In some embodiments, the bait oligonucleotides added to the plurality of nucleic acids. In some embodiments, bait oligonucleotides hybridize to a particular nucleic acid. In some embodiments, the hybridized bait oligonucleotide/nucleic acid molecule can be enriched (i.e., selectively increased in expression).

In some embodiments, a plurality of bait oligonucleotides can be used in any of the methods described herein to enrich one or more nucleic acids of interest from a plurality of nucleic acids. In some embodiments, the plurality of bait oligonucleotides are designed to enrich one or more nucleic acids that include all or a part of a sequence of an analyte of interest (e.g., one or more genes that function or are aberrantly expressed in a particular cellular state or pathway), or a complement thereof. For example, in some embodiments, a plurality of bait oligonucleotides can be used to enrich nucleic acids that include all or a part of a sequence of cancer-related transcripts, or complements thereof (e.g., as disclosed in U.S. Appl. No. 62/970,066 (Titled “Capturing Targeted Genetic Targets Using A Hybridization/Capture Approach”) and U.S. Appl. No. 62/929,686, (Titled “Capturing Targeted Genetic Targets Using A Hybridization/Capture Approach”), each of which is incorporated by reference in its entirety). In some embodiments, a plurality of bait oligonucleotides can be used to enrich nucleic acids that include all or a part of a sequence of immune-related transcripts, or complements thereof (e.g., as disclosed in U.S. Appl. No. 62/970,066 (Titled “Capturing Targeted Genetic Targets Using A Hybridization/Capture Approach”) and U.S. Appl. No. 62/929,686, (Titled “Capturing Targeted Genetic Targets Using A Hybridization/Capture Approach”), each of which is incorporated by reference in its entirety). In some embodiments, a plurality of bait oligonucleotides can be used to enrich nucleic acids that include all or a part of a sequence of pathway-specific transcripts, or complements thereof (e.g., as disclosed in U.S. Appl. No. 62/970,066 (Titled “Capturing Targeted Genetic Targets Using A Hybridization/Capture Approach”) and U.S. Appl. No. 62/929,686, (Titled “Capturing Targeted Genetic Targets Using A Hybridization/Capture Approach”), each of which is incorporated by reference in its entirety). In some embodiments, a plurality of bait oligonucleotides can be used to enrich nucleic acids that include all or a part of a sequence of neurological-specific transcripts, or complements thereof, as listed in Table 4 and in the accompanying sequence listing.

In some embodiments, after hybridization of one or more bait oligonucleotides to their target nucleic acids, the hybridized nucleic acids are enriched, creating a pool of nucleic acids that are enriched for particular nucleic acids of interest. In some embodiments, after hybridization of one or more bait oligonucleotides to their target nucleic acids, the un-hybridized nucleic acids are degraded (e.g., by a nuclease), thus enriching the hybridized nucleic acids. In some embodiments, after hybridization of one or more bait oligonucleotides, the hybridized nucleic acids are degraded (e.g., by a nuclease), thus enriching the un-hybridized nucleic acids; for example, this technique may be useful to decrease the amount of a high-abundance nucleic acid that is not of interest. In some embodiments, the bait oligonucleotides may not include a detectable moiety. In some embodiments, the enriched nucleic acids are purified. In some embodiments, the enriched nucleic acids are sample indexed (e.g., prior to sequencing).

In some embodiments, a bait oligonucleotide is hybridized with a nucleic acid at about 40° C., about 45° C., about 50° C., about 55° C., about 60° C., about 65° C., about 70° C., about 75° C., about 80° C. or higher. In some embodiments, the bait oligonucleotides are hybridized with a nucleic acid for at least 15 minutes, at least 30 minutes, at least 45 minutes, at least 1 hour, at least 90 minutes, at least 2 hours, at least 3 hours, at least 4 hours at least 5 hours, or longer.

In some embodiments, one or more detectable moieties can be associated (e.g., attached directly or indirectly) with a bait oligonucleotide. In some embodiments, the one or more detectable moieties can be used to detect (or enhance detection) of a bait oligonucleotide (e.g., a bait oligonucleotide hybridized to a nucleic acid).

In some embodiments, the enriched nucleic acid can be amplified. After amplification, the enriched and amplified nucleic acid can be used to generate a library of nucleic acids and sequenced using any method known in the art, including the exemplary sequencing methods described herein. In some embodiments, sequencing can include determining all or a portion of the sequence of the spatial barcode or the complement thereof in the nucleic acid. In some embodiments, sequencing includes determining all or a portion of the sequence of the analyte from the biological sample or a complement thereof in the nucleic acid. In some embodiments, sequencing includes high throughput sequencing.

In some embodiments, targeted spatial gene expression profiling using one or more panels as described herein (e.g., the cancer panel, the immune panel, the pathway panel, or the neuro panel) can enrich the target nucleic acids as compared to unbiased spatial profiling by about 1 fold, about 2 fold, about 3 fold, about 4 fold, about 5 fold, about 6 fold, about 7 fold, about 8 fold, about 9 fold, about 10 fold, about 20 fold, about 50 fold, about 100 fold or greater. In some embodiments, the targeted spatial gene expression profiling using one or more panels as described herein (e.g., the cancer panel, the immune panel, the pathway panel, or the neuro panel) can enrich the target nucleic acids per gene as compared to unbiased spatial profiling by about 1 fold, about 2 fold, about 3 fold, about 4 fold, about 5 fold, about 6 fold, about 7 fold, about 8 fold, about 9 fold, about 10 fold, about 20 fold, about 50 fold, about 100 fold or greater.

In some embodiments, the targeted spatial gene expression profiling using one or more panels as described herein (e.g., the cancer panel, the immune panel, the pathway panel, or the neuro panel) can increase on-target read percentage as compared to unbiased spatial profiling by about 1 fold, about 2 fold, about 3 fold, about 4 fold, about 5 fold, about 6 fold, about 7 fold, about 8 fold, about 9 fold, about 10 fold, about 20 fold, about 50 fold, about 100 fold or greater. In some embodiments, the targeted spatial gene expression profiling using the panel as described herein (e.g., the cancer panel, the immune panel, the pathway panel, or the neuro panel) can decrease off-target read percentage as compared to unbiased spatial profiling by about 1 fold, about 2 fold, about 3 fold, about 4 fold, about 5 fold, about 6 fold, about 7 fold, about 8 fold, about 9 fold, about 10 fold, about 20 fold, about 50 fold, about 100 fold or greater.

In some embodiments, the targeted spatial gene expression profiling using one or more panels as described herein (e.g., the cancer panel, the immune panel, the pathway panel, or the neuro panel) enriches detection of a target analyte. In some embodiments, the targeted spatial gene expression profiling using one or more panels as described herein (e.g., the cancer panel, the immune panel, the pathway panel, or the neuro panel) enriches detection of one or more target analytes. In some embodiments, enrichment includes an increase in the number of sequencing reads that are detected when the plurality nucleic acids and/or probes are sequenced. In some embodiments, enrichment includes an increase in the number of sequencing reads that are detected when the nucleic acids are sequenced after avidin-biotin pulldown described herein. In some embodiments, enrichment includes an increase in the number of sequencing reads that are detected when the nucleic acids are sequenced after streptavidin-biotin pulldown described herein. In some embodiments, the increase is about 50%, about 55%, about 60%, about 62%, about 65%, about 70%, about 75%, about 80%, about 85%, about 90%, about 95%, about 1 fold, about 2 fold, about 3 fold, about 4 fold, about 5 fold, about 6 fold, about 7 fold, about 8 fold, about 9 fold, about 10 fold, about 20 fold, about 50 fold, about 100 fold or greater enrichment of target analyte sequence reads compared to the number of sequence reads in the same target analyte when performing whole genome sequencing.

In some embodiments, the targeted spatial gene expression profiling using one or more panels as described herein (e.g., the cancer panel, the immune panel, the pathway panel, or the neuro panel) can have about 50%, about 55%, about 60%, about 62%, about 65%, about 70%, about 75%, about 80%, about 85%, about 90%, about 95%, or more of panel gene recovery relative to the unbiased spatial gene expression profiling.

In some embodiments, the targeted spatial gene expression profiling using one or more panels as described herein (e.g., the cancer panel, the immune panel, the pathway panel, or the neuro panel) can have about 50%, about 55%, about 60%, about 62%, about 65%, about 70%, about 75%, about 80%, about 85%, about 90%, about 95%, or more of panel UMI recovery relative to the unbiased spatial gene expression profiling.

In some embodiments, the targeted spatial gene expression profiling using one or more panels as described herein (e.g., the cancer panel, the immune panel, the pathway panel, or the neuro panel) can reduce gene or UMI targeting complexity (or complexity) to about 90%, about 80%, about 70%, about 60%, about 50%, about 40%, about 30%, about 20% or less of the unbiased spatial gene expression profiling.

In some embodiments, dual-indexed libraries can be used for targeted spatial gene expression profiling. In some embodiments, individually indexed libraries can be mixed before the enrichment step by hybridization/capture to generate a targeted library for downstream high-throughput sequencing. In some embodiments, individually indexed libraries can be mixed after the enrichment step by hybridization/capture to generate a targeted library for downstream high-throughput sequencing.

(2) Hybridization of Bait Oligonucleotides Directly to Analytes of Interest.

In some embodiments, a bait oligonucleotide (e.g., a set of bait oligonucleotides from one or more panels) can be added directly to an analyte. In some embodiment, the bait oligonucleotide is directly associated with (i.e., conjugated to) a detectable moiety at the 3′ end. In some embodiment, the bait oligonucleotide is directly associated with (i.e., conjugated to) a detectable moiety at the 5′ end. In some embodiments, the detectable moiety is a fluorescent marker as described herein.

In some embodiments, target-specific reactions are performed in the biological sample. In some embodiments, a nucleic acid analyte is denatured to create single-stranded analytes. In some embodiments, a bait oligonucleotide binds (e.g., hybridizes) directly to an analyte (e.g., a single-stranded analyte), and the detectable moiety (e.g., the fluorescent marker) can be identified using one or more imaging techniques disclosed herein. In some embodiments, target-specific reactions include in situ hybridization of the bait oligonucleotide to an analyte. In some embodiment, the in situ hybridization is fluorescence in situ hybridization (FISH). In some embodiments, the fluorescent marker can serve as a marker to purify an analyte of interest. In some embodiments, fluorescence microscopy can be used to locate the fluorescently-bound bait oligonucleotides. In some embodiments, analytes that are not hybridized to the bait oligonucleotide can be washed away. In some embodiments, the wash methods include isolating only those analytes that fluoresce. In some embodiments, the wash steps include any of the wash steps provided herein.

In some embodiments, a fluorophore (e.g., any of the fluorophores disclosed herein) can be directly associated to (e.g., conjugated to) the bait oligonucleotide. In some embodiments, the bait oligonucleotide can include a non-fluorescent moiety that is directly associated to (e.g., conjugated to) the bait oligonucleotide. In some embodiment, a fluorophore can bind to the non-fluorescent moiety, thereby enhancing detection of the analyte.

In some embodiments, the fluorescence in situ hybridization methods disclosed herein detect a particular set of transcripts. In some embodiments, fluorescence in situ hybridization methods disclosed herein detect cancer-related transcripts (e.g., as disclosed in U.S. Appl. No. 62/970,066 (Titled “Capturing Targeted Genetic Targets Using A Hybridization/Capture Approach”) and U.S. Appl. No. 62/929,686, (Titled “Capturing Targeted Genetic Targets Using A Hybridization/Capture Approach”), each of which is incorporated by reference in its entirety). In some embodiments, fluorescence in situ hybridization methods disclosed herein detect immune-related transcripts (e.g., as disclosed in U.S. Appl. No. 62/970,066 (Titled “Capturing Targeted Genetic Targets Using A Hybridization/Capture Approach”) and U.S. Appl. No. 62/929,686, (Titled “Capturing Targeted Genetic Targets Using A Hybridization/Capture Approach”), each of which is incorporated by reference in its entirety). In some embodiments, fluorescence in situ hybridization methods disclosed herein detect pathway-specific transcripts (e.g., as disclosed in U.S. Appl. No. 62/970,066 (Titled “Capturing Targeted Genetic Targets Using A Hybridization/Capture Approach”) and U.S. Appl. No. 62/929,686, (Titled “Capturing Targeted Genetic Targets Using A Hybridization/Capture Approach”), each of which is incorporated by reference in its entirety). In some embodiments, fluorescence in situ hybridization methods disclosed herein detect neurological-specific transcripts as described herein.

In some embodiments, the hybridized analyte/bait oligonucleotide can be cleaved from the substrate. In some embodiments, the spatially-barcoded array populated with capture probes can be contacted with a sample. The spatially-barcoded capture probes are cleaved and then interact with cells within the provided biological sample. In some embodiments, each capture probe can optionally include at least one cleavage domain. The cleavage domain represents the portion of the probe that is used to reversibly attach the probe to an array feature. Further, one or more segments or regions of the capture probe can optionally be released from the array feature by cleavage of the cleavage domain. As an example, spatial barcodes can be released by cleavage of the cleavage domain. In some embodiments, the capture probe can also include a cleavage site (e.g., a cleavage recognition site of a restriction endonuclease), a photolabile bond, a thermosensitive bond, or a chemical-sensitive bond. In some embodiments, once the spatially-barcoded capture probe is associated with a particular cell, the sample can be optionally removed for analysis.

In some embodiments, the cleaved analyte/bait oligonucleotide is purified for downstream steps (e.g., sequencing). Sequencing can be performed using any method known in the art, including sequencing methods described herein. In some embodiments, sequencing includes determining all or a portion of the sequence of the spatial barcode or the complement thereof. In some embodiments, sequencing includes determining all or a portion of the sequence of the analyte from the biological sample. In some embodiments, sequencing includes high throughput sequencing. In some embodiments, sequencing includes ligating an adapter to the nucleic acid.

In some embodiments, multiple bait oligonucleotides (e.g., two or more bait oligonucleotides) can be used to interrogate spatial gene expression in a biological sample via RNA-templated ligation.

(3) Downstream Application after Hybridization of Bait Oligonucleotides

In some embodiments of methods provided here, RNA-templated ligation is used to interrogate spatial gene expression in a biological sample (e.g., an FFPE tissue section). In some aspects, the steps of RNA-templated ligation include: (1) hybridization of pairs of bait oligonucleotides to a nucleic acid (e.g., a single-stranded cDNA or RNA molecule); (2) ligation of adjacently annealed probe pairs in situ; (3) RNase H treatment that (i) releases RNA-templated ligation products from the tissue (e.g., into solution) for downstream analysis and (ii) destroys unwanted DNA-templated ligation products; and optionally, (4) amplification of RNA—templated ligation products (e.g., by multiplex PCR).

In some aspects, RNA-templated ligation is used for detection of a target analyte, determination of sequence identity, and/or expression monitoring and transcript analysis. In some aspects, RNA-templated ligation allows for detection of a particular change in a nucleic acid (e.g., a mutation or single nucleotide polymorphism (SNP)), detection or expression of a particular nucleic acid, or detection or expression of a particular set of nucleic acids (e.g., in a similar cellular pathway or expressed in a particular pathology). In some embodiments, the methods that include RNA-templated ligation are used to analyze nucleic acids, e.g., by genotyping, quantitation of DNA copy number or RNA transcripts, localization of particular transcripts within samples, and the like. In some aspects, systems and methods provided herein that include RNA-templated ligation identify single nucleotide polymorphisms (SNPs). In some aspects, such systems and methods identify mutations.

In some aspects, disclosed herein are methods of detecting RNA expression that include bringing into contact a first bait oligonucleotide, a second bait oligonucleotide, and ligase (e.g., T4 RNA ligase). In some embodiments, the first bait oligonucleotide and the second bait oligonucleotide are designed to hybridize to a target sequence such that the 5′ end of the first bait oligonucleotide and the 3′ end of the second bait oligonucleotide are adjacent and can be ligated. After hybridization, a ligase (e.g., T4 RNA ligase) ligates the first bait oligonucleotide and the second bait oligonucleotide if the target sequence is present in the target sample, but does not ligate the first bait oligonucleotide and the second bait oligonucleotide if the target sequence is not present in the target sample. The presence or absence of the target sequence in the biological sample can be determined by determining whether or not the first and second bait oligonucleotides were ligated in the presence of ligase.

In some aspects, two or more RNA analytes are analyzed using methods that include RNA-templated ligation. In some aspects, when two or more analytes are analyzed, a first and second bait oligonucleotide that is specific for (e.g., specifically hybridizes to) each RNA analyte are used.

In some aspects, three or more bait oligonucleotides are used in RNA-templated ligation methods provided herein. In some embodiments, the three or more bait oligonucleotides are designed to hybridize to a target sequence such that the three or more probes hybridize adjacent to each other such that the 5′ and 3′ ends of adjacent probes can be ligated. In some embodiments, the presence or absence of the target sequence in the biological sample can be determined by determining whether or not the three or more bait oligonucleotide were ligated in the presence of ligase.

### III. Breast Cancer Biomarkers

Spatial transcriptomics technology has helped address the limitations of traditional pathological examination, combining the benefits of histological techniques and massive throughput of RNA-seq. Disclosed herein are methods of combining spatial transcriptomics technology with histological techniques to resolve the expression profile of a tumor. In particular, as disclosed herein are methods to capture an analyte of interest in an unbiased fashion. Then, imaging and sequencing data are processed together resulting in gene expression mapped to image position.

As disclosed herein, spatial patterns of gene expression that agreed with annotations from pathological examination were combined with immunohistochemical staining for tumor infiltrating lymphocytes, a hallmark of Triple Negative Breast Cancer (TNBC). By aggregating data from serial tissue sections, the delineation of gene expression patterns and cell-type identification were improved. These data were combined with 3′ single-nucleus RNA-seq from the same sample, generating cell-type expression profiles that were used to estimate the proportion of cell-types observed at a given position in the tissue section. Importantly, as disclosed herein, an enrichment strategy to select for cancer-associated genes was used. The enrichment strategy as shown herein showed concordance with the full transcriptome assay, suggesting that a targeted sequencing approach can be used where a fixed gene panel is appropriate.

(a) Biomarkers and Methods of Detecting Biomarkers in a Cancer Sample

Disclosed herein are methods of detecting biomarkers in a cancer sample. In some embodiments, the cancer is breast cancer. In some embodiments, the breast cancer is carcinoma. In some embodiments, the breast cancer is ductal cancer in situ or ductal carcinoma in situ (DCIS). DCIS is an overgrowth of abnormal cells in the milk ducts of the breast that has not spread beyond a milk duct into any normal surrounding breast tissue. In some embodiments, the sample is a tissue sample (e.g., a breast tissue sample). In some embodiments, the tissue sample includes cancer cells. In some embodiments, the sample is a section of a tissue (e.g., a frozen, fresh, or FFPE sample affixed to a slide).

The biomarkers detected herein are aberrantly expressed in cancer cells. In some embodiments, the biomarker is a nucleic acid. In some embodiments, the nucleic acid is mRNA. In some embodiments, the biomarker is one or more mutations in a DNA sample. In some embodiments, the biomarker is a protein. In some embodiments, the biomarkers are aberrantly expressed in breast cancer. In some embodiments, the biomarkers described herein are overexpressed in a cancer sample. In some embodiments, the biomarkers described herein are underexpressed in a cancer sample.

The biomarkers described herein can be detected in part of a tissue section. In some embodiments, the biomarkers disclosed herein are detected in some areas of a tissue section, but not detected in other areas of the tissue section. For example, a section can include an area identified as cancerous. In some embodiments, an area is annotated as cancerous by a pathologist. In some embodiments, an area is identified as cancerous by detection of a different biomarker using any method as described herein. In some embodiments, the cancerous area of a sample expresses (e.g., overexpresses) one or more biomarkers disclosed herein. In some embodiments, a biomarker is aberrantly expressed (e.g., overexpressed) in a particular type of cancer. For example, in some embodiments, a biomarker is aberrantly expressed is DCIS. In some embodiments, a biomarker is aberrantly expressed in a carcinoma.

In some embodiments, the biomarker is a transcript of CENPW, A2ML1, VLDLR, SCRG1, RCL1, FABP7, CCNE1, MIEN1, CDC37L1, ERMP1, TNFSF10, or a combination thereof. In some embodiments, the biomarker is a gene product (e.g., a protein) of CENPW, A2ML1, VLDLR, SCRG1, RCL1, FABP7, CCNE1, MIEN1, CDC37L1, ERMP1, TNFSF10, or a combination thereof. In some embodiments, a cell identified as a carcinoma cell aberrantly expresses CENPW, A2ML1, VLDLR, SCRG1, RCL1, FABP7, CCNE1, MIEN1, CDC37L1, ERMP1, TNFSF10, or a combination thereof.

In some embodiments, the biomarker is centromere protein W (CENPW). The gene product of CENPW is a component of the CENPA-NAC (nucleosome-associated) complex, a complex that plays a role in assembly of kinetochore proteins, mitotic progression and chromosome segregation. In some embodiments, CENPW is aberrantly expressed in cancer. In some embodiments, CENPW is overexpressed in cancer. In some embodiments, CENPW is overexpressed in breast cancer. In some embodiments, CENPW is overexpressed in a carcinoma cell.

In some embodiments, the biomarker is alpha-2-macroglobulin like 1 (A2ML1) (e.g., DNA, a transcript, or a protein). The gene product of A2ML1 inhibits proteinases using a trapping mechanism. The gene product of A2ML1 includes a peptide stretch, called the ‘bait region’ which contains specific cleavage sites for different proteinases. When a proteinase cleaves the bait region, a conformational change is induced in the protein which traps the proteinase. In some embodiments, A2ML1 is aberrantly expressed in cancer. In some embodiments, A2ML1 is overexpressed in cancer. In some embodiments, A2ML1 is overexpressed in breast cancer. In some embodiments, A2ML1 is overexpressed in a carcinoma cell.

In some embodiments, the biomarker is very low density lipoprotein receptor (VLDLR) (e.g., DNA, a transcript, or a protein). The low density lipoprotein receptor (LDLR) gene family consists of cell surface proteins involved in receptor-mediated endocytosis of specific ligands. This gene encodes a lipoprotein receptor that is a member of the LDLR family and plays important roles in VLDL-triglyceride metabolism and the reelin signaling pathway. In some embodiments, VLDLR is aberrantly expressed in cancer. In some embodiments, VLDLR is overexpressed in cancer. In some embodiments, VLDLR is overexpressed in breast cancer. In some embodiments, VLDLR is overexpressed in a carcinoma cell.

In some embodiments, the biomarker is SCRG1 (Scrapie-responsive protein 1, Scrapie-responsive gene 1 protein, or stimulator of chondrogenesis 1) (e.g., DNA, a transcript, or a protein). SCRG1 is associated with neurodegenerative changes observed in transmissible spongiform encephalopathies. It may play a role in host response to prion-associated infections. The scrapie responsive protein 1 may be partly included in the membrane or secreted by the cells due to its hydrophobic N-terminus. In addition, the encoded protein can interact with bone marrow stromal cell antigen 1 (BST1) to enhance the differentiation potentials of human mesenchymal stem cells during tissue and bone regeneration. In some embodiments, SCRG1 is aberrantly expressed in cancer. In some embodiments, SCRG1 is overexpressed in cancer. In some embodiments, SCRG1 is overexpressed in breast cancer. In some embodiments, SCRG1 is overexpressed in a carcinoma cell.

In some embodiments, the biomarker is RNA 3′-terminal phosphate cyclase-like protein (RCL1) (e.g., DNA, a transcript, or a protein). RCL1 plays a role in 40S-ribosomal-subunit biogenesis in the early pre-rRNA processing steps at sites A0, A1 and A2 that are required for proper maturation of the 18S RNA. In some embodiments, RCL1 is aberrantly expressed in cancer. In some embodiments, RCL1 is overexpressed in cancer. In some embodiments, RCL1 is overexpressed in breast cancer. In some embodiments, RCL1 is overexpressed in a carcinoma cell.

In some embodiments, the biomarker is fatty acid binding protein 7 (FABP7) (e.g., DNA, a transcript, or a protein). FABP7 is a cytoplasmic protein that bind long-chain fatty acids and other hydrophobic ligands. In some embodiments, FABP7 is aberrantly expressed in cancer. In some embodiments, FABP7 is overexpressed in cancer. In some embodiments, FABP7 is overexpressed in breast cancer. In some embodiments, FABP7 is overexpressed in a carcinoma cell.

In some embodiments, the biomarker is cyclin E1 (CCNE1) (e.g., DNA, a transcript, or a protein). CCNE1 is involved in control of the cell cycle at the G1/S (start) transition. In some embodiments, CCNE1 is aberrantly expressed in cancer. In some embodiments, CCNE1 is overexpressed in cancer. In some embodiments, CCNE1 is overexpressed in breast cancer. In some embodiments, CCNE1 is overexpressed in a carcinoma cell.

In some embodiments, the biomarker is migration and invasion enhancer 1 (MIEN1) (e.g., DNA, a transcript, or a protein). MIEN1 plays a role in cell migration. In some embodiments, MIEN1 is aberrantly expressed in cancer. In some embodiments, MIEN1 is overexpressed in cancer. In some embodiments, MIEN1 is overexpressed in breast cancer. In some embodiments, MIEN1 is overexpressed in a carcinoma cell.

In some embodiments, the biomarker is cell division cycle 37 like 1 (CDC37L1) (e.g., DNA, a transcript, or a protein). CDC37L1 is a cytoplasmic phosphoprotein that complexes with HSP90 as well as several other proteins involved in HSP90-mediated protein folding (Scholz et al., 2001). In some embodiments, CDC37L1 is aberrantly expressed in cancer. In some embodiments, CDC37L1 is overexpressed in cancer. In some embodiments, CDC37L1 is overexpressed in breast cancer. In some embodiments, CDC37L1 is overexpressed in a carcinoma cell.

In some embodiments, the biomarker is endoplasmic reticulum metallopeptidase 1 (ERMP1) (e.g., DNA, a transcript, or a protein). In some embodiments, ERMP1 is aberrantly expressed in cancer. In some embodiments, ERMP1 is overexpressed in cancer. In some embodiments, ERMP1 is overexpressed in breast cancer. In some embodiments, ERMP1 is overexpressed in a carcinoma cell.

In some embodiments, the biomarker is the cytokine tumor necrosis factor (ligand) superfamily, member 10 (TNFSF10) (e.g., DNA, a transcript, or a protein). In some embodiments, TNFSF10 is aberrantly expressed in cancer. In some embodiments, TNFSF10 is overexpressed in cancer. In some embodiments, TNFSF10 is overexpressed in breast cancer. In some embodiments, TNFSF10 is overexpressed in a carcinoma cell.

In some embodiments, the biomarker is a transcript of ACTG2, DMKN, CALML3, COL17A1, MAGED1, PTN, TMEM98, LY6D, TNC, RTN4IP1, or a combination thereof. In some embodiments, the biomarker is a gene product (e.g., a protein) of ACTG2, DMKN, CALML3, COL17A1, MAGED1, PTN, TMEM98, LY6D, TNC, RTN4IP1, or a combination thereof. In some embodiments, a cell identified as a DCIS cancer cell aberrantly expresses ACTG2, DMKN, CALML3, COL17A1, MAGED1, PTN, TMEM98, LY6D, TNC, RTN4IP1, or a combination thereof.

In some embodiments, the biomarker is actin gamma 2, smooth muscle (ACTG2). ACTG2 is a smooth muscle actin found in enteric tissues and is involved in cell motility. In some embodiments, ACTG2 is aberrantly expressed in cancer. In some embodiments, ACTG2 is overexpressed in cancer. In some embodiments, ACTG2 is overexpressed in breast cancer. In some embodiments, ACTG2 is overexpressed in a DCIS cancer cell.

In some embodiments, the biomarker is dermokine (DMKN). DMKN normally is expressed in the differentiated layers of skin. In some embodiments, DMKN is aberrantly expressed in cancer. In some embodiments, DMKN is overexpressed in cancer. In some embodiments, DMKN is overexpressed in breast cancer. In some embodiments, DMKN is overexpressed in a DCIS cancer cell.

In some embodiments, the biomarker is calmodulin like 3 (CALML3). In some embodiments, CALML3 is aberrantly expressed in cancer. In some embodiments, CALML3 is overexpressed in cancer. In some embodiments, CALML3 is overexpressed in breast cancer. In some embodiments, CALML3 is overexpressed in a DCIS cancer cell.

In some embodiments, the biomarker is collagen type XVII alpha 1 chain (COL17A1). COL17A1 is a transmembrane protein that is a structural component of hemidesmosomes, multiprotein complexes at the dermal-epidermal basement membrane zone that mediate adhesion of keratinocytes to the underlying membrane. In some embodiments, COL17A1 is aberrantly expressed in cancer. In some embodiments, COL17A1 is overexpressed in cancer. In some embodiments, COL17A1 is overexpressed in breast cancer. In some embodiments, COL17A1 is overexpressed in a DCIS cancer cell.

In some embodiments, the biomarker is MAGED1 (Melanoma-associated antigen D1; MAGE family member DD. MAGED1 is involved in the apoptotic response after nerve growth factor (NGF) binding in neuronal cells; MAGED1 inhibits cell cycle progression, and facilitates NGFR-mediated apoptosis. In some embodiments, MAGED1 is aberrantly expressed in cancer. In some embodiments, MAGED1 is overexpressed in cancer. In some embodiments, MAGED1 is overexpressed in breast cancer. In some embodiments, MAGED1 is overexpressed in a DCIS cancer cell.

In some embodiments, the biomarker is pleiotrophin (PTN). PTN is a secreted heparin-binding growth factor. PTN has significant roles in cell growth and survival, cell migration, angiogenesis and tumorigenesis. In some embodiments, PTN is aberrantly expressed in cancer. In some embodiments, PTN is overexpressed in cancer. In some embodiments, PTN is overexpressed in breast cancer. In some embodiments, PTN is overexpressed in a DCIS cancer cell.

In some embodiments, the biomarker is transmembrane protein 98 (TMEM98). TMEM98 functions as a negative regulator of MYRF in oligodendrocyte differentiation and myelination. TMEM98 interacts with the C-terminal of MYRF inhibiting MYRF self-cleavage and N-fragment nuclear translocation. In some embodiments, TMEM98 is aberrantly expressed in cancer. In some embodiments, TMEM98 is overexpressed in cancer. In some embodiments, TMEM98 is overexpressed in breast cancer. In some embodiments, TMEM98 is overexpressed in a DCIS cancer cell.

In some embodiments, the biomarker is lymphocyte antigen 6 family member D (LY6D). In some embodiments, LY6D is aberrantly expressed in cancer. In some embodiments, LY6D is overexpressed in cancer. In some embodiments, LY6D is overexpressed in breast cancer. In some embodiments, LY6D is overexpressed in a DCIS cancer cell.

In some embodiments, the biomarker is tenascin C (TNC). TNC encodes an extracellular matrix protein with a spatially and temporally restricted tissue distribution. TNC encodes a protein that is homohexameric with disulfide-linked subunits, and contains multiple EGF-like and fibronectin type-III domains. In some embodiments, TNC is aberrantly expressed in cancer. In some embodiments, TNC is overexpressed in cancer. In some embodiments, TNC is overexpressed in breast cancer. In some embodiments, TNC is overexpressed in a DCIS cancer cell.

In some embodiments, the biomarker is reticulon 4 interacting protein 1 (RTN4IP1). RTN4IP1 plays a role in the regulation of retinal ganglion cell (RGC) neurite outgrowth and in the development of the inner retina and optic nerve. In some embodiments, RTN4IP1 is aberrantly expressed in cancer. In some embodiments, RTN4IP1 is overexpressed in cancer. In some embodiments, RTN4IP1 is overexpressed in breast cancer. In some embodiments, RTN4IP1 is overexpressed in a DCIS cancer cell.

(b) Panel Capture Methods
(i) Selective Enrichment of Genes of Interest

In some embodiments, where the analyte is a nucleic acid analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by a polymerase. For example, one or more primer sequences with sequence complementarity to one or more RNAs of interest can be used to amplify the one or more RNAs of interest, thereby selectively enriching these RNAs. In some embodiments, an oligonucleotide with sequence complementarity (e.g. a probe) to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity using any of a variety of methods known to the field (e.g., streptavidin or avidin beads). Other non-nucleic acid affinity moieties are known in the art, for example, 2-(4-Hydroxyphenylazo)benzoic acid (HABA).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Subsequent application of the capture probes to the sample can result in improved capture of other types of RNA due to the reduction in non-specific RNA present in the sample.

In some embodiments, an analyte of interest includes one or more isoforms (e.g., a plurality of isoforms). In some embodiments, the plurality of isoforms includes three or more isoforms for the first genetic target. Isoforms of a genetic target refer to mRNA sequences that originate from the same genomic locus but comprise different mRNA sequences, including but not limited to transcription start sites (TSSs), protein coding DNA sequences (CDSs), and/or untranslated regions (UTRs). These differences are caused by alternative splicing, variable promoter usage, gene fusions or deletions, single nucleotide polymorphisms (SNPs), and/or other mutations or post-transcriptional modifications of specific genes. Isoforms of a genetic target may have different functional capacities due to the differences in mRNA sequence of the coding sequences and/or the cis-regulatory elements in the promoter sequences. Such alternative sequences may be recognized by alternative transcription factors and produce differential gene expression or behavior.

In some embodiments, the plurality of isoforms comprises five or more isoforms for the first genetic target. In some embodiments, the plurality of isoforms comprises 2, 3, 4, 5, 6, 7, 8, 9, 10 or more isoforms for the first genetic target. In some embodiments, the first genetic target is a gene and the plurality of isoforms are transcriptional isoforms of the first genetic target. In some embodiments, the first genetic target is a regulator element, such as a promotor, enhancer, or repressor sequence. In some embodiments, the exact genetic function of the genetic target is not known. In some embodiments, the first genetic target is an intro sequence or an exon sequence.

In some embodiments, the methods disclosed herein include obtaining the plurality of analytes, wherein the plurality of analytes comprises a first subset of analytes and second subset of analytes. Each respective sequence in the first subset of analytes maps to a first genetic target that is characterized by a plurality of isoforms. For instance, in some embodiments, the first genetic target includes a reference isoform and an alternative isoform and thus there are two isoforms. In some embodiments, there are more than two isoforms at any given genetic target. In some embodiments, there are two or more, three or more, four or more, or five or more isoforms at a genetic target. Each respective analyte in the second subset of analytes maps to an off-target portion of a reference genome. While the analytes of the second subset may also indeed map to regions of the genome that have multiple isoforms, what distinguishes the analytes of the second subset and makes them members of the second subset is that they do not map to the genetic target.

In some embodiments, the method further comprises exposing the plurality of analytes to a plurality of nucleic acid baits of length K residues, thereby forming a plurality of nucleic acid bait—analyte complexes. In some embodiments, K is between 25 and 1000. In some embodiments, K is between 50 and 500. In some embodiments, K is between 90 and 150. In some embodiments, K is between 95 and 130. In some embodiments, K is 100 or 120.

In some embodiments, each respective nucleic acid bait in the plurality of nucleic acid baits that hybridizes to an isoform in the plurality of isoforms of the genetic target (i) selectively hybridizes to a portion of a first isoform in the plurality of isoforms, or (ii) selectively hybridizes to a portion of an isoform in the plurality of isoforms, other than the first isoform, and is represented by a corresponding portion of a directed graph that is at least a threshold number of edges away (e.g., 1 edge, 2 edges, 3 edges, 4 edges) from any portion of the directed graph that represents a nucleic acid bait that selectively hybridizes to a portion of the first isoform of the genetic target as described in further detail below.

In some embodiments, the hybridization of each respective nucleic acid bait in the plurality of nucleic acid baits to an isoform in the plurality of isoforms is performed a priori. In some such embodiments, an index of candidate baits is generated by determining the length of the genetic target, the length of the respective baits K, and the desired coverage of the nucleic acid baits to the genetic target. In some such embodiments, the index of candidate baits is generated using input values provided by the user or customer. In some such embodiments, the index of candidate baits is modified by repositioning each respective nucleic acid bait by +/−10 base pairs along the genetic target. In some such embodiments, the respective attributes (e.g., melting temperature, Tm) of each respective nucleic acid bait is compared to established parameters. In some such embodiments, the established parameters for nucleic acid baits is obtained through inputs provided by the user or customer. In some embodiments, each respective nucleic acid bait in the plurality of nucleic acid baits that hybridizes to an isoform in the plurality of isoforms of the first genetic target has a Tm with respect to the isoform that is between a first threshold temperature and a second threshold temperature. In some such embodiments, the first threshold temperature is between 65° C. and 85° C. and the second threshold temperature is between 90° C. and 110° C. In some such embodiments, the first threshold temperature is 75° C. and the second threshold temperature is 100° C. In some embodiments, each respective nucleic acid bait in the plurality of nucleic acid baits that hybridizes to an isoform in the plurality of isoforms of the first genetic target that has a Tm below the first threshold temperature or above the second threshold temperature is filtered from the pool of candidate nucleic acid baits.

In some embodiments, the first isoform in the plurality of isoforms of the first genetic target is a principal isoform for the genetic target. For example, in some embodiments, the isoforms in the plurality of isoforms of the first genetic target are ranked prior to hybridization of the nucleic acid baits, where the highest ranked isoform is considered the principal isoform. In some such embodiments, isoform rank assignment is informed by annotations for known isoforms of the genetic target, where annotations are based on functional and/or evolutionary conservation of a respective isoform in the plurality of isoforms.

In some embodiments, a respective nucleic acid bait in a plurality of nucleic acid baits for a respective analyte in a plurality of analytes mapping to a first genetic target is located at a position that is at least a minimum threshold distance from the 3′ end of the respective analyte. In some such embodiments, off-target hybridization of a respective nucleic acid bait to a respective analyte can occur where there are unannotated poly-A sites or poly-A sequences present in the genomic exon and/or mRNA sequence that cause oligo-dT mispriming. As a result, in some such embodiments, the optimal position for nucleic acid bait hybridization to a corresponding analyte is located at a position that is at least a minimum threshold distance from the 3′ end. Non-limiting examples of a minimum threshold distance are from 100 to 200 base pairs (bp), from 200 to 300 bp, from 300 to 400 bp, from 400 to 500 bp, from 500 to 600 bp, from 600 to 700 bp, from 700 to 800 bp, from 800 to 900 bp, from 900 to 1000 bp, or more than 1000 bp. In some embodiments, the percentage of analytes that preferentially hybridize to nucleic acid baits at positions at least a minimum threshold distance away from the 3′ end is between 0% and 10%, between 10% and 20%, or between 20% and 30%. In addition, in some embodiments, nodes and/or edges corresponding to sequences (e.g., analytes or regions of an isoform of a first genetic target) comprising unannotated poly-A sites or poly-A sequences in the mRNA sequence are removed from the directed graph.

In some embodiments, each respective nucleic acid bait in the plurality of nucleic acid baits shares less than a threshold percentage of sequence identity to any other nucleic acid bait in the plurality of nucleic acid baits. For instance, in some embodiments, each respective nucleic acid bait in the plurality of nucleic acid baits shares less than 100 percent, less than 98 percent, less than 96 percent, less than 94 percent, less than 92 percent, less than 90 percent, less than 88 percent, less than 86 percent, less than 84 percent, less than 82 percent, less than 80, less than 70 percent, less than 60 percent, less than 50 percent, or less than 40 percent identity to any other nucleic acid bait in the plurality of nucleic acid baits. In some embodiments, the threshold percentage of sequence identity shared between each respective nucleic acid bait in the plurality of nucleic acid baits to any other nucleic acid bait in the plurality of nucleic acid baits is ten percent, twenty percent, thirty percent, or between five and fifty percent. In some embodiments, the threshold of shared sequence identity between a respective nucleic acid bait in the plurality of nucleic acid baits to any other nucleic acid bait in the plurality of nucleic acid baits determines the presence and abundance of cross-hybridization of each respective nucleic acid bait to off-target analytes.

In some embodiments, the method further comprises selectively capturing the plurality of nucleic acid bait—analyte complexes thereby filtering the plurality of analytes. In some such embodiments, the plurality of nucleic acid bait—analyte complexes are selectively captured to a solid support. In some such embodiments, the solid support comprises a bead. In some further such embodiments, each nucleic acid bait in the plurality of nucleic acid baits comprises a binding moiety. The solid support comprises a plurality of capture moieties. A respective nucleic acid bait—analyte complex is captured on the solid support through a reaction between a capture moiety in the plurality of capture moieties and the corresponding binding moiety of the respective nucleic acid bait—analyte complex. A capture moiety in the plurality of capture moieties comprises avidin and the corresponding binding moiety comprises biotin. A capture moiety in the plurality of capture moieties comprises streptavidin and the corresponding binding moiety comprises biotin. In some embodiments, the plurality of nucleic acid baits are directed to a member selected from the group consisting of: whole or partial exome capture whole or partial transcriptome capture, panel capture, targeted exon capture, anchored exome capture, anchored transcriptome capture, and tiled genomic region capture.

For example, in some such embodiments, a plurality of analytes comprised of nucleic acid analytes (e.g. RNA, mRNA, cDNA or genomic DNA) that are optionally amplified and/or library prepared with functional sequences (e.g. barcodes, spatial barcodes, UMIs, linkers, and/or sequencing adaptors) are hybridized to nucleic acid baits that have sequence complementarity to a plurality of analytes of interest. In some such embodiments, the nucleic acid baits are biotinylated. Due to the strong affinity of biotin to streptavidin or avidin, in some embodiments the nucleic acid bait—analyte complexes are captured by streptavidin-coated beads (e.g., pull-down). Due to the strong affinity of biotin to avidin, in some embodiments the nucleic acid bait
analyte complexes are captured by avidin-coated beads (e.g., pull-down). Analytes are dissociated from the captured nucleic acid bait—analyte complexes for amplification and/or sequencing analysis. In some embodiments, the capturing of the plurality of nucleic acid bait—analyte complexes occurs before amplification and/or library preparation for sequencing. In some embodiments, the capturing of the plurality of nucleic acid bait—analyte complexes occurs after amplification and/or library preparation for sequencing.

(ii) Additional Methods

Also disclosed herein are additional methods that can be combined with the panel capture methods described above. In some embodiments, the identity of one or more cells in a biological sample is determined. In some embodiments, the identity of one or more cells is annotated by a pathologist examining the sample. In some embodiments, a cell or area of a biological sample is determined to include cancer cells or not include cancer cells.

In some embodiments, one or more cells in a biological sample can be identified. In some embodiments, one or more cells in a biological sample are immune cells, For example, in some embodiments, the immune cells are one of a B-cell; a CD34+ cell; a T cell, including a CD4 T cell or a CD8 T cell; a dendritic cell; a monocyte; or a natural killer (NK) cell. In some embodiments, a biological sample is classified as having cancer cells (e.g., DCIS or invasive carcinoma); immune cells; fat, fibrous tissue, or normal glands.

In some embodiments, a one or more additional biomarkers can be detected. It is appreciated that biomarkers can be detected with or without enrichment of the target biomarker.

For example, in some instances, capture of analytes on an array can be performed indiscriminately (e.g., using a capture probe that hybridizes to a poly(A) tail of an mRNA molecule). In this instance, a biomarker can be identified after capture using methods disclosed herein (e.g., next generation sequencing methods). In some instances, bait oligonucleotides as disclosed herein that are designed to detect a biomarker can be used to enhance detection of the biomarker.

In instances in which a biomarker (i.e., an analyte) is captured on an array, sequencing of the analyte (or complement sequence) can be performed using methods known in the art, including but not limited to in situ methods as described herein.

In some embodiments, the one or more additional biomarkers are detected using any of the detection methods described herein. In some embodiments, the one or more additional biomarkers is detected using immunofluorescence. In some embodiments, the one or more additional biomarkers is detected using immunohistochemistry.

In some embodiments, disclosed herein are methods of identifying and detecting expression of a cluster of genes that are dysregulated in a breast cancer sample.

In some instances, the cluster of genes includes identifying and detecting upregulation of one or more of immunoglobulin lambda constant 2 (IGLC2), immunoglobulin heavy constant gamma 3 (IGHG3), immunoglobulin kappa constant (IGKC), immunoglobulin heavy constant gamma 1 (IGHG1), immunoglobulin lambda constant 3 (IGLC3), immunoglobulin heavy constant alpha 1 (IGHA1), Immunoglobulin Heavy Constant Gamma 2 (G2m Marker) (IGHG2), Immunoglobulin Heavy Constant Mu (IGHM), Immunoglobulin Heavy Constant Gamma 4 (G4m Marker) (IGHG4), and Joining Chain Of Multimeric IgA And IgM (JCHAIN); and downregulation of one or more of Inhibitor Of DNA Binding 3, HLH Protein (ID3), Class II Major Histocompatibility Complex Transactivator (CIITA), Cystatin F (CST7), Interferon Alpha Inducible Protein 27 Like 2 (IFI27L2), FYN Proto-Oncogene, Src Family Tyrosine Kinase (FYN), Microtubule Associated Monooxygenase, Calponin And LIM Domain Containing (MICAL1), Heme Oxygenase 1 (HMOX1), CD7 Molecule (CD7), Rho Guanine Nucleotide Exchange Factor 1 (ARHGEF1), and Complement Factor H (CFH). In some instances, this cluster is called cluster 0. In some instances, cluster 0 is detected in fibrous tissue of a breast cancer sample.

In some instances, the cluster of genes includes identifying and detecting upregulation of one or more of Metastasis Associated Lung Adenocarcinoma Transcript 1 (MALAT1), Cathepsin D (CTSD), Thymidine Phosphorylase (TYMP), SAM and HD Domain Containing Deoxynucleoside Triphosphate Triphosphohydrolase 1 (SAMHD1), Cytochrome B-245 Alpha Chain (CYBA), ISG15 Ubiquitin Like Modifier (ISG15), Complement C1q A Chain (C1QA), Ribosomal Protein S9 (RPS9), H2A.J Histone (H2AFJ), and Adipogenesis Regulatory Factor (ADIRF); and downregulation of one or more of Rho GDP Dissociation Inhibitor Alpha (ARHGDIA), Adenine Phosphoribosyltransferase (APRT), AE Binding Protein 1 (AEBP1), Plectin (PLEC), Apolipoprotein E (APOE), Fc Fragment Of IgG Receptor And Transporter (FCGRT), NADH:Ubiquinone Oxidoreductase Subunit B7 (NDUFB7), Methyl-CpG Binding Domain Protein 3 (MBD3), Elastin Microfibril Interfacer 1 (EMILIN1), and GADD45G Interacting Protein 1 (GADD45G1P1). In some instances, this cluster is called cluster 1. In some instances, cluster 1 is detected in fibrous tissue of a breast cancer sample.

In some instances, the cluster of genes includes identifying and detecting upregulation of one or more of C—X—C Motif Chemokine Ligand 14 (CXCL14), Tubulin Tyrosine Ligase Like 12 (TTLL12), GDNF Family Receptor Alpha 1 (GFRA1), Delta 4-Desaturase, Sphingolipid 1 (DEGS1), Anterior Gradient 2, Protein Disulphide Isomerase Family Member (AGR2), Acidic Residue Methyltransferase 1 (ARMT1), Cyclin D1 (CCND1), CAMP Regulated Phosphoprotein 21 (ARPP21), Carnitine 0-Acetyltransferase (CRAT), Protein Kinase CAMP-Activated Catalytic Subunit Beta (PRKACB); and downregulation of one or more of Nuclear Receptor Binding SET Domain Protein 3 (NSD3), Plasminogen Activator, Urokinase Receptor (PLAUR), Cyclin Dependent Kinase Inhibitor 2C (CDKN2C), Factor Interacting With PAPOLA And CPSF1 (FIP1L1), Transmembrane Protein 159 (TMEM159), Transmembrane Protein 141 (TMEM141), Lin-7 Homolog C, Crumbs Cell Polarity Complex Component (L1N7C), Rho Guanine Nucleotide Exchange Factor 39 (ARHGEF39), ARFGEF Family Member 3 (ARFGEF3), and Epithelial Membrane Protein 2 (EMP2). In some instances, this cluster is called cluster 2. In some instances, cluster 2 is detected in areas identified as having invasive carcinoma in a breast cancer sample.

In some instances, the cluster of genes includes identifying and detecting upregulation of one or more of Carboxypeptidase B1 (CPB1), Fc Fragment Of IgG Receptor IIIb (FCGR3B), Kelch Domain Containing 7B (KLHDC7B), Secretoglobin Family 1D Member 2 (SCGB1D2), Signal Peptide, CUB Domain And EGF Like Domain Containing 3 (SCUBE3), C—X—C Motif Chemokine Ligand 9 (CXCL9), Cytochrome C Oxidase Subunit 6C (COX6C), Complement Factor B (CFB), Secretoglobin Family 2A Member 2 (SCGB2A2), and Neuropeptide Y Receptor Y1 (NPY1R); and downregulation of one or more of ENSG00000262580 (AC087741.1), Guanylate Binding Protein 5 (GBP5), Intraflagellar Transport 27 (IFT27), Neuralized E3 Ubiquitin Protein Ligase 4 (NEURL4), Empty Spiracles Homeobox 1 (EMX1), Solute Carrier Family 13 Member 2 (SLC13A2), Family With Sequence Similarity 110 Member A (FAM110A), Signal Peptidase Complex Subunit 1 (SPCS1), Homogentisate 1,2-Dioxygenase (HGD), and Zinc Finger Protein 587 (ZNF587). In some instances, this cluster is called cluster 3. In some instances, cluster 3 is detected in areas identified as having invasive carcinoma in a breast cancer sample.

In some instances, the cluster of genes includes identifying and detecting upregulation of one or more of Cysteine Rich Secretory Protein 3 (CRISP3), SLIT And NTRK Like Family Member 6 (SLITRK6), Chromosome 6 Open Reading Frame 141 (C6orf141), V-Set Domain Containing T Cell Activation Inhibitor 1 (VTCN1), Serine Hydrolase Like 2 (SERHL2), CEA Cell Adhesion Molecule 6 (CEACAM6), ATP Binding Cassette Subfamily C Member 11 (ABCC11), Shisa Family Member 2 (SHISA2), Chromosome 2 open reading frame 54 (C2orf54), and PDZ and LIM Domain 1 (PDLIM1); and downregulation of one or more of Zinc Finger CCCH-Type Containing 12A (ZC3H12A), VPS37B Subunit Of ESCRT-I (VPS37B), Interferon Regulatory Factor 2 Binding Protein 2 (IRF2BP2), Ras Association (RalGDS/AF-6) And Pleckstrin Homology Domains 1 (RAPH1), NFKB Inhibitor Alpha (NFKBIA), Eukaryotic Translation Initiation Factor 2 Alpha Kinase 1 (EIF2AK1), Tripartite Motif Containing 33 (TRIM33), Splicing Factor Proline And Glutamine Rich (SFPQ), Coagulation Factor VII (F7), and Trafficking Protein Particle Complex 3 (TRAPPC3). In some instances, this cluster is called cluster 4. In some instances, cluster 4 is detected in areas identified as having invasive carcinoma in a breast cancer sample.

In some instances, the cluster of genes includes identifying and detecting upregulation of one or more of Long Intergenic Non-Protein Coding RNA 52 (LINC00052), Cytochrome C Oxidase Subunit 6C (COX6C), Synuclein Gamma (SNCG), WAP Four-Disulfide Core Domain 2 (WFDC2), Solute Carrier Family 39 Member 6 (SLC39A6), Microsomal Glutathione S-Transferase 1 (MGST1), Mitochondrial Coiled-Coil Domain 1 (MCCD1), Cystatin A (CSTA), Phosphodiesterase 5A (PDE5A), and Mitochondrially Encoded NADH:Ubiquinone Oxidoreductase Core Subunit 1 (MT-ND1); and downregulation of one or more of Signal Recognition Particle 14 (SRP14), Trafficking Protein Particle Complex 1 (TRAPPC1), Small Nuclear Ribonucleoprotein D3 Polypeptide (SNRPD3), Methionine Adenosyltransferase 2 (MAT2A), Solute Carrier Family 7 Member 8 (SLC7A8), RNA Polymerase II Subunit K (POLR2K), Transmembrane BAX Inhibitor Motif Containing 6 (TMBIM6), OCIA Domain Containing 1 (OCIAD1), Exosome Component 3 (EXOSC3), and Carbonic Anhydrase 1 (CA14). In some instances, this cluster is called cluster 5. In some instances, cluster 5 is detected in areas identified as having invasive carcinoma in a breast cancer sample.

In some instances, the cluster of genes includes identifying and detecting upregulation of one or more of Atypical Chemokine Receptor 1 (Duffy Blood Group) (ACKR1), Insulin Like Growth Factor Binding Protein 7 (IGFBP7), Aquaporin 1 (Colton Blood Group) (AQP1), Von Willebrand Factor (VWF), Metastasis Associated Lung Adenocarcinoma Transcript 1 (MALAT1), SPARC Like 1 (SPARCL1), Transgelin (TAGLN), C—C Motif Chemokine Ligand 21 (CCL21), Actin Alpha 2, Smooth Muscle (ACTA2), and Coiled-Coil Domain Containing 80 (CCDC₈₀); and downregulation of one or more of Cytochrome B5 Reductase 3 (CYB5R3), Plexin D1 (PLXND1), Dual Specificity Phosphatase 1 (DUSP1), Ribosomal Protein S6 (RPS6), Complement C1q A Chain (C1QA), Heart Development Protein with EGF Like Domains 1 (HEG1), ETS Proto-Oncogene 2, Transcription Factor (ETS2), A-Kinase Anchoring Protein 9 (AKAP9), Cell Division Cycle and Apoptosis Regulator 1 (CCAR1), and Tripartite Motif Containing 47 (TRIM47). In some instances, this cluster is called cluster 6. In some instances, cluster 6 is detected in fibrous tissue of a breast cancer sample.

In some instances, the cluster of genes includes identifying and detecting upregulation of one or more of Albumin (ALB), Matrix Gla Protein (MGP), ZNF350 Antisense RNA 1 (ZNF350-AS1), S100 Calcium Binding Protein G (S100G), Stanniocalcin 2 (STC2), CART Prepropeptide (CARTPT), Uncharacterized LOC102724957 (AC087379.2), Glypican 3 (GPC3), Endoplasmic Reticulum Protein 27 (ERP27), and Apolipoprotein D (APOD); and downregulation of one or more of CDV3 Homolog (CDV3), Triosephosphate Isomerase 1 (TPI1), TSPY Like 5 (TSPYL5), Phosphofructokinase, Platelet (PFKP), Cysteine Rich Transmembrane BMP Regulator 1 (CRIM1), Palmitoyl-Protein Thioesterase 1 (PPT1), ENSG00000259457 (AC100826.1), Aldolase, Fructose-Bisphosphate A (ALDOA), Leucine Rich Repeat Containing G Protein-Coupled Receptor 4 (LGR4), and Glutaredoxin 2 (GLRX2). In some instances, this cluster is called cluster 7. In some instances, cluster 7 is detected in areas identified as DCIS in a breast cancer sample.

In some instances, the cluster of genes includes identifying and detecting upregulation of one or more of Uncharacterized LOC102724957 (AC087379.2), S100 Calcium Binding Protein G (S100G), Secretoglobin Family 2A Member 2 (SCGB2A2), PGM5 Antisense RNA 1 (PGM5-AS1), Heme Binding Protein 1 (HEBP1), Adhesion Molecule With Ig Like Domain 2 (AMIGO2), PC-Esterase Domain Containing 1B (PCED1B), Secretoglobin Family 1D Member 2 (SCGB1D2), Inositol 1,4,5-Trisphosphate Receptor Type 1 (ITPR1), and Intraflagellar Transport 122 (IFT122); and downregulation of one or more of GABA Type A Receptor Associated Protein Like 1 (GABARAPL1), Glucose-6-Phosphatase Catalytic Subunit 3 (G6PC3), Inositol Polyphosphate-5-Phosphatase K (INPP5K), Stanniocalcin 2 (STC2), HEXIM P-TEFb Complex Subunit 2 (HEXIM2), Reactive Intermediate Imine Deaminase A Homolog (RIDA), LDL Receptor Related Protein 2 (LRP2), Hexokinase 2 (HK2), Interleukin 1 Receptor Type 2 (IL1R2), and Glutamic-Oxaloacetic Transaminase 2 (GOT2). In some instances, this cluster is called cluster 8. In some instances, cluster 8 is detected in areas identified as having invasive carcinoma in a breast cancer sample.

In some instances, the cluster of genes includes identifying and detecting upregulation of one or more of Albumin (ALB), Mitochondrially Encoded NADH:Ubiquinone Oxidoreductase Core Subunit 2 (MT-ND2), Mitochondrially Encoded NADH:Ubiquinone Oxidoreductase Core Subunit 1 (MT-ND1), Mitochondrially Encoded NADH:Ubiquinone Oxidoreductase Core Subunit 3 (MT-ND3), Mitochondrially Encoded ATP Synthase Membrane Subunit 6 (MT-ATP6), Mitochondrially Encoded NADH:Ubiquinone Oxidoreductase Core Subunit 4 (MT-ND4), Mitochondrially Encoded Cytochrome C Oxidase I (MT-CO1), Mitochondrially Encoded Cytochrome C Oxidase III (MT-CO3), Mitochondrially Encoded ATP Synthase Membrane Subunit 8 (MT-ATPS), Mitochondrially Encoded NADH:Ubiquinone Oxidoreductase Core Subunit 5 (MT-ND5); and downregulation of one or more of Glyoxalase I (GLO1), Abhydrolase Domain Containing 2 (ABHD2), Long Intergenic Non-Protein Coding RNA 52 (LINC00052), RAS Like Estrogen Regulated Growth Inhibitor (RERG), Stanniocalcin 2 (STC2), Metastasis Associated Lung Adenocarcinoma Transcript 1 (MALAT1), SRY-Box Transcription Factor 4 (SOX4), Carbonic Anhydrase 12 (CA12), Zinc Finger Protein 703 (ZNF703), and Mitochondrial Coiled-Coil Domain 1 (MCCD1). In some instances, this cluster is called cluster 9. In some instances, cluster 9 is detected in areas identified as DCIS in a breast cancer sample. In some instances, cluster 9 is detected in areas identified as having invasive carcinoma in a breast cancer sample. In some instances, cluster 9 is detected in areas identified as DCIS in a breast cancer sample and in areas identified as having invasive carcinoma in a breast cancer sample.

In some instances, the cluster of genes includes identifying and detecting upregulation of one or more of Long Intergenic Non-Protein Coding RNA 645 (LINC00645), Solute Carrier Family 30 Member 8 (SLC30A8), Mucin 5B, Oligomeric Mucus/Gel-Forming (MUC5B), Collectin Subfamily Member 12 (COLEC12), Parvalbumin (PVALB), Carboxypeptidase B1 (CPB1), Exocyst Complex Component 2 (EXOC2), ENSG00000278621 (AC037198.2), V-Set And Transmembrane Domain Containing 2A (VSTM2A), and Fibrous Sheath Interacting Protein 1 (FSIP1); and downregulation of one or more of Kinesin Family Member 16B (KIF16B), Spermatogenesis Associated 20 (SPATA20), Tetraspanin 9 (TSPAN9), Calcium Voltage-Gated Channel Auxiliary Subunit Beta 3 (CACNB3), Calcium/Calmodulin Dependent Protein Kinase II Inhibitor 1 (CAMK2N1), Intraflagellar Transport 27 (IFT27), Neuralized E3 Ubiquitin Protein Ligase 1 (NEURL1), Tripartite Motif Containing 3 (TRIM3), Solute Carrier Family 46 Member 1 (SLC46A1), and Enoyl-CoA Delta Isomerase 2 (ECI2). In some instances, this cluster is called cluster 10. In some instances, cluster 10 is detected in areas identified as having invasive carcinoma in a breast cancer sample.

In some instances, the cluster of genes includes identifying and detecting upregulation of one or more of Matrix Gla Protein (MGP), Trefoil Factor 1 (TFF1), Keratin 14 (KRT14), 5100 Calcium Binding Protein A9 (S100A9), Keratin 17 (KRT17), S100 Calcium Binding Protein G (S100G), 5100 Calcium Binding Protein A2 (S100A2), ZNF350 Antisense RNA 1 (ZNF350-AS1), Keratin 5 (KRT5), and 5100 Calcium Binding Protein A8 (S100A8); and downregulation of one or more of MN1 Proto-Oncogene, Transcriptional Regulator (MN1), Transmembrane Protein 45A (TMEM45A), Dynein Axonemal Light Intermediate Chain 1 (DNALI1), Chromosome 3 Open Reading Frame 14 (C3orf14), TSR1 Ribosome Maturation Factor (TSR1), ENSG00000233461 (AL445524.1) Semaphorin 3C (SEMA3C), NADH:Ubiquinone Oxidoreductase Complex Assembly Factor 2 (NDUFAF2), Achaete-Scute Family BHLH Transcription Factor 1 (ASCL1), and Grainyhead Like Transcription Factor 2 (GRHL2). In some instances, this cluster is called cluster 11. In some instances, cluster 11 is detected in areas identified as DCIS in a breast cancer sample.

In some instances, the cluster of genes includes identifying and detecting upregulation of one or more of Serum Amyloid A1 (SAA1), Fatty Acid Binding Protein 4 (FABP4), Glutathione Peroxidase 3 (GPX3), Pleckstrin Homology Domain Containing A8 (PIP), Alcohol Dehydrogenase 1B (Class I), Beta Polypeptide (ADH1B), Perilipin 1 (PLIN1), Collagen Type II Alpha 1 Chain (COL2A1), SH3 Domain Binding Glutamate Rich Protein Like (SH3BGRL), Adiponectin, C1Q And Collagen Domain Containing (ADIPOQ), and Perilipin 4 (PLIN4); and downregulation of one or more of Heterogeneous Nuclear Ribonucleoprotein A0 (HNRNPA0), Ribosomal Protein L11 (RPL11), Solute Carrier Family 40 Member 1 (SLC40A1), Ribosomal Protein Lateral Stalk Subunit P2 (RPLP2), CTD Small Phosphatase 2 (CTDSP2), Sortilin Related Receptor 1 (SORL1), Ribosomal Protein L31 (RPL31), Endothelin Converting Enzyme 1 (ECE1), Secreted Frizzled Related Protein 2 (SFRP2), and Cyclin D1 (CCND1). In some instances, this cluster is called cluster 12. In some instances, cluster 12 is detected in areas identified as having invasive carcinoma in a breast cancer sample.

In some instances, the cluster of genes includes identifying and detecting upregulation of one or more of Phosphodiesterase 5A (PDE5A), Matrix Gla Protein (MGP), WAP Four-Disulfide Core Domain 2 (WFDC2), MRPS30 Divergent Transcript (MRPS30-DT), RNA Binding Motif Protein 20 (RBM20), Mitochondrial Ribosomal Protein S30 (MRPS30), Autocrine Motility Factor Receptor (AMFR), Stanniocalcin 2 (STC2), Potassium Voltage-Gated Channel Subfamily E Regulatory Subunit 4 (KCNE4), and Discoidin Domain Receptor Tyrosine Kinase 1 (DDR1); and downregulation of one or more of Methylcrotonoyl-CoA Carboxylase 1 (MCCC1), Pyruvate Dehydrogenase Phosphatase Regulatory Subunit (PDPR), Archaelysin Family Metallopeptidase 2 (AMZ2), Chromosome 5 Open Reading Frame 15 (C5orf15), TBC1 Domain Family Member 9 (TBC1D9), SRSF Protein Kinase 1 (SRPK1), Long Intergenic Non-Protein Coding RNA 1488 (LINC01488), Tumor Susceptibility 101 (TSG101), Cytochrome C Oxidase Assembly Factor 3 (COA3), and NFKB Inhibitor Epsilon (NFKBIE). In some instances, this cluster is called cluster 13. In some instances, cluster 13 is detected in areas identified as having invasive carcinoma in a breast cancer sample.

In some embodiments, disclosed herein are methods of identifying and/or diagnosing a subject as having breast cancer, wherein the method comprises (a) determining an abundance of CENPW, or a byproduct or precursor or degradation product thereof; and (b) identifying a subject having increased abundance of CENPW, or a byproduct or precursor or degradation product thereof in the biological sample as compared to reference level(s) (e.g., presence or abundance) of the one or more of CENPW, or a byproduct or precursor or degradation product thereof as having breast cancer. In some embodiments, the breast cancer is breast cancer carcinoma.

In some embodiments, disclosed herein are methods of identifying and/or diagnosing a subject as having breast cancer, wherein the method comprises (a) determining an abundance of A2ML1, or a byproduct or precursor or degradation product thereof; and (b) identifying a subject having increased abundance of A2ML1, or a byproduct or precursor or degradation product thereof in the biological sample as compared to reference level(s) (e.g., presence or abundance) of the one or more of A2ML1, or a byproduct or precursor or degradation product thereof as having breast cancer. In some embodiments, the breast cancer is breast cancer carcinoma.

In some embodiments, disclosed herein are methods of identifying and/or diagnosing a subject as having breast cancer, wherein the method comprises (a) determining an abundance of VLDLR, or a byproduct or precursor or degradation product thereof; and (b) identifying a subject having increased abundance of VLDLR, or a byproduct or precursor or degradation product thereof in the biological sample as compared to reference level(s) (e.g., presence or abundance) of the one or more of VLDLR, or a byproduct or precursor or degradation product thereof as having breast cancer. In some embodiments, the breast cancer is breast cancer carcinoma.

In some embodiments, disclosed herein are methods of identifying and/or diagnosing a subject as having breast cancer, wherein the method comprises (a) determining an abundance of SCRG1, or a byproduct or precursor or degradation product thereof; and (b) identifying a subject having increased abundance of SCRG1, or a byproduct or precursor or degradation product thereof in the biological sample as compared to reference level(s) (e.g., presence or abundance) of the one or more of SCRG1, or a byproduct or precursor or degradation product thereof as having breast cancer. In some embodiments, the breast cancer is breast cancer carcinoma.

In some embodiments, disclosed herein are methods of identifying and/or diagnosing a subject as having breast cancer, wherein the method comprises (a) determining an abundance of RCL1, or a byproduct or precursor or degradation product thereof; and (b) identifying a subject having increased abundance of RCL1, or a byproduct or precursor or degradation product thereof in the biological sample as compared to reference level(s) (e.g., presence or abundance) of the one or more of RCL1, or a byproduct or precursor or degradation product thereof as having breast cancer. In some embodiments, the breast cancer is breast cancer carcinoma.

In some embodiments, disclosed herein are methods of identifying and/or diagnosing a subject as having breast cancer, wherein the method comprises (a) determining an abundance of FABP7, or a byproduct or precursor or degradation product thereof; and (b) identifying a subject having increased abundance of FABP7, or a byproduct or precursor or degradation product thereof in the biological sample as compared to reference level(s) (e.g., presence or abundance) of the one or more of FABP7, or a byproduct or precursor or degradation product thereof as having breast cancer. In some embodiments, the breast cancer is breast cancer carcinoma.

In some embodiments, disclosed herein are methods of identifying and/or diagnosing a subject as having breast cancer, wherein the method comprises (a) determining an abundance of CCNE1, or a byproduct or precursor or degradation product thereof; and (b) identifying a subject having increased abundance of CCNE1, or a byproduct or precursor or degradation product thereof in the biological sample as compared to reference level(s) (e.g., presence or abundance) of the one or more of CCNE1, or a byproduct or precursor or degradation product thereof as having breast cancer. In some embodiments, the breast cancer is breast cancer carcinoma.

In some embodiments, disclosed herein are methods of identifying and/or diagnosing a subject as having breast cancer, wherein the method comprises (a) determining an abundance of MIEN1, or a byproduct or precursor or degradation product thereof; and (b) identifying a subject having increased abundance of MIEN1, or a byproduct or precursor or degradation product thereof in the biological sample as compared to reference level(s) (e.g., presence or abundance) of the one or more of MIEN1, or a byproduct or precursor or degradation product thereof as having breast cancer. In some embodiments, the breast cancer is breast cancer carcinoma.

In some embodiments, disclosed herein are methods of identifying and/or diagnosing a subject as having breast cancer, wherein the method comprises (a) determining an abundance of CDC37L1, or a byproduct or precursor or degradation product thereof; and (b) identifying a subject having increased abundance of CDC37L1, or a byproduct or precursor or degradation product thereof in the biological sample as compared to reference level(s) (e.g., presence or abundance) of the one or more of CDC37L1, or a byproduct or precursor or degradation product thereof as having breast cancer. In some embodiments, the breast cancer is breast cancer carcinoma.

In some embodiments, disclosed herein are methods of identifying and/or diagnosing a subject as having breast cancer, wherein the method comprises (a) determining an abundance of ERMP1, or a byproduct or precursor or degradation product thereof; and (b) identifying a subject having increased abundance of ERMP1, or a byproduct or precursor or degradation product thereof in the biological sample as compared to reference level(s) (e.g., presence or abundance) of the one or more of ERMP1, or a byproduct or precursor or degradation product thereof as having breast cancer. In some embodiments, the breast cancer is breast cancer carcinoma.

In some embodiments, disclosed herein are methods of identifying and/or diagnosing a subject as having breast cancer, wherein the method comprises (a) determining an abundance of TNFSF10, or a byproduct or precursor or degradation product thereof; and (b) identifying a subject having increased abundance of TNFSF10, or a byproduct or precursor or degradation product thereof in the biological sample as compared to reference level(s) (e.g., presence or abundance) of the one or more of TNFSF10, or a byproduct or precursor or degradation product thereof as having breast cancer. In some embodiments, the breast cancer is breast cancer carcinoma.

In some embodiments, disclosed herein are methods of identifying and/or diagnosing a subject as having breast cancer, wherein the method comprises (a) determining an abundance of ACTG2, or a byproduct or precursor or degradation product thereof; and (b) identifying a subject having increased abundance of ACTG2, or a byproduct or precursor or degradation product thereof in the biological sample as compared to reference level(s) (e.g., presence or abundance) of the one or more of ACTG2, or a byproduct or precursor or degradation product thereof as having breast cancer. In some embodiments, the breast cancer is DCIS.

In some embodiments, disclosed herein are methods of identifying and/or diagnosing a subject as having breast cancer, wherein the method comprises (a) determining an abundance of DMKN, or a byproduct or precursor or degradation product thereof; and (b) identifying a subject having increased abundance of DMKN, or a byproduct or precursor or degradation product thereof in the biological sample as compared to reference level(s) (e.g., presence or abundance) of the one or more of DMKN, or a byproduct or precursor or degradation product thereof as having breast cancer. In some embodiments, the breast cancer is DCIS.

In some embodiments, disclosed herein are methods of identifying and/or diagnosing a subject as having breast cancer, wherein the method comprises (a) determining an abundance of CALML3, or a byproduct or precursor or degradation product thereof; and (b) identifying a subject having increased abundance of CALML3, or a byproduct or precursor or degradation product thereof in the biological sample as compared to reference level(s) (e.g., presence or abundance) of the one or more of CALML3, or a byproduct or precursor or degradation product thereof as having breast cancer. In some embodiments, the breast cancer is DCIS.

In some embodiments, disclosed herein are methods of identifying and/or diagnosing a subject as having breast cancer, wherein the method comprises (a) determining an abundance of COL17A1, or a byproduct or precursor or degradation product thereof; and (b) identifying a subject having increased abundance of COL17A1, or a byproduct or precursor or degradation product thereof in the biological sample as compared to reference level(s) (e.g., presence or abundance) of the one or more of COL17A1, or a byproduct or precursor or degradation product thereof as having breast cancer. In some embodiments, the breast cancer is DCIS.

In some embodiments, disclosed herein are methods of identifying and/or diagnosing a subject as having breast cancer, wherein the method comprises (a) determining an abundance of MAGED1, or a byproduct or precursor or degradation product thereof; and (b) identifying a subject having increased abundance of MAGED1, or a byproduct or precursor or degradation product thereof in the biological sample as compared to reference level(s) (e.g., presence or abundance) of the one or more of MAGED1, or a byproduct or precursor or degradation product thereof as having breast cancer. In some embodiments, the breast cancer is DCIS.

In some embodiments, disclosed herein are methods of identifying and/or diagnosing a subject as having breast cancer, wherein the method comprises (a) determining an abundance of PTN, or a byproduct or precursor or degradation product thereof; and (b) identifying a subject having increased abundance of PTN, or a byproduct or precursor or degradation product thereof in the biological sample as compared to reference level(s) (e.g., presence or abundance) of the one or more of PTN, or a byproduct or precursor or degradation product thereof as having breast cancer. In some embodiments, the breast cancer is DCIS.

In some embodiments, disclosed herein are methods of identifying and/or diagnosing a subject as having breast cancer, wherein the method comprises (a) determining an abundance of TMEM98, or a byproduct or precursor or degradation product thereof; and (b) identifying a subject having increased abundance of TMEM98, or a byproduct or precursor or degradation product thereof in the biological sample as compared to reference level(s) (e.g., presence or abundance) of the one or more of TMEM98, or a byproduct or precursor or degradation product thereof as having breast cancer. In some embodiments, the breast cancer is DCIS.

In some embodiments, disclosed herein are methods of identifying and/or diagnosing a subject as having breast cancer, wherein the method comprises (a) determining an abundance of LY6D, or a byproduct or precursor or degradation product thereof; and (b) identifying a subject having increased abundance of LY6D, or a byproduct or precursor or degradation product thereof in the biological sample as compared to reference level(s) (e.g., presence or abundance) of the one or more of LY6D, or a byproduct or precursor or degradation product thereof as having breast cancer. In some embodiments, the breast cancer is DCIS.

In some embodiments, disclosed herein are methods of identifying and/or diagnosing a subject as having breast cancer, wherein the method comprises (a) determining an abundance of TNC, or a byproduct or precursor or degradation product thereof; and (b) identifying a subject having increased abundance of TNC, or a byproduct or precursor or degradation product thereof in the biological sample as compared to reference level(s) (e.g., presence or abundance) of the one or more of TNC, or a byproduct or precursor or degradation product thereof as having breast cancer. In some embodiments, the breast cancer is DCIS.

In some embodiments, disclosed herein are methods of identifying and/or diagnosing a subject as having breast cancer, wherein the method comprises (a) determining an abundance of RTN4IP1, or a byproduct or precursor or degradation product thereof; and (b) identifying a subject having increased abundance of RTN4IP1, or a byproduct or precursor or degradation product thereof in the biological sample as compared to reference level(s) (e.g., presence or abundance) of the one or more of RTN4IP1, or a byproduct or precursor or degradation product thereof as having breast cancer. In some embodiments, the breast cancer is DCIS.

In some embodiments, disclosed herein are methods of identifying and/or diagnosing a subject as having breast cancer, wherein the method comprises (a) determining an abundance of ALB, or a byproduct or precursor or degradation product thereof; and (b) identifying a subject having increased abundance of ALB, or a byproduct or precursor or degradation product thereof in the biological sample as compared to reference level(s) (e.g., presence or abundance) of the one or more of ALB, or a byproduct or precursor or degradation product thereof as having breast cancer. In some embodiments, ALB is overexpressed in DCIS.

In some embodiments, disclosed herein are methods of identifying and/or diagnosing a subject as having breast cancer, wherein the method comprises (a) determining an abundance of CRISP3, or a byproduct or precursor or degradation product thereof; and (b) identifying a subject having increased abundance of CRISP3, or a byproduct or precursor or degradation product thereof in the biological sample as compared to reference level(s) (e.g., presence or abundance) of the one or more of CRISP3, or a byproduct or precursor or degradation product thereof as having breast cancer. In some embodiments, CRISP3 is overexpressed in invasive carcinoma (IC).

In some embodiments, disclosed herein are methods of identifying and/or diagnosing a subject as having breast cancer, wherein the method comprises (a) determining an abundance of IGLC2, or a byproduct or precursor or degradation product thereof; and (b) identifying a subject having increased abundance of IGLC2, or a byproduct or precursor or degradation product thereof in the biological sample as compared to reference level(s) (e.g., presence or abundance) of the one or more of IGLC2, or a byproduct or precursor or degradation product thereof as having breast cancer. In some embodiments, IGLC2 is overexpressed in fibrous tissue.

In some embodiments, disclosed herein are methods of identifying and/or diagnosing a subject as having breast cancer, wherein the method comprises (a) determining an abundance of IGHG3, or a byproduct or precursor or degradation product thereof; and (b) identifying a subject having increased abundance of IGHG3, or a byproduct or precursor or degradation product thereof in the biological sample as compared to reference level(s) (e.g., presence or abundance) of the one or more of IGHG3, or a byproduct or precursor or degradation product thereof as having breast cancer. In some embodiments, IGHG3 is overexpressed in fibrous tissue.

In some embodiments, disclosed herein are methods of identifying and/or diagnosing a subject as having breast cancer, wherein the method comprises (a) determining an abundance of IGKC, or a byproduct or precursor or degradation product thereof; and (b) identifying a subject having increased abundance of IGKC, or a byproduct or precursor or degradation product thereof in the biological sample as compared to reference level(s) (e.g., presence or abundance) of the one or more of IGKC, or a byproduct or precursor or degradation product thereof as having breast cancer. In some embodiments, IGKC is overexpressed in fibrous tissue.

In some embodiments, disclosed herein are methods of identifying and/or diagnosing a subject as having breast cancer, wherein the method comprises (a) determining an abundance of CXCL14, or a byproduct or precursor or degradation product thereof; and (b) identifying a subject having increased abundance of CXCL14, or a byproduct or precursor or degradation product thereof in the biological sample as compared to reference level(s) (e.g., presence or abundance) of the one or more of CXCL14, or a byproduct or precursor or degradation product thereof as having breast cancer. In some embodiments, CXCL14 is overexpressed in invasive carcinoma.

In some embodiments, disclosed herein are methods of identifying and/or diagnosing a subject as having breast cancer, wherein the method comprises (a) determining an abundance of IGHG1, or a byproduct or precursor or degradation product thereof; and (b) identifying a subject having increased abundance of IGHG1, or a byproduct or precursor or degradation product thereof in the biological sample as compared to reference level(s) (e.g., presence or abundance) of the one or more of IGHG1, or a byproduct or precursor or degradation product thereof as having breast cancer. In some embodiments, IGHG1 is overexpressed in fibrous tissue.

In some embodiments, disclosed herein are methods of identifying and/or diagnosing a subject as having breast cancer, wherein the method comprises (a) determining an abundance of IGLC3, or a byproduct or precursor or degradation product thereof; and (b) identifying a subject having increased abundance of IGLC3, or a byproduct or precursor or degradation product thereof in the biological sample as compared to reference level(s) (e.g., presence or abundance) of the one or more of IGLC3, or a byproduct or precursor or degradation product thereof as having breast cancer. In some embodiments, IGLC3 is overexpressed in fibrous tissue.

In some embodiments, disclosed herein are methods of identifying and/or diagnosing a subject as having breast cancer, wherein the method comprises (a) determining an abundance of MGP, or a byproduct or precursor or degradation product thereof; and (b) identifying a subject having increased abundance of MGP, or a byproduct or precursor or degradation product thereof in the biological sample as compared to reference level(s) (e.g., presence or abundance) of the one or more of MGP, or a byproduct or precursor or degradation product thereof as having breast cancer. In some embodiments, the MGP is expressed in DCIS.

In some embodiments, disclosed herein are methods of identifying and/or diagnosing a subject as having breast cancer, wherein the method comprises (a) determining an abundance of SLITRK6, or a byproduct or precursor or degradation product thereof; and (b) identifying a subject having increased abundance of SLITRK6, or a byproduct or precursor or degradation product thereof in the biological sample as compared to reference level(s) (e.g., presence or abundance) of the one or more of SLITRK6, or a byproduct or precursor or degradation product thereof as having breast cancer. In some embodiments, SLITRK6 is overexpressed in invasive carcinoma.

In some embodiments, disclosed herein are methods of identifying and/or diagnosing a subject as having breast cancer, wherein the method comprises (a) determining an abundance of CPB1, or a byproduct or precursor or degradation product thereof; and (b) identifying a subject having increased abundance of CPB1, or a byproduct or precursor or degradation product thereof in the biological sample as compared to reference level(s) (e.g., presence or abundance) of the one or more of CPB1, or a byproduct or precursor or degradation product thereof as having breast cancer. In some embodiments, CPB1 is overexpressed in invasive carcinoma.

In some embodiments, disclosed herein are methods of identifying and/or diagnosing a subject as having breast cancer, wherein the method comprises (a) determining an abundance of IGHA1, or a byproduct or precursor or degradation product thereof; and (b) identifying a subject having increased abundance of IGHA1, or a byproduct or precursor or degradation product thereof in the biological sample as compared to reference level(s) (e.g., presence or abundance) of the one or more of IGHA1, or a byproduct or precursor or degradation product thereof as having breast cancer. In some embodiments, IGHA1 is overexpressed in fibrous tissue.

In some embodiments, disclosed herein are methods of identifying and/or diagnosing a subject as having breast cancer, wherein the method comprises (a) determining an abundance of SRP14, or a byproduct or precursor or degradation product thereof; and (b) identifying a subject having increased abundance of SRP14, or a byproduct or precursor or degradation product thereof in the biological sample as compared to reference level(s) (e.g., presence or abundance) of the one or more of SRP14, or a byproduct or precursor or degradation product thereof as having breast cancer. In some embodiments, SRP14 is underexpressed in invasive carcinoma (IC).

In some embodiments, disclosed herein are methods of identifying and/or diagnosing a subject as having breast cancer, wherein the method comprises (a) determining an abundance of MCCC1, or a byproduct or precursor or degradation product thereof; and (b) identifying a subject having increased abundance of MCCC1, or a byproduct or precursor or degradation product thereof in the biological sample as compared to reference level(s) (e.g., presence or abundance) of the one or more of MCCC1, or a byproduct or precursor or degradation product thereof as having breast cancer. In some embodiments, MCCC1 is underexpressed in invasive carcinoma (IC).

In some embodiments, disclosed herein are methods of identifying and/or diagnosing a subject as having breast cancer, wherein the method comprises (a) determining an abundance of CDV3, or a byproduct or precursor or degradation product thereof; and (b) identifying a subject having increased abundance of CDV3, or a byproduct or precursor or degradation product thereof in the biological sample as compared to reference level(s) (e.g., presence or abundance) of the one or more of CDV3, or a byproduct or precursor or degradation product thereof as having breast cancer. In some embodiments, CDV3 is underexpressed in DCIS.

In some embodiments, disclosed herein are methods of identifying and/or diagnosing a subject as having breast cancer, wherein the method comprises (a) determining an abundance of KIF16B, or a byproduct or precursor or degradation product thereof; and (b) identifying a subject having increased abundance of KIF16B, or a byproduct or precursor or degradation product thereof in the biological sample as compared to reference level(s) (e.g., presence or abundance) of the one or more of KIF16B, or a byproduct or precursor or degradation product thereof as having breast cancer. In some embodiments, KIF16B is underexpressed in invasive carcinoma (IC).

In some embodiments, disclosed herein are methods of identifying and/or diagnosing a subject as having breast cancer, wherein the method comprises (a) determining an abundance of ID3, or a byproduct or precursor or degradation product thereof; and (b) identifying a subject having increased abundance of ID3, or a byproduct or precursor or degradation product thereof in the biological sample as compared to reference level(s) (e.g., presence or abundance) of the one or more of ID3, or a byproduct or precursor or degradation product thereof as having breast cancer. In some embodiments, ID3 is underexpressed in fibrous tissue.

In some embodiments, disclosed herein are methods of identifying and/or diagnosing a subject as having breast cancer, wherein the method comprises (a) determining an abundance of ZC3H12A, or a byproduct or precursor or degradation product thereof; and (b) identifying a subject having increased abundance of ZC3H12A, or a byproduct or precursor or degradation product thereof in the biological sample as compared to reference level(s) (e.g., presence or abundance) of the one or more of ZC3H12A, or a byproduct or precursor or degradation product thereof as having breast cancer. In some embodiments, ZC3H12A is underexpressed in invasive carcinoma (IC).

In some embodiments, disclosed herein are methods of identifying and/or diagnosing a subject as having breast cancer, wherein the method comprises (a) determining an abundance of TRAPPC1, or a byproduct or precursor or degradation product thereof; and (b) identifying a subject having increased abundance of TRAPPC1, or a byproduct or precursor or degradation product thereof in the biological sample as compared to reference level(s) (e.g., presence or abundance) of the one or more of TRAPPC1, or a byproduct or precursor or degradation product thereof as having breast cancer. In some embodiments, TRAPPC1 is underexpressed in invasive carcinoma (IC).

In some embodiments, disclosed herein are methods of identifying and/or diagnosing a subject as having breast cancer, wherein the method comprises (a) determining an abundance of NSD3, or a byproduct or precursor or degradation product thereof; and (b) identifying a subject having increased abundance of NSD3, or a byproduct or precursor or degradation product thereof in the biological sample as compared to reference level(s) (e.g., presence or abundance) of the one or more of NSD3, or a byproduct or precursor or degradation product thereof as having breast cancer. In some embodiments, NSD3 is underexpressed in invasive carcinoma (IC).

In some embodiments, disclosed herein are methods of identifying and/or diagnosing a subject as having breast cancer, wherein the method comprises (a) determining an abundance of HNRNPA01, or a byproduct or precursor or degradation product thereof; and (b) identifying a subject having increased abundance of HNRNPA01, or a byproduct or precursor or degradation product thereof in the biological sample as compared to reference level(s) (e.g., presence or abundance) of the one or more of HNRNPA01, or a byproduct or precursor or degradation product thereof as having breast cancer. In some embodiments, the HNRNPA01 is underexpressed in invasive carcinoma (IC).

In some embodiments, disclosed herein are methods of identifying and/or diagnosing a subject as having breast cancer, wherein the method comprises (a) determining an abundance of SPATA20, or a byproduct or precursor or degradation product thereof; and (b) identifying a subject having increased abundance of SPATA20, or a byproduct or precursor or degradation product thereof in the biological sample as compared to reference level(s) (e.g., presence or abundance) of the one or more of SPATA20, or a byproduct or precursor or degradation product thereof as having breast cancer. In some embodiments, SPATA20 is underexpressed in invasive carcinoma (IC).

In some embodiments, disclosed herein are methods of identifying and/or diagnosing a subject as having breast cancer, wherein the method comprises (a) determining an abundance of PDPR, or a byproduct or precursor or degradation product thereof; and (b) identifying a subject having increased abundance of PDPR, or a byproduct or precursor or degradation product thereof in the biological sample as compared to reference level(s) (e.g., presence or abundance) of the one or more of PDPR, or a byproduct or precursor or degradation product thereof as having breast cancer. In some embodiments, PDPR is underexpressed in invasive carcinoma (IC).

In some embodiments, disclosed herein are methods of identifying and/or diagnosing a subject as having breast cancer, wherein the method comprises (a) determining an abundance of GABARAPL1, or a byproduct or precursor or degradation product thereof; and (b) identifying a subject having increased abundance of GABARAPL1, or a byproduct or precursor or degradation product thereof in the biological sample as compared to reference level(s) (e.g., presence or abundance) of the one or more of GABARAPL1, or a byproduct or precursor or degradation product thereof as having breast cancer. In some embodiments, GABARAPL1 is underexpressed in invasive carcinoma (IC).

In some embodiments, a reference abundance (e.g., amount of transcript) is taken from a sample whose transcript abundance is measured as the amount a transcript (e.g., mRNA) from a sample. In some embodiments, the sample is from a different, non-cancerous location in a tissue sample. In some embodiments, reference sample is from a similar tissue obtained from a subject not identified or suspected of having breast cancer. In some embodiments, a reference sample is taken from a sample that is from a cancerous location in a tissue sample. In some embodiments, the reference abundance or presence of a transcript is from a cancerous sample whose abundance is known. In this instance, the known abundance of the transcript is from the cancerous sample can be used as a reference against a test sample.

In some embodiments, the methods disclosed herein include confirming a diagnosis of breast cancer in the subject by obtaining an image of the subject's breast. In some embodiments, the methods disclosed herein include administering a treatment to the breast cancer based on the results of the diagnostic test and/or based on imaging. For example, a subject identified as having breast cancer can be treated with one or more of an endocrine therapy, chemotherapy a hormonal therapy, or a surgical resection. In some embodiments, the endocrine therapy includes tamoxifen, raloxifene, megestrol, or toremifene. In some embodiments, the subject is treated with an aromatase inhibitor such as anastrozole, letrozole, or exemestane. In some embodiments, the chemotherapy is adjuvant chemotherapy. In some embodiments, the chemotherapy is neoadjuvant chemotherapy such as a taxane derivative, such as docetaxel and/or paclitaxel, and/or other anti-cancer agents, such as, members of the anthracycline class of anti-cancer agents, doxorubicin, or topoisomerase inhibitors. In some embodiments, the surgical resection is surgery for breast tissue and/or lymph node tissue. In some embodiments, the breast tissue surgery is selected from the group comprising lumpectomy, quadrantectomy, partial mastectomy, segmental mastectomy, complete mastectomy, and the lymph node tissue surgery is selected from the group consisting of sentinel lymph node biopsy and axillary lymph node dissection.

In some embodiments, disclosed herein are methods of identifying a subject as having an increased likelihood of developing breast cancer, wherein the method comprises (a) determining an abundance of one or more of (1) CENPW, or a byproduct or precursor or degradation product thereof; (2) A2ML1, or a byproduct or precursor or degradation product thereof; (3) VLDLR, or a byproduct or precursor or degradation product thereof; (4) SCRG1, or a byproduct or precursor or degradation product thereof; (5) RCL1, or a byproduct or precursor or degradation product thereof; (6) FABP7, or a byproduct or precursor or degradation product thereof; (7) CCNE1, or a byproduct or precursor or degradation product thereof; (8) MIEN1, or a byproduct or precursor or degradation product thereof; (9) CDC37L1, or a byproduct or precursor or degradation product thereof; (10) ERMP1, or a byproduct or precursor or degradation product thereof; (11) TNFSF10, or a byproduct or precursor or degradation product thereof; (12) ACTG2, or a byproduct or precursor or degradation product thereof; (13) DMKN, or a byproduct or precursor or degradation product thereof; (14) CALML3, or a byproduct or precursor or degradation product thereof; (15) COL17A1, or a byproduct or precursor or degradation product thereof; (16) MAGED1, or a byproduct or precursor or degradation product thereof; (17) PTN, or a byproduct or precursor or degradation product thereof; (18) TMEM98, or a byproduct or precursor or degradation product thereof; (19) LY6D, or a byproduct or precursor or degradation product thereof; (20) TNC, or a byproduct or precursor or degradation product thereof; and (21) RTN4IP1, or a byproduct or precursor or degradation product thereof; and (b) identifying a subject having increased abundance of one or more of (1)-(21), in the biological sample as compared to reference level(s) (e.g., presence or abundance) of the one or more of (1)-(21), as having an increased likelihood of developing breast cancer.

In some embodiments, disclosed herein are methods of identifying a subject as having an increased likelihood of developing breast cancer, wherein the method comprises (a) determining an abundance of one or more of (1) ALB, or a byproduct or precursor or degradation product thereof; (2) CRISP3, or a byproduct or precursor or degradation product thereof; (3) IGLC2, or a byproduct or precursor or degradation product thereof; (4) IGHG3, or a byproduct or precursor or degradation product thereof; (5) IGKC, or a byproduct or precursor or degradation product thereof; (6) CXCL14, or a byproduct or precursor or degradation product thereof; (7) IGHGL, or a byproduct or precursor or degradation product thereof; (8) IGLC3, or a byproduct or precursor or degradation product thereof; (9) MGP, or a byproduct or precursor or degradation product thereof; (10) SLITRK6, or a byproduct or precursor or degradation product thereof; (11) CPB1, or a byproduct or precursor or degradation product thereof; (12) IGHA1, or a byproduct or precursor or degradation product thereof; and (b) identifying a subject having increased abundance of one or more of (1)-(12), in the biological sample as compared to reference level(s) (e.g., presence or abundance) of the one or more of (1)-(12), as having an increased likelihood of developing breast cancer.

In some embodiments, disclosed herein are methods of identifying a subject as having an increased likelihood of developing breast cancer, wherein the method comprises (a) determining an abundance of one or more of the genes in cluster 0, cluster 1, cluster 2, cluster 3, cluster 4, cluster 5, cluster 6, cluster 7, cluster 8, cluster 9, cluster 10, cluster 11, cluster 12, cluster 13 as disclosed in Section VII(b)(ii) herein. In some instance, the methods further include identifying a subject having dysregulated abundance of one or more of clusters 0-13, in the biological sample as compared to reference level(s) (e.g., presence or abundance) of the one or more of clusters 0-13, as having an increased likelihood of developing breast cancer.

In some embodiments, disclosed herein are methods of identifying a subject as having an increased likelihood of developing breast cancer, wherein the method comprises (a) determining an abundance of one or more of (13) SRP14, or a byproduct or precursor or degradation product thereof; (14) MCCC1, or a byproduct or precursor or degradation product thereof; (15) CDV3, or a byproduct or precursor or degradation product thereof; (16) KIF16B, or a byproduct or precursor or degradation product thereof; (17) ID3, or a byproduct or precursor or degradation product thereof; (18) ZC3H12A, or a byproduct or precursor or degradation product thereof; (19) TRAPPC1, or a byproduct or precursor or degradation product thereof; (20) NSD3, or a byproduct or precursor or degradation product thereof; (21) HNRNPA01, or a byproduct or precursor or degradation product thereof; (22) SPATA20, or a byproduct or precursor or degradation product thereof; (23) PDPR, or a byproduct or precursor or degradation product thereof; and (24) GABARAPL1, or a byproduct or precursor or degradation product thereof; and (b) identifying a subject having decreased abundance of one or more of (13)-(24), in the biological sample as compared to reference level(s) (e.g., presence or abundance) of the one or more of (13)-(24), as having an increased likelihood of developing breast cancer. In some embodiments, the method further comprises monitoring the identified subject for the development of symptoms of breast cancer. In some embodiments, the method further comprises recording in the identified subject's clinical record that the subject has an increased likelihood of developing breast cancer. In some embodiments, the method further comprises notifying the subject's family that the subject has an increased likelihood of developing breast cancer. In some embodiments, the method further comprises administering to the subject a treatment for decreasing the likelihood of developing breast cancer. In some embodiments, the methods further include obtaining the biological sample from the subject. In some embodiments, the abundance is an amount of protein or a byproduct or precursor or degradation product thereof. In some embodiments, the abundance is an abundance of mRNA or a fragment thereof.

In some embodiments, the methods disclosed herein include methods of monitoring risk of developing breast cancer in a subject over time, wherein the method includes (a) determining a first abundance of one or more of (1) CENPW, or a byproduct or precursor or degradation product thereof; (2) A2ML1, or a byproduct or precursor or degradation product thereof; (3) VLDLR, or a byproduct or precursor or degradation product thereof; (4) SCRG1, or a byproduct or precursor or degradation product thereof; (5) RCL1, or a byproduct or precursor or degradation product thereof; (6) FABP7, or a byproduct or precursor or degradation product thereof; (7) CCNE1, or a byproduct or precursor or degradation product thereof; (8) MIEN1, or a byproduct or precursor or degradation product thereof; (9) CDC37L1, or a byproduct or precursor or degradation product thereof; (10) ERMP1, or a byproduct or precursor or degradation product thereof; (11) TNFSF10, or a byproduct or precursor or degradation product thereof; (12) ACTG2, or a byproduct or precursor or degradation product thereof; (13) DMKN, or a byproduct or precursor or degradation product thereof; (14) CALML3, or a byproduct or precursor or degradation product thereof; (15) COL17A1, or a byproduct or precursor or degradation product thereof; (16) MAGED1, or a byproduct or precursor or degradation product thereof; (17) PTN, or a byproduct or precursor or degradation product thereof; (18) TMEM98, or a byproduct or precursor or degradation product thereof; (19) LY6D, or a byproduct or precursor or degradation product thereof; (20) TNC, or a byproduct or precursor or degradation product thereof; and (21) RTN4IP1, or a byproduct or precursor or degradation product thereof, in a first biological sample obtained from a subject at a first time point. In some embodiments, the methods include determining a second abundance of one or more of (1) CENPW, or a byproduct or precursor or degradation product thereof; (2) A2ML1, or a byproduct or precursor or degradation product thereof; (3) VLDLR, or a byproduct or precursor or degradation product thereof; (4) SCRG1, or a byproduct or precursor or degradation product thereof; (5) RCL1, or a byproduct or precursor or degradation product thereof; (6) FABP7, or a byproduct or precursor or degradation product thereof; (7) CCNE1, or a byproduct or precursor or degradation product thereof; (8) MIEN1, or a byproduct or precursor or degradation product thereof; (9) CDC37L1, or a byproduct or precursor or degradation product thereof; (10) ERMP1, or a byproduct or precursor or degradation product thereof; (11) TNFSF10, or a byproduct or precursor or degradation product thereof; (12) ACTG2, or a byproduct or precursor or degradation product thereof; (13) DMKN, or a byproduct or precursor or degradation product thereof; (14) CALML3, or a byproduct or precursor or degradation product thereof; (15) COL17A1, or a byproduct or precursor or degradation product thereof; (16) MAGED1, or a byproduct or precursor or degradation product thereof; (17) PTN, or a byproduct or precursor or degradation product thereof; (18) TMEM98, or a byproduct or precursor or degradation product thereof; (19) LY6D, or a byproduct or precursor or degradation product thereof; (20) TNC, or a byproduct or precursor or degradation product thereof; and (21) RTN4IP1, or a byproduct or precursor or degradation product thereof, in a second biological sample obtained from the subject at a second time point. Then, in some embodiments, the methods further include identifying: (i) a subject having an increased second abundance of one or more of (1) through (21) above as compared to the first abundance of the one or more of (1)-(21) as having an increasing risk of developing breast cancer. In some embodiment, a subject having about the same or lowered second abundance of one or more of (1) through (21) as compared to the first abundance of the one or more of (1) through (21), as having about the same or a decreasing risk of developing breast cancer.

In some embodiments, the methods disclosed herein include methods of monitoring risk of developing breast cancer in a subject over time, wherein the method includes (a) determining a first abundance of one or more of (1) ALB, or a byproduct or precursor or degradation product thereof; (2) CRISP3, or a byproduct or precursor or degradation product thereof; (3) IGLC2, or a byproduct or precursor or degradation product thereof; (4) IGHG3, or a byproduct or precursor or degradation product thereof; (5) IGKC, or a byproduct or precursor or degradation product thereof; (6) CXCL14, or a byproduct or precursor or degradation product thereof; (7) IGHGL, or a byproduct or precursor or degradation product thereof; (8) IGLC3, or a byproduct or precursor or degradation product thereof; (9) MGP, or a byproduct or precursor or degradation product thereof; (10) SLITRK6, or a byproduct or precursor or degradation product thereof; (11) CPB1, or a byproduct or precursor or degradation product thereof; (12) IGHA1, or a byproduct or precursor or degradation product thereof; (13) SRP14, or a byproduct or precursor or degradation product thereof; (14) MCCC1, or a byproduct or precursor or degradation product thereof; (15) CDV3, or a byproduct or precursor or degradation product thereof; (16) KIF16B, or a byproduct or precursor or degradation product thereof; (17) ID3, or a byproduct or precursor or degradation product thereof; (18) ZC3H12A, or a byproduct or precursor or degradation product thereof; (19) TRAPPC1, or a byproduct or precursor or degradation product thereof; (20) NSD3, or a byproduct or precursor or degradation product thereof; (21) HNRNPA01, or a byproduct or precursor or degradation product thereof; (22) SPATA20, or a byproduct or precursor or degradation product thereof; (23) PDPR, or a byproduct or precursor or degradation product thereof; and (24) GABARAPL1, or a byproduct or precursor or degradation product thereof in a second biological sample obtained from the subject at a second time point. Then, in some embodiments, the methods further include identifying: (i) a subject having (A) an increased second abundance of one or more of (1) through (12) above or (B) a decreased second abundance of one or more of (13) through (24) as compared to the first abundance of the one or more of (1)-(24) as having an increasing risk of developing breast cancer. In some embodiment, a subject having (A) about the same or lowered second abundance of one or more of (1) through (12) or (B) about the same or elevated second abundance of one or more of (13) through (24) as compared to the first abundance of the one or more of (1) through (24), as having about the same or a decreasing risk of developing breast cancer.

In some embodiments, disclosed herein are methods of methods of monitoring risk of developing breast cancer in a subject over time, wherein the method includes determining a first abundance of one or more of the genes in cluster 0, cluster 1, cluster 2, cluster 3, cluster 4, cluster 5, cluster 6, cluster 7, cluster 8, cluster 9, cluster 10, cluster 11, cluster 12, cluster 13, or any combination thereof, as disclosed in Section VII(b)(ii) herein. In some instance, the methods further include identifying a subject having dysregulated abundance of one or more of clusters 0-13, in the biological sample as compared to reference level(s) (e.g., presence or abundance) of the one or more of clusters 0-13, as having an increased likelihood of developing breast cancer. Then, in some embodiments, the methods further include identifying: (i) a subject having dysregulated abundance(s) in one or more clusters as compared to the first abundance of the one or more of clusters as having an increasing risk of developing breast cancer.

In some embodiments, the methods disclosed herein further include identifying a subject as having an increasing risk of developing breast cancer. In some embodiments, the method further includes administering a treatment for reducing risk of developing breast cancer to the subject or increasing the dose of a treatment for reducing risk of developing breast cancer to be administered to the subject. In some embodiments, the method further comprises recording in the subject's clinical record that the subject has an increasing risk of developing breast cancer. In some embodiments, the method comprises identifying a subject as having about the same or a decreasing risk of developing breast cancer. In some embodiments, the method further comprises recording in the subject's clinical record that the subject as having about the same or a decreasing risk of developing breast cancer.

In some embodiments, disclosed herein is a method of determining efficacy of a treatment for reducing the risk of developing breast cancer in a subject. In some embodiments, the method includes determining a first abundance of one or more of (1) CENPW, or a byproduct or precursor or degradation product thereof; (2) A2ML1, or a byproduct or precursor or degradation product thereof; (3) VLDLR, or a byproduct or precursor or degradation product thereof; (4) SCRG1, or a byproduct or precursor or degradation product thereof; (5) RCL1, or a byproduct or precursor or degradation product thereof; (6) FABP7, or a byproduct or precursor or degradation product thereof; (7) CCNE1, or a byproduct or precursor or degradation product thereof; (8) MIEN1, or a byproduct or precursor or degradation product thereof; (9) CDC37L1, or a byproduct or precursor or degradation product thereof; (10) ERMP1, or a byproduct or precursor or degradation product thereof; (11) TNFSF10, or a byproduct or precursor or degradation product thereof; (12) ACTG2, or a byproduct or precursor or degradation product thereof; (13) DMKN, or a byproduct or precursor or degradation product thereof; (14) CALML3, or a byproduct or precursor or degradation product thereof; (15) COL17A1, or a byproduct or precursor or degradation product thereof; (16) MAGED1, or a byproduct or precursor or degradation product thereof; (17) PTN, or a byproduct or precursor or degradation product thereof; (18) TMEM98, or a byproduct or precursor or degradation product thereof; (19) LY6D, or a byproduct or precursor or degradation product thereof; (20) TNC, or a byproduct or precursor or degradation product thereof; and (21) RTN4IP1, or a byproduct or precursor or degradation product thereof, in a first biological sample obtained from a subject at a first time point. In some embodiments, the methods also include determining a second abundance of the one or more of (1) CENPW, or a byproduct or precursor or degradation product thereof; (2) A2ML1, or a byproduct or precursor or degradation product thereof; (3) VLDLR, or a byproduct or precursor or degradation product thereof; (4) SCRG1, or a byproduct or precursor or degradation product thereof; (5) RCL1, or a byproduct or precursor or degradation product thereof; (6) FABP7, or a byproduct or precursor or degradation product thereof; (7) CCNE1, or a byproduct or precursor or degradation product thereof; (8) MIEN1, or a byproduct or precursor or degradation product thereof; (9) CDC37L1, or a byproduct or precursor or degradation product thereof; (10) ERMP1, or a byproduct or precursor or degradation product thereof; (11) TNFSF10, or a byproduct or precursor or degradation product thereof; (12) ACTG2, or a byproduct or precursor or degradation product thereof; (13) DMKN, or a byproduct or precursor or degradation product thereof; (14) CALML3, or a byproduct or precursor or degradation product thereof; (15) COL17A1, or a byproduct or precursor or degradation product thereof; (16) MAGED1, or a byproduct or precursor or degradation product thereof; (17) PTN, or a byproduct or precursor or degradation product thereof; (18) TMEM98, or a byproduct or precursor or degradation product thereof; (19) LY6D, or a byproduct or precursor or degradation product thereof; (20) TNC, or a byproduct or precursor or degradation product thereof; and (21) RTN4IP1, or a byproduct or precursor or degradation product thereof, in a second biological sample obtained from the subject at a second time point, wherein the subject is administered one or more doses of a treatment for reducing the risk of developing breast cancer between the first and second time points. In some embodiments, the treatment is effective if the subject has about the same or decreased second abundance(s) of one or more of (1) through (21) above, as compared to the first abundance(s) of the one or more of (1) through (21) above.

In some embodiments, disclosed herein is a method of determining efficacy of a treatment for reducing the risk of developing breast cancer in a subject. In some embodiments, the method includes determining a first abundance of one or more of (1) ALB, or a byproduct or precursor or degradation product thereof; (2) CRISP3, or a byproduct or precursor or degradation product thereof; (3) IGLC2, or a byproduct or precursor or degradation product thereof; (4) IGHG3, or a byproduct or precursor or degradation product thereof; (5) IGKC, or a byproduct or precursor or degradation product thereof; (6) CXCL14, or a byproduct or precursor or degradation product thereof; (7) IGHGL, or a byproduct or precursor or degradation product thereof; (8) IGLC3, or a byproduct or precursor or degradation product thereof; (9) MGP, or a byproduct or precursor or degradation product thereof; (10) SLITRK6, or a byproduct or precursor or degradation product thereof; (11) CPB1, or a byproduct or precursor or degradation product thereof; (12) IGHA1, or a byproduct or precursor or degradation product thereof; (13) SRP14, or a byproduct or precursor or degradation product thereof; (14) MCCC1, or a byproduct or precursor or degradation product thereof; (15) CDV3, or a byproduct or precursor or degradation product thereof; (16) KIF16B, or a byproduct or precursor or degradation product thereof; (17) ID3, or a byproduct or precursor or degradation product thereof; (18) ZC3H12A, or a byproduct or precursor or degradation product thereof; (19) TRAPPC1, or a byproduct or precursor or degradation product thereof; (20) NSD3, or a byproduct or precursor or degradation product thereof; (21) HNRNPA01, or a byproduct or precursor or degradation product thereof; (22) SPATA20, or a byproduct or precursor or degradation product thereof; (23) PDPR, or a byproduct or precursor or degradation product thereof; and (24) GABARAPL1, or a byproduct or precursor or degradation product thereof, in a second biological sample obtained from the subject at a second time point. In some instances, the subject is administered one or more doses of a treatment for reducing the risk of developing breast cancer between the first and second time points. In some embodiments, the treatment is effective if the subject has (A) about the same or decreased second abundance(s) of one or more of (1) through (12) above, as compared to the first abundance(s) of the one or more of (1) through (12) above or (B) about the same or increased second abundance(s) of one or more of (13) through (24) above, as compared to the first abundance(s) of the one or more of (13) through (24) above.

In some embodiments, method of determining efficacy of a treatment for reducing the risk of developing breast cancer in a subject. In some embodiments, the method includes determining a first abundance of one or more of the genes in cluster 0, cluster 1, cluster 2, cluster 3, cluster 4, cluster 5, cluster 6, cluster 7, cluster 8, cluster 9, cluster 10, cluster 11, cluster 12, cluster 13, or any combination thereof, as disclosed in Section VII(b)(ii) herein. In some instance, the methods further include identifying a subject having dysregulated abundance of one or more of clusters 0-13, in the biological sample as compared to reference level(s) (e.g., presence or abundance) of the one or more of clusters 0-13, as having an increased likelihood of developing breast cancer.

The methods disclosed herein further include identifying a subject as having an increasing risk of developing breast cancer. In some embodiments, the method further includes administering a treatment for reducing risk of developing breast cancer to the subject or increasing the dose of a treatment for reducing risk of developing breast cancer to be administered to the subject. In some embodiments, the method further comprises recording in the subject's clinical record that the subject has an increasing risk of developing breast cancer. In some embodiments, the method comprises identifying a subject as having about the same or a decreasing risk of developing breast cancer. In some embodiments, the method further comprises recording in the subject's clinical record that the subject as having about the same or a decreasing risk of developing breast cancer.

In some embodiments, the methods disclosed herein include identifying the treatment as being effective in the subject. In some embodiments, the method further includes selecting additional doses of the treatment for the subject. In some embodiments, the method further includes administering additional doses of the treatment to the subject. In some embodiments, the method further includes recording in the subject's clinical record that the treatment is effective in the subject. In some embodiments, the method includes identifying the treatment as not being effective in the subject. In some embodiments, the method further includes selecting a different treatment for the subject. In some embodiments, the method further includes administering a different treatment to the subject. In some embodiments, the method further includes increasing the dose of the treatment to be administered to the subject. In some embodiments, the method further includes administering one or more additional doses of the treatment to the subject in combination with an additional treatment. In some embodiments, the methods further include obtaining the first and second biological samples from the subject. In some embodiments, each of the first and second abundance is an abundance of protein or a byproduct or precursor or degradation product thereof. In some embodiments, each of the first and second abundance is an abundance of mRNA or a fragment thereof.

In some embodiments, also disclosed herein is a method of identifying a patient subpopulation for which a treatment for reducing the risk of developing breast cancer is effective, the method comprising (a) administering a treatment for reducing the risk of developing breast cancer to a patient subpopulation; (b) determining (i) a first abundance of one or more of: (1) CENPW, or a byproduct or precursor or degradation product thereof; (2) A2ML1, or a byproduct or precursor or degradation product thereof; (3) VLDLR, or a byproduct or precursor or degradation product thereof; (4) SCRG1, or a byproduct or precursor or degradation product thereof; (5) RCL1, or a byproduct or precursor or degradation product thereof; (6) FABP7, or a byproduct or precursor or degradation product thereof; (7) CCNE1, or a byproduct or precursor or degradation product thereof; (8) MIEN1, or a byproduct or precursor or degradation product thereof; (9) CDC37L1, or a byproduct or precursor or degradation product thereof; (10) ERMP1, or a byproduct or precursor or degradation product thereof; (11) TNFSF10, or a byproduct or precursor or degradation product thereof; (12) ACTG2, or a byproduct or precursor or degradation product thereof; (13) DMKN, or a byproduct or precursor or degradation product thereof; (14) CALML3, or a byproduct or precursor or degradation product thereof; (15) COL17A1, or a byproduct or precursor or degradation product thereof; (16) MAGED1, or a byproduct or precursor or degradation product thereof; (17) PTN, or a byproduct or precursor or degradation product thereof; (18) TMEM98, or a byproduct or precursor or degradation product thereof; (19) LY6D, or a byproduct or precursor or degradation product thereof; (20) TNC, or a byproduct or precursor or degradation product thereof; and (21) RTN4IP1, or a byproduct or precursor or degradation product thereof, in a first biological sample obtained from a patient subpopulation at a first time point, and (ii) second abundance(s) of the one or more of (1) through (21), in a second biological sample obtained from the patient population at a second time point, wherein the patient subpopulation is administered one or more doses of a treatment for reducing the risk of developing breast cancer between the first and second time points; and (c) determining a correlation between efficacy of the treatment for reducing the risk of developing breast cancer and the second abundance(s) from the patient subpopulation as compared to abundance(s) in a sample obtained from an untreated patient, wherein lower second abundance(s) in the samples from the patient subpopulation as compared to the abundance(s) in the sample from the untreated patient is indicative that the treatment is effective for reducing risk of developing breast cancer in the patient subpopulation. In some embodiments, the therapeutic treatment is an antagonist of one or more of (1) through (21).

In some embodiments, also disclosed herein is a method of identifying a patient subpopulation for which a treatment for reducing the risk of developing breast cancer is effective, the method comprising (a) administering a treatment for reducing the risk of developing breast cancer to a patient subpopulation; (b) determining (i) a first abundance of one or more of: (1) ALB, or a byproduct or precursor or degradation product thereof; (2) CRISP3, or a byproduct or precursor or degradation product thereof; (3) IGLC2, or a byproduct or precursor or degradation product thereof; (4) IGHG3, or a byproduct or precursor or degradation product thereof; (5) IGKC, or a byproduct or precursor or degradation product thereof; (6) CXCL14, or a byproduct or precursor or degradation product thereof; (7) IGHGL, or a byproduct or precursor or degradation product thereof; (8) IGLC3, or a byproduct or precursor or degradation product thereof; (9) MGP, or a byproduct or precursor or degradation product thereof; (10) SLITRK6, or a byproduct or precursor or degradation product thereof; (11) CPB1, or a byproduct or precursor or degradation product thereof; (12) IGHA1, or a byproduct or precursor or degradation product thereof; (13) SRP14, or a byproduct or precursor or degradation product thereof; (14) MCCC1, or a byproduct or precursor or degradation product thereof; (15) CDV3, or a byproduct or precursor or degradation product thereof; (16) KIF16B, or a byproduct or precursor or degradation product thereof; (17) ID3, or a byproduct or precursor or degradation product thereof; (18) ZC3H12A, or a byproduct or precursor or degradation product thereof; (19) TRAPPC1, or a byproduct or precursor or degradation product thereof; (20) NSD3, or a byproduct or precursor or degradation product thereof; (21) HNRNPA01, or a byproduct or precursor or degradation product thereof; (22) SPATA20, or a byproduct or precursor or degradation product thereof; (23) PDPR, or a byproduct or precursor or degradation product thereof; and (24) GABARAPL1, or a byproduct or precursor or degradation product thereof, or (25) one or more of cluster 0, cluster 1, cluster 2, cluster 3, cluster 4, cluster 5, cluster 6, cluster 7, cluster 8, cluster 9, cluster 10, cluster 11, cluster 12, cluster 13, or any combination thereof, as disclosed in Section VII(b)(ii) herein, in a first biological sample obtained from a patient subpopulation at a first time point, and (ii) second abundance(s) of the one or more of (1) through (25), in a second biological sample obtained from the patient population at a second time point, wherein the patient subpopulation is administered one or more doses of a treatment for reducing the risk of developing breast cancer between the first and second time points; and (c) determining a correlation between efficacy of the treatment for reducing the risk of developing breast cancer and the second abundance(s) from the patient subpopulation as compared to abundance(s) in a sample obtained from an untreated patient, wherein lower second abundance(s) of one or more of (1) through (12); increased second abundance(s) of one or more of (13) through (24); or differential expression in (25) in the samples from the patient subpopulation as compared to the abundance(s) in the sample from the untreated patient is indicative that the treatment is effective for reducing risk of developing breast cancer in the patient subpopulation. In some embodiments, the therapeutic treatment is an antagonist of one or more of (1) through (25).

In some embodiments, disclosed herein are methods of identifying a patient subpopulation for which a treatment for reducing the risk of developing breast cancer is effective. In some embodiments, the methods include: (a) administering a treatment for reducing the risk of developing breast cancer to a patient subpopulation; (b) determining (i) a first abundance of one or more of: (1) CENPW, or a byproduct or precursor or degradation product thereof; (2) A2ML1, or a byproduct or precursor or degradation product thereof; (3) VLDLR, or a byproduct or precursor or degradation product thereof; (4) SCRG1, or a byproduct or precursor or degradation product thereof; (5) RCL1, or a byproduct or precursor or degradation product thereof; (6) FABP7, or a byproduct or precursor or degradation product thereof; (7) CCNE1, or a byproduct or precursor or degradation product thereof; (8) MIEN1, or a byproduct or precursor or degradation product thereof; (9) CDC37L1, or a byproduct or precursor or degradation product thereof; (10) ERMP1, or a byproduct or precursor or degradation product thereof; (11) TNFSF10, or a byproduct or precursor or degradation product thereof; (12) ACTG2, or a byproduct or precursor or degradation product thereof; (13) DMKN, or a byproduct or precursor or degradation product thereof; (14) CALML3, or a byproduct or precursor or degradation product thereof; (15) COL17A1, or a byproduct or precursor or degradation product thereof; (16) MAGED1, or a byproduct or precursor or degradation product thereof; (17) PTN, or a byproduct or precursor or degradation product thereof; (18) TMEM98, or a byproduct or precursor or degradation product thereof; (19) LY6D, or a byproduct or precursor or degradation product thereof; (20) TNC, or a byproduct or precursor or degradation product thereof; and (21) RTN4IP1, or a byproduct or precursor or degradation product thereof, in a first biological sample obtained from a patient subpopulation at a first time point, and (ii) second abundance(s) of the one or more of (1) through (21), in a second biological sample obtained from the patient population at a second time point, wherein the patient subpopulation is administered one or more doses of a treatment for reducing the risk of developing breast cancer between the first and second time points; and (c) determining a correlation between efficacy of the treatment for reducing the risk of developing breast cancer and the second abundance(s) from the patient subpopulation as compared to abundance(s) in a sample obtained from an untreated patient, wherein decreased second abundance(s) in the samples from the patient subpopulation as compared to the abundance(s) in the sample from the untreated patient is indicative that the treatment is effective for reducing risk of developing breast cancer in the patient subpopulation. In some embodiments, the therapeutic treatment is an agonist of one or more of (1) through (21).

In some embodiments, disclosed herein are methods of identifying a patient subpopulation for which a treatment for reducing the risk of developing breast cancer is effective. In some embodiments, the methods include: (a) administering a treatment for reducing the risk of developing breast cancer to a patient subpopulation; (b) determining (i) a first abundance of one or more of: (1) ALB, or a byproduct or precursor or degradation product thereof; (2) CRISP3, or a byproduct or precursor or degradation product thereof; (3) IGLC2, or a byproduct or precursor or degradation product thereof; (4) IGHG3, or a byproduct or precursor or degradation product thereof; (5) IGKC, or a byproduct or precursor or degradation product thereof; (6) CXCL14, or a byproduct or precursor or degradation product thereof; (7) IGHGL, or a byproduct or precursor or degradation product thereof; (8) IGLC3, or a byproduct or precursor or degradation product thereof; (9) MGP, or a byproduct or precursor or degradation product thereof; (10) SLITRK6, or a byproduct or precursor or degradation product thereof; (11) CPB1, or a byproduct or precursor or degradation product thereof; (12) IGHA1, or a byproduct or precursor or degradation product thereof; (13) SRP14, or a byproduct or precursor or degradation product thereof; (14) MCCC1, or a byproduct or precursor or degradation product thereof; (15) CDV3, or a byproduct or precursor or degradation product thereof; (16) KIF16B, or a byproduct or precursor or degradation product thereof; (17) ID3, or a byproduct or precursor or degradation product thereof; (18) ZC3H12A, or a byproduct or precursor or degradation product thereof; (19) TRAPPC1, or a byproduct or precursor or degradation product thereof; (20) NSD3, or a byproduct or precursor or degradation product thereof; (21) HNRNPA01, or a byproduct or precursor or degradation product thereof; (22) SPATA20, or a byproduct or precursor or degradation product thereof; (23) PDPR, a byproduct or precursor or degradation product thereof; and (24) GABARAPL1, or a byproduct or precursor or degradation product thereof, or (25) one or more of cluster 0, cluster 1, cluster 2, cluster 3, cluster 4, cluster 5, cluster 6, cluster 7, cluster 8, cluster 9, cluster 10, cluster 11, cluster 12, cluster 13, or any combination thereof, as disclosed in Section VII(b)(ii) herein, in a first biological sample obtained from a patient subpopulation at a first time point, and (ii) second abundance(s) of the one or more of (1) through (25), in a second biological sample obtained from the patient population at a second time point, wherein the patient subpopulation is administered one or more doses of a treatment for reducing the risk of developing breast cancer between the first and second time points; and (c) determining a correlation between efficacy of the treatment for reducing the risk of developing breast cancer and the second abundance(s) from the patient subpopulation as compared to abundance(s) in a sample obtained from an untreated patient, wherein decreased second abundances of any one of (1) through (12); elevated second abundance(s) of any one of (13) through (24); or differential expression in (25) in the samples from the patient subpopulation as compared to the abundance(s) in the sample from the untreated patient is indicative that the treatment is effective for reducing risk of developing breast cancer in the patient subpopulation. In some embodiments, the therapeutic treatment is an agonist of one or more of (1) through (25).

In some embodiments, disclosed herein are methods of modifying treatment for reducing the risk of developing breast cancer in a subject. In some embodiments, the methods include: (a) administering a treatment for reducing the risk of developing breast cancer to a subject; (b) determining (i) pre-treatment abundance(s) of one or more of: (1) CENPW, or a byproduct or precursor or degradation product thereof; (2) A2ML1, or a byproduct or precursor or degradation product thereof; (3) VLDLR, or a byproduct or precursor or degradation product thereof; (4) SCRG1, or a byproduct or precursor or degradation product thereof; (5) RCL1, or a byproduct or precursor or degradation product thereof; (6) FABP7, or a byproduct or precursor or degradation product thereof; (7) CCNE1, or a byproduct or precursor or degradation product thereof; (8) MIEN1, or a byproduct or precursor or degradation product thereof; (9) CDC37L1, or a byproduct or precursor or degradation product thereof; (10) ERMP1, or a byproduct or precursor or degradation product thereof; (11) TNFSF10, or a byproduct or precursor or degradation product thereof; (12) ACTG2, or a byproduct or precursor or degradation product thereof; (13) DMKN, or a byproduct or precursor or degradation product thereof; (14) CALML3, or a byproduct or precursor or degradation product thereof; (15) COL17A1, or a byproduct or precursor or degradation product thereof; (16) MAGED1, or a byproduct or precursor or degradation product thereof; (17) PTN, or a byproduct or precursor or degradation product thereof; (18) TMEM98, or a byproduct or precursor or degradation product thereof; (19) LY6D, or a byproduct or precursor or degradation product thereof; (20) TNC, or a byproduct or precursor or degradation product thereof; and (21) RTN4IP1, or a byproduct or precursor or degradation product thereof, in a pre-treatment sample obtained from a patient before treatment and (ii) post-treatment level(s) of the one or more of (1) through (21), in a post-treatment sample obtained from the patient after treatment, wherein decreased level(s) of (1) through (21), in the post-treatment sample, as compared to the level(s) of one or more of (1) through (21), in a pre-treatment sample, is indicative of the responsiveness to treatment with the treatment for reducing the risk of developing breast cancer; and (c) increasing the amount of the treatment for reducing the risk of developing breast cancer administered to the patient based on the higher level(s) of the one or more of (1) through (21) in the post-treatment sample as compared to the level(s) of one or more of (1) through (21) in the pre-treatment sample. In some embodiments, the treatment for reducing the risk of developing breast cancer is an antagonist of one or more of (1) through (21).

In some embodiments, disclosed herein are methods of modifying treatment for reducing the risk of developing breast cancer in a subject. In some embodiments, the methods include: (a) administering a treatment for reducing the risk of developing breast cancer to a subject; (b) determining (i) pre-treatment abundance(s) of one or more of: (1) ALB, or a byproduct or precursor or degradation product thereof; (2) CRISP3, or a byproduct or precursor or degradation product thereof; (3) IGLC2, or a byproduct or precursor or degradation product thereof; (4) IGHG3, or a byproduct or precursor or degradation product thereof; (5) IGKC, or a byproduct or precursor or degradation product thereof; (6) CXCL14, or a byproduct or precursor or degradation product thereof; (7) IGHGL, or a byproduct or precursor or degradation product thereof; (8) IGLC3, or a byproduct or precursor or degradation product thereof; (9) MGP, or a byproduct or precursor or degradation product thereof; (10) SLITRK6, or a byproduct or precursor or degradation product thereof; (11) CPB1, or a byproduct or precursor or degradation product thereof; (12) IGHA1, or a byproduct or precursor or degradation product thereof; (13) SRP14, or a byproduct or precursor or degradation product thereof; (14) MCCC1, or a byproduct or precursor or degradation product thereof; (15) CDV3, or a byproduct or precursor or degradation product thereof; (16) KIF16B, or a byproduct or precursor or degradation product thereof; (17) ID3, or a byproduct or precursor or degradation product thereof; (18) ZC3H12A, or a byproduct or precursor or degradation product thereof; (19) TRAPPC1, or a byproduct or precursor or degradation product thereof; (20) NSD3, or a byproduct or precursor or degradation product thereof; (21) HNRNPA01, or a byproduct or precursor or degradation product thereof; (22) SPATA20, or a byproduct or precursor or degradation product thereof; (23) PDPR, or a byproduct or precursor or degradation product thereof; (24) GABARAPL1, or a byproduct or precursor or degradation product thereof, and (25) one or more of cluster 0, cluster 1, cluster 2, cluster 3, cluster 4, cluster 5, cluster 6, cluster 7, cluster 8, cluster 9, cluster 10, cluster 11, cluster 12, cluster 13, or any combination thereof, as disclosed in Section VII(b)(ii) herein, in a pre-treatment sample obtained from a patient before treatment and (ii) post-treatment level(s) of the one or more of (1) through (25), in a post-treatment sample obtained from the patient after treatment, wherein decreased level(s) of (1) through (12); increased levels of (13) through (24); or differential expression of (25), in the post-treatment sample, as compared to the level(s) of one or more of (1) through (25), in a pre-treatment sample, is indicative of the responsiveness to treatment with the treatment for reducing the risk of developing breast cancer; and (c) increasing the amount of the treatment for reducing the risk of developing breast cancer administered to the patient based on the abundance(s) of the one or more of (1) through (25) in the post-treatment sample as compared to the abundance(s) of one or more of (1) through (25) in the pre-treatment sample. In some embodiments, the treatment for reducing the risk of developing breast cancer is an antagonist of one or more of (1) through (25).

Also disclosed herein are methods of modifying treatment for reducing the risk of developing breast cancer in a subject. In some embodiments, the methods include: (a) administering a treatment for reducing the risk of developing breast cancer to a subject; (b) determining (i) pre-treatment level(s) of one or more of: (1) CENPW, or a byproduct or precursor or degradation product thereof; (2) A2ML1, or a byproduct or precursor or degradation product thereof; (3) VLDLR, or a byproduct or precursor or degradation product thereof; (4) SCRG1, or a byproduct or precursor or degradation product thereof; (5) RCL1, or a byproduct or precursor or degradation product thereof; (6) FABP7, or a byproduct or precursor or degradation product thereof; (7) CCNE1, or a byproduct or precursor or degradation product thereof; (8) MIEN1, or a byproduct or precursor or degradation product thereof; (9) CDC37L1, or a byproduct or precursor or degradation product thereof; (10) ERMP1, or a byproduct or precursor or degradation product thereof; (11) TNFSF10, or a byproduct or precursor or degradation product thereof; (12) ACTG2, or a byproduct or precursor or degradation product thereof; (13) DMKN, or a byproduct or precursor or degradation product thereof; (14) CALML3, or a byproduct or precursor or degradation product thereof; (15) COL17A1, or a byproduct or precursor or degradation product thereof; (16) MAGED1, or a byproduct or precursor or degradation product thereof; (17) PTN, or a byproduct or precursor or degradation product thereof; (18) TMEM98, or a byproduct or precursor or degradation product thereof; (19) LY6D, or a byproduct or precursor or degradation product thereof; (20) TNC, or a byproduct or precursor or degradation product thereof; and (21) RTN4IP1, or a byproduct or precursor or degradation product thereof, in a pre-treatment sample obtained from a patient before treatment and (ii) post-treatment level. In some embodiments, the methods include: (a) administering a treatment for reducing the risk of developing breast cancer to a subject; (b) determining (i) pre-treatment abundance(s) of one or more of: (1) CENPW, or a byproduct or precursor or degradation product thereof; (2) A2ML1, or a byproduct or precursor or degradation product thereof; (3) VLDLR, or a byproduct or precursor or degradation product thereof; (4) SCRG1, or a byproduct or precursor or degradation product thereof; (5) RCL1, or a byproduct or precursor or degradation product thereof; (6) FABP7, or a byproduct or precursor or degradation product thereof; (7) CCNE1, or a byproduct or precursor or degradation product thereof; (8) MIEN1, or a byproduct or precursor or degradation product thereof; (9) CDC37L1, or a byproduct or precursor or degradation product thereof; (10) ERMP1, or a byproduct or precursor or degradation product thereof; (11) TNFSF10, or a byproduct or precursor or degradation product thereof; (12) ACTG2, or a byproduct or precursor or degradation product thereof; (13) DMKN, or a byproduct or precursor or degradation product thereof; (14) CALML3, or a byproduct or precursor or degradation product thereof; (15) COL17A1, or a byproduct or precursor or degradation product thereof; (16) MAGED1, or a byproduct or precursor or degradation product thereof; (17) PTN, or a byproduct or precursor or degradation product thereof; (18) TMEM98, or a byproduct or precursor or degradation product thereof; (19) LY6D, or a byproduct or precursor or degradation product thereof; (20) TNC, or a byproduct or precursor or degradation product thereof; and (21) RTN4IP1, or a byproduct or precursor or degradation product thereof, in a pre-treatment sample obtained from a patient before treatment and (ii) post-treatment abundance(s) of the one or more of (1) through (21), in a post-treatment sample obtained from the patient after treatment, wherein decreased abundance(s) of (1) through (21), in the post-treatment sample, as compared to the abundance(s) of one or more of (1) through (21), in a pre-treatment sample, is indicative of the responsiveness to treatment with the treatment for reducing the risk of developing breast cancer; and (c) increasing the amount of the treatment for reducing the risk of developing breast cancer administered to the patient based on the decreased abundance(s) of the one or more of (1) through (21) in the post-treatment sample as compared to the abundance(s) of one or more of (1) through (21) in the pre-treatment sample. In some embodiments, the treatment for reducing the risk of developing breast cancer is an agonist of one or more of (1) through (21).

Also disclosed herein are methods of modifying treatment for reducing the risk of developing breast cancer in a subject. In some embodiments, the methods include: (a) administering a treatment for reducing the risk of developing breast cancer to a subject; (b) determining (i) pre-treatment level(s) of one or more of: (1) ALB, or a byproduct or precursor or degradation product thereof; (2) CRISP3, or a byproduct or precursor or degradation product thereof; (3) IGLC2, or a byproduct or precursor or degradation product thereof; (4) IGHG3, or a byproduct or precursor or degradation product thereof; (5) IGKC, or a byproduct or precursor or degradation product thereof; (6) CXCL14, or a byproduct or precursor or degradation product thereof; (7) IGHGL, or a byproduct or precursor or degradation product thereof; (8) IGLC3, or a byproduct or precursor or degradation product thereof; (9) MGP, or a byproduct or precursor or degradation product thereof; (10) SLITRK6, or a byproduct or precursor or degradation product thereof; (11) CPB1, or a byproduct or precursor or degradation product thereof; (12) IGHA1, or a byproduct or precursor or degradation product thereof; (13) SRP14, or a byproduct or precursor or degradation product thereof; (14) MCCC1, or a byproduct or precursor or degradation product thereof; (15) CDV3, or a byproduct or precursor or degradation product thereof; (16) KIF16B, or a byproduct or precursor or degradation product thereof; (17) ID3, or a byproduct or precursor or degradation product thereof; (18) ZC3H12A, or a byproduct or precursor or degradation product thereof; (19) TRAPPC1, or a byproduct or precursor or degradation product thereof; (20) NSD3, or a byproduct or precursor or degradation product thereof; (21) HNRNPA01, or a byproduct or precursor or degradation product thereof; (22) SPATA20, or a byproduct or precursor or degradation product thereof; (23) PDPR, or a byproduct or precursor or degradation product thereof; (24) GABARAPL1, or a byproduct or precursor or degradation product thereof, and (25) one or more of cluster 0, cluster 1, cluster 2, cluster 3, cluster 4, cluster 5, cluster 6, cluster 7, cluster 8, cluster 9, cluster 10, cluster 11, cluster 12, cluster 13, or any combination thereof, as disclosed in Section VII(b)(ii) herein, in a pre-treatment sample obtained from a patient before treatment and (ii) post-treatment abundance(s) of the one or more of (1) through (25), in a post-treatment sample obtained from the patient after treatment, wherein decreased abundance(s) of one or more of (1) through (12); increased abundance(s) of one or more of (13) through (24); or differential expression of (25), in the post-treatment sample, as compared to the abundance(s) of one or more of (1) through (25), in a pre-treatment sample, is indicative of the responsiveness to treatment with the treatment for reducing the risk of developing breast cancer; and (c) increasing the amount of the treatment for reducing the risk of developing breast cancer administered to the patient based on the differential abundance(s) of the one or more of (1) through (25) in the post-treatment sample as compared to the abundance(s) of one or more of (1) through (25) in the pre-treatment sample. In some embodiments, the treatment for reducing the risk of developing breast cancer is an agonist of one or more of (1) through (25).

Also disclosed herein are methods of modifying treatment for reducing the risk of developing breast cancer in a subject. In some embodiments, the methods include: (a) administering a treatment for reducing the risk of developing breast cancer to a subject; (b) determining (i) pre-treatment level(s) of one or more of: (1) ALB, or a byproduct or precursor or degradation product thereof; (2) CRISP3, or a byproduct or precursor or degradation product thereof; (3) IGLC2, or a byproduct or precursor or degradation product thereof; (4) IGHG3, or a byproduct or precursor or degradation product thereof; (5) IGKC, or a byproduct or precursor or degradation product thereof; (6) CXCL14, or a byproduct or precursor or degradation product thereof; (7) IGHGL, or a byproduct or precursor or degradation product thereof; (8) IGLC3, or a byproduct or precursor or degradation product thereof; (9) MGP, or a byproduct or precursor or degradation product thereof; (10) SLITRK6, or a byproduct or precursor or degradation product thereof; (11) CPB1, or a byproduct or precursor or degradation product thereof; (12) IGHA1, or a byproduct or precursor or degradation product thereof, in a pre-treatment sample obtained from a patient before treatment and (ii) post-treatment abundance(s) of the one or more of (1) through (12), in a post-treatment sample obtained from the patient after treatment, wherein decreased abundance(s) of (1) through (12), in the post-treatment sample, as compared to the abundance(s) of one or more of (1) through (12), in a pre-treatment sample, is indicative of the responsiveness to treatment with the treatment for reducing the risk of developing breast cancer; and (c) increasing the amount of the treatment for reducing the risk of developing breast cancer administered to the patient based on the decreased abundance(s) of the one or more of (1) through (12) in the post-treatment sample as compared to the abundance(s) of one or more of (1) through (12) in the pre-treatment sample. In some embodiments, the treatment for reducing the risk of developing breast cancer is an agonist of one or more of (1) through (12).

Also disclosed herein are methods of modifying treatment for reducing the risk of developing breast cancer in a subject. In some embodiments, the methods include: (a) administering a treatment for reducing the risk of developing breast cancer to a subject; (b) determining (i) pre-treatment level(s) of one or more of: (13) SRP14, or a byproduct or precursor or degradation product thereof; (14) MCCC1, or a byproduct or precursor or degradation product thereof; (15) CDV3, or a byproduct or precursor or degradation product thereof; (16) KIF16B, or a byproduct or precursor or degradation product thereof; (17) ID3, or a byproduct or precursor or degradation product thereof; (18) ZC3H12A, or a byproduct or precursor or degradation product thereof; (19) TRAPPC1, or a byproduct or precursor or degradation product thereof; (20) NSD3, or a byproduct or precursor or degradation product thereof; (21) HNRNPA01, or a byproduct or precursor or degradation product thereof; (22) SPATA20, or a byproduct or precursor or degradation product thereof; (23) PDPR, or a byproduct or precursor or degradation product thereof; and (24) GABARAPL1, or a byproduct or precursor or degradation product thereof, in a pre-treatment sample obtained from a patient before treatment and (ii) post-treatment abundance(s) of the one or more of (13) through (24), in a post-treatment sample obtained from the patient after treatment, wherein increased abundance(s) of (13) through (24), in the post-treatment sample, as compared to the abundance(s) of one or more of (13) through (24), in a pre-treatment sample, is indicative of the responsiveness to treatment with the treatment for reducing the risk of developing breast cancer; and (c) increasing the amount of the treatment for reducing the risk of developing breast cancer administered to the patient based on the increased abundance(s) of the one or more of (13) through (24) in the post-treatment sample as compared to the abundance(s) of one or more of (13) through (24) in the pre-treatment sample. In some embodiments, the treatment for reducing the risk of developing breast cancer is an agonist of one or more of (13) through (24).

Also disclosed herein are methods of modifying treatment for reducing the risk of developing breast cancer in a subject. In some embodiments, the methods include: (a) administering a treatment for reducing the risk of developing breast cancer to a subject; (b) determining (i) pre-treatment and post-treatment abundances(s) of one or more of: cluster 0, cluster 1, cluster 2, cluster 3, cluster 4, cluster 5, cluster 6, cluster 7, cluster 8, cluster 9, cluster 10, cluster 11, cluster 12, cluster 13, or any combination thereof, as disclosed in Section VII(b)(ii) herein, (c) modifying the amount of the treatment for reducing the risk of developing breast cancer administered to the patient based on the differential abundance(s) of the one or more of clusters 0-13 in the post-treatment sample as compared to the abundance(s) of one or more of clusters 0-13 in the pre-treatment sample. In some embodiments, the treatment for reducing the risk of developing breast cancer is an agonist of one or more genes in clusters 0-13.

Also disclosed herein are methods of monitoring relapse for developing recurrent breast cancer in a subject. In some embodiments, the methods include: (a) determining (i) pre-treatment level(s) of one or more of: (1) CENPW, or a byproduct or precursor or degradation product thereof; (2) A2ML1, or a byproduct or precursor or degradation product thereof; (3) VLDLR, or a byproduct or precursor or degradation product thereof; (4) SCRG1, or a byproduct or precursor or degradation product thereof; (5) RCL1, or a byproduct or precursor or degradation product thereof; (6) FABP7, or a byproduct or precursor or degradation product thereof; (7) CCNE1, or a byproduct or precursor or degradation product thereof; (8) MIEN1, or a byproduct or precursor or degradation product thereof; (9) CDC37L1, or a byproduct or precursor or degradation product thereof; (10) ERMP1, or a byproduct or precursor or degradation product thereof; (11) TNFSF10, or a byproduct or precursor or degradation product thereof; (12) ACTG2, or a byproduct or precursor or degradation product thereof; (13) DMKN, or a byproduct or precursor or degradation product thereof; (14) CALML3, or a byproduct or precursor or degradation product thereof; (15) COL17A1, or a byproduct or precursor or degradation product thereof; (16) MAGED1, or a byproduct or precursor or degradation product thereof; (17) PTN, or a byproduct or precursor or degradation product thereof; (18) TMEM98, or a byproduct or precursor or degradation product thereof; (19) LY6D, or a byproduct or precursor or degradation product thereof; (20) TNC, or a byproduct or precursor or degradation product thereof; and (21) RTN4IP1, or a byproduct or precursor or degradation product thereof, in a pre-treatment sample obtained from a patient before treatment and (ii) post-treatment level. In some embodiments, the methods include: (a) administering a treatment for reducing the risk of developing breast cancer to a subject; (b) determining (i) pre-treatment abundance(s) of one or more of: (1) CENPW, or a byproduct or precursor or degradation product thereof; (2) A2ML1, or a byproduct or precursor or degradation product thereof; (3) VLDLR, or a byproduct or precursor or degradation product thereof; (4) SCRG1, or a byproduct or precursor or degradation product thereof; (5) RCL1, or a byproduct or precursor or degradation product thereof; (6) FABP7, or a byproduct or precursor or degradation product thereof; (7) CCNE1, or a byproduct or precursor or degradation product thereof; (8) MIEN1, or a byproduct or precursor or degradation product thereof; (9) CDC37L1, or a byproduct or precursor or degradation product thereof; (10) ERMP1, or a byproduct or precursor or degradation product thereof; (11) TNFSF10, or a byproduct or precursor or degradation product thereof; (12) ACTG2, or a byproduct or precursor or degradation product thereof; (13) DMKN, or a byproduct or precursor or degradation product thereof; (14) CALML3, or a byproduct or precursor or degradation product thereof; (15) COL17A1, or a byproduct or precursor or degradation product thereof; (16) MAGED1, or a byproduct or precursor or degradation product thereof; (17) PTN, or a byproduct or precursor or degradation product thereof; (18) TMEM98, or a byproduct or precursor or degradation product thereof; (19) LY6D, or a byproduct or precursor or degradation product thereof; (20) TNC, or a byproduct or precursor or degradation product thereof; and (21) RTN4IP1, or a byproduct or precursor or degradation product thereof, in a pre-treatment sample obtained from a patient before treatment and (ii) post-treatment abundance(s) of the one or more of (1) through (21), in a post-treatment sample obtained from the patient after treatment. The methods further include monitoring the abundance(s) of one or more of (1) through (21) over time (e.g., multiple times over a period of weeks, months, or years) after treatment. Relapse for developing recurrent breast cancer in a subject can be determined if there is an increased abundance of any one of (1) through (21) as compared to the abundance(s) of one or more of (1) through (21), in a post-treatment sample. In some instances, the method further includes increasing the amount of the treatment for reducing the risk of developing breast cancer administered to the patient based on the increased abundance(s) of the one or more of (1) through (21) as compared to the abundance(s) of one or more of (1) through (21) in the post-treatment sample. In some embodiments, the treatment for reducing the risk of developing breast cancer is an agonist of one or more of (1) through (21).

Also disclosed herein are methods of monitoring relapse for developing recurrent breast cancer in a subject. In some embodiments, the methods include: (a) determining (i) pre-treatment level(s) of one or more of: (1) ALB, or a byproduct or precursor or degradation product thereof; (2) CRISP3, or a byproduct or precursor or degradation product thereof; (3) IGLC2, or a byproduct or precursor or degradation product thereof; (4) IGHG3, or a byproduct or precursor or degradation product thereof; (5) IGKC, or a byproduct or precursor or degradation product thereof; (6) CXCL14, or a byproduct or precursor or degradation product thereof; (7) IGHGL, or a byproduct or precursor or degradation product thereof; (8) IGLC3, or a byproduct or precursor or degradation product thereof; (9) MGP, or a byproduct or precursor or degradation product thereof; (10) SLITRK6, or a byproduct or precursor or degradation product thereof; (11) CPB1, or a byproduct or precursor or degradation product thereof; (12) IGHA1, or a byproduct or precursor or degradation product thereof; (13) SRP14, or a byproduct or precursor or degradation product thereof; (14) MCCC1, or a byproduct or precursor or degradation product thereof; (15) CDV3, or a byproduct or precursor or degradation product thereof; (16) KIF16B, or a byproduct or precursor or degradation product thereof; (17) ID3, or a byproduct or precursor or degradation product thereof; (18) ZC3H12A, or a byproduct or precursor or degradation product thereof; (19) TRAPPC1, or a byproduct or precursor or degradation product thereof; (20) NSD3, or a byproduct or precursor or degradation product thereof; (21) HNRNPA01, or a byproduct or precursor or degradation product thereof; (22) SPATA20, or a byproduct or precursor or degradation product thereof; (23) PDPR, or a byproduct or precursor or degradation product thereof; and (24) GABARAPL1, or a byproduct or precursor or degradation product thereof, in a pre-treatment sample obtained from a patient before treatment and (ii) post-treatment abundance(s) of the one or more of (1) through (24), in a post-treatment sample obtained from the patient after treatment. The methods further include monitoring the abundance(s) of one or more of (1) through (24) over time (e.g., multiple times over a period of weeks, months, or years) after treatment. Relapse for developing recurrent breast cancer in a subject can be determined if there is an increased abundance of any one of (1) through (12) or an decreased abundance of any one of (13) through (24), as compared to the abundance(s) of one or more of (1) through (24), in a post-treatment sample. In some instances, the method further includes increasing the amount of the treatment for reducing the risk of developing breast cancer administered to the patient based on the increased abundance(s) of the one or more of (1) through (12) or the decreased abundance(s) of (13) through (24) in the relapsed sample as compared to the abundance(s) of one or more of (1) through (24) in the post-treatment sample. In some embodiments, the treatment for reducing the risk of developing breast cancer is an agonist of one or more of (1) through (24).

(c) Integration of Single Nucleus RNA-Seq with Spatial Gene Expression Capture

Spatial gene expression as described herein provides a wealth of information regarding the location and abundance of a sample. In some instances, these methods are augmented by single-cell cell methods to further determine the cell type at a particular location in a sample. In some instances, the methods disclosed herein examine single cells in a first section of a tissue to identify cell make-up in the sample. Then, a second serial section is imaged and subjected to the spatial analysis methods disclosed herein (e.g., spatial detection using an array having capture probes; selective enrichment using one or more bait oligonucleotides disclosed herein). Using biomarkers associated with cell types, one can combine the cell make-up data with the spatial information to determine cell type in the sample. Combining spatial analysis with single cell analysis provides the ability to understanding microenvironments and cellular structure, including allowing the ability to understand the spatial distribution of cellular components.

In some instances, the methods of analyzing individual cells (i.e., individual nuclei) include steps of separating cells from a biological sample, isolating RNA from each cell, barcoding RNA from each cell, cDNA amplification and cleanup, and sequencing.

The methods disclosed herein allow for profiling of 500-100,000 individual cells per sample. In some instances, a pool of ˜3,500,000 barcodes are sampled separately to index each cell's transcriptome by partitioning thousands of cells into nanoliter-scale beads-in-emulsion, where all generated cDNA share a common barcode sequence. Libraries are generated and sequenced from the cDNA and barcodes are used to associate individual reads back to the individual partitions (i.e., cell). See e.g., Chromium Next GEM Single Cell 3′ Reagent Kits v3.1 User Guide, Document CG000204, November 2019, which is incorporated by reference in its entirety.

In some instances, the biological sample is a tissue. In some instances, the biological sample is a section of a tissue. In instances where Single Nucleus RNA-sequencing is used with spatial methods, serial sections of a tissue are used, one for each method. In some instances, the section is about 10 μm. In some instances, the section is about 5 μm to about 20 μm; about 5 μm to about 15 μm; or about 5 μm to about 10 μm.

In a single nucleus RNA-sequencing sample, the number of isolated nuclei can vary. In some instances the number of isolated nuclei range from about 500 nuclei to about 100,000 nuclei (e.g., about 500, about 1,000, about 2,000, about 3,000, about 4,000, about 5,000, about 6,000, about 7,000, about 8,000, about 9,000, 10,000, about 20,000, about 30,000, about 40,000, about 50,000, about 60,000, about 70,000, about 80,000, about 90,000, or about 1,000,000, nuclei).

The methods disclosed herein allow for identification of cell types based on known biomarkers or biomarkers disclosed herein. In some instances, single cells can be identified include one or more of T cells, B cells, cancer or tumor cells, tumor stem cells, CD49f^{hi} mammary stem cells, CD163+ macrophages, CD80+ macrophages, alveolar secretory cells, luminal progenitor cells, stromal cells, plasmacytoid dendritic cells, mast cells, myoepithelial cells, mature luminal cells, endothelial lymphatic cells, and ductal cells.

Because one spot on an array can have multiple cells, it is necessary to deconvolute the spot in order to determine which cell type(s) are in each spot. In some instances, each spot includes about 1 to 10 cells (e.g., 1 to 5, 2 to 5, 2 to 4). Further, because each spot contains more than one cell it can be useful to estimate the proportion of a given cell type at each spot. This gives insight into spatial organization of cells as well as how different cell types might interact in space. Multiple methods of deconvolution of spots are known in the art. See Elosua et al., SPOTlight: Seeded NMF regression to Deconvolute Spatial Transcriptomics Spots with Single-Cell Transcriptomes; Maaskola et al., Charting Tissue Expression Anatomy by Spatial Transcriptome Deconvolution; and MuSiC.

### EXAMPLES

### Example 1: Targeted Spatial Gene Expression Workflow

A slide is prepared and kept for storage at −80° C. for less than a week. After being thawed, the biological sample is fixed by methanol, stained by H&E (Haemotoxylin and Eosin), and then imaged in bright field. Next, the fixed sample is permeabilized. Next, the first strand cDNA is synthesized and then denatured, followed by the second cDNA synthesis and denaturation. Next, the cDNA is transferred off the slide and quantified by qPCR. Next, the cDNA is amplified followed by quality control (QC). Next, the cDNA is modified by fragmentation, end repair and A-tailing, followed by adapter ligation. Next, sample index PCR (SI-PCR) is performed and the quality of the resulting library is checked by QC. The library as peppered above is dried down and hybridized with biotinylated baits. After the hybridization, the biotinylated baits is captured with streptavidin or avidin beads. The beads is washed and the retaining library is re-amplified as a new library. The quality of the new library is checked and used for downstream steps.

In an alternative way, the steps including hybridization using biotinylated baits, capture with streptavidin or avidin beads and washing is performed prior to the adapter ligation and SI-PCR steps.

### Example 2: Selective Enrichment of Genes of Interest in Triple Negative Breast Cancer Sample

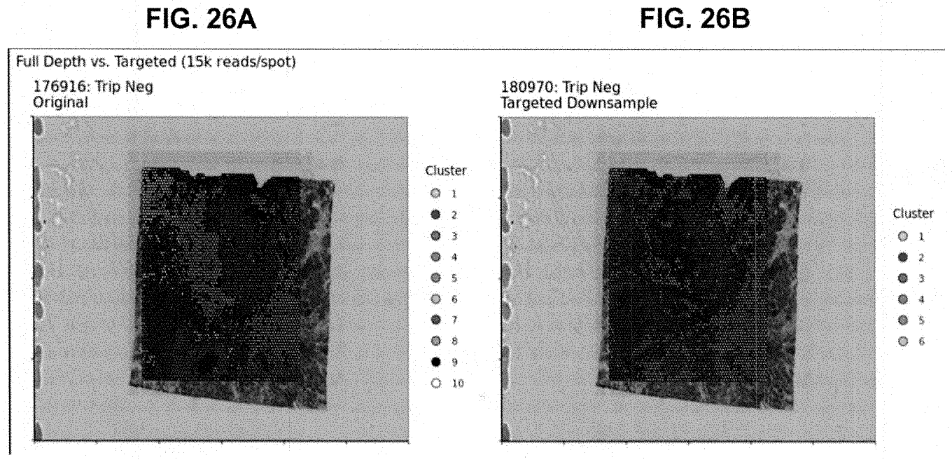

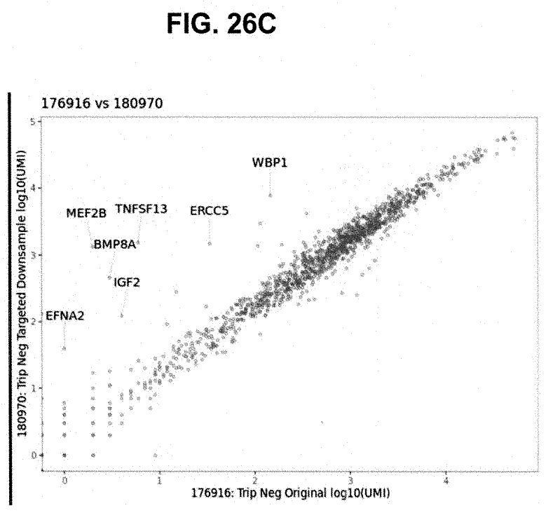

A triple negative breast cancer sample was prepared according to the steps in Example 1. Identification of genes of interest were determined using a cancer-specific panel looking at 1141 genes known to be involved in cancer biology (FIG. 26B) compared to a non-biased approach using probes to cover the entire transcriptome (FIG. 26A). Whole Transcriptome methods were used to sequence at ˜50k reads/spot. As shown in FIG. 26C, there was strong concordance between each group, demonstrating the comparative capability of using the cancer-specific panel instead of probes for the entire transcriptome. Each point in FIG. 26C represented the log 10 sum UMI of each 1141 pan cancer genes.

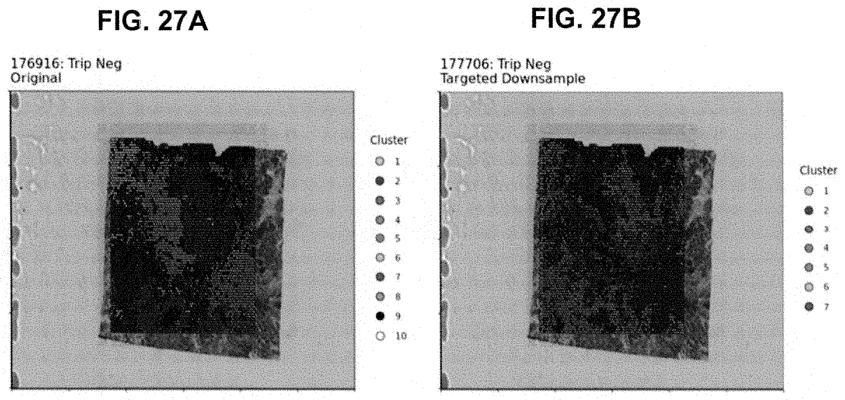

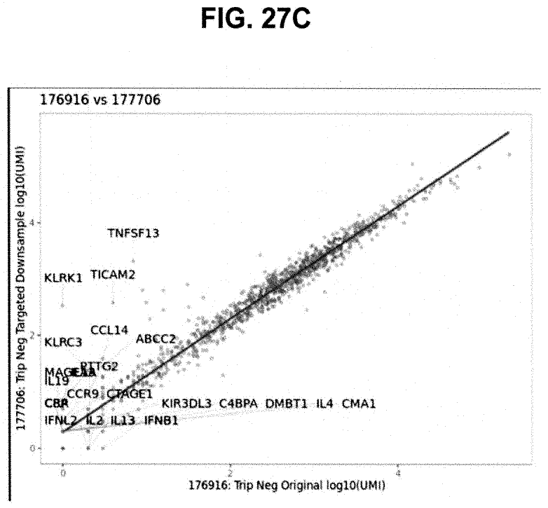

Next, identification of genes of interest were determined in the triple negative breast cancer sample using an immune-specific panel (FIG. 27B) compared to a non-biased approach using probes to cover the entire transcriptome (FIG. 27A). As shown in FIG. 27C, there was strong concordance between each group, demonstrating the comparative capability of using the cancer-specific panel instead of probes for the entire transcriptome.

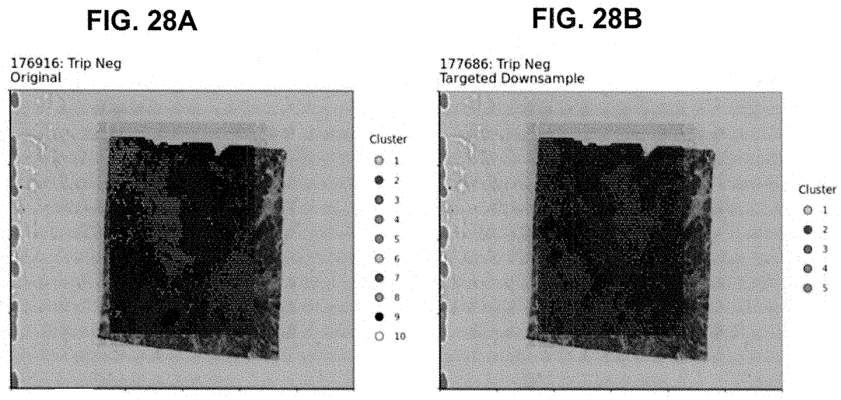

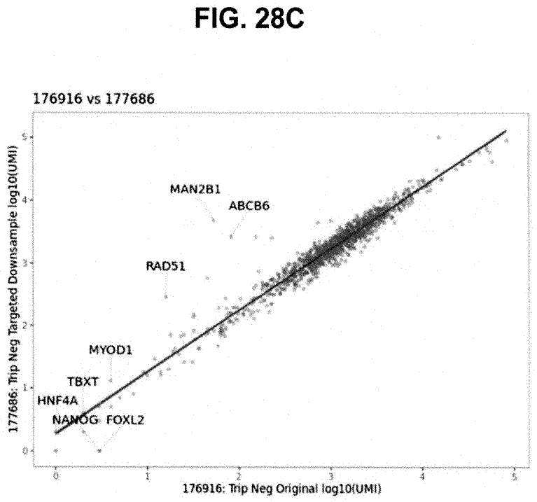

Next, identification of genes of interest were determined in the triple negative breast cancer sample using a pathway (i.e., transcription factor) specific panel (FIG. 28B) compared to a non-biased approach using probes to cover the entire transcriptome (FIG. 28A). As shown in FIG. 28C, there was strong concordance between each group, demonstrating the comparative capability of using the cancer-specific panel instead of probes for the entire transcriptome.

The bait oligonucleotides and target analytes for an exemplary cancer, immune, and pathway panels were disclosed in U.S. Appl. No. 62/970,066 (Titled “Capturing Targeted Genetic Targets Using A Hybridization/Capture Approach”) and U.S. Appl. No. 62/929,686, (Titled “Capturing Targeted Genetic Targets Using A Hybridization/Capture Approach”), each of which is incorporated by reference in its entirety.

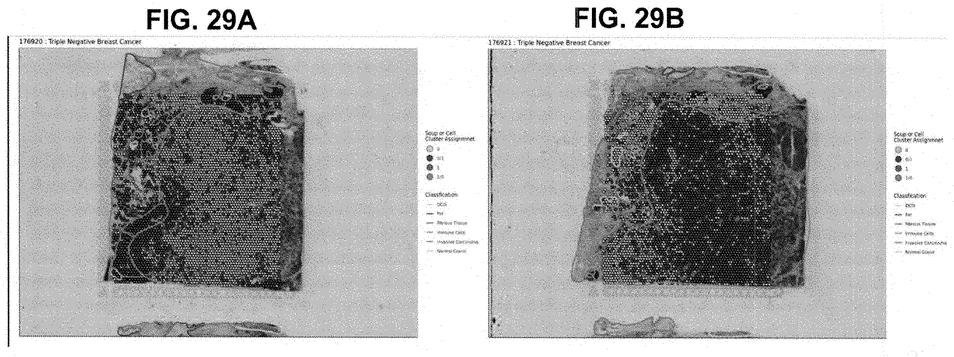

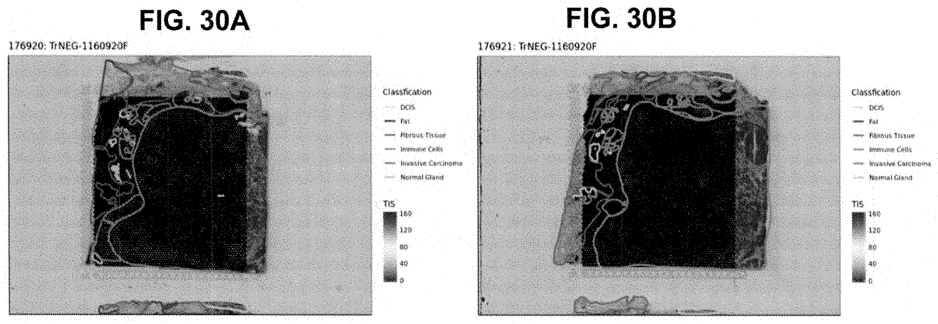

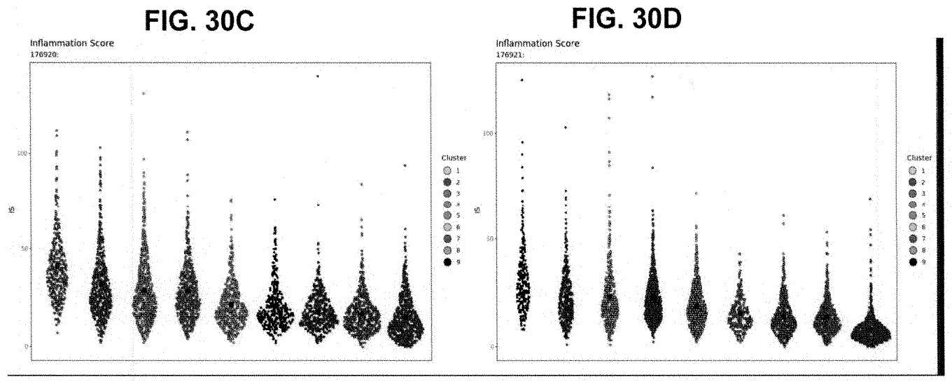

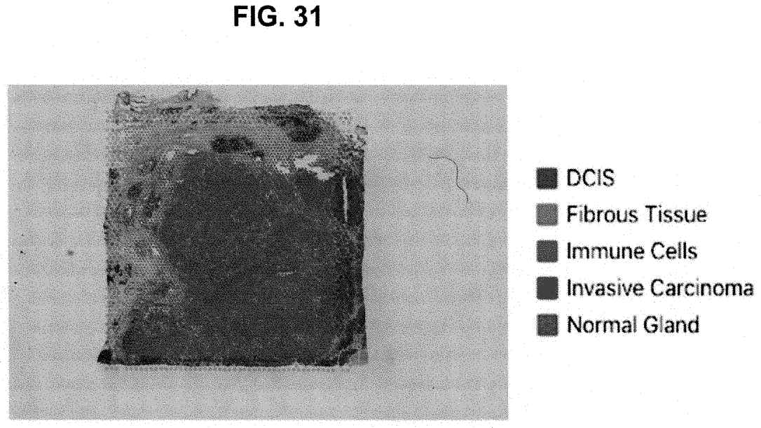

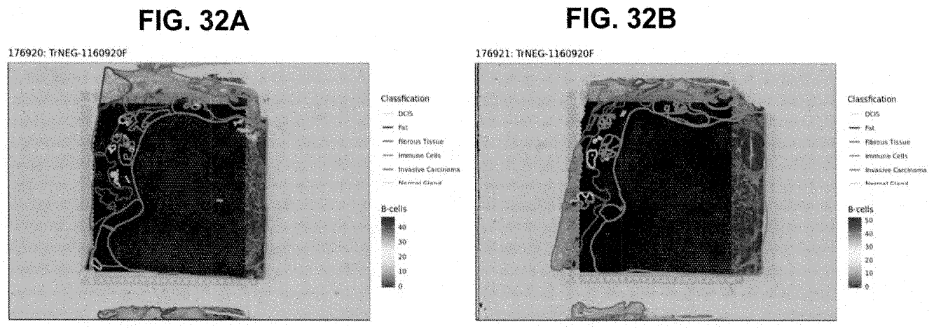

The triple negative breast cancer sample was examined by a pathologist, who identified areas of the sample as having areas that were cancerous or non-cancerous. See FIGS. 29A-32B. Based on the experiments, it was identified that CENPW, A2ML1, VLDLR, SCRG1, RCL1, FABP7, CCNE1, MIEN1, CDC37L1, ERMP1, and TNFSF10 were overexpressed in carcinoma cells. And, it was identified that ACTG2, DMKN, CALML3, COL17A1, MAGED1, PTN, TMEM98, LY6D, TNC, RTN4IP1 were overexpressed in DCIS cancer cells. See, e.g., Table 2.

TABLE 2
Transcripts with increased expression
(increased expression is shown in parentheses
relative to all other cells in same sample.

| Invasive Carcinoma vs. | | Ductal Carcinoma | |
|---|---|---|---|
| All | | In Situ vs. All | |
| FABP7 | (2.25) | ACTG2 | (5.09) |
| CENPW | (1.88) | DMKN | (3.84) |
| A2ML1 | (1.69) | CALML3 | (3.73) |
| SCRG1 | (1.68) | COL17A1 | (3.65) |
| VLDLR | (1.63) | MAGED1 | (3.49) |
| CDC37L1 | (1.55) | PTN | (3.25) |
| RCL1 | (1.53) | TMEM98 | (3.14) |
| ERMP1 | (1.53) | LY6D | (3.06) |
| TNFSF10 | (1.52) | TNC | (2.92) |

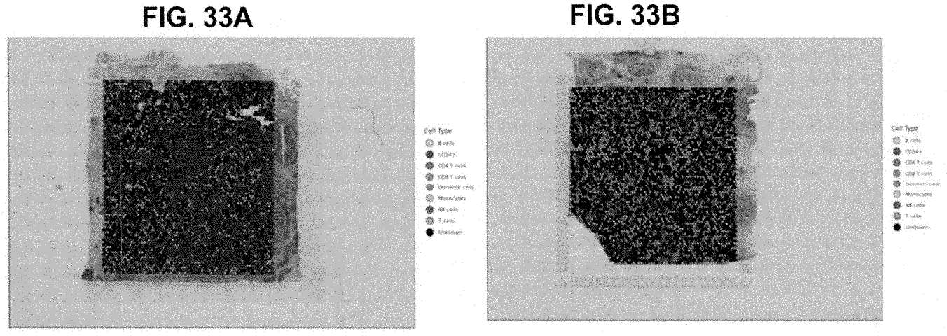

In addition, based on immune clustering, immune cells were identified. See FIGS. 33A-33B.

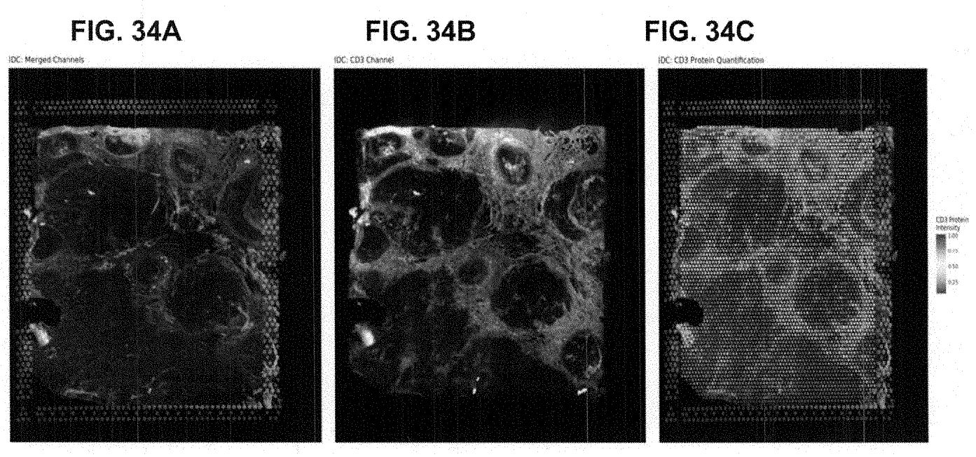

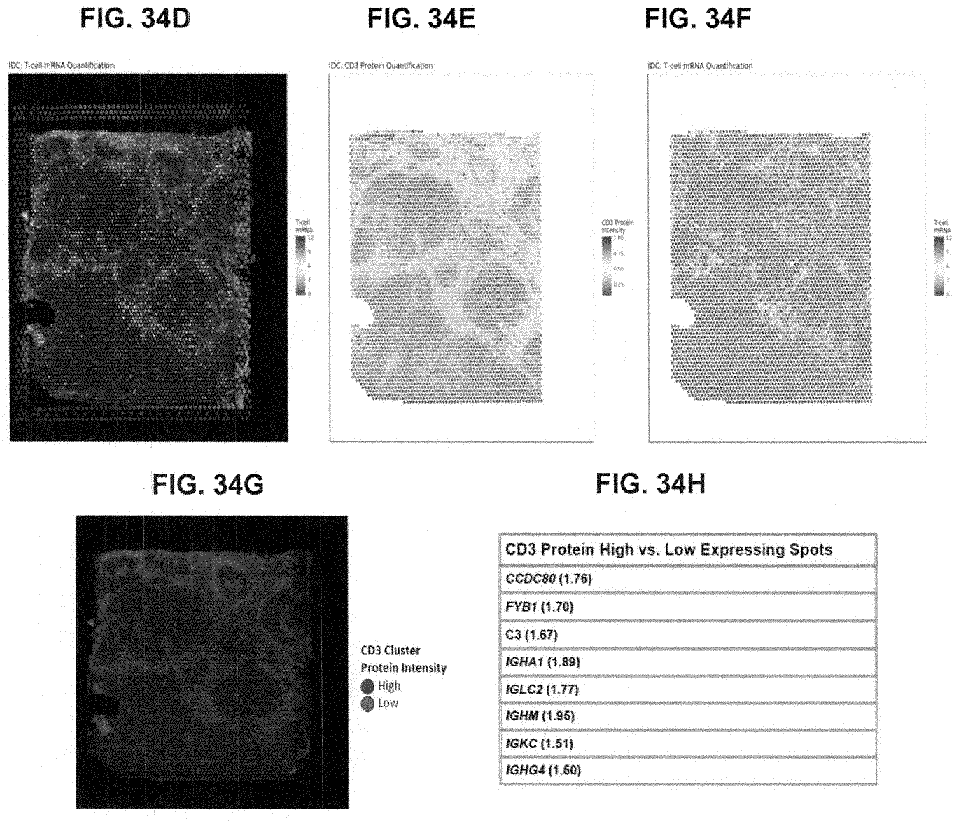

In addition to identifying analytes of interest using the enrichment methods described herein, additional methods were performed on the samples. For example, detection of CD3 was determined using immunofluorescence. As shown in FIGS. 34A-34H, CD3 detection was observed in samples that underwent enrichment analysis, demonstrating that one can overlap detection of a marker of interest with enrichment analysis.

### Example 3: Detection of Genes of Interest in DCIS Breast Cancer Sample

To further identify genes that are differentially expressed in a breast cancer sample, spatial gene expression was analyzed. A breast cancer tissue sample was serially sectioned and placed onto a gene expression slide. The sample was then fixed, stained, and permeabilized, releasing mRNA. The released mRNA hybridized to spatially barcoded capture probes on the slide, allowing for the capture of global gene expression information. cDNA was then synthesized from captured mRNA and sequencing libraries were prepared. The libraries were then sequenced and further analyzed.

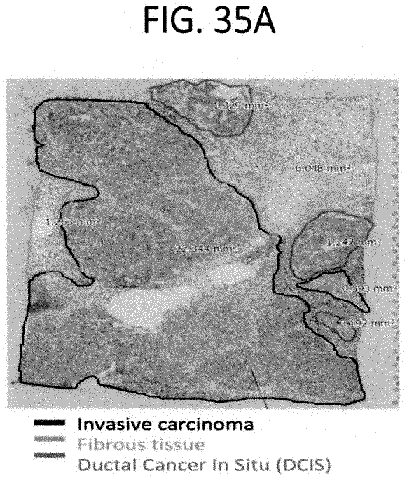

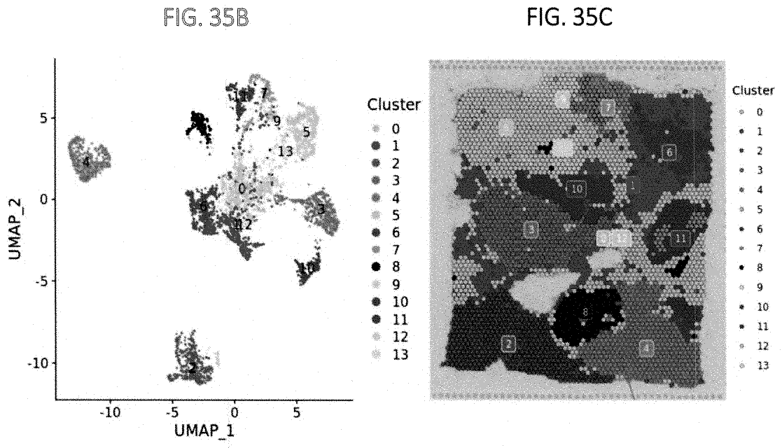

A pathologist annotated areas of the breast cancer tissue sample as invasive carcinoma, fibrous tissue, or ductal cancer in situ (DCIS). FIG. 35A. Gene expression clusters were identified using Seurat, which is an R package designed for quality control, analysis, and exploration of single-cell RNA-sequencing data. Seurat aims to enable users to identify and interpret sources of heterogeneity from single-cell transcriptomic measurements and to integrate diverse types of single-cell data. See Stuart et al. Cell, 2019 Jun. 13; 177(7):1888-1902.e21; Butler et al., Nat Biotechnol., 2018 June; 36(5):411-420, each of which is incorporated by reference in its entirety.

The sequencing data from cDNA libraries were further analyzed. Using Seurat, 14 clusters (number 0-13) were identified as containing differentially expressed genes compared to other areas of the breast cancer tissue shown in FIG. 35A. Gene expression of the 14 clusters was compared and plotted using Uniform Manifold Approximation and Projection (UMAP). FIG. 35B. See McInnes and Healy, UMAP: Uniform Manifold Approximation and Projection for Dimension Reduction, ArXiv e-prints 1802.03426, 2018, which is incorporated by reference in its entirety. Further, spatial cluster expression was determined. See FIG. 35C. Based on a comparison between pathology annotations (FIG. 35A) and tissue sample cluster expression (FIG. 35C), clusters 7 and 11 were detected in DCIS tissue; clusters 2, 3, 4, 5, 8, 10, 12, and 13 were detected in invasive carcinoma (IC); and clusters 0, 1, and 6 were detected in fibrous tissue. Cluster 9 was detected in areas identified as DCIS or IC. Consistent with the spatial expression identifying cluster 9 as either DCIS or IC, a UMAP graph places cluster 9 between clusters 7 and 11 (detected in DCIS tissue) and clusters 13 and 5 (detected in IC tissue), respectively. FIG. 35B. These data demonstrate that clustered gene expression can be used to identify areas of a breast cancer tissue such as IC, fibrous tissue, or DCIS tissue, and can be used to identify heterogenous expression in sub-areas within each pathologist-identified area.

Lists of the most differentiated genes in each cluster are provided in Tables 3-16 (Clusters 0-13) below. Upregulated genes are designated by an increase in the average log fold change (avg_logFC), whereas downregulated genes are designated by a decrease in the ave_logFC.

TABLE 3
Most differentially expressed genes in Cluster 0.

| Cluster | Gene | avg_logFC (Increase) | p_val | pct. 1 | pct. 2 | p_val_adj |
|---|---|---|---|---|---|---|
| 0 | IGLC2 | 2.237698576 | 9.874E−207 | 1 | 0.97 | 1.9502E−202 |
| 0 | IGHG3 | 1.969278877 | 6.4947E−207 | 1 | 1 | 1.2828E−202 |
| 0 | IGKC | 1.927099555 | 4.2908E−208 | 1 | 1 | 8.4747E−204 |
| 0 | IGHG1 | 1.871963119 | 8.4732E−226 | 1 | 0.987 | 1.6735E−221 |
| 0 | IGLC3 | 1.8685086 | 7.1717E−160 | 0.949 | 0.617 | 1.4165E−155 |
| 0 | IGHA1 | 1.659076716 | 7.9596E−138 | 0.998 | 0.95 | 1.5721E−133 |
| 0 | IGHG2 | 1.609732309 | 6.45414E−90 | 0.731 | 0.433 | 1.27476E−85 |
| 0 | IGHM | 1.578956691 | 7.9731E−126 | 0.978 | 0.804 | 1.5748E−121 |
| 0 | IGHG4 | 1.445300704 | 8.1275E−196 | 1 | 1 | 1.6053E−191 |
| 0 | JCHAIN | 1.442206837 | 4.7992E−116 | 0.738 | 0.389 | 9.4789E−112 |
| Cluster | Gene | avg_logFC (Decrease) | p_val | pct. 1 | pct. 2 | p_val_adj |
| 0 | ID3 | 0.250178206 | 2.04457E−15 | 0.599 | 0.511 | 4.03822E−11 |
| 0 | CIITA | 0.250796336 | 1.22165E−18 | 0.42 | 0.267 | 2.41288E−14 |
| 0 | CST7 | 0.251487743 | 2.18694E−27 | 0.323 | 0.147 | 4.31943E−23 |
| 0 | IFI27L2 | 0.251944559 | 2.48422E−16 | 0.565 | 0.453 | 4.90658E−12 |
| 0 | FYN | 0.25199912 | 3.63133E−23 | 0.345 | 0.18 | 7.17225E−19 |
| 0 | MICAL1 | 0.252555589 | 2.10911E−16 | 0.501 | 0.367 | 4.1657E−12 |
| 0 | HMOX1 | 0.252966034 | 1.05706E−10 | 0.418 | 0.318 | 2.0878E−06 |
| 0 | CD7 | 0.253105009 | 1.58297E−16 | 0.409 | 0.272 | 3.12652E−12 |
| 0 | ARHGEF1 | 0.254803199 | 5.90394E−20 | 0.816 | 0.769 | 1.16609E−15 |
| 0 | CFH | 0.25502568 | 5.29489E−21 | 0.453 | 0.291 | 1.04579E−16 |

TABLE 4
Most differentially expressed genes in Cluster 1.

| Cluster | Gene | avg_logFC (Increase) | p_val | pct. 1 | pct. 2 | p_val_adj |
|---|---|---|---|---|---|---|
| 1 | MALAT1 | 0.76555696 | 1.9285E−132 | 1 | 0.998 | 3.809E−128 |
| 1 | CTSD | 0.322061763 | 1.06506E−67 | 1 | 1 | 2.10361E−63 |
| 1 | TYMP | 0.309075856 | 2.21042E−42 | 1 | 0.988 | 4.3658E−38 |
| 1 | SAMHD1 | 0.300996766 | 3.4535E−21 | 0.896 | 0.652 | 6.82102E−17 |
| 1 | CYBA | 0.2885973 | 6.78103E−35 | 0.998 | 0.874 | 1.33932E−30 |
| 1 | ISG15 | 0.288174446 | 9.97411E−37 | 1 | 1 | 1.96999E−32 |
| 1 | C1QA | 0.281999783 | 2.05697E−40 | 1 | 0.903 | 4.06272E−36 |
| 1 | RPS9 | 0.27786914 | 2.17818E−70 | 1 | 1 | 4.30212E−66 |
| 1 | H2AFJ | 0.276362387 | 3.23165E−26 | 1 | 0.999 | 6.38283E−22 |
| 1 | ADIRF | 0.276352352 | 9.25273E−41 | 1 | 1 | 1.82751E−36 |
| Cluster | Gene | avg_logFC (Decrease) | p_val | pct. 1 | pct. 2 | p_val_adj |
| 1 | ARHGDIA | 0.252646375 | 1.09333E−27 | 1 | 0.999 | 2.15943E−23 |
| 1 | APRT | 0.252736884 | 2.08263E−36 | 1 | 1 | 4.1134E−32 |
| 1 | AEBP1 | 0.254118619 | 2.90888E−37 | 1 | 0.94 | 5.74533E−33 |
| 1 | PLEC | 0.2577431 | 2.73281E−25 | 1 | 0.94 | 5.39757E−21 |
| 1 | APOE | 0.262450453 | 1.98698E−44 | 1 | 1 | 3.92448E−40 |
| 1 | FCGRT | 0.263904163 | 3.83161E−23 | 1 | 0.91 | 7.56782E−19 |
| 1 | NDUFB7 | 0.266525453 | 4.21567E−31 | 1 | 0.999 | 8.32636E−27 |
| 1 | MBD3 | 0.267243753 | 2.20384E−15 | 0.95 | 0.758 | 4.3528E−11 |
| 1 | EMILIN1 | 0.269909237 | 4.98053E−28 | 0.921 | 0.63 | 9.83704E−24 |
| 1 | GADD45GIP1 | 0.270198314 | 3.5038E−23 | 0.998 | 0.976 | 6.92036E−19 |

TABLE 5
Most differentially expressed genes in Cluster 2.

| Cluster | Gene | avg_logFC (Increase) | p_val | pct. 1 | pct. 2 | p_val_adj |
|---|---|---|---|---|---|---|
| 2 | CXCL14 | 1.873297518 | 1.2773E−247 | 1 | 0.959 | 2.5228E−243 |
| 2 | TTLL12 | 1.445474956 | 9.8846E−233 | 0.995 | 0.871 | 1.9523E−228 |
| 2 | GFRA1 | 1.317657382 | 4.4333E−235 | 1 | 0.945 | 8.7562E−231 |
| 2 | DEGS1 | 1.213369592 | 7.1204E−212 | 1 | 0.928 | 1.4064E−207 |
| 2 | AGR2 | 1.127044122 | 2.2901E−222 | 1 | 0.996 | 4.5233E−218 |
| 2 | ARMT1 | 1.116280085 | 3.4403E−150 | 0.965 | 0.771 | 6.795E−146 |
| 2 | CCND1 | 1.08106123 | 7.3788E−241 | 1 | 1 | 1.4574E−236 |
| 2 | ARPP21 | 1.076105815 | 0 | 0.826 | 0.104 | 0 |
| 2 | CRAT | 0.9897182 | 3.7078E−203 | 0.981 | 0.528 | 7.3233E−199 |
| 2 | PRKACB | 0.983798671 | 3.7861E−193 | 0.984 | 0.573 | 7.478E−189 |
| Cluster | Gene | avg_logFC (Decrease) | p_val | pct. 1 | pct. 2 | p_val_adj |
| 2 | NSD3 | 0.250253912 | 4.50449E−25 | 0.81 | 0.646 | 8.89681E−21 |
| 2 | PLAUR | 0.250509089 | 1.18007E−30 | 0.588 | 0.301 | 2.33075E−26 |
| 2 | CDKN2C | 0.25052229 | 7.03066E−45 | 0.431 | 0.153 | 1.38862E−40 |
| 2 | FIP1L1 | 0.250833324 | 1.02448E−26 | 0.613 | 0.35 | 2.02344E−22 |
| 2 | TMEM159 | 0.2509697 | 1.38051E−27 | 0.667 | 0.394 | 2.72665E−23 |
| 2 | TMEM141 | 0.25121772 | 5.44499E−30 | 0.984 | 0.953 | 1.07544E−25 |
| 2 | LIN7C | 0.251234768 | 9.94912E−31 | 0.688 | 0.389 | 1.96505E−26 |
| 2 | ARHGEF39 | 0.251245333 | 5.73091E−53 | 0.456 | 0.148 | 1.13191E−48 |
| 2 | ARFGEF3 | 0.251555275 | 1.43128E−29 | 0.681 | 0.388 | 2.82691E−25 |
| 2 | EMP2 | 0.251573293 | 1.63626E−24 | 0.85 | 0.714 | 3.23178E−20 |

TABLE 6
Most differentially expressed genes in Cluster 3.

| Cluster | Gene | avg_logFC (Increase) | p_val | pct. 1 | pct. 2 | p_val_adj |
|---|---|---|---|---|---|---|
| 3 | CPB1 | 1.813546977 | 4.8082E−198 | 1 | 0.928 | 9.4967E−194 |
| 3 | FCGR3B | 1.312602264 | 2.3751E−183 | 0.861 | 0.262 | 4.6911E−179 |
| 3 | KLHDC7B | 0.963524765 | 9.7325E−120 | 0.913 | 0.518 | 1.9223E−115 |
| 3 | SCGB1D2 | 0.881685998 | 3.22688E−83 | 0.97 | 0.782 | 6.37341E−79 |
| 3 | SCUBE3 | 0.797313921 | 2.1631E−170 | 0.837 | 0.231 | 4.2724E−166 |
| 3 | CXCL9 | 0.790154947 | 1.21794E−60 | 0.903 | 0.7 | 2.40555E−56 |
| 3 | COX6C | 0.788284816 | 1.6234E−137 | 1 | 1 | 3.2064E−133 |
| 3 | CFB | 0.767466081 | 9.9425E−127 | 0.995 | 0.966 | 1.9638E−122 |
| 3 | SCGB2A2 | 0.747428171 | 2.95969E−71 | 0.988 | 0.821 | 5.84569E−67 |
| 3 | NPY1R | 0.736002145 | 5.3061E−109 | 0.47 | 0.092 | 1.048E−104 |
| Cluster | Gene | avg_logFC (Decrease) | p_val | pct. 1 | pct. 2 | p_val_adj |
| 3 | AC087741.1 | 0.250441692 | 1.25487E−31 | 0.502 | 0.236 | 2.47849E−27 |
| 3 | GBP5 | 0.250653298 | 1.09264E−21 | 0.433 | 0.222 | 2.15807E−17 |
| 3 | IFT27 | 0.2506927 | 1.77701E−27 | 0.71 | 0.417 | 3.50977E−23 |
| 3 | NEURL4 | 0.251015027 | 8.51238E−35 | 0.584 | 0.278 | 1.68128E−30 |
| 3 | EMX1 | 0.251397736 | 1.72136E−47 | 0.359 | 0.104 | 3.39985E−43 |
| 3 | SLC13A2 | 0.251482828 | 1.26552E−38 | 0.413 | 0.151 | 2.49954E−34 |
| 3 | FAM110A | 0.251756928 | 5.76693E−22 | 0.946 | 0.911 | 1.13903E−17 |
| 3 | SPCS1 | 0.25179759 | 1.57136E−34 | 0.998 | 0.989 | 3.1036E−30 |
| 3 | HGD | 0.251819872 | 4.74587E−38 | 0.446 | 0.17 | 9.37357E−34 |
| 3 | ZNF587 | 0.251876129 | 2.46484E−31 | 0.594 | 0.293 | 4.86832E−27 |

TABLE 7
Most differentially expressed genes in Cluster 4.

| Cluster | Gene | avg_logFC (Increase) | p_val | pct. 1 | pct. 2 | p_val_adj |
|---|---|---|---|---|---|---|
| 4 | CRISP3 | 2.606820862 | 2.548E−239 | 1 | 0.801 | 5.0325E−235 |
| 4 | SLITRK6 | 1.815692524 | 7.0593E−276 | 0.995 | 0.369 | 1.3943E−271 |
| 4 | C6orf141 | 1.429567269 | 1.9098E−236 | 0.977 | 0.39 | 3.772E−232 |
| 4 | VTCN1 | 1.205762478 | 1.1731E−195 | 0.992 | 0.565 | 2.3169E−191 |
| 4 | SERHL2 | 1.035361703 | 8.2259E−177 | 0.997 | 0.952 | 1.6247E−172 |
| 4 | CEACAM6 | 0.869552995 | 2.2032E−169 | 0.914 | 0.304 | 4.3516E−165 |
| 4 | ABCC11 | 0.858101263 | 8.065E−171 | 0.818 | 0.216 | 1.5929E−166 |
| 4 | SHISA2 | 0.841892886 | 1.8502E−101 | 0.924 | 0.596 | 3.65428E−97 |
| 4 | C2orf54 | 0.840209125 | 7.6291E−179 | 0.858 | 0.236 | 1.5068E−174 |
| 4 | PDLIM1 | 0.827500664 | 4.4222E−135 | 0.98 | 0.787 | 8.7343E−131 |
| Cluster | Gene | avg_logFC (Decrease) | p_val | pct. 1 | pct. 2 | p_val_adj |
| 4 | ZC3H12A | 0.25019439 | 2.6266E−34 | 0.559 | 0.261 | 5.18781E−30 |
| 4 | VPS37B | 0.250812072 | 7.14498E−28 | 0.608 | 0.333 | 1.41121E−23 |
| 4 | IRF2BP2 | 0.250861762 | 2.73864E−30 | 0.995 | 0.987 | 5.40908E−26 |
| 4 | RAPH1 | 0.251221523 | 4.552E−31 | 0.572 | 0.284 | 8.99066E−27 |
| 4 | NFKBIA | 0.251297742 | 1.34827E−16 | 0.954 | 0.958 | 2.66297E−12 |
| 4 | EIF2AK1 | 0.251322666 | 2.57398E−27 | 0.934 | 0.868 | 5.08388E−23 |
| 4 | TRIM33 | 0.2514734 | 2.19479E−30 | 0.592 | 0.301 | 4.33493E−26 |
| 4 | SFPQ | 0.251550205 | 1.28212E−27 | 0.987 | 0.958 | 2.53231E−23 |
| 4 | F7 | 0.251667544 | 3.4573E−30 | 0.605 | 0.308 | 6.8285E−26 |
| 4 | TRAPPC3 | 0.251990477 | 7.40789E−23 | 0.762 | 0.553 | 1.46313E−18 |

TABLE 8
Most differentially expressed genes in Cluster 5.

| Cluster | Gene | avg_logFC (Increase) | p_val | pct. 1 | pct. 2 | p_val_adj |
|---|---|---|---|---|---|---|
| 5 | LINC00052 | 0.932352783 | 2.0054E−164 | 0.609 | 0.092 | 3.9609E−160 |
| 5 | COX6C | 0.931214106 | 4.2849E−142 | 1 | 1 | 8.463E−138 |
| 5 | SNCG | 0.919464458 | 4.6901E−123 | 0.997 | 0.74 | 9.2633E−119 |
| 5 | WFDC2 | 0.850299518 | 5.0658E−115 | 0.972 | 0.655 | 1.0005E−110 |
| 5 | SLC39A6 | 0.842272163 | 2.1966E−139 | 1 | 1 | 4.3385E−135 |
| 5 | MGST1 | 0.792992516 | 5.35745E−55 | 0.668 | 0.324 | 1.05815E−50 |
| 5 | MCCD1 | 0.783075139 | 6.3641E−103 | 1 | 0.728 | 1.25697E−98 |
| 5 | CSTA | 0.762385432 | 1.31863E−91 | 1 | 0.929 | 2.60443E−87 |
| 5 | PDE5A | 0.745956824 | 2.48765E−97 | 0.818 | 0.328 | 4.91336E−93 |
| 5 | MT-ND1 | 0.714131921 | 2.0471E−110 | 1 | 1 | 4.0432E−106 |
| Cluster | Gene | avg_logFC (Decrease) | p_val | pct. 1 | pct. 2 | p_val_adj |
| 5 | SRP14 | 0.250014586 | 9.9687E−30 | 1 | 1 | 1.96892E−25 |
| 5 | TRAPPC1 | 0.250231971 | 1.21924E−21 | 0.966 | 0.925 | 2.40812E−17 |
| 5 | SNRPD3 | 0.250640943 | 4.05507E−24 | 0.982 | 0.949 | 8.00917E−20 |
| 5 | MAT2A | 0.251201295 | 7.11429E−22 | 0.994 | 0.98 | 1.40514E−17 |
| 5 | SLC7A8 | 0.251686529 | 4.91604E−12 | 0.763 | 0.66 | 9.70967E−08 |
| 5 | POLR2K | 0.251970379 | 2.92774E−25 | 0.997 | 0.993 | 5.78257E−21 |
| 5 | TMBIM6 | 0.25203555 | 3.23036E−46 | 1 | 1 | 6.38028E−42 |
| 5 | OCIAD1 | 0.25256478 | 1.80539E−19 | 0.917 | 0.894 | 3.56584E−15 |
| 5 | EXOSC3 | 0.253204453 | 1.69102E−15 | 0.745 | 0.578 | 3.33994E−11 |
| 5 | CA14 | 0.255572019 | 2.4818E−30 | 0.372 | 0.135 | 4.90181E−26 |

TABLE 9
Most differentially expressed genes in Cluster 6.

| Cluster | Gene | avg_logFC (Increase) | p_val | pct. 1 | pct. 2 | p_val_adj |
|---|---|---|---|---|---|---|
| 6 | ACKR1 | 1.164188559 | 6.20534E−72 | 0.551 | 0.158 | 1.22562E−67 |
| 6 | IGFBP7 | 1.155549296 | 4.2857E−101 | I | 0.972 | 8.46469E−97 |
| 6 | AQP1 | 1.148352788 | 8.43379E−59 | 0.745 | 0.418 | 1.66576E−54 |
| 6 | VWF | 1.132634271 | 1.72739E−68 | 0.955 | 0.62 | 3.41178E−64 |
| 6 | MALAT1 | 1.097207191 | 4.7557E−100 | 1 | 0.999 | 9.39293E−96 |
| 6 | SPARCL1 | 1.03827919 | 1.90465E−71 | 0.992 | 0.711 | 3.76188E−67 |
| 6 | TAGLN | 1.036652912 | 3.55202E−70 | 1 | 0.799 | 7.01559E−66 |
| 6 | CCL21 | 1.008295494 | 1.49472E−39 | 0.409 | 0.131 | 2.95222E−35 |
| 6 | ACTA2 | 0.996706517 | 2.74918E−49 | 0.968 | 0.687 | 5.42991E−45 |
| 6 | CCDC80 | 0.983680244 | 7.12231E−81 | 0.976 | 0.567 | 1.40673E−76 |
| Cluster | Gene | avg_logFC (Decrease) | p_val | pct. 1 | pct. 2 | p_val_adj |
| 6 | CYB5R3 | 0.25163237 | 6.42024E−09 | 0.992 | 0.889 | 0.000126806 |
| 6 | PLXND1 | 0.251694121 | 3.32911E−08 | 0.992 | 0.87 | 0.000657533 |
| 6 | DUSP1 | 0.251949699 | 3.42806E−08 | 1 | 0.965 | 0.000677075 |
| 6 | RPS6 | 0.252683686 | 4.58295E−29 | 1 | 1 | 9.05179E−25 |
| 6 | C1QA | 0.253357634 | 1.09397E−15 | 1 | 0.91 | 2.1607E−11 |
| 6 | HEG1 | 0.254800252 | 0.000174538 | 0.279 | 0.213 | 1 |
| 6 | ETS2 | 0.25641834 | 0.001070649 | 0.32 | 0.273 | 1 |
| 6 | AKAP9 | 0.257293084 | 0.003696097 | 0.409 | 0.415 | 1 |
| 6 | CCAR1 | 0.257652478 | 2.56255E−10 | 0.826 | 0.633 | 5.0613E−06 |
| 6 | TRIM47 | 0.257710346 | 1.9105E−10 | 0.887 | 0.704 | 3.77342E−06 |

TABLE 10
Most differentially expressed genes in Cluster 7.

| Cluster | Gene | avg_logFC (Increase) | p_val | pct. 1 | pct. 2 | p_val_adj |
|---|---|---|---|---|---|---|
| 7 | ALB | 3.018020845 | 3.7726E−68 | 0.91 | 0.596 | 7.45127E−64 |
| 7 | MGP | 1.857551944 | 2.92965E−93 | 1 | 1 | 5.78636E−89 |
| 7 | ZNF350-AS1 | 1.654270953 | 5.7211E−109 | 0.862 | 0.231 | 1.13E−104 |
| 7 | S100G | 1.516630368 | 1.66337E−81 | 1 | 0.882 | 3.28532E−77 |
| 7 | STC2 | 1.197048816 | 1.755E−75 | 0.994 | 0.913 | 3.4663E−71 |
| 7 | CARTPT | 1.161620086 | 4.75215E−74 | 0.641 | 0.146 | 9.38598E−70 |
| 7 | AC087379.2 | 1.128126745 | 8.79613E−53 | 0.731 | 0.267 | 1.73732E−48 |
| 7 | GPC3 | 1.077283133 | 5.18464E−74 | 0.862 | 0.353 | 1.02402E−69 |
| 7 | ERP27 | 1.063082306 | 1.04333E−67 | 0.808 | 0.306 | 2.06068E−63 |
| 7 | APOD | 1.037935962 | 1.24707E−49 | 0.934 | 0.692 | 2.46309E−45 |
| Cluster | Gene | avg_logFC (Decrease) | p_val | pct. 1 | pct. 2 | p_val_adj |
| 7 | CDV3 | 0.250134338 | 5.36348E−10 | 0.91 | 0.871 | 1.05934E−05 |
| 7 | TPI1 | 0.25129609 | 1.36497E−13 | 1 | 0.999 | 2.69596E−09 |
| 7 | TSPYL5 | 0.251830782 | 2.86966E−11 | 0.844 | 0.718 | 5.66786E−07 |
| 7 | PFKP | 0.252016961 | 7.62543E−08 | 0.629 | 0.474 | 0.001506099 |
| 7 | CRIM1 | 0.252263553 | 1.53415E−11 | 0.515 | 0.286 | 3.03009E−07 |
| 7 | PPT1 | 0.253111642 | 2.97806E−13 | 1 | 0.962 | 5.88198E−09 |
| 7 | AC100826.1 | 0.254598773 | 4.48212E−26 | 0.365 | 0.102 | 8.85264E−22 |
| 7 | ALDOA | 0.255033207 | 3.97131E−09 | 0.85 | 0.767 | 7.84374E−05 |
| 7 | LGR4 | 0.255225468 | 7.34236E−22 | 0.413 | 0.142 | 1.45019E−17 |
| 7 | GLRX2 | 0.255424409 | 2.3313E−10 | 0.605 | 0.387 | 4.60455E−06 |

TABLE 11
Most differentially expressed genes in Cluster 8.

| Cluster | Gene | avg_logFC (Increase) | p_val | pct. 1 | pct. 2 | p_val_adj |
|---|---|---|---|---|---|---|
| 8 | AC087379.2 | 1.522104743 | 2.0576E−137 | 0.982 | 0.256 | 4.064E−133 |
| 8 | S100G | 1.306401389 | 1.23714E−85 | 1 | 0.882 | 2.44348E−81 |
| 8 | SCGB2A2 | 1.19728421 | 2.00029E−58 | 0.994 | 0.831 | 3.95076E−54 |
| 8 | PGM5-AS1 | 1.122790247 | 9.8375E−210 | 0.768 | 0.068 | 1.943E−205 |
| 8 | HEBP1 | 1.047481665 | 8.87729E−78 | 1 | 0.952 | 1.75335E−73 |
| 8 | AMIGO2 | 1.028239612 | 6.52522E−84 | 0.933 | 0.369 | 1.2888E−79 |
| 8 | PCED1B | 0.998411713 | 9.52857E−78 | 0.982 | 0.711 | 1.88199E−73 |
| 8 | SCGB1D2 | 0.95963851 | 2.65838E−43 | 0.97 | 0.794 | 5.25056E−39 |
| 8 | ITPR1 | 0.939019125 | 9.19629E−70 | 0.982 | 0.723 | 1.81636E−65 |
| 8 | IFT122 | 0.93503288 | 3.00484E−79 | 1 | 0.963 | 5.93486E−75 |
| Cluster | Gene | avg_logFC (Decrease) | p_val | pct. 1 | pct. 2 | p_val_adj |
| 8 | GABARAPL1 | 0.250341138 | 2.71991E−11 | 0.549 | 0.315 | 5.3721E−07 |
| 8 | G6PC3 | 0.250432894 | 2.34275E−13 | 0.988 | 0.954 | 4.62717E−09 |
| 8 | INPP5K | 0.250631086 | 1.42594E−11 | 0.817 | 0.583 | 2.81637E−07 |
| 8 | STC2 | 0.251084053 | 3.03925E−09 | 0.951 | 0.915 | 6.00282E−05 |
| 8 | HEXIM2 | 0.251279917 | 4.76458E−09 | 0.726 | 0.521 | 9.41052E−05 |
| 8 | RIDA | 0.252173971 | 5.80131E−12 | 0.713 | 0.463 | 1.14582E−07 |
| 8 | LRP2 | 0.252242655 | 2.33188E−16 | 0.518 | 0.233 | 4.6057E−12 |
| 8 | HK2 | 0.252549276 | 2.65516E−12 | 0.902 | 0.752 | 5.24421E−08 |
| 8 | IL1R2 | 0.252817886 | 2.29597E−24 | 0.378 | 0.112 | 4.53477E−20 |
| 8 | GOT2 | 0.253194125 | 8.64267E−11 | 0.951 | 0.933 | 1.70701E−06 |

TABLE 12
Most differentially expressed genes in Cluster 9.

| Cluster | Gene | avg_logFC (Increase) | p_val | pct. 1 | pct. 2 | p_val_adj |
|---|---|---|---|---|---|---|
| 9 | ALB | 1.128772536 | 3.09084E−38 | 0.859 | 0.598 | 6.10471E−34 |
| 9 | MT-ND2 | 0.833165922 | 1.09533E−76 | 1 | 1 | 2.16338E−72 |
| 9 | MT-ND1 | 0.710992385 | 1.19906E−72 | 1 | 1 | 2.36826E−68 |
| 9 | MT-ND3 | 0.697529604 | 7.52593E−74 | 1 | 1 | 1.48645E−69 |
| 9 | MT-ATP6 | 0.689210803 | 2.78756E−74 | 1 | 1 | 5.5057E−70 |
| 9 | MT-ND4 | 0.622259532 | 7.89828E−72 | 1 | 1 | 1.55999E−67 |
| 9 | MT-CO1 | 0.608445612 | 2.58326E−60 | 1 | 1 | 5.10219E−56 |
| 9 | MT-CO3 | 0.564176502 | 2.38763E−68 | 1 | 1 | 4.71581E−64 |
| 9 | MT-ATP8 | 0.553967653 | 2.34899E−26 | 0.675 | 0.371 | 4.6395E−22 |
| 9 | MT-ND5 | 0.543556469 | 1.60679E−32 | 1 | 0.972 | 3.17357E−28 |
| Cluster | Gene | avg_logFC (Decrease) | p_val | pct. 1 | pct. 2 | p_val_adj |
| 9 | GLO1 | 0.256088408 | 5.38322E−09 | 1 | 0.967 | 0.000106324 |
| 9 | ABHD2 | 0.256260458 | 1.20616E−12 | 1 | 0.931 | 2.38229E−08 |
| 9 | LINC00052 | 0.256816809 | 1.63578E−13 | 0.325 | 0.128 | 3.23082E−09 |
| 9 | RERG | 0.258917705 | 1.91148E−11 | 0.951 | 0.811 | 3.77536E−07 |
| 9 | STC2 | 0.273505647 | 3.20466E−11 | 0.988 | 0.914 | 6.32952E−07 |
| 9 | MALAT1 | 0.276485039 | 2.92227E−17 | 1 | 0.999 | 5.77177E−13 |
| 9 | SOX4 | 0.279350546 | 9.12168E−08 | 0.988 | 0.927 | 0.001801623 |
| 9 | CA12 | 0.280510067 | 2.33579E−11 | 0.982 | 0.915 | 4.61342E−07 |
| 9 | ZNF703 | 0.296272389 | 1.89071E−21 | 1 | 1 | 3.73434E−17 |
| 9 | MCCD1 | 0.299751994 | 7.81129E−13 | 0.957 | 0.742 | 1.54281E−08 |

TABLE 13
Most differentially expressed genes in Cluster 10.

| Cluster | Gene | avg_logFC (Increase) | p_val | pct. 1 | pct. 2 | p_val_adj |
|---|---|---|---|---|---|---|
| 10 | LINC00645 | 1.554766862 | 2.4267E−152 | 0.968 | 0.197 | 4.7929E−148 |
| 10 | SLC30A8 | 1.15206813 | 2.66089E−91 | 0.885 | 0.273 | 5.25551E−87 |
| 10 | MUC5B | 0.970379983 | 6.16705E−74 | 1 | 0.913 | 1.21805E−69 |
| 10 | COLEC12 | 0.914745725 | 3.87756E−57 | 0.968 | 0.718 | 7.65858E−53 |
| 10 | PVALB | 0.900229898 | 2.43034E−70 | 1 | 0.871 | 4.80017E−66 |
| 10 | CPB1 | 0.864886628 | 8.42462E−28 | 1 | 0.933 | 1.66395E−23 |
| 10 | EXOC2 | 0.861629448 | 9.11902E−63 | 0.974 | 0.736 | 1.8011E−58 |
| 10 | AC037198.2 | 0.752617236 | 5.18903E−69 | 0.731 | 0.199 | 1.02489E−64 |
| 10 | VSTM2A | 0.737447794 | 2.17124E−45 | 0.891 | 0.477 | 4.28842E−41 |
| 10 | FSIP1 | 0.721439645 | 6.21151E−60 | 0.801 | 0.288 | 1.22683E−55 |
| Cluster | Gene | avg_logFC (Decrease) | p_val | pct. 1 | pct. 2 | p_val_adj |
| 10 | KIF16B | 0.250136535 | 2.97876E−09 | 0.545 | 0.336 | 5.88335E−05 |
| 10 | SPATA20 | 0.250274461 | 1.06422E−12 | 0.929 | 0.866 | 2.10195E−08 |
| 10 | TSPAN9 | 0.250994922 | 1.4495E−11 | 0.859 | 0.627 | 2.86291E−07 |
| 10 | CACNB3 | 0.251004717 | 9.29225E−10 | 0.615 | 0.381 | 1.83531E−05 |
| 10 | CAMK2N1 | 0.251422988 | 1.88546E−10 | 0.891 | 0.789 | 3.72398E−06 |
| 10 | IFT27 | 0.251458321 | 1.52644E−10 | 0.679 | 0.438 | 3.01486E−06 |
| 10 | NEURL1 | 0.252584022 | 2.52458E−10 | 0.84 | 0.637 | 4.98629E−06 |
| 10 | TRIM3 | 0.252765807 | 4.58074E−09 | 0.756 | 0.591 | 9.04741E−05 |
| 10 | SLC46A1 | 0.252878017 | 2.57216E−13 | 0.699 | 0.402 | 5.08028E−09 |
| 10 | ECI2 | 0.253214426 | 2.3343E−08 | 0.846 | 0.77 | 0.000461048 |

TABLE 14
Most differentially expressed genes in Cluster 11.

| Cluster | Gene | avg_logFC (Increase) | p_val | pct. 1 | pct. 2 | p_val_adj |
|---|---|---|---|---|---|---|
| 11 | MGP | 1.783159997 | 3.85543E−83 | 1 | 1 | 7.61487E−79 |
| 11 | TFF1 | 1.227959562 | 7.47452E−57 | 1 | 0.976 | 1.47629E−52 |
| 11 | KRT14 | 1.225131728 | 2.72598E−91 | 0.752 | 0.152 | 5.38409E−87 |
| 11 | S100A9 | 1.131511529 | 7.93036E−22 | 0.846 | 0.665 | 1.56633E−17 |
| 11 | KRT17 | 1.125163636 | 3.86663E−88 | 0.698 | 0.134 | 7.63697E−84 |
| 11 | S100G | 1.024339965 | 8.86712E−41 | 0.993 | 0.883 | 1.75135E−36 |
| 11 | S100A2 | 0.979776639 | 5.5328E−108 | 0.664 | 0.094 | 1.0928E−103 |
| 11 | ZNF350-AS1 | 0.969905765 | 2.40754E−45 | 0'705 | 0.24 | 4.75514E−41 |
| 11 | KRT5 | 0.965368643 | 2.5431E−127 | 0.698 | 0.085 | 5.0229E−123 |
| 11 | S100A8 | 0.964816133 | 3.96337E−34 | 0.597 | 0.208 | 7.82804E−30 |
| Cluster | Gene | avg_logFC (Decrease) | p_val | pct. 1 | pct. 2 | p_val_adj |
| 11 | MN1 | 0.250383023 | 1.3128E−14 | 0.456 | 0.198 | 2.59292E−10 |
| 11 | TMEM45A | 0.251362007 | 4.87257E−25 | 0.369 | 0.098 | 9.62382E−21 |
| 11 | DNALI1 | 0.251438063 | 2.77364E−17 | 0.55 | 0.237 | 5.47822E−13 |
| 11 | C3orf14 | 0.252462456 | 1.6936E−11 | 0.644 | 0.376 | 3.34502E−07 |
| 11 | TSR1 | 0.252566899 | 3.76623E−10 | 0.624 | 0.388 | 7.43868E−06 |
| 11 | AL445524.1 | 0.252748869 | 5.63616E−12 | 0.544 | 0.288 | 1.1132E−07 |
| 11 | SEMA3C | 0.252987332 | 8.68902E−06 | 0.725 | 0.578 | 0.171616801 |
| 11 | NDUFAF2 | 0.253508185 | 3.96142E−10 | 0.685 | 0.421 | 7.82421E−06 |
| 11 | ASCL1 | 0.253569127 | 1.55004E−07 | 0.456 | 0.263 | 0.003061487 |
| 11 | GRHL2 | 0.253639823 | 3.9779E−12 | 0.617 | 0.348 | 7.85675E−08 |

TABLE 15
Most differentially expressed genes in Cluster 12.

| Cluster | Gene | avg_logFC (Increase) | p_val | pct. 1 | pct. 2 | p_val_adj |
|---|---|---|---|---|---|---|
| 12 | SAA1 | 1.627887297 | 3.39544E−69 | 0.889 | 0.188 | 6.70634E−65 |
| 12 | FABP4 | 1.436847865 | 2.49417E−73 | 0.691 | 0.098 | 4.92624E−69 |
| 12 | GPX3 | 1.235701869 | 3.44158E−29 | 0.802 | 0.354 | 6.79746E−25 |
| 12 | PIP | 1.049914368 | 2.86315E−19 | 0.667 | 0.286 | 5.65501E−15 |
| 12 | ADH1B | 0.899800048 | 1.29817E−61 | 0.58 | 0.076 | 2.56402E−57 |
| 12 | PLIN1 | 0.784480331 | 1.32243E−53 | 0.543 | 0.077 | 2.61193E−49 |
| 12 | COL2A1 | 0.783492947 | 5.22017E−12 | 0.778 | 0.628 | 1.03104E−07 |
| 12 | SH3BGRL | 0.768594814 | 4.86726E−12 | 0.827 | 0.699 | 9.61333E−08 |
| 12 | AD1POQ | 0.687320225 | 8.2589E−109 | 0.494 | 0.027 | 1.6312E−104 |
| 12 | PLIN4 | 0.65682665 | 5.41747E−33 | 0.407 | 0.068 | 1.07001E−28 |
| Cluster | Gene | avg_logFC (Decrease) | p_val | pct. 1 | pct. 2 | p_val_adj |
| 12 | HNRNPA0 | 0.250265112 | 2.70326E−05 | 0.975 | 0.957 | 0.533920821 |
| 12 | RPL11 | 0.250877761 | 7.1194E−12 | 1 | 1 | 1.40615E−07 |
| 12 | SLC40A1 | 0.252618406 | 0.000200052 | 0.605 | 0.432 | 1 |
| 12 | RPLP2 | 0.253394982 | 4.98006E−18 | 1 | 1 | 9.83611E−14 |
| 12 | CTDSP2 | 0.253415479 | 5.19057E−06 | 0.938 | 0.844 | 0.102518854 |
| 12 | SORL1 | 0.254054156 | 0.000137998 | 0.494 | 0.326 | 1 |
| 12 | RPL31 | 0.255440813 | 1.14535E−09 | 1 | 1 | 2.26219E−05 |
| 12 | ECE1 | 0.258177708 | 0.00017553 | 0.765 | 0.72 | 1 |
| 12 | SFRP2 | 0.259944446 | 3.32806E−05 | 0.914 | 0.847 | 0.65732505 |
| 12 | CCND1 | 0.2602566 | 0.000364464 | 0.988 | 1 | 1 |

TABLE 16
Most differentially expressed genes in Cluster 13.

| Cluster | Gene | avg_logFC (Increase) | p_val | pct. 1 | pct. 2 | p_val_adj |
|---|---|---|---|---|---|---|
| 13 | PDE5A | 1.439985157 | 3.24289E−22 | 0.967 | 0.365 | 6.40503E−18 |
| 13 | MGP | 1.222167551 | 9.68174E−16 | 1 | 1 | 1.91224E−11 |
| 13 | WFDC2 | 1.091487434 | 5.91075E−17 | 1 | 0.679 | 1.16743E−12 |
| 13 | MRPS30-DT | 1.081270562 | 4.90793E−34 | 0.9 | 0.157 | 9.69365E−30 |
| 13 | RBM20 | 1.079620292 | 3.3553E−29 | 0.967 | 0.227 | 6.62704E−25 |
| 13 | MRPS30 | 1.056911819 | 3.60223E−19 | 1 | 0.667 | 7.11477E−15 |
| 13 | AMFR | 1.046976995 | 1.25599E−16 | 1 | 0.94 | 2.4807E−12 |
| 13 | STC2 | 0.9846206 | 5.37678E−17 | 1 | 0.916 | 1.06197E−12 |
| 13 | KCNE4 | 0.922004616 | 1.41051E−17 | 0.9 | 0.325 | 2.7859E−13 |
| 13 | DDR1 | 0.915241601 | 7.09634E−18 | 1 | 0.997 | 1.4016E−13 |
| Cluster | Gene | avg_logFC (Decrease) | p_val | pct. 1 | pct. 2 | p_val_adj |
| 13 | MCCC1 | 0.25012036 | 0.008672717 | 0.533 | 0.329 | 1 |
| 13 | PDPR | 0.250283653 | 0.001475067 | 0.867 | 0.636 | 1 |
| 13 | AMZ2 | 0.251051569 | 0.00140596 | 0.9 | 0.741 | 1 |
| 13 | C5orf15 | 0.251417578 | 0.001008343 | 0.9 | 0.833 | 1 |
| 13 | TBC1D9 | 0.251419806 | 0.00067258 | 1 | 0.971 | 1 |
| 13 | SRPK1 | 0.251720616 | 0.004445033 | 0.6 | 0.368 | 1 |
| 13 | LINC01488 | 0.252824252 | 0.000284945 | 0.3 | 0.102 | 1 |
| 13 | TSG101 | 0.253298185 | 0.00115588 | 0.867 | 0.7 | 1 |
| 13 | COA3 | 0.253593567 | 0.001172954 | 1 | 0.993 | 1 |
| 13 | NFKBIE | 0.253780521 | 0.000400873 | 0.667 | 0.343 | 1 |

Further, using Seurat, the most differentially-expressed genes in the breast cancer sample from FIG. 35A were identified by examining the average log fold change in a particular gene compared to the average expression of all genes in the breast cancer tissue sample. As shown in FIG. 36 and Table 17 below, the most upregulated genes in the breast cancer tissue sample include ALB, CRISP3, IGLC2, IGHG3, IGKC, CXCL14, IGHGL, IGLC3, MGP, SLITRK6, CPB1, and IGHA1.

TABLE 17
Most upregulated genes in a breast cancer sample.

| Cluster | Gene | avg_logFC (Increase) | p_val | pct. 1 | pct. 2 | p_val_adj |
|---|---|---|---|---|---|---|
| 7 | ALB | 3.0180208 | 3.77E−68 | 0.91 | 0.596 | 7.45E−64 |
| 4 | CRISP3 | 2.6068209 | 2.55E−239 | 1 | 0.801 | 5.03E−235 |
| 0 | IGLC2 | 2.2376986 | 9.87E−207 | 1 | 0.97 | 1.95E−202 |
| 0 | IGHG3 | 1.9692789 | 6.49E−207 | 1 | 1 | 1.28E−202 |
| 0 | IGKC | 1.9270996 | 4.29E−208 | 1 | 1 | 8.47E−204 |
| 2 | CXCL14 | 1.8732975 | 1.28E−247 | 1 | 0.959 | 2.52E−243 |
| 0 | IGHG1 | 1.8719631 | 8.47E−226 | 1 | 0.987 | 1.67E−221 |
| 0 | IGLC3 | 1.8685086 | 7.17E−160 | 0.949 | 0.617 | 1.42E−155 |
| 7 | MGP | 1.8575519 | 2.93E−93 | 1 | 1 | 5.79E−89 |
| 4 | SLITRK6 | 1.8156925 | 7.06E−276 | 0.995 | 0.369 | 1.39E−271 |

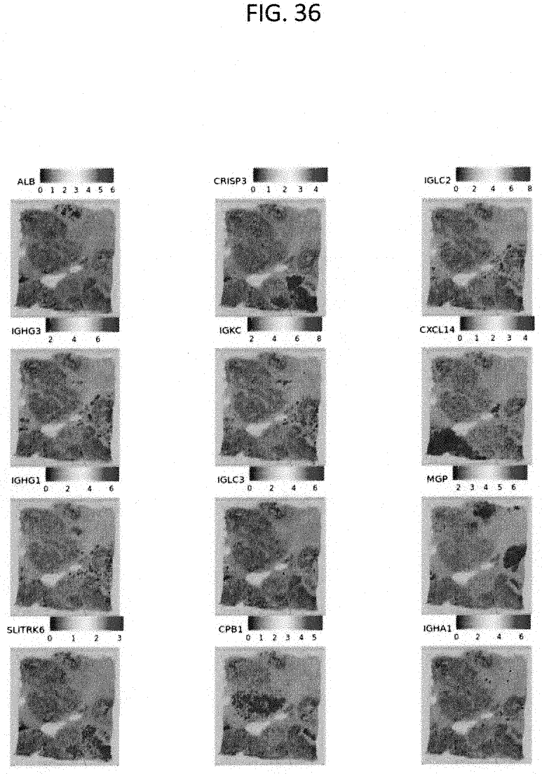

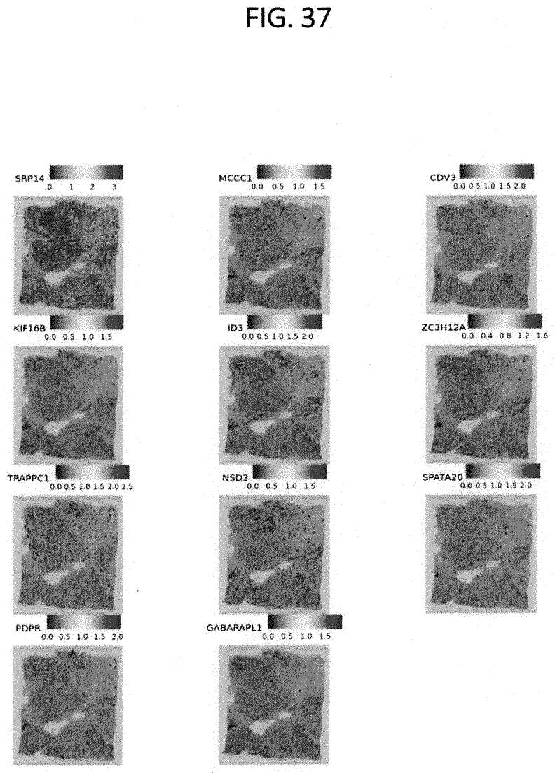

As shown in FIG. 37 and Table 18 below, the most downregulated genes in the breast cancer tissue sample include SRP14, MCCC1, CDV3, KIF16B, ID3, ZC3H12A, TRAPPC1, NSD3, HNRNPA01, SPATA20, PDPR, and GABARAPL1.

TABLE 18
Most downregulated genes in a breast cancer sample.

| Cluster | Gene | avg_logFC (Decrease) | p_val | pct. 1 | pct. 2 | p_val_adj |
|---|---|---|---|---|---|---|
| 5 | SRP14 | 0.2500146 | 9.97E−30 | 1 | 1 | 1.97E−25 |
| 13 | MCCC1 | 0.2501204 | 8.67E−03 | 0.533 | 0.329 | 1.00E+00 |
| 7 | CDV3 | 0.25013413 | 5.36E−10 | 0.91 | 0.871 | 1.06E−05 |
| 10 | KIF16B | 0.2501365 | 2.98E−09 | 0.545 | 0.336 | 5.88E−05 |
| 0 | ID3 | 0.2501782 | 2.04E−15 | 0.599 | 0.511 | 4.04E−11 |
| 4 | ZC3H12A | 0.2501944 | 2.63E−34 | 0.559 | 0.261 | 5.19E−30 |
| 5 | TRAPPC1 | 0.250232 | 1.13E−21 | 0.966 | 0.925 | 2.41E−17 |
| 2 | NSD3 | 0.2502539 | 4.50E−25 | 0.81 | 0.646 | 8.90E−21 |
| 12 | HNRNPA01 | 0.2502651 | 2.70E−05 | 0.975 | 0.957 | 5.34E−01 |
| 10 | SPATA20 | 0.2502745 | 1.06E−12 | 0.929 | 0.866 | 2.10E−08 |

The above data provide both individual gene markers and clusters of gene markers that are differentially expressed in a breast cancer sample. The individual gene markers and clusters of gene markers can be utilized to identify particular types of breast cancer (e.g., invasive carcinoma; DCIS) in a tissue sample, aiding in both diagnostic and therapeutic methods.

### Example 4: Spatially-Resolved Gene Expression and Clustering in Invasive Ductal Carcinoma

The spatial gene expression of invasive ductal carcinoma tissue from a female patient (ER+, PR−, HER2+) was profiled (BioIVT: Asterand—Case ID 66320; Specimen ID 116899F). As a control, the healthy tissue sections adjacent to the tumor were obtained. 4 replicates were used for each tissue type.

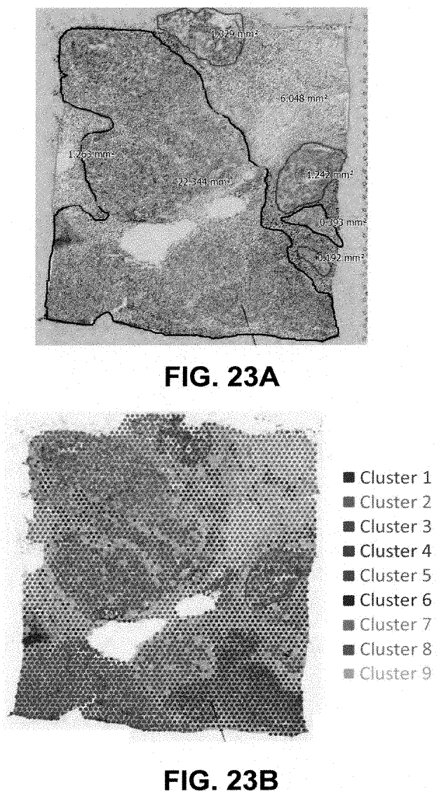

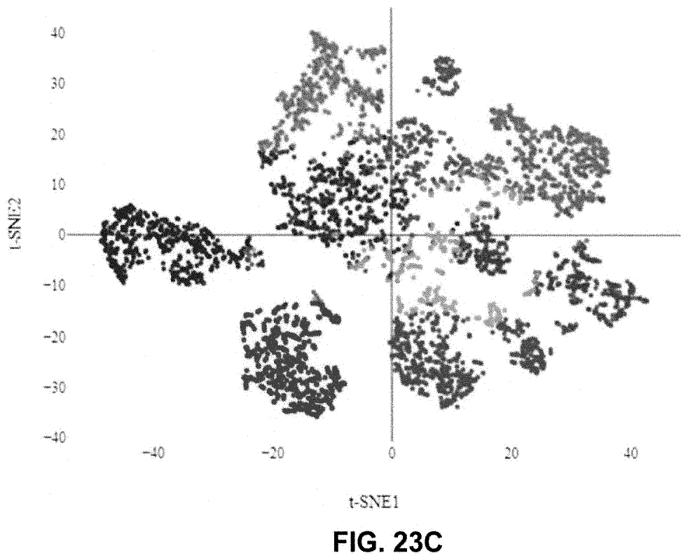

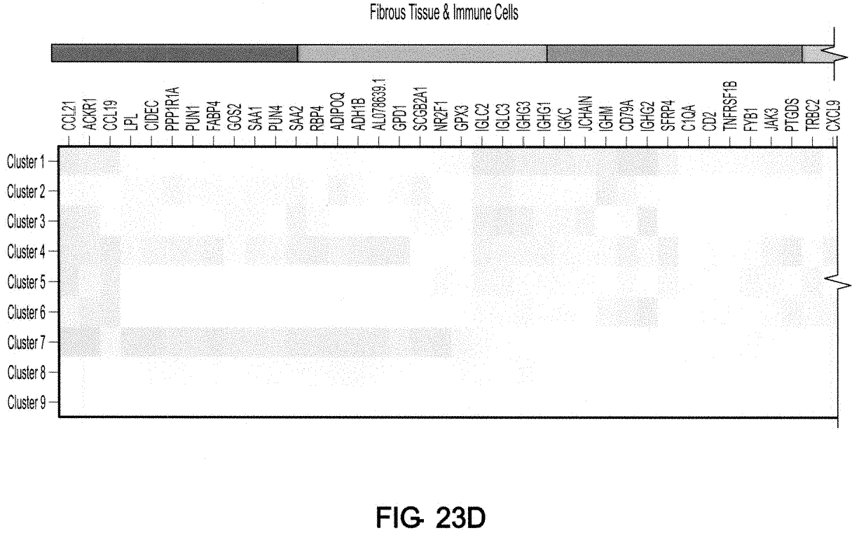

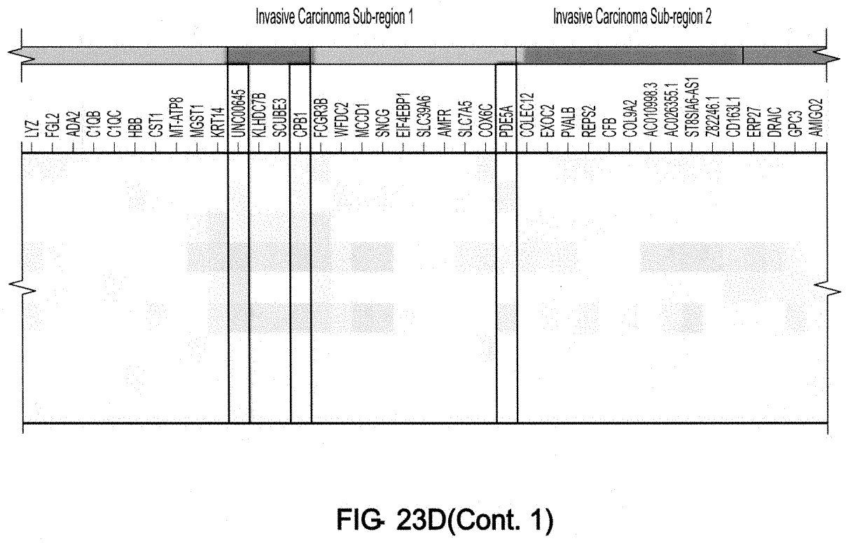

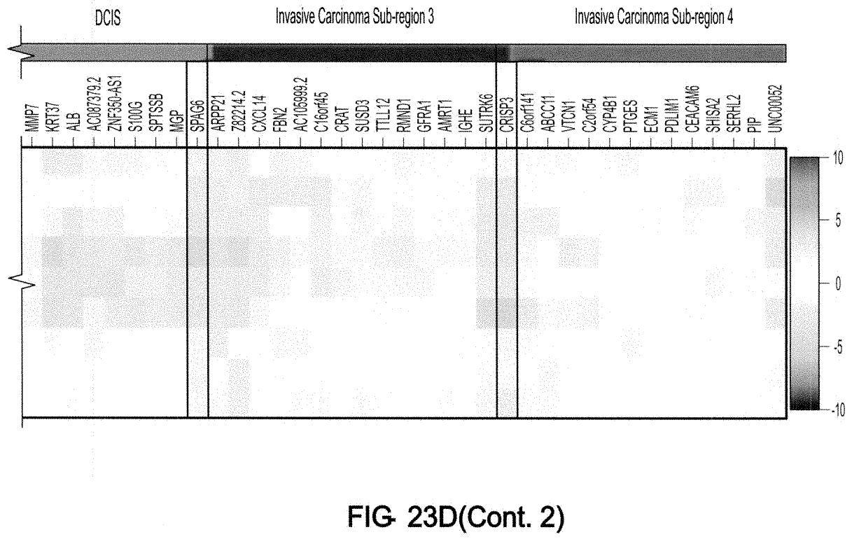

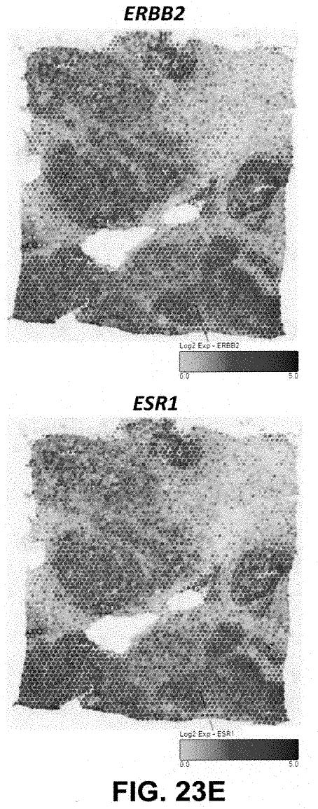

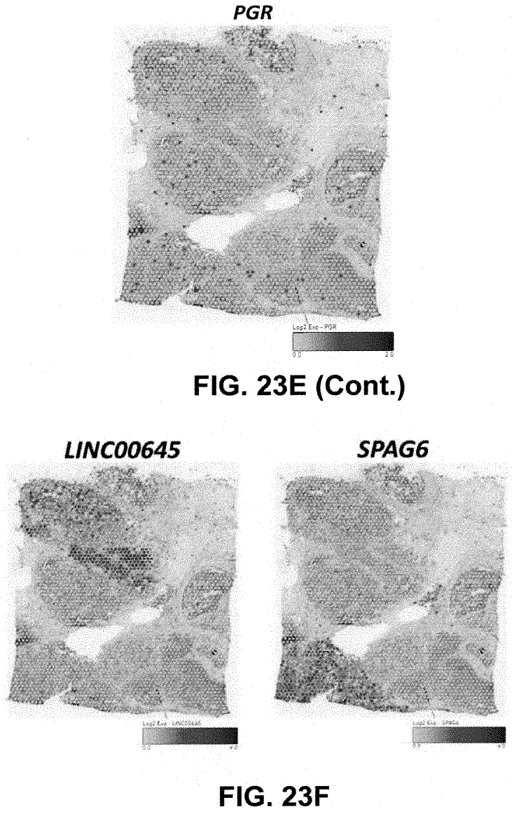

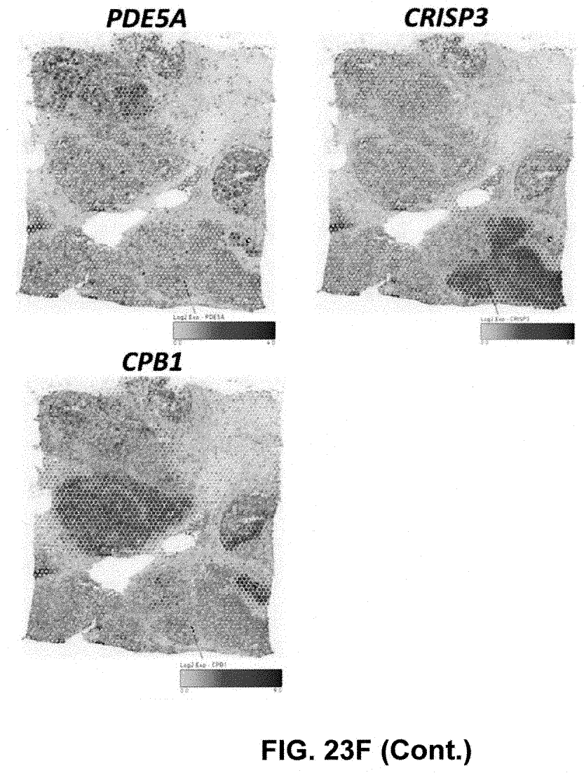

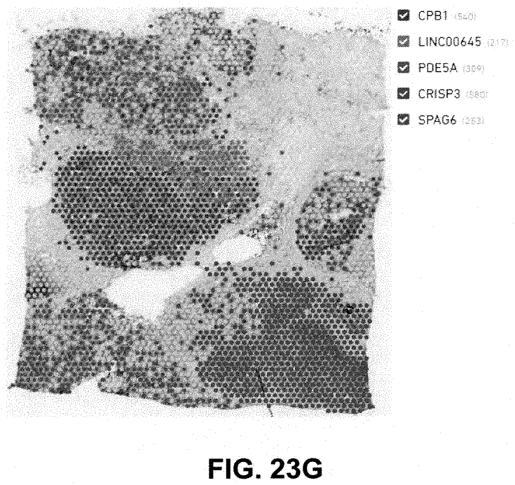

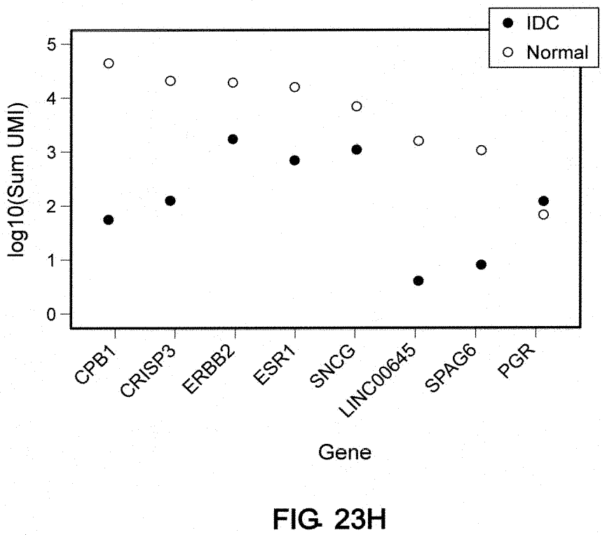

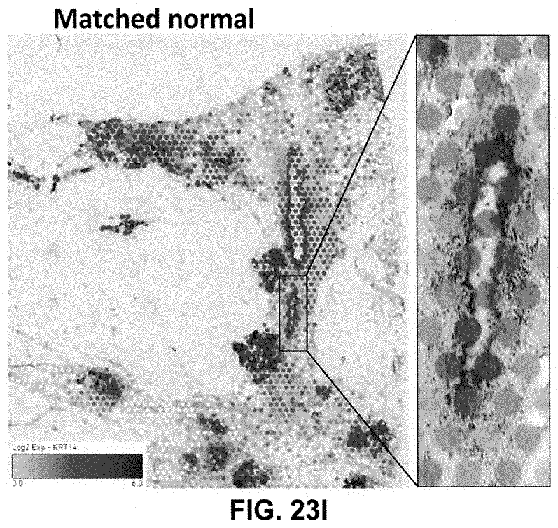

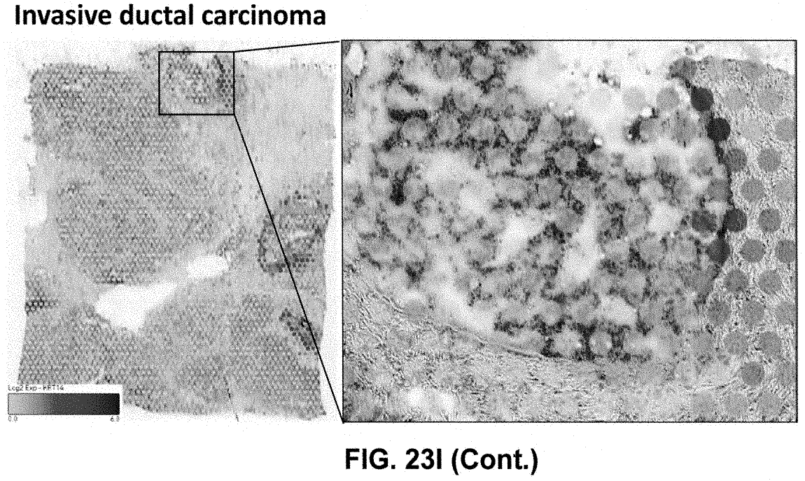

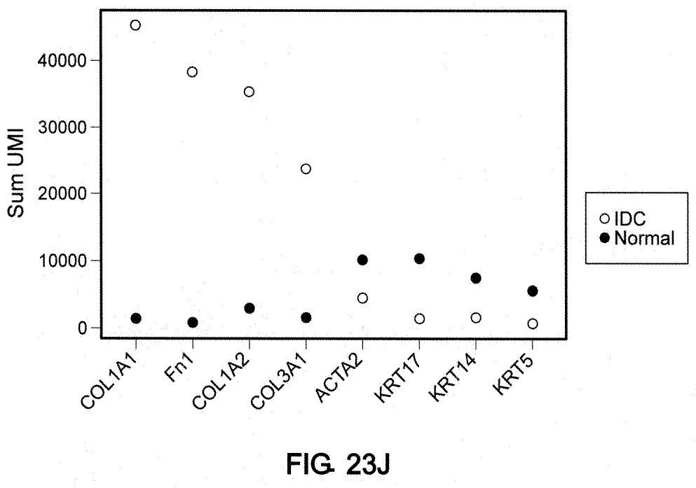

Spatially-resolved gene expression and clustering in invasive ductal carcinoma reveal intra-tumor heterogeneity is shown in FIGS. 23A-H. FIG. 23A shows a histological section of an invasive ductal carcinoma annotated by a pathologist. The section contains a large proportion of invasive carcinoma (outlined in black), three separate ductal cancer in situ regions, and fibrous tissue. FIG. 23B shows a tissue plot with spots colored by unsupervised clustering of transcripts. FIG. 23C shows a t-SNE plot of spots colored by unsupervised clustering of transcripts. FIG. 23D shows a gene expression heat map of the most variable genes between the 9 identified clusters. The region defined as fibrous tissue mostly corresponds to clusters 1, 7, and 8. Interestingly, a large region annotated as invasive carcinoma by a pathologist contained spatial spots that were assigned to DCIS (cluster 5). In addition, four subtypes of invasive carcinoma with distinct molecular properties (clusters 2, 3, 4, and 6) were identified, revealing intra-tumor heterogeneity.

The expression levels of genes corresponding to human epidermal growth factor receptor 2 (Her2 or ERBB2), estrogen receptor (ER or ESR1), and progesterone receptor (PGR) in the tissue section are shown in FIG. 23E. It is clearly visible that ERBB2 and ESR1 are highly expressed in the invasive carcinoma and DCIS regions while the expression of PR is absent, consistent with the patient's diagnosis. One of the top differentially expressed genes from each cluster in the invasive carcinoma region was selected (rectangular boxes in FIG. 23D), and its expression levels are located in the tissue as shown in FIG. 23F and overlapped in one plot as shown in FIG. 23G. With the exception of PGR, all of these genes were highly up-regulated in the carcinoma tissue compared to the adjacent normal tissue (FIG. 23H). Analysis revealed that all of these up-regulated genes have implication in cancer progression. Interestingly, in the subset of cluster 3, a long non-coding RNA, of which abnormal expression has recently been implicated in tumor development (see, e.g., Chang T, et al. Long Non-Coding RNA and Breast Cancer. Technol Cancer Res Treat. 2019, 18, 1533033819843889, incorporated herein by reference in its entirety), is one of the top differentially expressed genes. In glioblastoma, LINC00645 promotes epithelial-to-mesenchymal transition by inducing TGF-β (see, e.g., Li, C. et al. Long non-coding RNA linc00645 promotes TGF-β-induced epithelial—mesenchymal transition by regulating miR-205-3p-ZEB1 axis in glioma. Cell Death & Dis. 2019, 10, 272, incorporated herein by reference in its entirety).

During breast cancer progression, the myoepithelial cells, which continue to surround preinvasive in situ carcinoma, gradually disappear (see, e.g., Gudjonsson, T. et al. Myoepithelial Cells: Their Origin and Function in Breast Morphogenesis and Neoplasia. J. Mammary Gland Biol. Neoplasia. 2009, 10, 261, incorporated herein by reference in its entirety). This phenomenon is clearly visualized in FIGS. 23I and 23J where KRT14 (a gene signature of myoepithelial cells) was highly expressed around the lining of the duct in the normal tissue while it was disappearing in the DCIS region in IDC tissue (FIG. 23I). The extracellular matrix genes such as COL1A1 and FN1, key genes associated with invasion and metastasis, were highly upregulated while smooth muscles and basal keratin were down-regulated in IDC (FIG. 23J).

### Example 5: Identification of Heterogeneity in Triple Negative Breast Cancer Sample Using Spatial Gene Expression Methods

Extensive heterogeneity exists in the breast cancer tumor microenvironment. However, clinical characterization has been primarily limited to pathologist annotation and tissue staining for three key genes: estrogen receptor (ER), progesterone receptor (PR), and human epidermal growth factor receptor 2 (HER2). Triple negative breast cancer (TNBC) expresses none of the usual breast cancer therapeutic targets, including ER, PR, and HER2, making it unresponsive to either hormone therapy or targeted anti-HER2 drugs like trastuzumab.

Here, heterogeneity was examined using spatial methods. Briefly, human tissue specimens from triple negative breast cancer biopsy were obtained. 10 μm fresh-frozen tissue was sectioned using a cryostat. The sectioned tissues for each sample were placed onto a gene expression slide having approximately 5,000 gene expression spots, each of which contains spatially barcoded probes that bind mRNA from the cells in the tissue section above it.

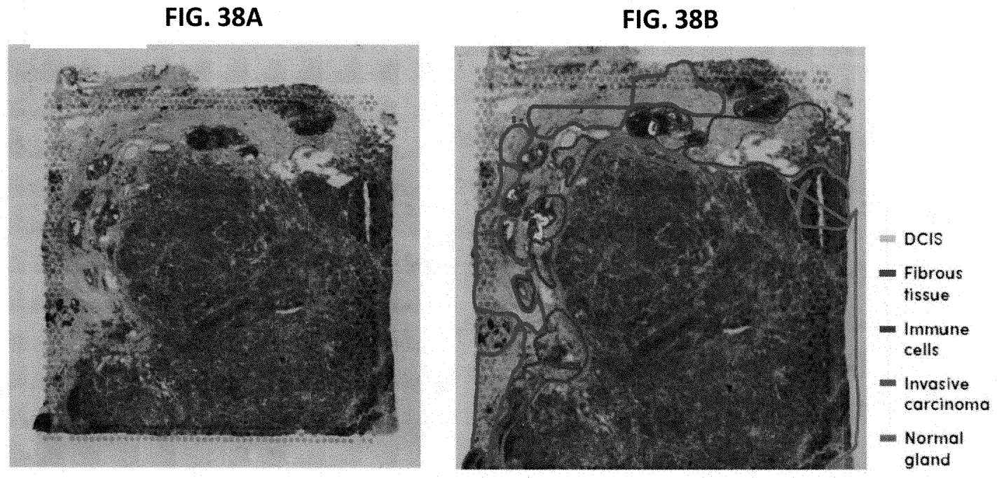

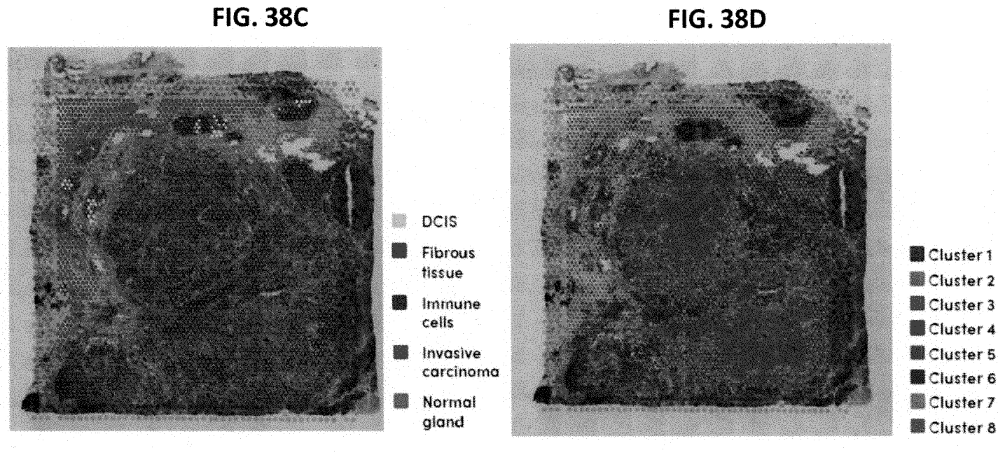

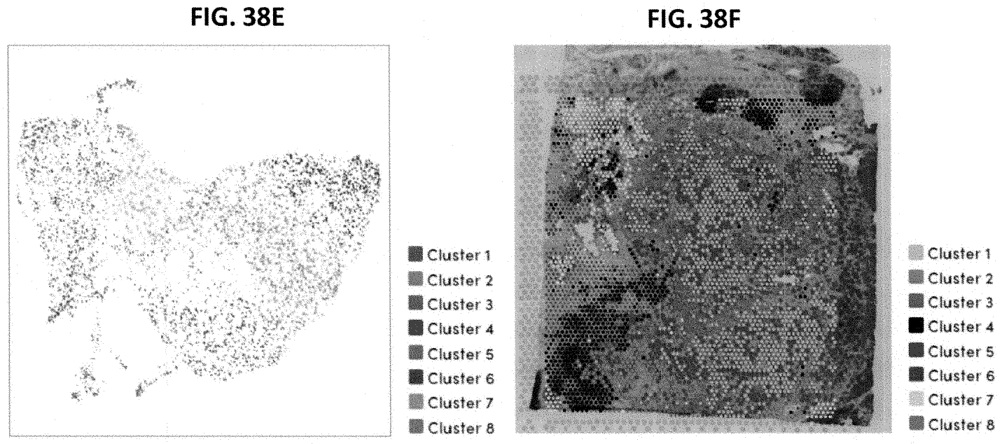

The samples were fixed with acetone, and were stained with hematoxylin and eosin (H&E) according established protocols. H&E-stained tissues were images at 20× magnification using brightfield settings (see FIG. 38A) and manually annotated by a pathologist. As shown in FIG. 38B, annotation of the sample outlined areas of DCIS, fibrous tissue, immune cells, invasive carcinoma, and normal gland. The outlined areas identified by the pathologist were filled in, showing the five annotations. As shown in FIG. 38C, the bulk of the section was annotated as invasive carcinoma, meaning the cancer cells have spread beyond the milk duct and invaded the surrounding breast tissue, and have the potential to spread to other parts of the body. Surrounding the invasive carcinoma are immune cells, which are interspersed with discrete regions of ductal carcinoma in situ (DCIS), areas of non-invasive cancer where atypical cells line the milk ducts. Along the upper left periphery lies fibrous tissue, predominantly made up of extracellular matrix. Normal gland, is also present.

After imaging, the tissue was prepared for spatial detection of gene expression. First, the tissue was permeabilized, releasing mRNA that bound to spatially barcoded capture probes on the slide. Briefly, tissues were washed and permeabilized using Proteinase K, incubated at 37° C. for at least 5 minutes and then washed to remove the protease.

After permeabilization, the released mRNA are allowed to hybridize to the capture domain on the capture probe immobilized on the spatial array via the polyA tail on the mRNA. The captured mRNA molecules were copied, using the capture probe as a template and the extension product was released from the spatial array. Briefly, the tissues were incubated with a second strand extension mix comprising Kapa Hifi DNA polymerase (Roche) for 25 minutes at 53° C. Following incubation, the extension mix was removed from the tissues and the tissues were washed with SSC. A solution of KOH was added to each of the tissue wells, the tissues were incubated at room temperature for 10 minutes to release the extension product from the spatial array and the supernatant from each tissue well was transferred for quantitation, library preparation and sequencing on the Illumina NextSeq sequencing instrument.

Sequencing results were analyzed and groups (i.e., clusters) of co-segregated genes were identified. Clusters were visualized spatially, with reference to the tissue section (FIG. 38D), or as UMAP projections showing the distance between gene expression-based clusters (FIG. 38E). While a traditional single cell UMAP is made up of dots representing single cells, with spatial gene expression each dot represents a tissue-covered spot that may include mRNA from one to ten cells as shown in FIG. 38E. A list of select differentially expressed genes for all eight graph-based clusters is provided in Table 19.

In general, there was high correlation between pathologist annotations and graph-based clusters determined by gene expression, particularly at region boundaries. For instance, as shown in FIG. 38D, graph-based Cluster 3 primarily overlapped with the pathologist-annotated region labeled as fibrous tissue, and Clusters 6 and 7 overlapped largely with immune cells. Cluster 8 showed consistent localization with DCIS. Using Spatial Gene Expression, additional heterogeneity can be identified within regions, particularly for invasive carcinoma, which is made up of four distinct gene expression-based clusters, including Cluster 1, Cluster 2, Cluster 4, and Cluster 5.

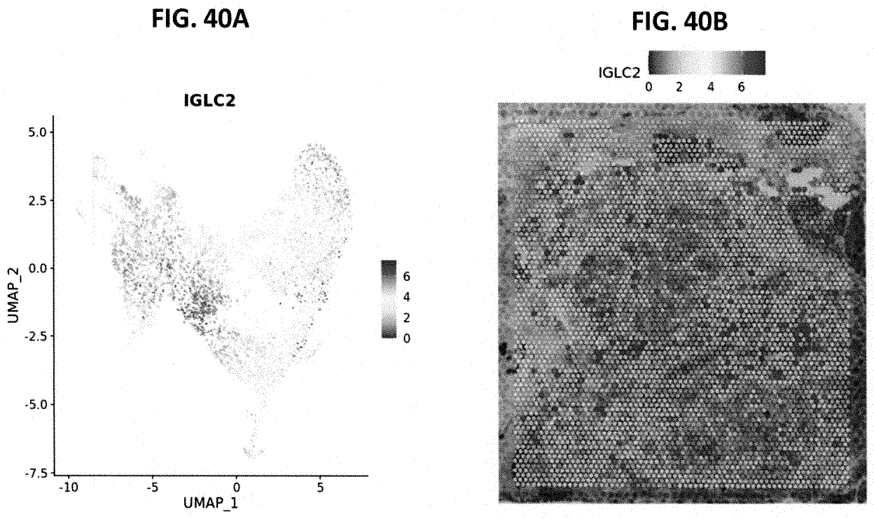

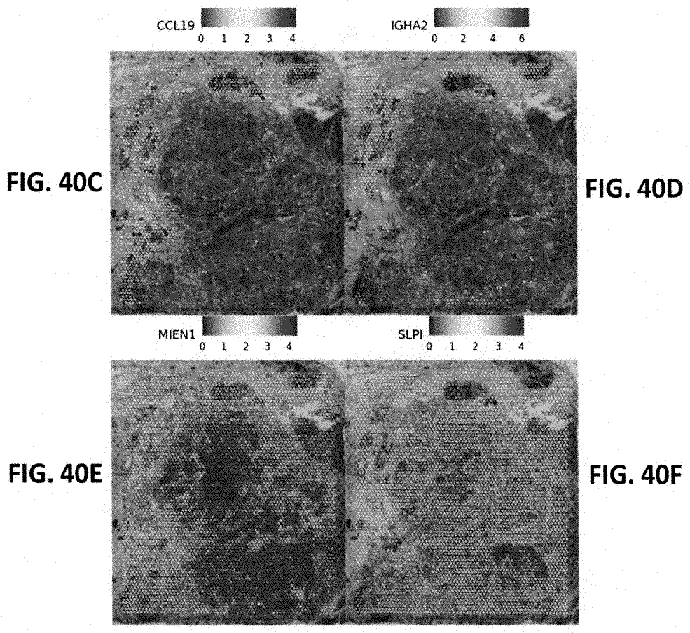

In the same sample used herein, these methods were used to identify expression of individual genes. For example, immunoglobulin lambda constant 2 (IGLC2; FIGS. 40A-40B); C—C motif chemokine ligand 19 (CCL19, FIG. 40C); immunoglobulin heavy constant alpha 2 (A2m marker) (IGHA2; FIG. 40D); migration and invasion enhancer 1 (MIEN1; FIG. 40E), and secretory leukocyte peptidase inhibitor (SLPI, FIG. 40F) were identified.

Next, using the cancer panel described herein, similar cluster patterns and transcriptional profiles were resolved on a second section of the same TNBC sample. See FIG. 38F.

Taken together, these data demonstrate that, while whole transcriptome analysis is ideal for discovery and full characterization of graph-based clusters, targeted transcriptome analysis can provide exceptional value in the disambiguation of spatial heterogeneity and validation of preliminary findings.

TABLE 19
Select differentially expressed genes for spatially defined clusters

| Cluster | Gene | Name | Summary |
|---|---|---|---|
| 1 | MMP7 | Matrix metallopeptidase 7 | Exhibits elevated expression in |
| | | | several types of human cancer |
| 1 | FYB1 | FYN binding protein 1 | Involved in platelet activation and IL- |
| | | | 2 expression |
| 2 | FDCSP | Follicular dendritic cell | Secreted protein that binds to |
| | | secreted protein | activated B cells to regulate antibody |
| | | | responses may promote cancer cell |
| | | | invasion and migration |
| 2 | BCL2A1 | BCL2 related protein Al | Reduces release of pro-apoptotic |
| | | | cytochrome c, and serves as a direct |
| | | | transcription factor of NK-kB in |
| | | | response to inflammatory mediators |
| 3 | TNXB | Tenascin XB | Extracellular matrix glycoprotein |
| | | | with anti-adhesive effects |
| 3 | CLDN5 | Claudin 5 | Integral membrane protein and |
| | | | component of tight junctions |
| 4 | KLK6 | Kallikrein related | Implicated in carcinogenesis and may |
| | | peptidase 6 | serve as a biomarker for multiple |
| | | | cancer types |
| 4 | CENPW | Centromere protein W | Putative oncogene |
| 5 | CST1 | Cystatin SN | Favorable prognostic marker in |
| | | | breast cancer |
| 5 | POSTN | Periostin | Secreted extracellular matrix protein |
| | | | that plays a role in cancer stem cell |
| | | | maintenance and metastasis |
| 6 | CXCL14 | C-X-C motif chemokine | Chemoattractant for neutrophils and |
| | | ligand 14 | expressed by myeloid dendritic cells |
| 6 | CD79B | CD79b molecule | Part of B lymphocyte antigen |
| | | | receptor |
| 7 | IGLC3 | Immunoglobulin lambda | Part of the constant region of B-cell |
| | | constant 3 (Kern-Oz + | antibodies |
| | | marker) | |
| 7 | MZB1 | Marginal zone B and B1 | Marker of plasma B cells |
| | | cell specific protein | |
| 8 | PIGR | Polymeric | Favorable prognostic marker in |
| | | immunoglobulin receptor | breast cancer |
| 8 | CLU | Clusterin | Secreted chaperone protein that can |
| | | | protect against apoptosis |

### Example 6: Integration of Single Nucleus RNA-Seq with Spatial Gene Expression Integration

Spatial gene expression spots have a diameter of about 55 microns and typically capture 1-10 cells per spot, depending on tissue thickness and cell density. To increase resolution of spatially resolved gene expression to the single cell level, single nuclei RNA-sequencing (snRNA-seq) of two TNBC samples was used to identify cellular subtypes within the tissue.

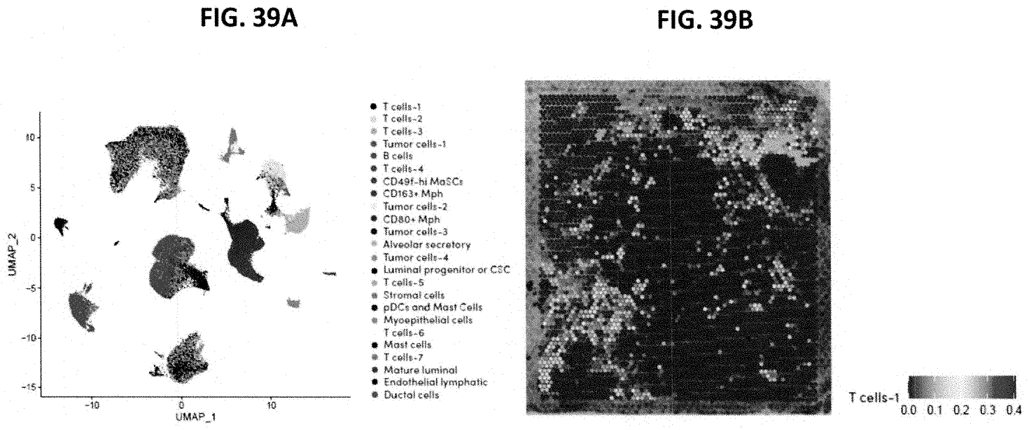

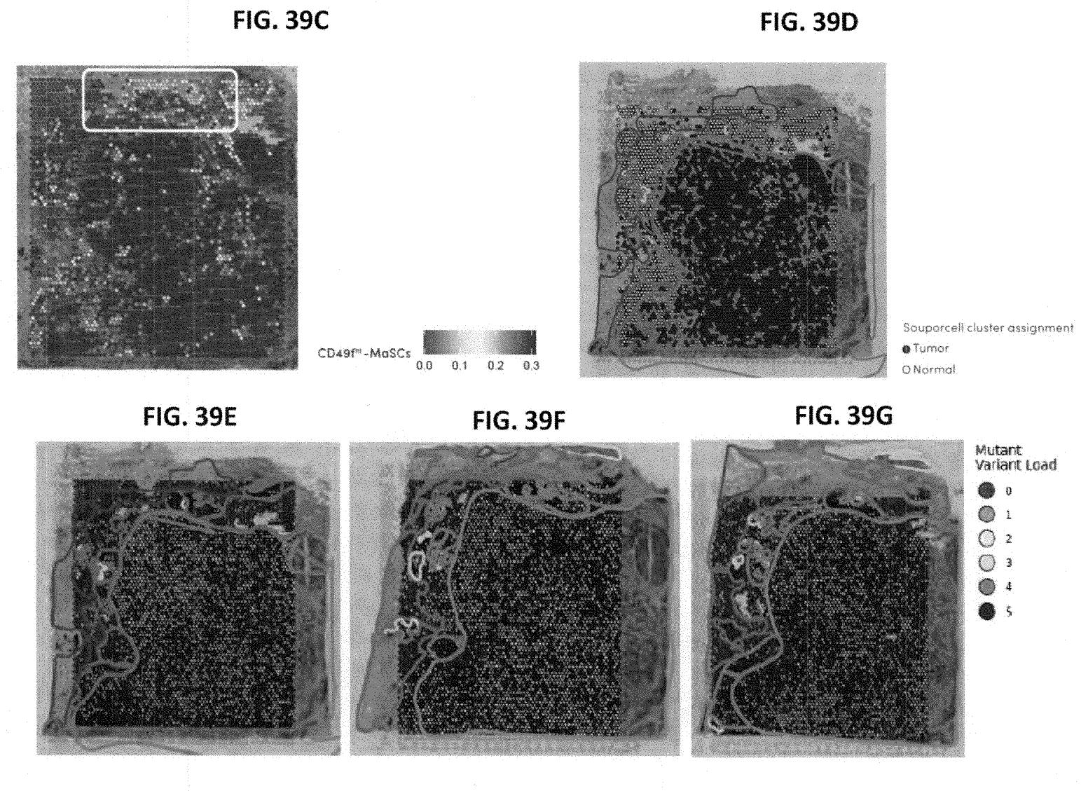

The methods of sectioning a tissue, staining and imaging the section, and capturing and amplification of mRNA was performed as described in Example 5. In addition, two additional fresh-frozen serial TNBC samples were sectioned, and individual nuclei were isolated using methods described previously. See Chromium Next GEM Single Cell 3′ Reagent Kits v3.1 User Guide, Document Number CG000204, Revision Date: November 2019. The resulting data for 86,249 single nuclei were aggregated before clustering and annotated manually using a marker gene approach. See FIG. 39A. Gene expression profiles for both tissues were processed in Seurat and batch corrected using Harmony. See e.g., Korsunsky et al., Fast, Sensitive and Accurate Integration of Single-Cell Data with Harmony. Nat. Methods. 16, 1289-1296 (2019), which is incorporated by reference in its entirety. Cell-type anchors determined by snRNA-seq were matched to Visium Gene Expression spots where these cell types were abundant. For additional detail on how to generate and match cell-type anchors, consult the “Analysis, visualization, and integration of spatial datasets with Seurat” vignette (Analysis, visualization, and integration of spatial datasets with Seurat. Satija Lab. satijalab.org/seurat/v3.2/spatial_vignette.html, which is incorporated by reference in its entirety).

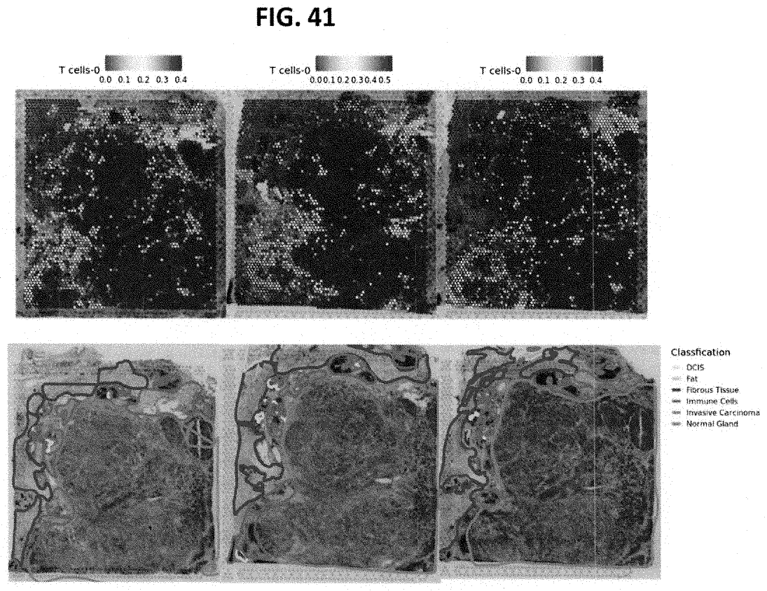

The extent and composition of immune cell infiltration in a tumor informs prognosis. Thus, combining snRNA-seq with spatial gene expression provides a unique view into both the identity of infiltrating cells as well as the extent of penetration into the cancerous region. By leveraging paired snRNA-seq data, spots containing specific subtypes of T cells were identified. As shown in FIG. 39B and FIG. 41, T cells were found throughout the immune cell-annotated region, as well as within the invasive carcinoma region, well beyond the exterior histological boundary. This cluster, T cells-1, expressed PD-1, perforin, granzyme, and other checkpoint inhibitors, suggesting an exhausted T-cell phenotype and limited anti-tumor efficacy.

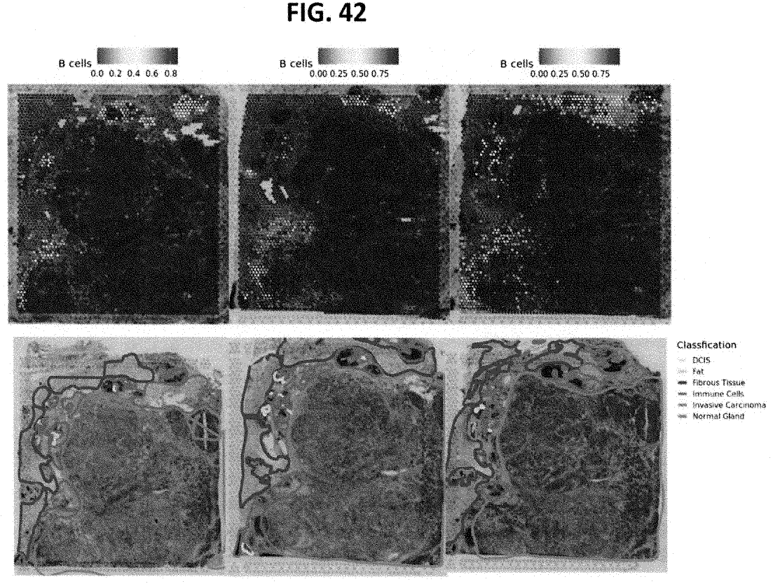

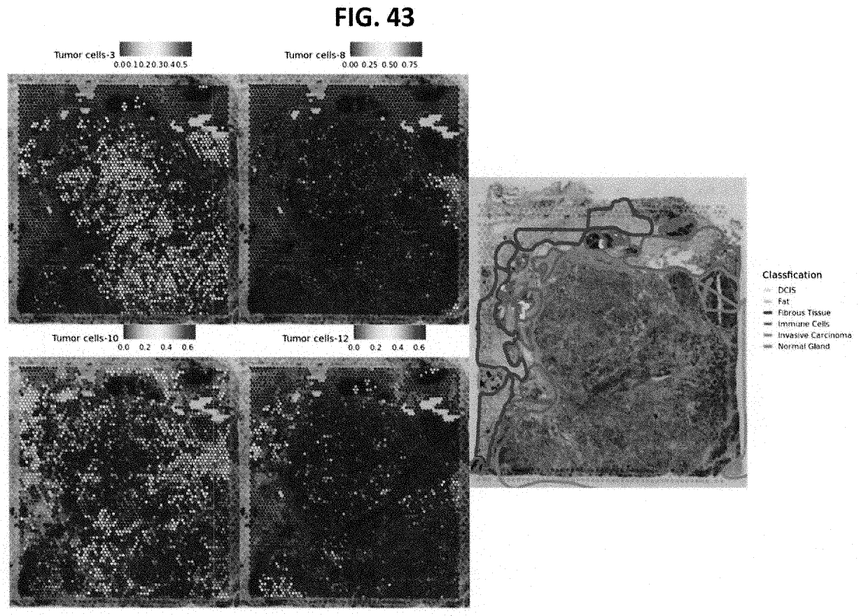

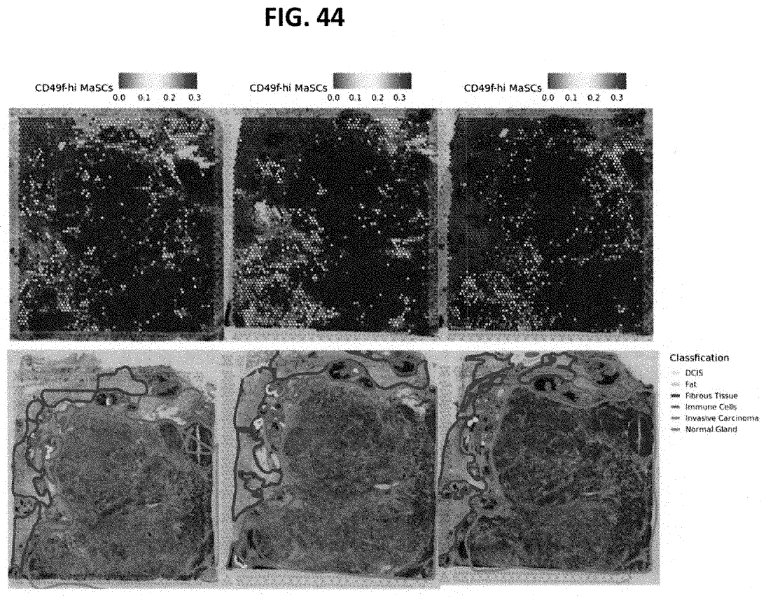

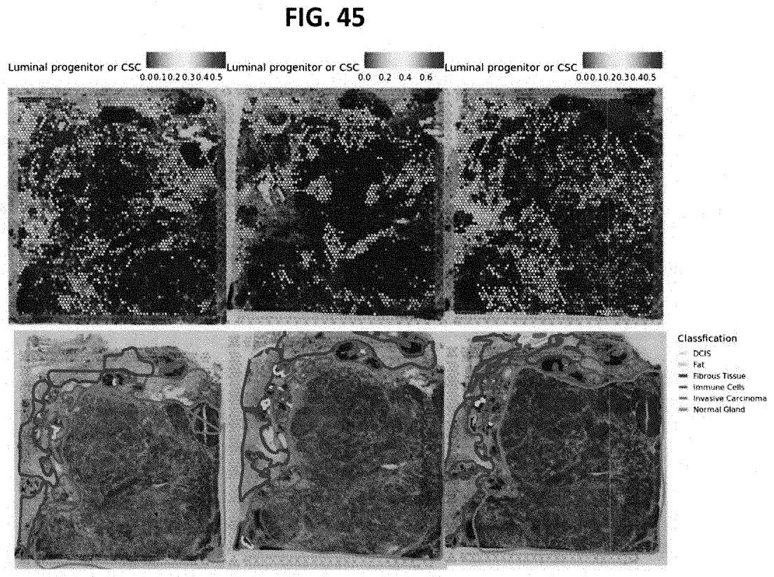

In the absence of snRNA-seq data, the predominant cell type in graph-based clusters can also be inferred based on gene expression. For example, as shown in FIG. 38D, Cluster 7, along the periphery of the pathologist-annotated invasive carcinoma region, expressed numerous immunoglobulin genes, along with MZB1 and JCHAIN, both markers of plasma B cells, suggesting that in this patient, a novel B-cell response has been mounted against the tumor. Analysis of paired, full-length Ig receptors in B cells from this sample could provide additional prognostic value and potential tumor-specific antibody sequences. But, by coupling snRNA-seq and spatial analysis, CD49f^{hi} mammary stem cells (MaSCs), a potential tumor stem cell population, were identified and localized predominantly within the fibrous tissue (FIGS. 39C and 44). Transformation of MaSCs into breast cancer stem cells may result in CD49f^{hi} cells within a breast tumor, the presence of which have been associated with increased chance of cancer recurrence. Given the identification of CD49f^{hi} cells in this TNBC section, knowing whether the cells are tumorigenic could help inform prognosis. Additional cells were identified using the methods disclosed herein. For example, B cells (FIG. 42), subsets of tumor cells (FIG. 43) and cancer stem cells (i.e., luminal progenitor cells) (FIG. 45) were identified in the sample.

To discriminate between tumor and normal cells, spots were individually genotyped. Serial sections of TNBC individually genotyped by souporcell variant identification pipeline. See Heaton et al., bioRxiv 2019. doi.org/10.1101/699637. Variants called in bulk and genotyped using VarTrix6. Clustering (k=2) for identification of major distinct spot clusters shows refinement of tumor/normal boundaries. As shown in FIG. 39D, it was possible to differentiate between putative tumor spots and non-transformed normal spots as long as the divergent alleles were expressed. For example, in the sample in FIG. 39D, the regions containing CD49f^{hi} MaSCs appear genetically normal, suggesting these cells have not yet transformed into breast cancer stem cells, though they may be driven towards a cancer phenotype, given their gene expression profiles. Finally, alternate allele genotype load identified by selection of nonsense (stop gain) and missense (stop loss) mutations were enriched in tumor samples. As shown in FIGS. 39E-39G, Mutant Variant Load represents the number of deleterious alternate allele was observed for a given spot.

### Example 7: Selective Enrichment of Genes of Interest in Breast Invasive Ductal Carcinoma Sample

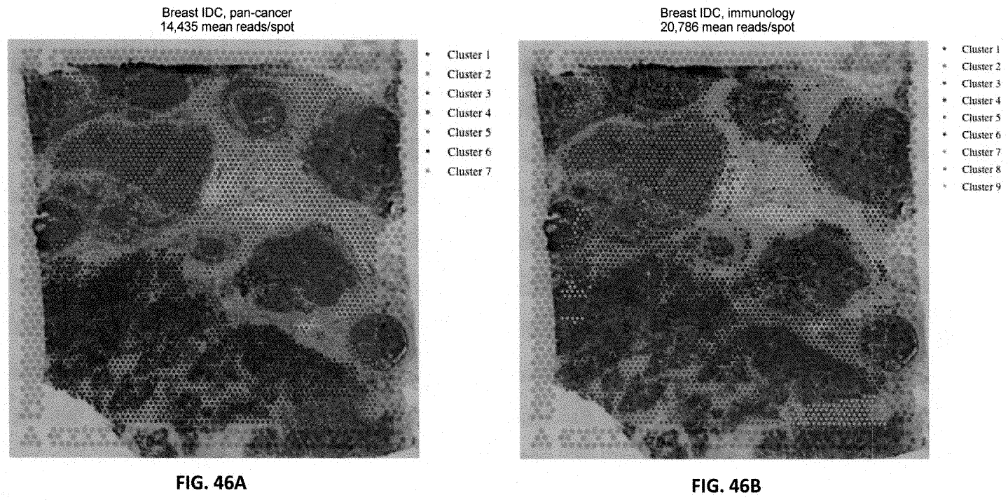

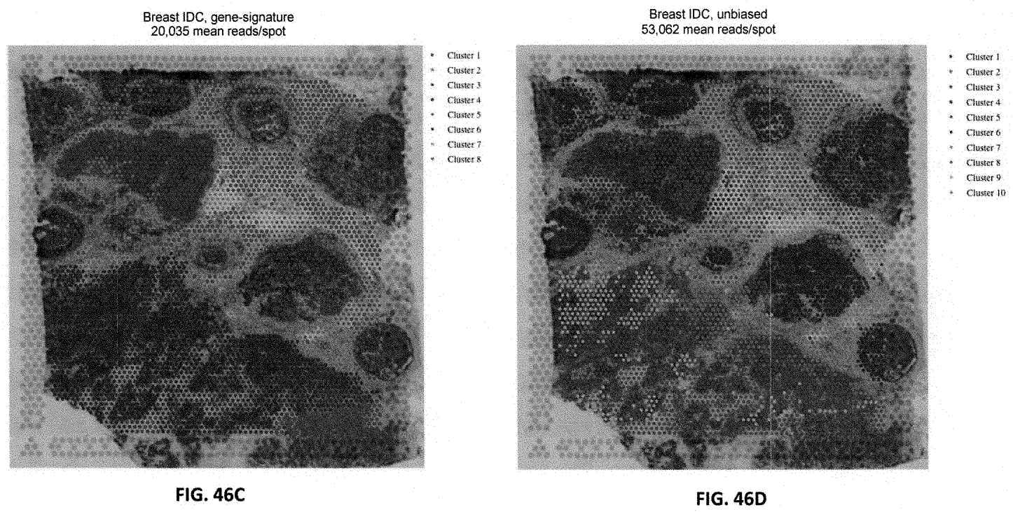

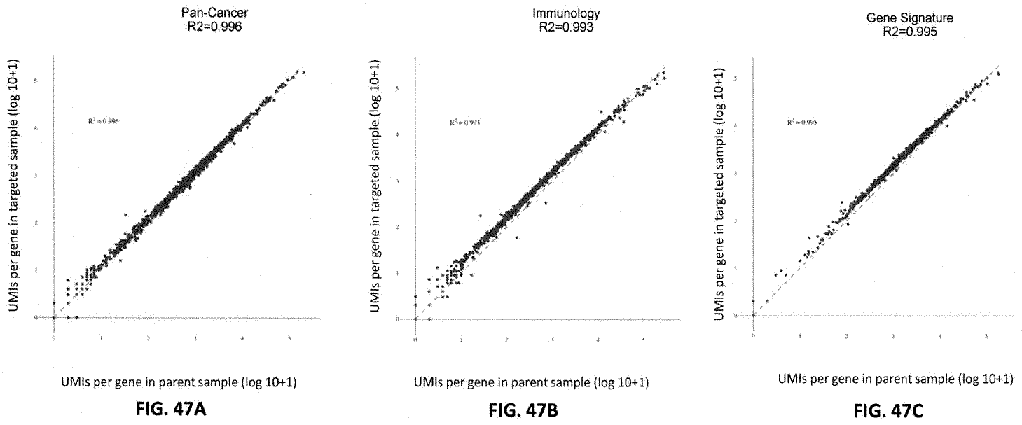

An invasive ductal carcinoma tissue sample from a female patient (ER+, PR−, HER2+) (BioIVT: Asterand—Case ID 66320; Specimen ID 1168993F) was processed as described according to the steps in Example 1. Identification of genes of interest were determined using a cancer-specific panel (FIG. 46A), immune-specific panel (FIG. 46B), and gene-signature (transcription factor) specific panel (FIG. 46C) compared to a non-biased approach using probes to cover the entire transcriptome (FIG. 46D). As shown in FIGS. 47A-47C, there was strong concordance between each group, demonstrating the comparative capability of using a gene-specific panel instead of probes for the entire transcriptome.

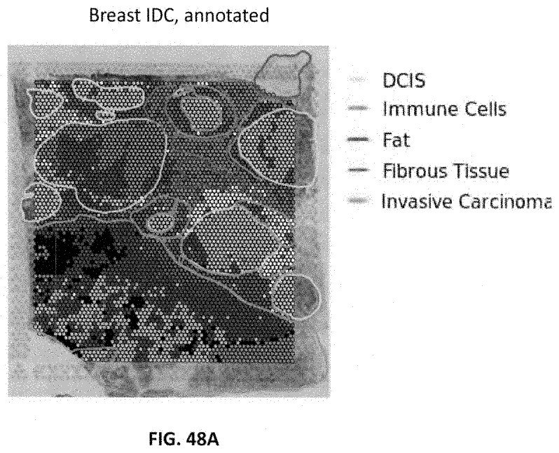

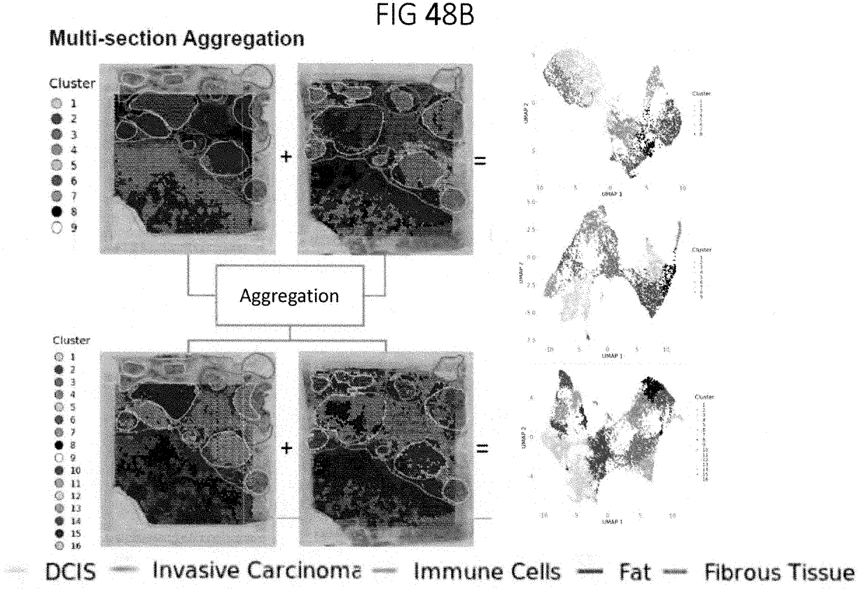

The invasive ductal carcinoma sample was examined by a pathologist, who identified areas of the sample as having areas that were cancerous or non-cancerous. See FIG. 48A. Individual TNBC sections were processed and expression in two different tissue and gene expression spaces was determined; after aggregation of RNA-seq data, clusters were mapped back to their original tissue space but now share expression space (UMAP). See FIG. 48B. Based on the pan-cancer targeted sample, it was identified that ALB, SOCS2, CSTA, BAMBI, WIPI1, PRLR, BCL2, ZNF03, and IL6ST were overexpressed in DCIS cancer cells. And, it was identified that PRKACB, SPP1, CCND1, FGFR1, VEGFA, FN1, HES6, S100A11, H2AFX, were overexpressed in invasive carcinoma cancer cells. See, e.g., Table 20.

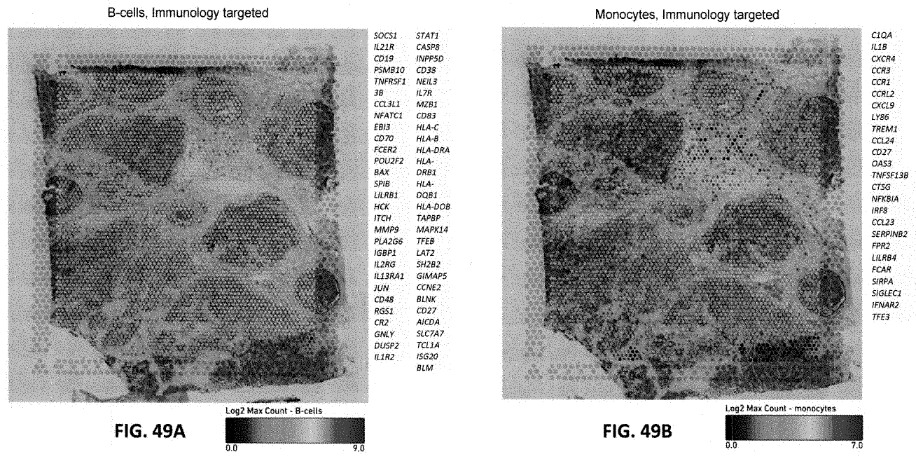

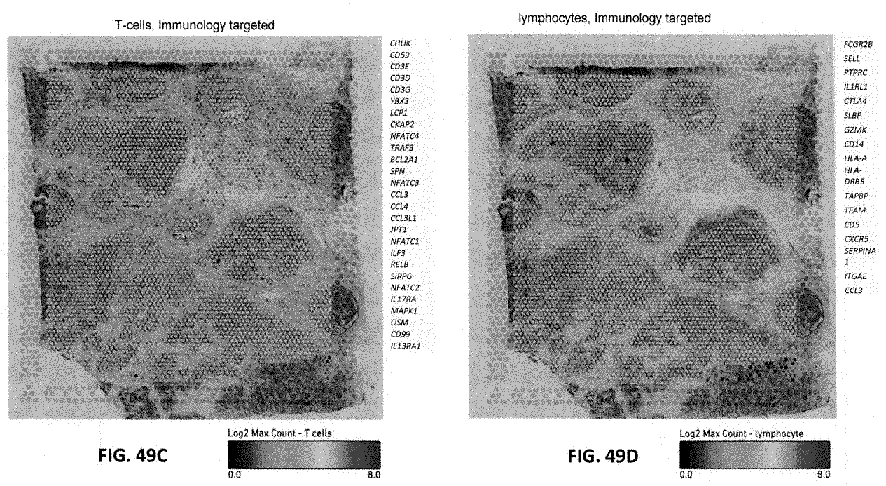

In addition, based on immune clustering, immune cells were identified. See FIGS. 49A-49D.

TABLE 20
Transcripts with increased expression (increased
expression is shown in parentheses relative to all other
cells in same sample), taken from pan-cancer
targeted invasive ductal carcinoma sample.

| DCIS vs. All | | Invasive Carcinoma vs. All | |
|---|---|---|---|
| (Log fold change) | | (Log fold change) | |
| ALB | (3.51) | PRKACB | (2.32) |
| SOCS2 | (2.88) | SPP1 | (2.24) |
| CSTA | (2.69) | CCND1 | (1.52) |
| BAMBI | (2.33) | FGFR1 | (1.47) |
| WIPI1 | (2.19) | VEGFA | (1.45) |
| PRLR | (1.84) | FN1 | (1.41) |
| BCL2 | (1.57) | HES6 | (1.36) |
| ZNF703 | (1.57) | S100A11 | (1.14) |
| IL6ST | (1.55) | H2AFX | (1.11) |

### Example 8: Spot Deconvolution with scRNA-Seq

The snRNA-seq sample of Example 6 is further analyzed to identify the cell types in each spot on an array. Here, a first section of a sample is placed on an array and analyte capture as described herein. A second section of tissue undergoes cellular dissociation, creating a sample with isolated cells that can be analyzed. Briefly, a tissue is minced into small pieces and treated with lysis buffer to homogenize the sample. The homogenous resultant is filtered and centrifuged to collect a pellet of nuclei. The nuclei is resuspended and used for single cell analysis. Multiple methods of deconvolution of spots are known in the art. See Elosua et al., SPOTlight: Seeded NMF regression to Deconvolute Spatial Transcriptomics Spots with Single-Cell Transcriptomes; Maaskola et al., Charting Tissue Expression Anatomy by Spatial Transcriptome Deconvolution; and MuSiC.

Data captured from the second section (i.e., the single nuclei data) is combined with the data from the first section (i.e., the whole tissue data) to gain a higher cell type understanding and potentially deconvolve the cell type identity within each spot on the array. Additional methods of single cell isolation is found in Hu et al., Mol Cell. 2017 Dec. 7; 68(5):1006-1015.e7; Habib et al., Science, 2016 Aug. 26; 353(6302):925-8; Habib et al., Nat Methods, 2017 October; 14(10):955-958; Lake et al., Science, 2016 Jun. 24; 352(6293):1586-90; and Lacar et al., Nat Commun, 2016 Apr. 19; 7:11022; each of which is incorporated by reference in its entirety.

1-67. (canceled)

68. A method of determining the abundance of two or more analytes in a breast cancer sample from a subject, wherein the method comprises
(a) determining an abundance of two or more analytes selected from the group consisting of: (1) CENPW, (2) A2ML1, (3) VLDLR, (4) SCRG1, (5) RCL1, (6) FABP7, (7) CCNE1, (8) MIEN1, (9) CDC37L1, (10) ERMP1, (11) TNFSF10, (12) ACTG2, (13) DMKN, (14) CALML3, (15) COL17A1, (16) MAGED1, (17) PTN, (18) TMEM98, (19) LY6D, (20) TNC, and (21) RTN4IP1, and byproducts, precursors, and degradation products thereof in a biological sample obtained from the subject; and
(b) identifying a subject having increased abundance(s) of the two or more analytes (1)-(21) and byproducts, precursors, and degradation products thereof, in the biological sample as compared to reference abundance(s) of the two or more analytes (1)-(21) and byproducts, precursors, and degradation products thereof, as having breast cancer; thereby determining the abundance of two or more analytes in the breast cancer sample from the subject.

69. The method of claim 68, wherein a subject is further treated for (a) invasive carcinoma, when the subject has increased abundance(s) of two or more analytes (1)-(11) and byproducts, precursors, and degradation products thereof, or (b) ductal carcinoma, when the subject has increased abundance(s) of two or more analytes (12)-(21) and byproducts, precursors, and degradation products thereof.

70. (canceled)

71. A method of determining the abundance of two or more analytes in a breast cancer sample from a subject, wherein the method comprises
(a) determining an abundance of two or more analytes selected from the group consisting of: ALB, SOCS2, CSTA, BAMBI, WIPI1, PRLR, BCL2, ZNF03, IL6ST, PRKACB, SPP1, CCND1, FGFR1, VEGFA, FN1, HES6, S100A11, H2AFX, and byproducts, precursors, and degradation products thereof in a biological sample obtained from the subject; and
(b) identifying a subject having increased abundance(s) of two or more analytes ALB, SOCS2, CSTA, BAMBI, WIPI1, PRLR, BCL2, ZNF03, IL6ST, PRKACB, SPP1, CCND1, FGFR1, VEGFA, FN1, HES6, S100A11, H2AFX, and byproducts, precursors, and degradation products thereof, in the biological sample as compared to reference abundance(s) of the two or more analytes ALB, SOCS2, CSTA, BAMBI, WIPI1, PRLR, BCL2, ZNF03, IL6ST, PRKACB, SPP1, CCND1, FGFR1, VEGFA, FN1, HES6, S100A11, H2AFX, and byproducts, precursors, and degradation products thereof, as having breast cancer; thereby determining the abundance of two or more analytes in the breast cancer sample from the subject.

72. The method of claim 71, wherein a subject is further treated for (a) ductal carcinoma, when the subject has increased abundance(s) of two or more analytes ALB, SOCS2, CSTA, BAMBI, WIPI1, PRLR, BCL2, ZNF03, IL6ST, and byproducts, precursors, and degradation products thereof or (b) invasive carcinoma, when the subject has increased abundance(s) of two or more analytes PRKACB, SPP1, CCND1, FGFR1, VEGFA, FN1, HES6, S100A11, and H2AFX, and byproducts, precursors, and degradation products thereof.

73. (canceled)

74. A method of determining the abundance of two or more analytes in a breast cancer sample from a subject, wherein the method comprises
(a) determining in a biological sample obtained from the subject an abundance of one or more of:
(i) two or more analytes selected from the group consisting of IGLC2, IGHG3, IGKC, IGHG1, IGLC3, IGHA1, IGHG2, IGHM, IGHG4, JCHAIN, ID3, CIITA, CST7, IFI27L2, FYN, MICAL1, HMOX1, CD7, ARHGEF1, and CFH and byproducts, precursors, and degradation products thereof;
(ii) two or more analytes selected from the group consisting of MALAT1, CTSD, TYMP, SAMHD1, CYBA, ISG15, C1QA, RPS9, H2AFJ, ADIRF, ARHGDIA, APRT, AEBP1, PLEC, APOE, FCGRT, NDUFB7, MBD3, EMILIN1, and GADD45GIP1 and byproducts, precursors, and degradation products thereof;
(iii) two or more analytes selected from the group consisting of CXCL14, TTLL12, GFRA1, DEGS1, AGR2, ARMT1, CCND1, ARPP21, CRAT, PRKACB, NSD3, PLAUR, CDKN2C, FIP1L1, TMEM159, TMEM141, LIN7C, ARHGEF39, ARFGEF3, and EMP2 and byproducts, precursors, and degradation products thereof;
(iv) two or more analytes selected from the group consisting of CPB1, FCGR3B, KLHDC7B, SCGB1D2, SCUBE3, CXCL9, COX6C, CFB, SCGB2A2, NPY1R, AC087741.1, GBP5, IFT27, NEURL4, EMX1, SLC13A2, FAM110A, SPCS1, HGD, and ZNF587 and byproducts, precursors, and degradation products thereof;
(v) two or more analytes selected from the group consisting of CRISP3, SLITRK6, C6orf141, VTCN1, SERHL2, CEACAM6, ABCC11, SHISA2, C2orf54, PDLIM1, ZC3H12A, VPS37B, IRF2BP2, RAPH1, NFKBIA, EIF2AK1, TRIM33, SFPQ, F7, and TRAPPC3 and byproducts, precursors, and degradation products thereof;
(vi) two or more analytes selected from the group consisting of LINC00052, COX6C, SNCG, WFDC2, SLC39A6, MGST1, MCCD1, CSTA, PDE5A, MT-ND1, SRP14, TRAPPC1, SNRPD3, MAT2A, SLC7A8, POLR2K, TMBIM6, OCIAD1, EXOSC3, and CA14 and byproducts, precursors, and degradation products thereof;
(vii) two or more analytes selected from the group consisting of ACKR1, IGFBP7, AQP1, VWF, MALAT1, SPARCL1, TAGLN, CCL21, ACTA2, CCDC₈₀, CYB5R3, PLXND1, DUSP1, RPS6, C1QA, HEG1, ETS2, AKAP9, CCAR1, and TRIM47 and byproducts, precursors, and degradation products thereof;
(viii) two or more analytes selected from the group consisting of ALB, MGP, ZNF350-AS1, S100G, STC2, CARTPT, AC087379.2, GPC3, ERP27, APOD, CDV3, TPI1, TSPYL5, PFKP, CRIM1, PPT1, AC100826.1, ALDOA, LGR4, and GLRX2 and byproducts, precursors, and degradation products thereof;
(ix) two or more analytes selected from the group consisting of AC087379.2, S100G, SCGB2A2, PGM5-AS1, HEBP1, AMIGO2, PCED1B, SCGB1D2, ITPR1, IFT122, GABARAPL1, G6PC3, INPP5K, STC2, HEXIM2, RIDA, LRP2, HK2, IL1R2, and GOT2 and byproducts, precursors, and degradation products thereof;
(x) two or more analytes selected from the group consisting of ALB, MT-ND2, MT-ND1, MT-ND3, MT-ATP6, MT-ND4, MT-CO1, MT-CO3, MT-ATPS, MT-ND5, GLO1, ABHD2, LINC00052, RERG, STC2, MALAT1, SOX4, CA12, ZNF703, and MCCD1 and byproducts, precursors, and degradation products thereof;
(xi) two or more analytes selected from the group consisting of LINC00645, SLC30A8, MUC5B, COLEC12, PVALB, CPB1, EXOC2, AC037198.2, VSTM2A, FSIP1, KIF16B, SPATA20, TSPAN9, CACNB3, CAMK2N1, IFT27, NEURL1, TRIM3, SLC46A1, and ECI2 and byproducts, precursors, and degradation products thereof;
(xii) two or more analytes selected from the group consisting of MGP, TFF1, KRT14, S100A9, KRT17, S100G, S100A2, ZNF350-AS1, KRT5, S100A8, MN1, TMEM45A, DNALI1, C3orf14, TSR1, AL445524.1, SEMA3C, NDUFAF2, ASCL1, and GRHL2 and byproducts, precursors, and degradation products thereof;
(xiii) two or more analytes selected from the group consisting of SAA1, FABP4, GPX3, PIP, ADH1B, PLIN1, COL2A1, SH3BGRL, ADIPOQ, PLIN4, HNRNPA0, RPL11, SLC40A1, RPLP2, CTDSP2, SORL1, RPL31, ECE1, SFRP2, and CCND1 and byproducts, precursors, and degradation products thereof; or
(xiv) two or more analytes selected from the group consisting of PDE5A, MGP, WFDC2, MRPS30-DT, RBM20, MRPS30, AMFR, STC2, KCNE4, DDR1, MCCC1, PDPR, AMZ2, C5orf15, TBC1D9, SRPK1, LINC01488, TSG101, COA3, and NFKBIE and byproducts, precursors, and degradation products thereof; and
(b) identifying a subject having dysregulated abundance(s) of one or more of (i)-(xiv) in the biological sample as compared to reference abundance(s) of the one or more of as having breast cancer; thereby determining the abundance of two or more analytes in the breast cancer sample from the subject.

75. The method of claim 74, wherein the method comprises:
(a) determining an abundance of one or more of:
(i) two or more analytes selected from the group consisting of IGLC2, IGHG3, IGKC, IGHG1, IGLC3, IGHA1, IGHG2, IGHM, IGHG4, JCHAIN, and byproducts, precursors, and degradation products thereof;
(ii) two or more analytes selected from the group consisting of MALAT1, CTSD, TYMP, SAMHD1, CYBA, ISG15, C1QA, RPS9, H2AFJ, ADIRF, and byproducts, precursors, and degradation products thereof;
(iii) two or more analytes selected from the group consisting of CXCL14, TTLL12, GFRA1, DEGS1, AGR2, ARMT1, CCND1, ARPP21, CRAT, PRKACB, and byproducts, precursors, and degradation products thereof;
(iv) two or more analytes selected from the group consisting of CPB1, FCGR3B, KLHDC7B, SCGB1D2, SCUBE3, CXCL9, COX6C, CFB, SCGB2A2, NPY1R, and byproducts, precursors, and degradation products thereof;
(v) two or more analytes selected from the group consisting of CRISP3, SLITRK6, C6orf141, VTCN1, SERHL2, CEACAM6, ABCC11, SHISA2, C2orf54, PDLIM1, and byproducts, precursors, and degradation products thereof;
(vi) two or more analytes selected from the group consisting of LINC00052, COX6C, SNCG, WFDC2, SLC39A6, MGST1, MCCD1, CSTA, PDE5A, MT-ND1, and byproducts, precursors, and degradation products thereof;
(vii) two or more analytes selected from the group consisting of ACKR1, IGFBP7, AQP1, VWF, MALAT1, SPARCL1, TAGLN, CCL21, ACTA2, CCDC₈₀, and byproducts, precursors, and degradation products thereof;
(viii) two or more analytes selected from the group consisting of ALB, MGP, ZNF350-AS1, S100G, STC2, CARTPT, AC087379.2, GPC3, ERP27, APOD, and byproducts, precursors, and degradation products thereof;
(ix) two or more analytes selected from the group consisting of AC087379.2, S100G, SCGB2A2, PGM5-AS1, HEBP1, AMIGO2, PCED1B, SCGB1D2, ITPR1, IFT122, and byproducts, precursors, and degradation products thereof;
(x) two or more analytes selected from the group consisting of ALB, MT-ND2, MT-ND1, MT-ND3, MT-ATP6, MT-ND4, MT-CO1, MT-CO3, MT-ATPS, MT-ND5, and byproducts, precursors, and degradation products thereof;
(xi) two or more analytes selected from the group consisting of LINC00645, SLC30A8, MUC5B, COLEC12, PVALB, CPB1, EXOC2, AC037198.2, VSTM2A, FSIP1, and byproducts, precursors, and degradation products thereof;
(xii) two or more analytes selected from the group consisting of MGP, TFF1, KRT14, S100A9, KRT17, S100G, S100A2, ZNF350-AS1, KRT5, S100A8, and byproducts, precursors, and degradation products thereof;
(xiii) two or more analytes selected from the group consisting of SAA1, FABP4, GPX3, PIP, ADH1B, PLIN1, COL2A1, SH3BGRL, ADIPOQ, PLIN4, and byproducts, precursors, and degradation products thereof; or
(xiv) two or more analytes selected from the group consisting of PDE5A, MGP, WFDC2, MRPS30-DT, RBM20, MRPS30, AMFR, STC2, KCNE4, DDR1, and byproducts, precursors, and degradation products thereof; and
(b) identifying a subject having increased abundance(s) of one or more of (i)-(xiv), in the biological sample as compared to reference abundance(s) of the one or more of (i)-(xiv), as having breast cancer.

76. The method of claim 74, wherein the method comprises:
(a) determining an abundance of one or more of:
(i) two or more analytes selected from the group consisting of ID3, CIITA, CST7, IFI27L2, FYN, MICAL1, HMOX1, CD7, ARHGEF1, and CFH and byproducts, precursors, and degradation products thereof;
(ii) two or more analytes selected from the group consisting of ARHGDIA, APRT, AEBP1, PLEC, APOE, FCGRT, NDUFB7, MBD3, EMILIN1, and GADD45GIP1 and byproducts, precursors, and degradation products thereof;
(iii) two or more analytes selected from the group consisting of NSD3, PLAUR, CDKN2C, FIP1L1, TMEM159, TMEM141, LIN7C, ARHGEF39, ARFGEF3, and EMP2 and byproducts, precursors, and degradation products thereof;
(iv) two or more analytes selected from the group consisting of AC087741.1, GBP5, IFT27, NEURL4, EMX1, SLC13A2, FAM110A, SPCS1, HGD, and ZNF587 and byproducts, precursors, and degradation products thereof;
(v) two or more analytes selected from the group consisting of ZC3H12A, VPS37B, IRF2BP2, RAPH1, NFKBIA, EIF2AK1, TRIM33, SFPQ, F7, and TRAPPC3 and byproducts, precursors, and degradation products thereof;
(vi) two or more analytes selected from the group consisting of SRP14, TRAPPC1, SNRPD3, MAT2A, SLC7A8, POLR2K, TMBIM6, OCIAD1, EXOSC3, and CA14 and byproducts, precursors, and degradation products thereof;
(vii) two or more analytes selected from the group consisting of CYB5R3, PLXND1, DUSP1, RPS6, C1QA, HEG1, ETS2, AKAP9, CCAR1, and TRIM47 and byproducts, precursors, and degradation products thereof;
(viii) two or more analytes selected from the group consisting of CDV3, TPI1, TSPYL5, PFKP, CRIM1, PPT1, AC100826.1, ALDOA, LGR4, and GLRX2 and byproducts, precursors, and degradation products thereof;
(ix) two or more analytes selected from the group consisting of GABARAPL1, G6PC3, INPP5K, STC2, HEXIM2, RIDA, LRP2, HK2, IL1R2, and GOT2 and byproducts, precursors, and degradation products thereof;
(x) two or more analytes selected from the group consisting of GLO1, ABHD2, LINC00052, RERG, STC2, MALAT1, SOX4, CA12, ZNF703, and MCCD1 and byproducts, precursors, and degradation products thereof;
(xi) two or more analytes selected from the group consisting of KIF16B, SPATA20, TSPAN9, CACNB3, CAMK2N1, IFT27, NEURL1, TRIM3, SLC46A1, and ECI2 and byproducts, precursors, and degradation products thereof;
(xii) two or more analytes selected from the group consisting of MN1, TMEM45A, DNALI1, C3orf14, TSR1, AL445524.1, SEMA3C, NDUFAF2, ASCL1, and GRHL2 and byproducts, precursors, and degradation products thereof;
(xiii) two or more analytes selected from the group consisting of HNRNPA0, RPL11, SLC40A1, RPLP2, CTDSP2, SORL1, RPL31, ECE1, SFRP2, and CCND1 and byproducts, precursors, and degradation products thereof; or
(xiv) two or more analytes selected from the group consisting of MCCC1, PDPR, AMZ2, C5orf15, TBC1D9, SRPK1, LINC01488, TSG101, COA3, and NFKBIE and byproducts, precursors, and degradation products thereof; and
(b) identifying a subject having decreased abundance(s) of one or more of (i)-(xiv), in the biological sample as compared to reference abundance(s) of the one or more of (i)-(xiv), as having breast cancer.

77-78. (canceled)

79. The method of claim 68, further comprising administering a treatment of breast cancer to the subject, adjusting a dosage of a treatment of breast cancer for the subject, or adjusting a treatment of breast cancer for the subject.

80. The method of claim 79, wherein the treatment comprises administering one or more therapies selected from the group consisting of an endocrine therapy, a chemotherapy a hormonal therapy, and a surgical resection.

81. The method of claim 80, wherein the endocrine therapy is one or more agents selected from the group consisting of tamoxifen, raloxifene, megestrol, toremifene, and an aromatase inhibitor; the chemotherapy is neoadjuvant chemotherapy; and the surgical resection is surgery for breast tissue and/or lymph node tissue.

82. The method of claim 81, wherein the aromatase inhibitor is one or more agents selected from the group consisting of anastrozole, letrozole, or exemestane; and the neoadjuvant chemotherapy is one or more agents selected from a taxane derivative, an anthracycline derivative, and a topoisomerase inhibitors.

83-84. (canceled)

85. The method of claim 82, wherein the taxane derivative is one or more agents selected from docetaxel and paclitaxel; and the anthracycline derivative is doxorubicin.

86. (canceled)

87. The method of claim 81, wherein the breast tissue surgery is selected from the group comprising lumpectomy, quadrantectomy, partial mastectomy, segmental mastectomy, complete mastectomy, and the lymph node tissue surgery is selected from the group consisting of sentinel lymph node biopsy and axillary lymph node dissection.

88. The method of claim 68, wherein the two or more analytes are mRNA molecules.

89. The method of claim 88, wherein the determining step comprises determining the abundance and location of the two or more analytes, the method comprising:
(a) contacting the biological sample with a substrate comprising a plurality of attached capture probes, wherein a capture probe of the plurality comprises (i) a spatial barcode and (ii) a capture domain that binds to a sequence present in the analyte;
(b) hybridizing the two or more analytes to the capture domain;
(c) extending a 3′ end of the capture probe using the analyte that is bound to the capture domain as a template to generate an extended capture probe;
(d) amplifying the extended capture probe; and
(e) determining (i) all or a portion of the sequence of the spatial barcode or the complement thereof, and (ii) all or a portion of the sequence of the analyte from the biological sample; and using the determined sequences of (i) and (ii) to identify the location of the analyte in the biological sample, thereby determining the abundance and location of the two or more analytes.

90. The method of claim 68, wherein the two or more analytes are proteins.

91. The method of claim 90, wherein the determining step comprises determining the abundance and location of the two or more analytes, the method comprising:
(a) attaching the biological sample with a plurality of analyte capture agents, wherein an analyte capture agent of the plurality of analyte capture agents comprises:
(i) an analyte binding moiety that binds to the two or more analytes;
(ii) an analyte binding moiety barcode that uniquely identifies an interaction between the two or more analytes and the analyte binding moiety; and
(iii) an analyte capture sequence, wherein the analyte capture sequence binds to a capture domain;
(b) contacting the biological sample with a substrate, wherein the substrate comprises a plurality of capture probes, wherein a capture probe of the plurality of capture probes comprises (i) the capture domain and (ii) a spatial barcode;
(c) hybridizing the two or more analytes to the capture probe; and
(d) determining (i) all or a part of a sequence corresponding to the analyte binding moiety barcode, and (ii) all or a part of a sequence corresponding to the spatial barcode, or a complement thereof, and using the determined sequence of (i) and (ii) to identify the abundance and spatial location of the two or more analytes in the biological sample.

92. The method of claim 68, wherein the biological sample from the subject comprises more than one biological sample from the subject from a plurality of time points and determining the abundance of the two or more analytes in the two or more biological samples from the plurality of time points from the subject.

93. The method of claim 68, wherein the biological sample is a solid tissue sample, wherein the solid tissue sample is a formalin fixed paraffin embedded tissue sample or a frozen tissue sample.

94. The method of claim 68, wherein the biological sample is a breast tissue sample from a subject suspected of having a breast carcinoma of any one of: a ductal carcinoma in situ, a triple negative breast cancer, an estrogen receptor positive breast cancer, a progesterone receptor negative breast cancer, and a human epidermal growth factor receptor 2 positive breast cancer.
